(19) United States
(12) Patent Application Publication
(10) Pub. No.: US 2025/0197409 A1
(43) Pub. Date: Jun. 19, 2025

(54) BIFUNCTIONAL COMPOUND AND PHARMACEUTICAL COMPOSITION COMPRISING THE BIFUNCTIONAL COMPOUND, AND METHOD FOR TREATING ANDROGEN RECEPTOR RELATED DISEASE BY USING THE SAME

(71) Applicant: ANHORN MEDICINES CO., LTD., Taipei City (TW)

(72) Inventors: Chu-Chiang Lin, Taipei City (TW); Hung-Chuan Chen, New Taipei City (TW); Pei-Chin Cheng, Changhua County (TW); Chih-Chang Chou, Taoyuan City (TW)

(21) Appl. No.: 18/879,351

(22) PCT Filed: Jun. 29, 2023
(86) PCT No.: PCT/CN2023/103576

§ 371 Date: Dec. 27, 2024

Related U.S. Application Data
(60) Provisional application No. 63/357,054, filed on Jun. 30, 2022.

(51) Int. Cl. C07D 487/04; A61K 31/4545; A61K 31/496; A61K 31/497; A61K 31/501; A61K 31/506; A61K 31/551; A61K 47/55; A61P 17/14; C07D 495/04; C07D 519/00

(52) U.S. Cl. CPC C07D 487/04; A61K 31/4545; A61K 31/496; A61K 31/497; A61K 31/501; A61K 31/506; A61K 31/551; A61K 47/55; A61P 17/14; C07D 495/04; C07D 519/00

(57) ABSTRACT

Provided is a bifunctional compound, or a pharmaceutically acceptable salt, hydrate, solvate, metabolite or prodrug thereof, wherein the bifunctional compound is represented by Formula (I):

ABM-L-CLM (I);
wherein:
ABM is an androgen receptor binding moiety;
-L- is a linking moiety; and
CLM is a cereblon E3 ubiquitin ligase binding moiety represented by Formula (II)-1:

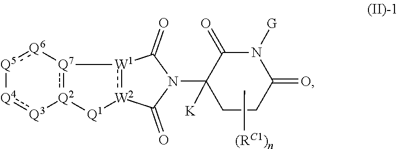

wherein one end of the -L- is covalently joined to Q₃, Q₄, Q₅ or Q₆; and the other end of the -L- is covalently joined to the ABM. Also provided are a pharmaceutical composition comprising the bifunctional compound and a method for treating an androgen receptor related disease or disorder by administering the bifunctional compound.

### BACKGROUND OF THE INVENTION

### 1. Field of the Invention

The present disclosure relates to a bifunctional compound; and a pharmaceutical composition comprising the bifunctional compound and a method for treating an androgen receptor related disease or disorder by administering the bifunctional compound.

### 2. Description of the Prior Arts

Protein-protein interactions are difficult to be targeted using small molecules because proteins have large contact surfaces and the shallow grooves or flat interfaces thereon may get involved in the interactions. On the other hand, tagging the pathogenic protein with ubiquitin and the eventual degradation of the pathogenic protein by the 26S proteasome system has demonstrated that this modality can provide an extended and thorough removal of the cause of disease (Sun et al., Signal Transduct. Target Ther. 2019, 4:64). In addition to the E1 and E2 enzymes, E3 ubiquitin ligases (also known as E3 ligases) and their substrate recognition proteins confer the substrate specificity for ubiquitination. They are the critical components for specific and selective degradation of target protein substrates. Recent development of targeted protein degradation indicated E3 ligases such as cereblon (CRBN) E3 ligase, von Hippel-Lindau disease tumor suppressor (VHL) E3 ligase, double minute 2 protein (MDM2) E3 ligase, and cell inhibitor of apoptosis protein (cIAP) E3 ligase have been utilized successfully for small molecule protein degrader design. These molecules are likely to become therapeutic candidates (Wang et al., Acta Pharm Sin B. 2020 February; 10(2):207-238).

One E3 ligase with therapeutic potential is cereblon E3 ligase, a protein in humans that is encoded by the CRBN gene. CRBN orthologs are highly conserved from plants to humans. Cereblon forms an E3 ubiquitin ligase complex with damaged DNA binding protein 1 (DDB1), Cullin-4A (CUL4A), and Regulator of Cullins 1 (ROC 1). This complex ubiquitinates a number of other proteins (Vriend et al., Front Mol Biosci. 2018, 5:19).

Androgen receptor (AR) belongs to a nuclear hormone receptor family that is activated by androgens, such as testosterone and dihydrotestosterone. Upon binding of androgen, AR is translocated into the nucleus where it acts as a transcription factor to promote gene expression responsible for male sexual characteristics. While AR is responsible for development of male sexual characteristics, it also drives the growth and survival of prostate cancer cells (Salami et al., Commun. Biol. 2018, 1:100).

AR signaling suppression is a common strategy for treating prostate cancer. Prostate cancer is the second most diagnosed cancer and the fifth leading cause of death in men worldwide. The 5-year survival rate for most men with local or regional prostate cancer is nearly 100%. For men diagnosed with prostate cancer that has spread to other parts of the body, the 5-year survival rate is 31%. Based on GLOBOCAN estimates, 1.41 million new cases of prostate cancer were reported in 2020 with 375 thousand deaths worldwide. For decades, the androgen deprivation therapy (ADT), by either surgical or chemical castration, has been the standard treatment for prostate cancer management. However, a castration-resistant form of prostate cancer eventually develops, whereby tumor cell proliferation resumes despite sub-castration levels of serum testosterone. With the approval of second-generation antiandrogen drugs such as abiraterone acetate (ABI), enzalutamide (ENZ), and recently approved apalutamide (APA) and darolutamide (DARO), overall survival rate of castration-resistant prostate cancer (CRPC) patients was improved. But, as frequently seen in cancer chemotherapy, drug resistant escape mutations eventually appear while the disease progresses (Zhao et al., Mol Cancer Ther. 2020, 19(8):1708-1718). In addition, it is also found that androgen and AR involve in many skin conditions, such as androgenetic alopecia, acne, hirsutism, atopic dermatitis, and the like. In the skin, significant amounts of sexual hormones are synthesized, and androgen affects hair growth, epidermal barrier homeostasis, wound healing, sebaceous gland growth, differentiation and so on. Nowadays, antiandrogen drugs such as clascoterone, cyproterone acetate and flutamide have been used for treating these skin conditions. The level of androgen and AR may play important roles in the development of the diseases involving AR signaling. (Zhou et al., Journal of Biosciences and Medicines 2022, 10:180-200).

The present disclosure provides an alternative approach via proteolysis targeting chimeric (PROTAC) bifunctional compounds for the treatment of AR-mediated or AR-dependent diseases or disorders. Related PROTAC patent applications are disclosed in WO2018071606, WO2018144649 and WO2021143816. However, compounds having better effects for treating androgen receptor related diseases or disorders are still needed.

### SUMMARY OF THE INVENTION

The present disclosure provides proteolysis targeting chimeric (PROTAC) bifunctional compounds comprising both a cereblon (CRBN) E3 ubiquitin ligase binding moiety and an androgen receptor binding moiety, which redirect a ubiquitin proteasome degradation system to degrade androgen receptors, and degrade the androgen receptors and/or otherwise inhibit the androgen receptors. Unexpectedly, these bifunctional compounds demonstrate higher potency in the degradation of androgen receptors.

In one aspect, the present disclosure provides a bifunctional compound, or a pharmaceutically acceptable salt, hydrate, solvate, metabolite or prodrug thereof, wherein the bifunctional compound is represented by Formula (I):

$$ABM - L - CLM ; \qquad ( I )$$

wherein:
ABM is an androgen receptor binding moiety;
-L- is a linking moiety; and
CLM is a cereblon E3 ubiquitin ligase binding moiety represented by Formula (II)-1:

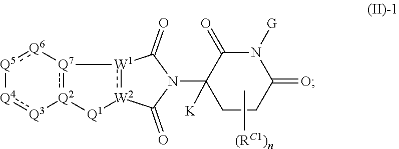

wherein:
[symbol] represents a single bond or a double bond;
one end of the -L- is covalently joined to Q₃, Q₄, Q₅ or Q₆; and the other end of the -L- is covalently joined to the ABM;
W¹ and W² are each independently CR^{C2} or N when a single bond is present between W¹ and W²; or, W¹ and W² are each C when a double bond is present between W¹ and W²;
G is selected from the group consisting of —H, —OH, —CH₂OH, —R^{C3}OC(═O)OR^{C4}, —R^{C3}OC(═O)NR^{C4}R^{C5}, and 2-(trimethylsilyl)ethoxymethyl group;
Q¹ is O, S or NR^{C6};
Q² and Q⁷ are each independently N or CR^{C2} when a single bond is present between Q² and Q⁷; or, Q² and Q⁷ are each C when a double bond is present between Q² and Q⁷;
when the one end of the -L- is covalently joined to any one atom selected from Q₃, Q₄, Q₅ and Q₆; the atom covalently joined with the -L- is CR^{C2} when two single bonds are each independently present between the C atom of CR^{C2} and its two adjacent atoms on the ring, or the atom covalently joined with the -L- is C when a double bond is present between the C atom and one of its two adjacent atoms on the ring; and, the other atoms selected from Q³, Q⁴, Q⁵ and Q⁶ which are not covalently joined with the -L- are each independently O, S, C(R^{C2})₂ or NR^{C2} when two single bonds are each independently present between the O atom, S atom, C atom of C(R^{C2})₂ or N atom of NR^{C2} and its two adjacent atoms on the ring, or the other atoms selected from Q³, Q⁴, Q⁵ and Q⁶ which are not covalently joined with the -L- are each independently CR^{C2} when a double bond is present between the C atom of CR^{C2} and one of its two adjacent atoms on the ring;
K is selected from the group consisting of —H, an unsubstituted alkyl group, an alkyl group substituted by R^{C7}, an unsubstituted cycloalkyl group, and a cycloalkyl group substituted by R^{C7}; K is bound to the 6-membered ring with a stereospecific bond or a non-stereospecific bond;
R^{C1} is selected from the group consisting of an unsubstituted alkyl group, an alkyl group substituted by R^{C8}, an unsubstituted aryl group, an aryl group substituted by R^{C8}, an unsubstituted alkyl-aryl group, an alkyl-aryl group substituted by R^{C8}, an unsubstituted alkoxyl group, and an alkoxyl group substituted by R^{C8};
R^{C2} is selected from the group consisting of —H, -D, a halo group, —CH₂OH, —NR^{C4}R^{C5}, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, and an aryl group substituted by one or more halo groups;
R^{C3} is selected from the group consisting of an unsubstituted alkylene group, and an alkylene group substituted by R^{C7};
R^{C4} and R^{C5} are each independently selected from the group consisting of —H, an unsubstituted alkyl group, an alkyl group substituted by R^{C9}, an unsubstituted cycloalkyl group, a cycloalkyl group substituted by R^{C9}, an unsubstituted heterocyclyl group, a heterocyclyl group substituted by R^{C9}, an unsubstituted aryl group, an aryl group substituted by R^{C9}, an unsubstituted heteroaryl group, and a heteroaryl group substituted by R^{C9};
R^{C6} is selected from the group consisting of —H, a halo group, —CH₂OH, 2-(trimethylsilyl)ethoxymethyl, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C7} is selected from the group consisting of a halo group, —CH₂OH, —NR^{C4}R^{C5}, 2-(trimethylsilyl)ethoxymethyl, an alkoxyl group, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C8} is selected from the group consisting of a halo group, —CH₂OH, —NR^{C4}R^{C5}, 2-(trimethylsilyl)ethoxymethyl, an unsubstituted cycloalkyl group, a cycloalkyl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C9} is selected from the group consisting of a halo group, —CH₂OH, 2-(trimethylsilyl)ethoxymethyl, and an alkoxyl group;
n is 0, 1, 2, 3 or 4; and
the heteroatom is selected from N, O and S.

In another aspect, the present disclosure provides a pharmaceutical composition comprising an effective amount of the above-mentioned compound, or the pharmaceutically acceptable salt, hydrate, solvate, metabolite or prodrug thereof; and a pharmaceutically acceptable carrier.

In yet another aspect, the present disclosure provides a method for treating an androgen receptor related disease or disorder in a subject in need thereof, comprising administering an effective amount of the above-mentioned compound, or the pharmaceutically acceptable salt, hydrate, solvate, metabolite or prodrug thereof, or the above-mentioned pharmaceutical composition to the subject.

In some embodiments, the CLM is represented by Formula (II)-2:

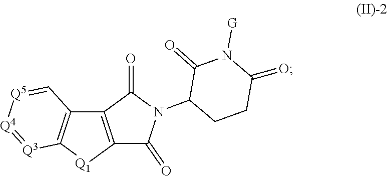

wherein
one end of the -L- is covalently joined to Q₃, Q₄ or Q₅;
G is selected from the group consisting of —H, —OH, —CH₂OH and 2-(trimethylsilyl)ethoxymethyl group;
Q¹ is O, S or NR^{C6};
when the one end of the -L- is covalently joined to any one atom selected from Q₃, Q₄ and Q₅; the atom attached with the -L- is C; and, the other atoms selected from Q³, Q⁴ and Q⁵ which are not attached with the -L- are each independently CR^{C2};
R^{C2} is selected from the group consisting of —H, -D, a halo group, an unsubstituted alkyl group, and an alkyl group substituted by one or more halo groups; and
R^{C6} is selected from the group consisting of —H, —CH₂OH, 2-(trimethylsilyl)ethoxymethyl, an unsubstituted C₁₋₆ alkyl group, and a C₁₋₆ alkyl group substituted by one or more halo groups.

In some embodiments, the heteroatoms in Formula (II)-1 are each independently selected from N, O and S.

In some embodiments, G is selected from the group consisting of —H, —OH, —CH₂OH, —CH₂OCOOCH₃ and 2-(trimethylsilyl)ethoxymethyl group.

In some embodiments, the 2-(trimethylsilyl)ethoxymethyl group is also abbreviated as an SEM group.

In some embodiments, K is bound to the 6-membered ring with a stereospecific bond:

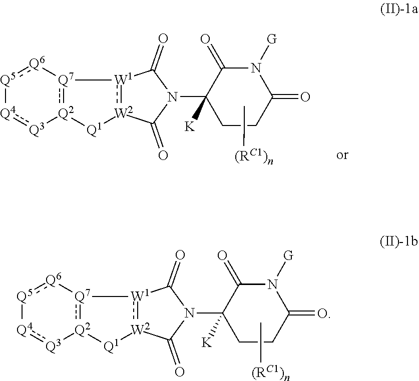

In the present invention, the carbon on the 6-membered ring which is attached with K is a chiral carbon center, and the bifunctional compound, or a pharmaceutically acceptable salt, hydrate, solvate, metabolite or prodrug thereof may exist two stereoisomers having a CLM represented by Formula (II)-1a and Formula (II)-1b. The bifunctional compound, or a pharmaceutically acceptable salt, hydrate, solvate, metabolite or prodrug thereof may have one above-mentioned stereoisomer or both stereoisomers.

In some embodiments, K is selected from the group consisting of —H, an unsubstituted C₁₋₆ alkyl group, a C₁₋₆ alkyl group substituted by R^{C7}, and a C₃₋₈ cycloalkyl group. In some embodiments, K may be an unsubstituted C₁₋₃ alkyl group, or a C₁₋₃ alkyl group substituted by R^{C7}.

In some embodiments, K is an alkyl selected from the group consisting of a linear alkyl and a branched alkyl, each of which is unsubstituted or substituted by R^{C7}. In some embodiments, K is an alkyl selected from the group consisting of a linear C₁₋₆ alkyl and a branched C₁₋₆ alkyl, each of which is unsubstituted or substituted by R^{C7}. In some embodiments, K is an alkyl selected from the group consisting of a linear C₁₋₃ alkyl and a branched C₁₋₃ alkyl, each of which is unsubstituted or substituted by R^{C7}.

In some embodiments, R^{C1} may be an unsubstituted C₁₋₆ alkyl group, a C₁₋₆ alkyl group substituted by R^{C8}, an unsubstituted C₃₋₈ aryl group, a C₃₋₈ aryl group substituted by R^{C8}, an unsubstituted C₃₋₈ alkyl-aryl group, an C₃₋₈ alkyl-aryl group substituted by R^{C8}, an unsubstituted C₁₋₆ alkoxyl group, or a C₁₋₆ alkoxyl group substituted by R^{C8}. In some embodiments, R^{C1} may be an unsubstituted C₁₋₃ alkyl group, a C₁₋₃ alkyl group substituted by R^{C8}, an unsubstituted C₁₋₃ alkoxyl group, or a C₁₋₃ alkoxyl group substituted by R^{C8}.

In some embodiments, R^{C1} is an alkyl selected from the group consisting of a linear alkyl and a branched alkyl, each of which is unsubstituted or substituted by R^{C8}. In some embodiments, R^{C1} is an alkyl selected from the group consisting of a linear C₁₋₆ alkyl and a branched C₁₋₆ alkyl, each of which is unsubstituted or substituted by R^{C8}. In some embodiments, R^{C1} is an alkyl selected from the group consisting of a linear C₁₋₃ alkyl and a branched C₁₋₃ alkyl, each of which is unsubstituted or substituted by R^{C8}.

In the present disclosure, a halo group may be F, Cl, Br or I. In the present disclosure, a halo group may be F or Cl. In the present disclosure, a halo group is F.

In the present disclosure, R^{C2} is selected from the group consisting of —H, -D (deuterium), a halo group, an unsubstituted C₁₋₆ alkyl group, and a C₁₋₆ alkyl group substituted by one or more halo groups. In the present disclosure, R^{C2} is selected from the group consisting of —H, -D, —F, —Cl, an unsubstituted C₁₋₃ alkyl group, and a C₁₋₃ alkyl group substituted by one or more halo groups.

In the present disclosure, R^{C4} and R^{C5} are each independently selected from the group consisting of an unsubstituted C₁₋₆ alkyl group, a C₁₋₆ alkyl group substituted by R^{C9}, an unsubstituted C₃₋₈ cycloalkyl group, a C₃₋₈ cycloalkyl group substituted by R^{C9}, an unsubstituted C₃₋₈ heterocyclyl group, a C₃₋₈ heterocyclyl group substituted by R^{C9}, a C₃₋₈ aryl group, and a C₃₋₈ heteroaryl group. In the present disclosure, R^{C4} and R^{C5} are each independently selected from the group consisting of an unsubstituted C₁₋₃ alkyl group, and a C₁₋₃ alkyl group substituted by R^{C9}. In the present disclosure, R^{C4} may be methyl, ethyl, n-propyl or isopropyl.

In some embodiments, R^{C4} and R^{C5} are each independently an alkyl selected from the group consisting of a linear alkyl and a branched alkyl, each of which is unsubstituted or substituted by R^{C9}. In some embodiments, R^{C4} and R^{C5} are each independently an alkyl selected from the group consisting of a linear C₁₋₆ alkyl and a branched C₁₋₆ alkyl, each of which is unsubstituted or substituted by R^{C9}. In some embodiments, R^{C4} and R^{C5} are each independently an alkyl selected from the group consisting of a linear C₁₋₃ alkyl and a branched C₁₋₃ alkyl, each of which is unsubstituted or substituted by R^{C9}.

In some embodiments, the CLM is represented by Formula (II)-1 or Formula (II)-2, wherein Q¹ is NR^{C6}; and R^{C6} is —H, an unsubstituted C₁₋₆ alkyl group, or a C₁₋₆ alkyl group substituted by one or more halo groups. In some embodiments, Q¹ is NR^{C6}; and R^{C6} is H, an unsubstituted C₁₋₃ alkyl group, or a C₁₋₃ alkyl group substituted by one or more halo groups. In some embodiments, Q¹ is NR^{C6}; and R^{C6} is an unsubstituted C₁₋₃ alkyl group selected from methyl, ethyl, n-propyl, and isopropyl. In some embodiments, Q¹ is NR^{C6}; and R^{C6} is a C₁₋₃ alkyl group selected from methyl, ethyl, n-propyl, isopropyl, which is substituted by one or more halo groups independently selected from F, Cl and Br.

In some embodiments, one end of the -L- is covalently joined to Q₄ or Q₅. In some embodiments, one end of the -L- is covalently joined to Q₄.

In some embodiments, the -L- is a linking moiety represented by Formula (III):

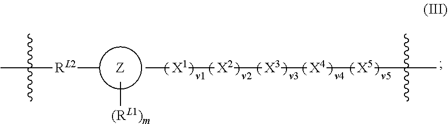

wherein
Z is selected from the group consisting of a 3- to 8-membered monocyclic ring, a 5- to 12-membered bicyclic ring, a 8- to 15-membered tricyclic ring and a 6- to 12-membered spiro bicyclic ring, each independently having 0 to 4 heteroatoms and 0 or 1 double bond;
R^{L1} is selected from the group consisting of an unsubstituted C₁₋₆ alkyl group, a C₁₋₆ alkyl group substituted by a C₁₋₆ alkoxyl group, a C₁₋₆ alkyl group substituted by one or more halo groups, a halo group, an unsubstituted C₁₋₆ alkoxyl group, a keto group, and an oxide group;
R^{L2} is a bond or an ethynylene group;
X¹ is a methylene group or an ethylene group, each of which is unsubstituted or substituted by an alkyl or a cycloalkyl;
X² and X⁵ are each independently selected from the group consisting of an unsubstituted methylene group, a methylene group substituted by an alkyl or a cycloalkyl, CO, COCO, CONH, NHCO, NHCONR^{L3}, NHCOCONR^{L3}—, NHSO₂NR^{L3}, SO₂NH, CHR^{L3}, NR^{L4}, O and S;
X³ is selected from the group consisting of a C₁₋₈ alkylene group, a C₁₋₈ heteroalkylene group, a 5- to 8-membered arylene group, a 5- to 8-membered heteroarylene group having 1 to 3 hetero atoms, a 3- to 7-membered cyclic alkylene group, a 3- to 7-membered heterocyclic alkylene group having 1 to 2 hetero atoms, and a 6- to 12-membered spiro bicyclic ring having 0 to 4 heteroatoms, each of which is unsubstituted or substituted by an alkyl or a cycloalkyl;
X⁴ is an unsubstituted C₁₋₃ alkylene group, or a C₁₋₆ alkylene group substituted by an alkyl or a cycloalkyl;
R^{L3} and R^{L4} are each independently selected from the group consisting of —H, an unsubstituted C₁₋₆ alkyl group, a C₁₋₆ alkyl group substituted by a C₁₋₆ alkoxyl group, and a C₁₋₆ alkyl group substituted by one or more halo groups;
m is 0, 1, 2, 3, 4, 5 or 6; v1 is 0 or 1; v2 is 0 or 1; v3 is 1; v4 is 0 or 1; and v5 is 1.

In some embodiments, the heteroatoms in Formula (III) are each independently selected from N, O and S. In some embodiments, the Z ring comprises one or two heteroatoms selected from N, O and S.

In some embodiments, the -L- is a linking moiety represented by Formula (III), wherein
Z is selected from the group consisting of a 3- to 8-membered monocyclic ring, a 6- to 10-membered bicyclic ring and a 8- to 11-membered spiro bicyclic ring, each independently having 1 to 2 heteroatoms and 0 or 1 double bond;
R^{L1} is selected from the group consisting of an unsubstituted linear C₁₋₃ alkyl group, and a keto group;
R^{L2} is a bond or an ethynylene group;
X¹ is methylene group or an ethylene group, each of which is unsubstituted or substituted by an alkyl or a cycloalkyl;
X² is selected from the group consisting of an unsubstituted methylene group, a methylene group substituted by an alkyl or a cycloalkyl, CO, NHCO, NR^{L4}, and O;
X³ is selected from the group consisting of a C₁₋₆ alkylene group, a 3- to 7-membered cyclic alkylene group, and a 3- to 7-membered heterocyclic alkylene group having 0 to 2 hetero atoms, each of which is unsubstituted or substituted by an alkyl or a cycloalkyl;
X⁴ is an unsubstituted C₁₋₃ alkylene group, or a C₁₋₃ alkylene group substituted by an alkyl or a cycloalkyl;
X⁵ is selected from the group consisting of an unsubstituted methylene group, a methylene group substituted by an alkyl or a cycloalkyl, CONH and O;
R^{L4} is hydrogen;
m is 0, 1, 2 or 3; v1 is 0 or 1; v2 is 0 or 1; v3 is 1; v4 is 0 or 1; and v5 is 1.

In some embodiments, R^{L1} is selected from the group consisting of H, an unsubstituted C₁₋₃ alkyl group, a C₁₋₃ alkyl group substituted by a C₁₋₃ alkoxyl group, a C₁₋₃ alkyl group substituted by one or more halo groups, a halo group, an unsubstituted C₁₋₃ alkoxyl group, a keto group, and an oxide group. In some embodiments, R^{L1} is an oxide group which is attached to one heteroatom N on the Z ring to form an N-oxide group (N⁺—O⁻).

In some embodiments, R^{L1} is an alkyl selected from the group consisting of a linear C₁₋₆ alkyl and a branched C₁₋₆ alkyl, each of which is unsubstituted, or substituted by a C₁₋₆ alkoxyl group, or substituted by one or more halo groups. In some embodiments, R^{L1} is an alkyl selected from the group consisting of a linear C₁₋₃ alkyl and a branched C₁₋₃ alkyl, each of which is unsubstituted, or substituted by a C₁₋₆ alkoxyl group, or substituted by one or more halo groups. In some embodiments, R^{L1} is an unsubstituted C₁₋₃ alkyl group selected from methyl, ethyl, n-propyl and isopropyl.

In some embodiments, X³ is selected from the group consisting of a C₁₋₆ alkylene group, a 3- to 7-membered cyclic alkylene group, and a 3- to 7-membered heterocyclic alkylene group having 0 to 2 hetero atoms, each of which is unsubstituted or substituted by a C₁₋₆ alkyl or a C₃₋₆ cycloalkyl. In some embodiments, X³ is an unsubstituted C₁₋₆ alkylene group selected from a methylene group, an ethylene group, a propylene group, a butylene group, a pentylene group and a hexylene group.

In some embodiments, X⁴ is an unsubstituted C₁₋₃ alkylene group selected from a methylene group, an ethylene group and a propylene group.

In some embodiments, X⁵ is selected from the group consisting of a methylene group, CONH and O.

In some embodiments, R^{L3} and R^{L4} are each independently an alkyl selected from the group consisting of a linear C₁₋₆ alkyl and a branched C₁₋₆ alkyl, each of which is unsubstituted, or substituted by a C₁₋₆ alkoxyl group, or substituted by one or more halo groups. In some embodiments, R^{L3} and R^{L4} are each independently an alkyl selected from the group consisting of a linear C₁₋₃ alkyl and a branched C₁₋₃ alkyl, each of which is unsubstituted, or substituted by a C₁₋₄ alkoxyl group, or substituted by one or more halo groups.

In some embodiments, the -L- is selected from the group consisting of

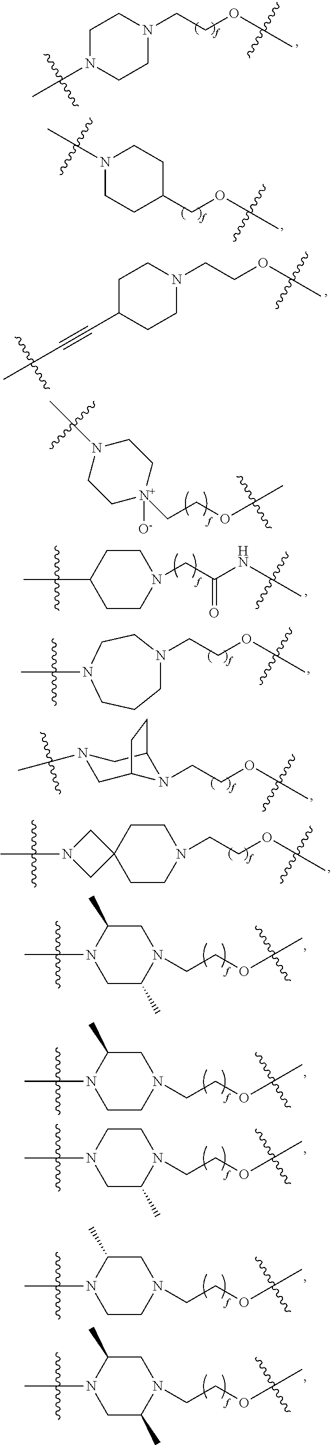

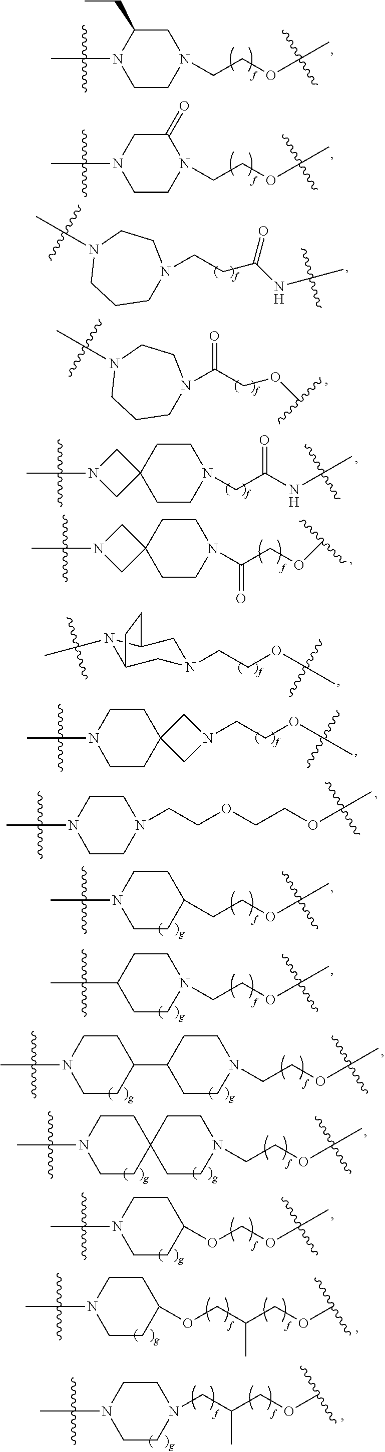

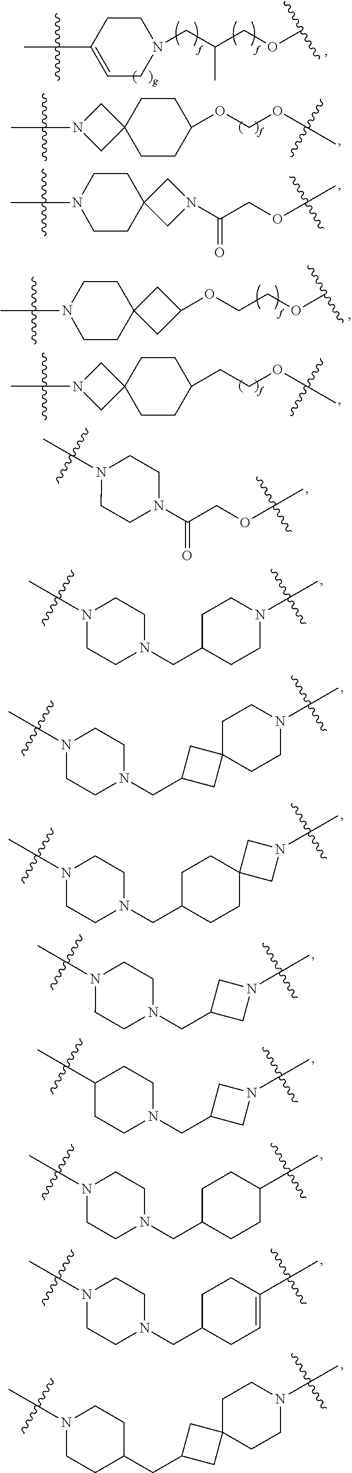

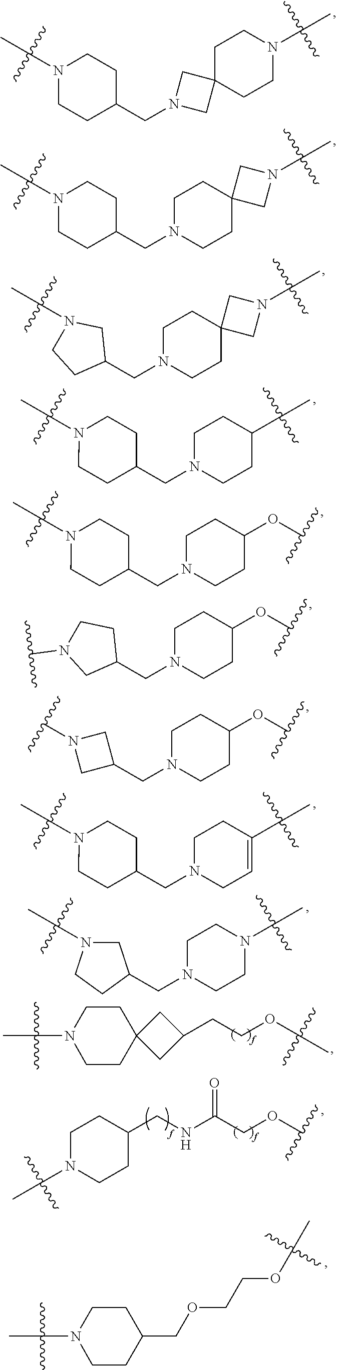

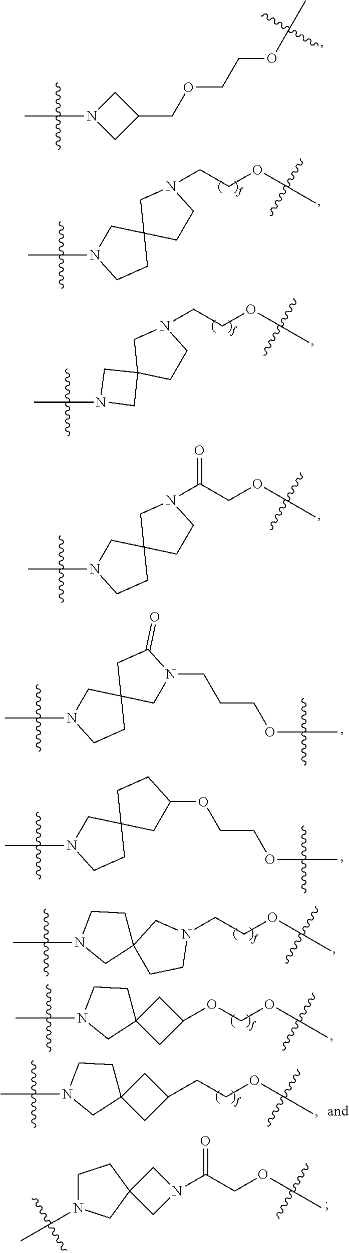

wherein f is an integer of 0, 1, 2, 3 or 4; and
g is an integer of 0, 1, 2 or 3.

In some embodiments, the ABM is an androgen receptor binding moiety represented by

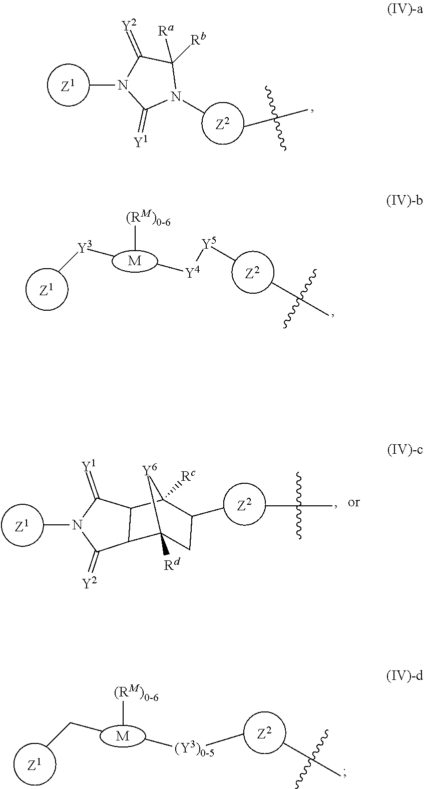

wherein:
Z¹ is selected from the group consisting of an aryl group, a heteroaryl group, a bicyclic group, or a bi-heterocyclic group, each independently substituted by one or more substituents independently selected from the group consisting of a halo group, a hydroxyl group, a nitro group, —CN, —C≡CH, an unsubstituted C₁₋₆ alkyl group, a C₁₋₆ alkyl group substituted by a C₁₋₆ alkoxyl group, a C₁₋₆ alkyl group substituted by one or more halo groups, an unsubstituted C₁₋₆ alkoxyl group, a C₁₋₆ alkoxyl group substituted by one or more halo groups, an unsubstituted C₂₋₆ alkenyl, a C₂₋₆ alkenyl substituted by one or more halo groups, an unsubstituted C₂₋₆ alkynyl, a C₃₋₆ alkynyl substituted by one or more halo groups, and any combinations thereof;
Y¹ and Y² are each independently NR^{Y1}, O or S;
Y³, Y⁴ and Y⁵ are each independently selected from the group consisting of a bond, —O—, —NR^{Y2}—, —C(—R^{Y1})(—R^{Y2})—, —C(═O)—, —C(═S)—, —S(═O)—, —SO₂—, a heteroarylene group, and an arylene group;
Y⁶ is —N(—R^{Y1})—, —O— or —S—;
M is a 3- to 6-membered ring having 0 to 4 heteroatoms, which is unsubstituted or substituted by 0 to 6 R^{M} groups;
each R^{M} group is independently selected from the group consisting of an unsubstituted C₁₋₆ alkyl group, a C₁₋₆ alkyl group substituted by a C₁₋₆ alkoxyl group, a C₁₋₆ alkyl group substituted by one or more halo groups, a halo group, and a C₁₋₆ alkoxyl group; or two R^{M} groups are taken together with the atom they attach to and form a 3- to 8-membered ring system containing 0 to 2 heteroatoms;
R^{a}, R^{b}, R^{c}, R^{d}, R^{Y1} and R^{Y2} are each independently selected from the group consisting of —H, an unsubstituted C₁₋₆ alkyl group, a C₁₋₆ alkyl group substituted by a C₁₋₆ alkoxyl group, a C₁₋₆ alkyl group substituted by one or more halo groups, a halo group, a C₁₋₆ alkoxyl group, a cyclic group, and a heterocyclic group; or R^{a}, R^{b} are taken together with the atom they attach to and form a 3- to 8-membered ring system containing 0 to 2 heteroatoms;
Z² is selected from the group consisting of a bond, a C₁₋₆ alkylene group, a C₁₋₆ heteroalkylene group, —O—, an arylene group, a heteroarylene group, an alicyclic bivalent group, a heterocyclic bivalent group, a heterobicyclic bivalent group, a bicyclic arylene group, and a bicyclic heteroarylene group, each or which is unsubstituted or substituted by 1 to 10 R^{Z2} groups;
each R^{Z2} group is independently selected from the group consisting of —H, a halo group, an unsubstituted C₁₋₆ alkyl group, a C₁₋₆ alkyl group substituted by one or more —F, —OR^{Z2A}, a C₃₋₆ cycloalkyl group, a C₄₋₆ cycloheteroalkyl group, an unsubstituted C₁₋₆ alkyl group, a C₁₋₆ alkyl group substituted by a C₁₋₃ alkyl group, a C₁₋₆ alkyl group substituted by a C₁₋₆ alkoxyl group, a C₁₋₆ alkyl group substituted by one or more halo groups, an unsubstituted heterocyclic group, a heterocyclic group substituted by a C₁₋₃ alkyl group, a heterocyclic group substituted by a C₁₋₆ alkoxyl group, a heterocyclic group substituted by one or more halo groups, an unsubstituted aryl group, an aryl group substituted by a C₁₋₃ alkyl group, an aryl group substituted by a C₁₋₆ alkoxyl group, an aryl group substituted by one or more halo groups, an unsubstituted heteroaryl group, a heteroaryl group substituted by a C₁₋₃ alkyl group, a heteroaryl group substituted by a C₁₋₆ alkoxyl group, a heteroaryl group substituted by one or more halo groups, a bicyclic heteroaryl group, an unsubstituted C₁₋₃ alkoxyl group, and a C₁₋₃ alkoxyl substituted by one or more groups selected from —F, —OH, —NH₂, —NR^{Y1}R^{Y2} and —CN; and
R^{Z2A} is selected from the group consisting of H, a C₁₋₆ alkyl group, and a C₁₋₆ heteroalkyl group, each of which is unsubstituted or substituted by a cycloalkyl group, a cycloheteroalkyl group, an aryl group, a heterocyclic group, a heteroaryl group, a halo group, or a C₁₋₃ alkoxyl group.

In some embodiments, the heteroatoms in Formula (IV)-a, (IV)-b, (IV)-c or (IV)-d are each independently selected from N, O and S.

In some embodiments, Z¹ is selected from the group consisting of

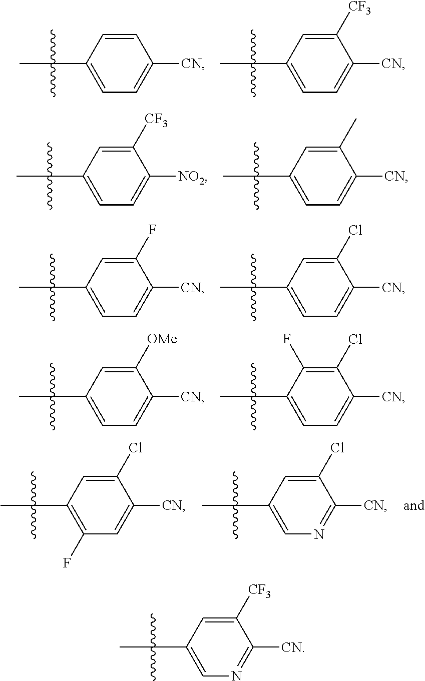

In some embodiments, Z¹ is selected from the group consisting of:

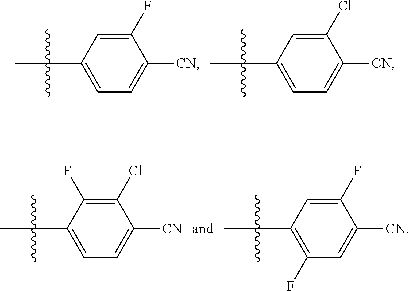 [SMILES: *c1ccc(C#N)c(F)c1 ; *c1ccc(C#N)c(Cl)c1 ; *c1ccc(C#N)c(Cl)c1F ; *c1cc(F)c(C#N)cc1F]

In some embodiments, Z² is selected from the group consisting of

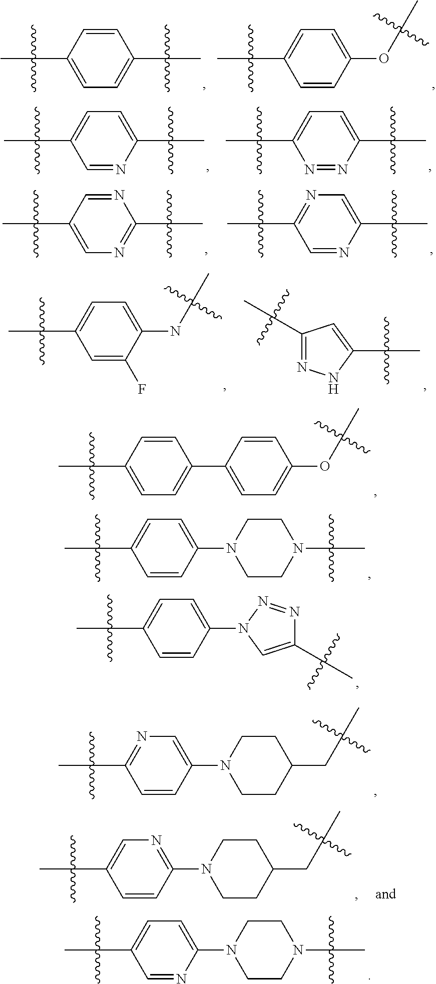

In some embodiments, M is

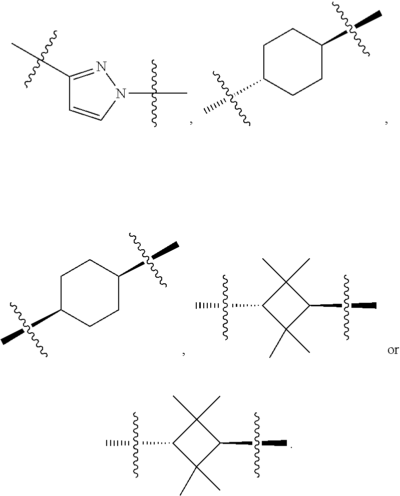

In some embodiments, each R^{M} group is independently an alkyl selected from the group consisting of a linear C₁₋₆ alkyl and a branched C₁₋₆ alkyl, each of which is unsubstituted, or substituted by a C₁₋₆ alkoxyl group, or substituted by one or more halo groups. In some embodiments, each R^{M} group is independently an alkyl selected from the group consisting of a linear C₁₋₃ alkyl and a branched C₁₋₃ alkyl, each of which is unsubstituted, or substituted by a C₁₋₆ alkoxyl group, or substituted by one or more halo groups. In some embodiments, each R^{M} group is independently methyl, ethyl, n-propyl or isopropyl.

In some embodiments, R^{a}, R^{b}, R^{c}, R^{d}, R^{Y1} and R^{Y2} are each independently an alkyl selected from the group consisting of a linear C₁₋₆ alkyl and a branched C₁₋₆ alkyl, each of which is unsubstituted, or substituted by a C₁₋₆ alkoxyl group, or substituted by one or more halo groups. In some embodiments, R^{a}, R^{b}, R^{c}, R^{d}, R^{Y1} and R^{Y2} are each independently an alkyl selected from the group consisting of a linear C₁₋₃ alkyl and a branched C₁₋₃ alkyl, each of which is unsubstituted, or substituted by a C₁₋₆ alkoxyl group, or substituted by one or more halo groups. In some embodiments, R^{a}, R^{b}, R^{c}, R^{d}, R^{Y1} and R^{Y2} are independently methyl, ethyl, n-propyl or isopropyl.

In some embodiments, (Y³)₀₋₅ shown in Formula (IV)-d is

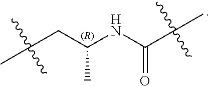

The mark (R) indicates that the carbon has R (rectus) configuration.

In some embodiments, each R^{Z2} group is independently an alkyl selected from the group consisting of a linear C₁₋₆ alkyl and a branched C₁₋₆ alkyl, each of which is unsubstituted or substituted by one or more halo groups. In some embodiments, each R^{Z2} group is independently an alkyl selected from the group consisting of a linear C₁₋₃ alkyl and a branched C₁₋₃ alkyl, each of which is unsubstituted or substituted by one or more halo groups.

In some embodiments, R^{Z2A} is an alkyl selected from the group consisting of a linear C₁₋₆ alkyl and a branched C₁₋₆ alkyl. In some embodiments, R^{Z2A} is an alkyl selected from the group consisting of a linear C₁₋₃ alkyl and a branched C₁₋₃ alkyl.

In some embodiments, R^{Z2A} is a heteroalkyl selected from the group consisting of a linear C₁₋₆ heteroalkyl and a branched C₁₋₆ heteroalkyl. In some embodiments, R^{Z2A} is a heteroalkyl selected from the group consisting of a linear C₁₋₃ heteroalkyl and a branched C₁₋₃ heteroalkyl.

In some embodiments, the ABM is selected from the group consisting of

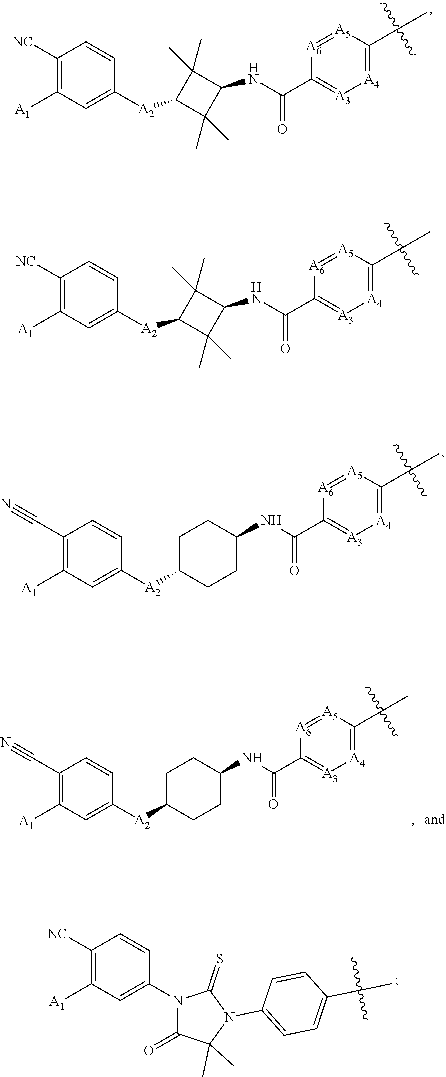

wherein:
A₁ is selected from —Cl, —F, —Br or —CF₃;
A₂ is selected from —O—, —NH—, —NCH₃— or —NCH₂CH₃—; and
A₃, A₄, A₅ and A₆ are each independently CH or N.

In certain embodiments, the ABM is an androgen receptor binding moiety represented by Formula (IV)-b, wherein M is a 4-membered alicyclic ring having 0 to 2 heteroatoms, which is unsubstituted or optionally substituted by 1 to 6 R^{M}. Other groups have the same meaning as indicated above.

In certain embodiments, the ABM is an androgen receptor binding moiety represented by Formula (IV)-b, wherein Z² is a bond, a C₁₋₆ alkylene group, a C₁₋₆ heteroalkylene group, —O—, an arylene group, a heteroarylene group, an alicyclic bivalent group, a heterocyclic bivalent group, a heterobicyclic bivalent group, a bicyclic arylene group, and a bicyclic heteroarylene group, each of which is substituted by 1, 2 or 3 R^{Z2} groups. Other groups have the same meaning as indicated above. Other groups have the same meaning as indicated above.

In certain embodiments, the ABM is an androgen receptor binding moiety represented by Formula (IV)-b, wherein
Z¹ is

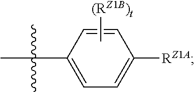

R^{ZA1} is —H or —CN;
each R^{Z1B} is independently —H, a halo group, or —CF₃; t is 0, 1, 2, 3, or 4;
Y³ is —O— or —NR^{Y2}—;
M is a 4- to 6-membered ring, which is unsubstituted or substituted with 1 to 4 R^{M} groups, and each R^{M} is independently —H or methyl;
Y⁴ is —NH—;
Y⁵ is —C(═O)—;
Z² is independently selected from the group consisting of an unsubstituted C₅₋₆ aryl group, a C₅₋₆ aryl group substituted by 1, 2 or 3 R^{Z2} groups, a C₅₋₆ heteroaryl group having 1 to 2 heteroatoms, and a C₅₋₆ heteroaryl group having 1 heteroatom and substituted by 1, 2 or 3 R^{Z2} groups, and a C₅₋₆ heteroaryl group having 2 heteroatoms and substituted by 1 or 2 R^{Z2} groups;
R^{Y2} is —H or an unsubstituted C₁₋₃ alkyl group selected from methyl, ethyl, n-propyl or isopropyl;
each R^{Z2A} group is an alkyl selected from the group consisting of a linear C₁₋₆ alkyl and a branched C₁₋₆ alkyl. In some embodiments, R^{Z2A} is an alkyl selected from the group consisting of a linear C₁₋₃ alkyl and a branched C₁₋₃ alkyl. Other groups have the same meaning as indicated above. Other groups have the same meaning as indicated above.

In certain embodiments, the ABM is an androgen receptor binding moiety represented by Formula (IV)-d, wherein
Z¹ is an aryl substituted by one or more halo groups, or an aryl substituted by —CN; or an aryl independently substituted by —CN and one or more halo groups;
Y³ is a bond, —NR^{Y2}, —CR^{Y1}R^{Y2}, or —C(═O)—;
M is a 5-membered aromatic ring having 1 or 2 heteroatoms;
R^{Y1} and R^{Y2} are each independently —H, or a C₁₋₆ alkyl group;
Z² is a bond, an aryl, or heteroaryl, each optionally substituted by 1, 2 or 3 R^{w2}; and each R^{w2} is independently —H, a halo group, C₁₋₆ alkyl (optionally substituted by 1 or more —F), C₁₋₃ alkoxyl (optionally substituted by 1 or more —F);
R^{W2} group is independently selected from the group consisting of —H, a halo group, a 6-membered alicyclic group having 1 or 2 heteroatoms, or a 5-membered aromatic group having 1 or 2 or 3 heteroatoms. Other groups have the same meaning as indicated above.

In certain embodiments, the ABM is an androgen receptor binding moiety represented by Formula (IV)-d, wherein
Z¹ is

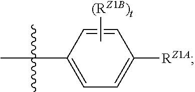

wherein each R^{Z1A} is a halo group or CN; and each R^{Z1B} is independently H or halo. Other groups have the same meaning as indicated above.

In some embodiments, ABM is selected from the group consisting of

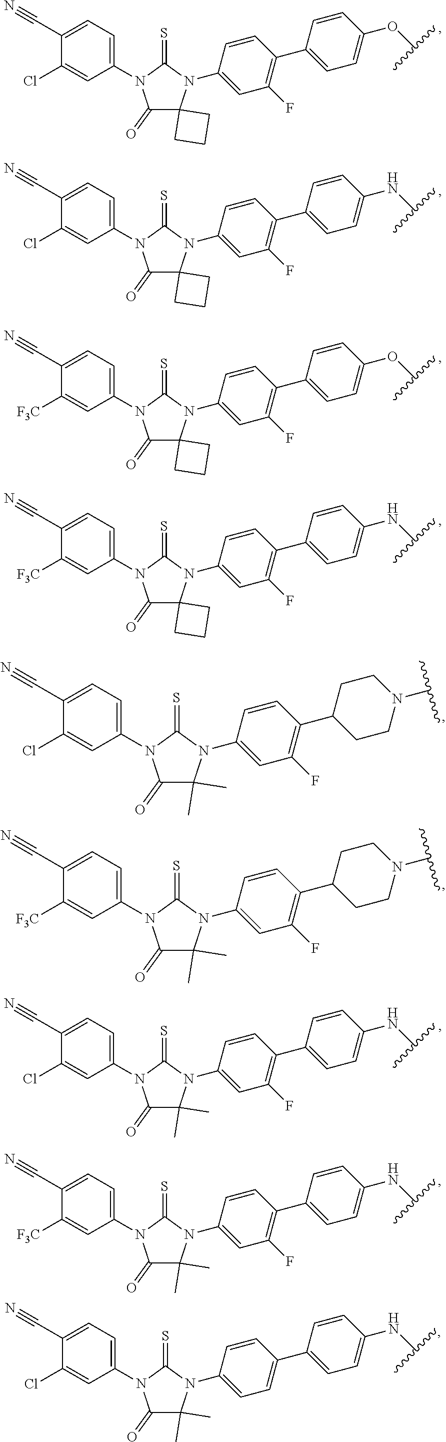

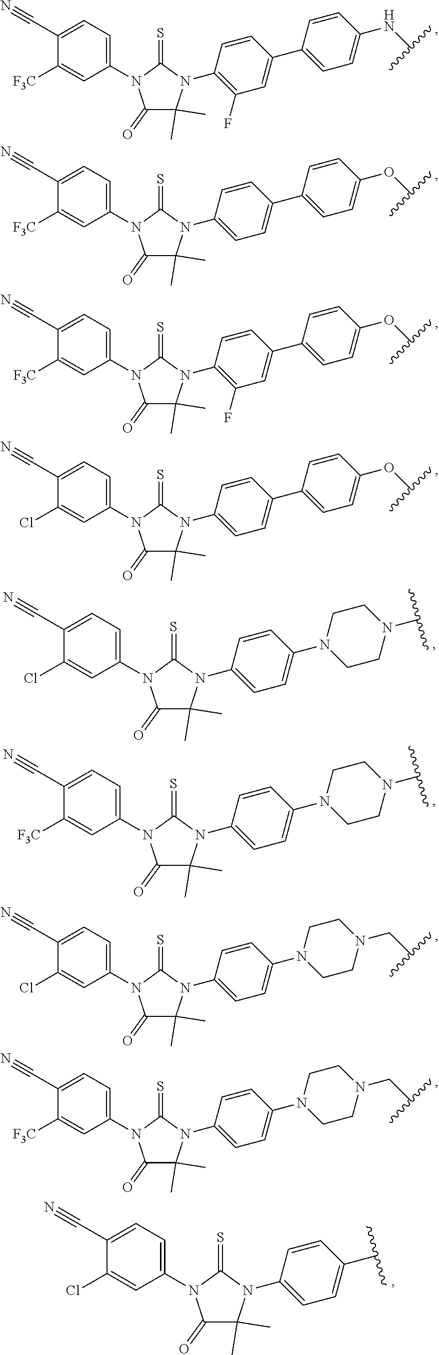

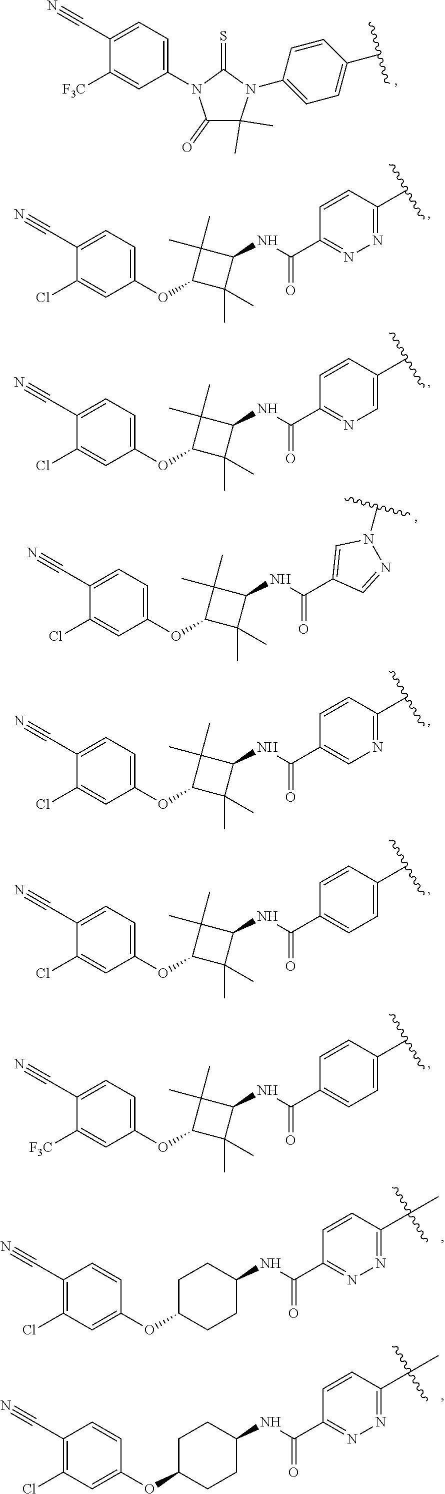

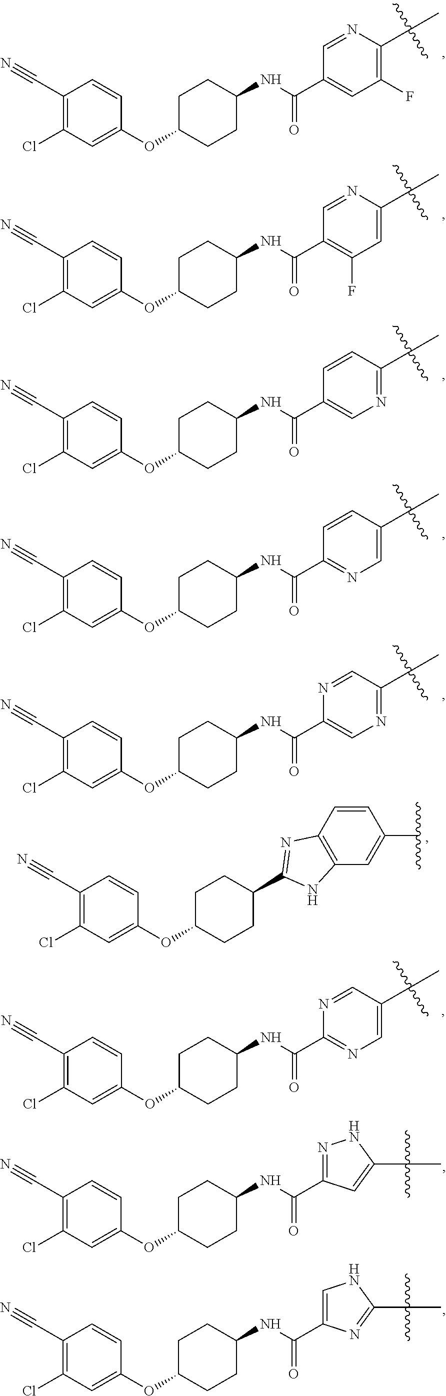

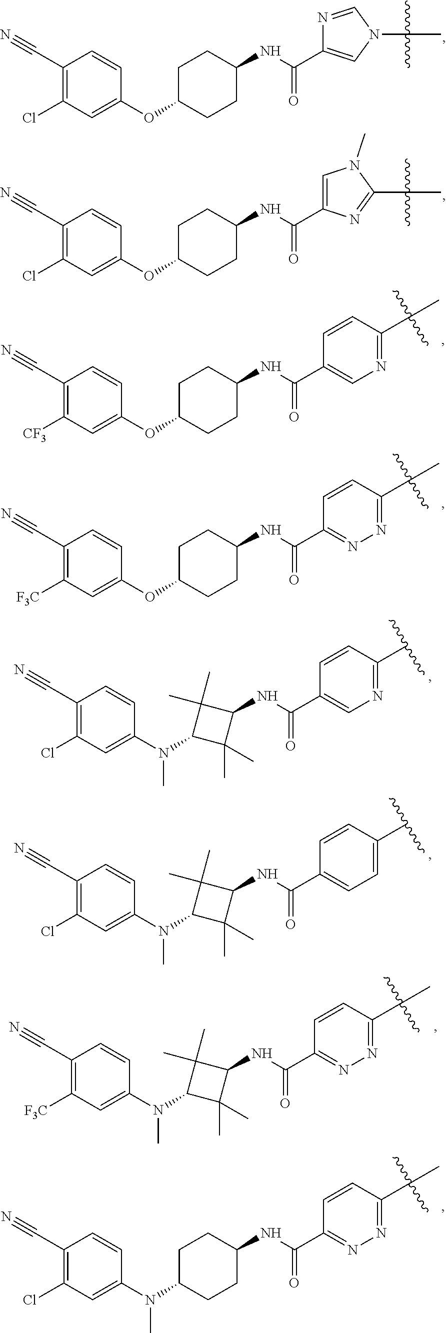

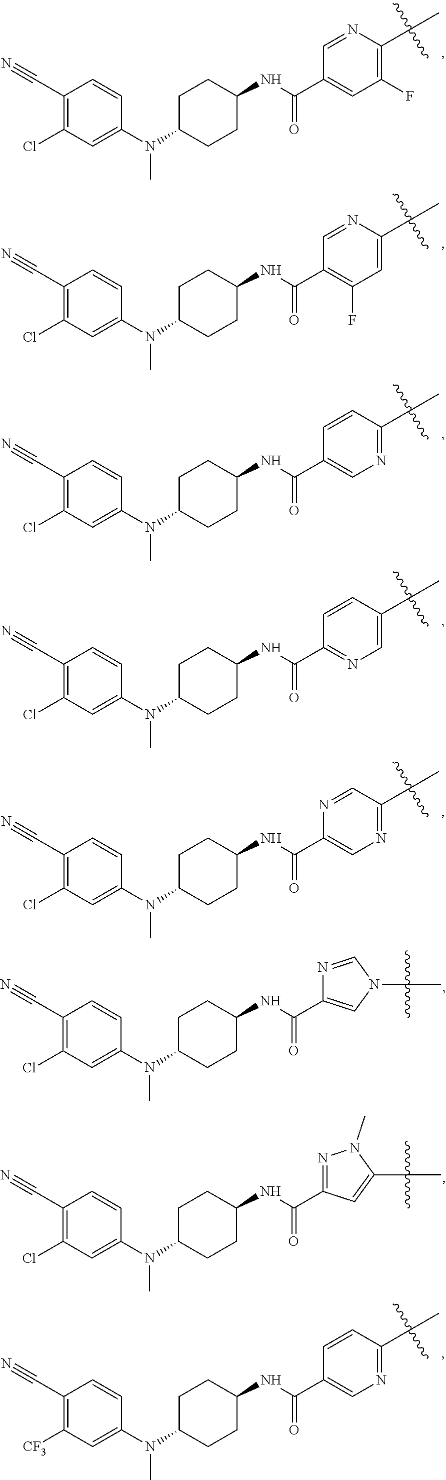

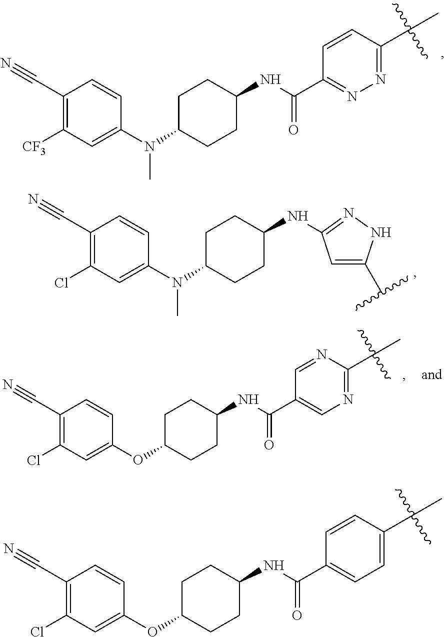 [SMILES: *c1ccc(C(=O)N[C@H]2CC[C@H](N(C)c3ccc(C#N)c(C(F)(F)F)c3)CC2)nn1 ; *c1cc(N[C@H]2CC[C@H](N(C)c3ccc(C#N)c(Cl)c3)CC2)n[nH]1 ; *c1ncc(C(=O)N[C@H]2CC[C@H](Oc3ccc(C#N)c(Cl)c3)CC2)cn1 ; *c1ccc(C(=O)N[C@H]2CC[C@H](Oc3ccc(C#N)c(Cl)c3)CC2)cc1]

In some embodiments, the pharmaceutical composition of the present invention further comprises a second therapeutic agent.

In some embodiments, the androgen receptor related disease is an androgen receptor related cancer or an androgen receptor related skin disease.

In some embodiments, the androgen receptor related cancer is breast cancer or prostate cancer. In some embodiments, the cancer is breast cancer. In some embodiments, the cancer is prostate cancer. In some embodiments, the prostate cancer is castration-resistant prostate cancer.

In some embodiments, the androgen receptor related skin disease is androgenetic alopecia, acne, hidradenitis suppurativa, hirsutism, or atopic dermatitis.

In some embodiments, the method for treating an androgen receptor related disease further comprises administering an effective amount of a second therapeutic agent.

In some embodiments, the second therapeutic agent may be an androgen receptor inhibitor. Examples of the androgen receptor inhibitor may be enzalutamide.

In some embodiments, the second therapeutic agent may be an antitumor agent conventionally used in the art. Examples of conventional antitumor agents include Docetaxel, Flutamide, Goserelin acetate, Leuprolide acetate, colchicine, Leuprolide acetate, Mitoxantrone hydrochloride, 5-fluorouracil, and Olaparib. Examples thereof include one or more of cyclophosphamide, mitomycin C and the like.

In some embodiments, the second therapeutic agent may be a therapeutic agent for treating an AR related skin disease conventionally used in the art. Examples of conventional antitumor agents include Clascoterone, ASC-J9, Spironolactone, Flutamide, Finasteride, dutasteride, Cyproterone acetate, Pyrilutamide, Minoxidil, Ketoconazole and the like.

As used herein in the specification and in the claims, the articles “a” and “an” are used to refer to one or to more than one (i.e., to at least one) of the grammatical object of the article unless the context clearly indicates otherwise. By way of example, “an element” means one element or more than one element.

As used herein in the specification and in the claims, the phrase “and/or,” should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “effective” can mean, but is in no way limited to, that amount/dose of the active pharmaceutical ingredient, which, when used in the context of its intended use, effectuates or is sufficient to prevent, inhibit the occurrence, ameliorate, delay or treat (alleviate a symptom to some extent, preferably all) the symptoms of a condition, disorder or disease state in a subject in need of such treatment or receiving such treatment. The term “effective” subsumes all other effective amount or effective concentration terms, e.g., “effective amount/dose,” “pharmaceutically effective amount/dose” or “therapeutically effective amount/dose,” which are otherwise described or used in the present application.

The effective amount depends on the type and severity of disease, the composition used, the route of administration, the type of mammal being treated, the physical characteristics of the specific mammal under consideration, concurrent medication, and other factors which those skilled in the medical arts will recognize. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other steroisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described.

It is understood by those of ordinary skill in the art that molecules which are described herein are stable compounds as generally described hereunder. When the bond [symbol] is shown, both a double bond and single bond are represented or understood within the context of the compound shown and well-known rules for valence interactions.

As used herein, an “alkoxyl group” refers to an alkyl group which is singularly bonded to oxygen; such as methoxy (—O—CH₃) and ethoxy (—O—CH₂CH₃).

As used herein, “derivatives” can mean compositions formed from the native compounds either directly, by modification, or by partial substitution. As used herein, “analogs” can mean compositions that have a structure similar to, but not identical to, the native compound.

The term “ubiquitin ligase” refers to a family of proteins that facilitate the transfer of ubiquitin to a specific substrate protein, targeting the substrate protein for degradation. For example, cereblonE3 ubiquitin ligase, alone or in combination with E2 ubiquitin-conjugating enzyme, causes the attachment of ubiquitin to lysine on a target protein, and subsequently targets the specific protein substrates for degradation by the proteasome. Thus, E3 ubiquitin ligase, alone or in complex with E2 ubiquitin conjugating enzyme, is responsible for the transfer of ubiquitin to target proteins. In general, the ubiquitin ligases are involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further, E3 ubiquitin ligase promotes the formation of poly-ubiquitin chains through the lysine residues on ubiquitin. Lys48-linked chains are the predominant type of poly-ubiquitination, which can target proteins to proteasome for degradation.

The term “patient” or “subject” is used throughout the specification to describe a cell, tissue, or animal, preferably a mammal, e.g., a human or a domesticated animal, to whom treatment, including prophylactic treatment, with the compositions according to the present disclosure is provided. For treatment of those infections, conditions or disease states which are specific for a specific animal such as a human patient, the term “patient” refers to that specific animal, including a domesticated animal such as a dog or cat or a farm animal such as a horse, cow, sheep, etc. In general, in the present disclosure, the term “patient” refers to a human patient unless otherwise stated or implied from the context of the use of the term.

Understand that in all cases disclosed herein, the description of an integer range of any variable describes the description range, all individual members of the range, and all possible subranges of that variable. For example, the description that n is an integer from 0 to 4 is 0, 1, 2, 3 and 4 are described as being in the range of individual selectable values. In addition, the description that n is an integer from 0 to 4 also describes each and all subranges, each of which n is 0-4, 0-3, 0-2, 0-1, 1-4, 1-3, 1-2, 2-4, 2-3 and 3-4. In addition, the term “C₁₋₆ alkyl” indicates an alkyl having a carbon number of 1 to 6.

The following is a detailed description provided to aid those skilled in the art in practicing the present invention. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

### BRIEF DESCRIPTION OF THE DRAWINGS

FIG. 1 is the color chart of the skin color scores 1 to 8 used in the hair regrowth experiment of C57BL/6 mice.

FIG. 2 is the photographs of the photographs of the Corn oil+Vehicle group and the DHT+Vehicle group.

FIG. 3 is a line graph representing the hair regrowth efficacy of the compounds of the present invention.

### DETAILED DESCRIPTION OF THE PREFERRED EMBODIMENTS

Shown in Table 1 below are exemplary of the bifunctional compounds with the structure ABM-L-CLM of the present invention:

TABLE 1

| | |
|---|---|
| 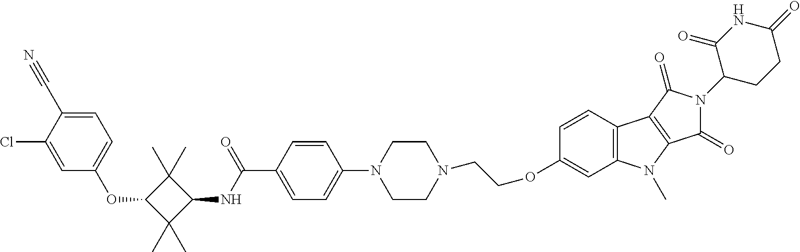 [SMILES: Cn1c2c(c3ccc(OCCN4CCN(c5ccc(C(=O)N[C@H]6C(C)(C)[C@H](Oc7ccc(C#N)c(Cl)c7)C6(C)C)cc5)CC4)cc31)C(=O)N(C1CCC(=O)NC1=O)C2=O] | Com- pound 1 |
| 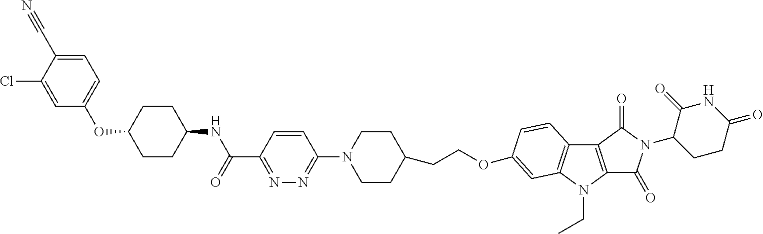 [SMILES: CCn1c2c(c3ccc(OCCC4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 2 |
| 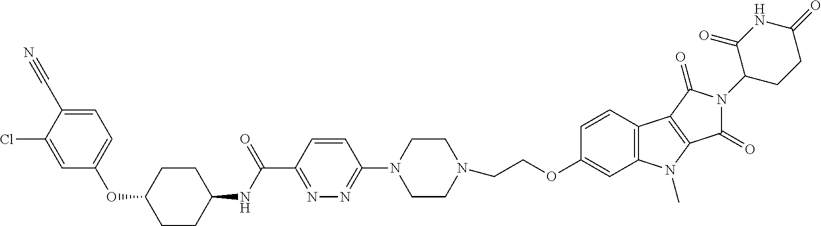 [SMILES: Cn1c2c(c3ccc(OCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 3 |
| 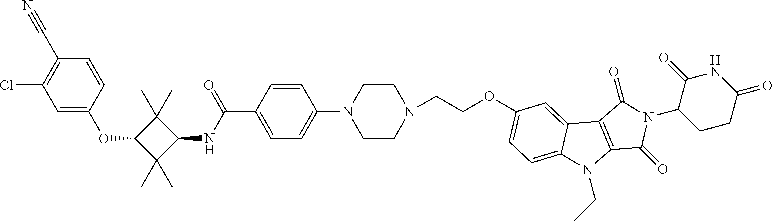 [SMILES: CCn1c2c(c3cc(OCCN4CCN(c5ccc(C(=O)N[C@H]6C(C)(C)[C@H](Oc7ccc(C#N)c(Cl)c7)C6(C)C)cc5)CC4)ccc31)C(=O)N(C1CCC(=O)NC1=O)C2=O] | Com- pound 4 |
| 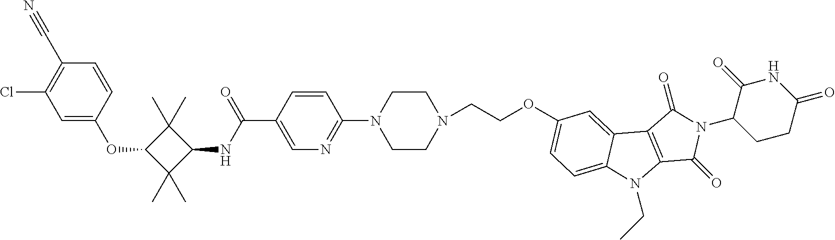 [SMILES: CCn1c2c(c3cc(OCCN4CCN(c5ccc(C(=O)N[C@H]6C(C)(C)[C@H](Oc7ccc(C#N)c(Cl)c7)C6(C)C)cn5)CC4)ccc31)C(=O)N(C1CCC(=O)NC1=O)C2=O] | Com- pound 5 |
| 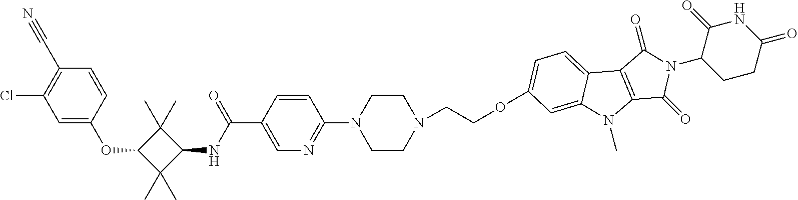 [SMILES: Cn1c2c(c3ccc(OCCN4CCN(c5ccc(C(=O)N[C@H]6C(C)(C)[C@H](Oc7ccc(C#N)c(Cl)c7)C6(C)C)cn5)CC4)cc31)C(=O)N(C1CCC(=O)NC1=O)C2=O] | Com- pound 6 |
| 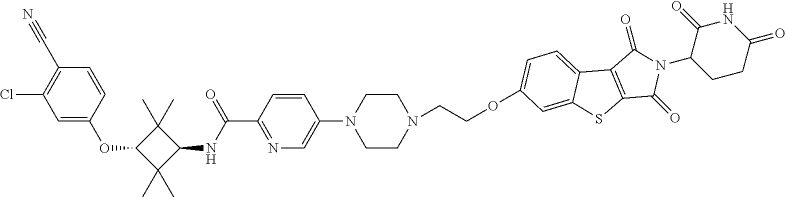 [SMILES: CC1(C)[C@H](NC(=O)c2ccc(N3CCN(CCOc4ccc5c6c(sc5c4)C(=O)N(C4CCC(=O)NC4=O)C6=O)CC3)cn2)C(C)(C)[C@H]1Oc1ccc(C#N)c(Cl)c1] | Com- pound 7 |
| 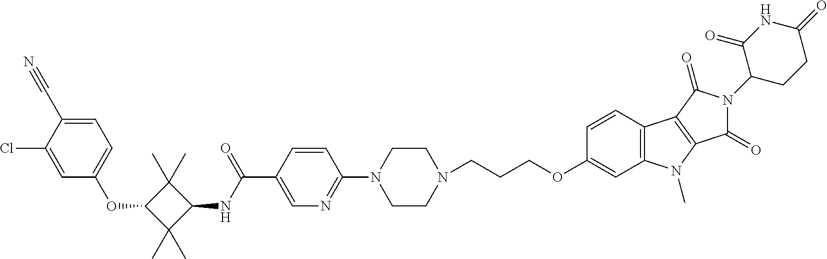 [SMILES: Cn1c2c(c3ccc(OCCCN4CCN(c5ccc(C(=O)N[C@H]6C(C)(C)[C@H](Oc7ccc(C#N)c(Cl)c7)C6(C)C)cn5)CC4)cc31)C(=O)N(C1CCC(=O)NC1=O)C2=O] | Com- pound 8 |
| 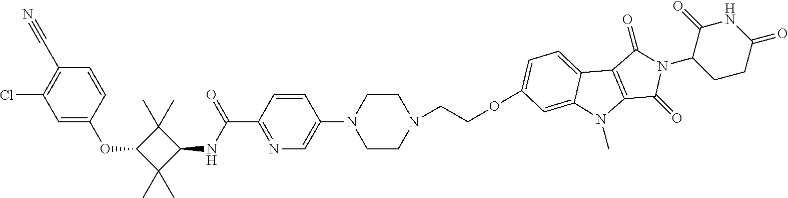 [SMILES: Cn1c2c(c3ccc(OCCN4CCN(c5ccc(C(=O)N[C@H]6C(C)(C)[C@H](Oc7ccc(C#N)c(Cl)c7)C6(C)C)nc5)CC4)cc31)C(=O)N(C1CCC(=O)NC1=O)C2=O] | Com- pound 9 |
| 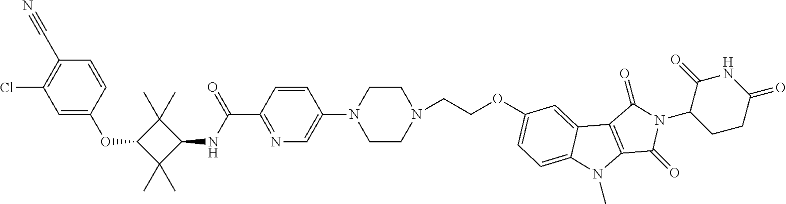 [SMILES: Cn1c2c(c3cc(OCCN4CCN(c5ccc(C(=O)N[C@H]6C(C)(C)[C@H](Oc7ccc(C#N)c(Cl)c7)C6(C)C)nc5)CC4)ccc31)C(=O)N(C1CCC(=O)NC1=O)C2=O] | Com- pound 10 |
| 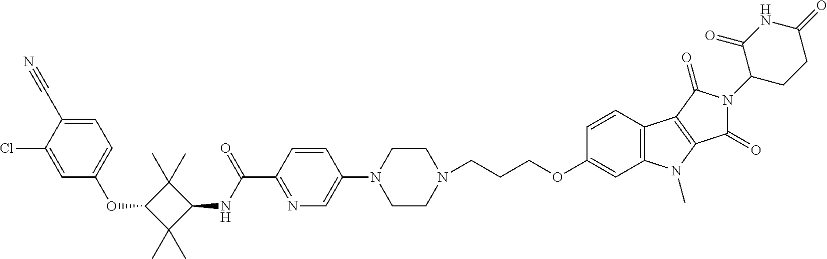 [SMILES: Cn1c2c(c3ccc(OCCCN4CCN(c5ccc(C(=O)N[C@H]6C(C)(C)[C@H](Oc7ccc(C#N)c(Cl)c7)C6(C)C)nc5)CC4)cc31)C(=O)N(C1CCC(=O)NC1=O)C2=O] | Com- pound 11 |
| 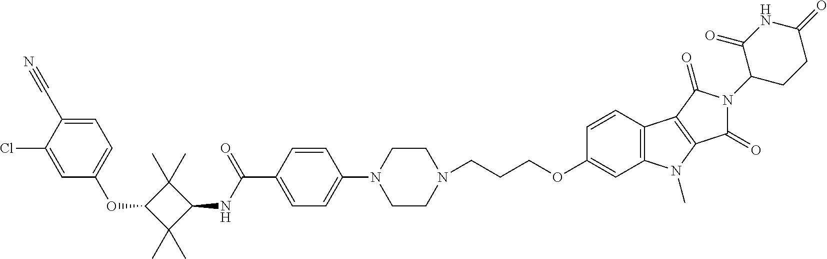 [SMILES: Cn1c2c(c3ccc(OCCCN4CCN(c5ccc(C(=O)N[C@H]6C(C)(C)[C@H](Oc7ccc(C#N)c(Cl)c7)C6(C)C)cc5)CC4)cc31)C(=O)N(C1CCC(=O)NC1=O)C2=O] | Com- pound 12 |
| 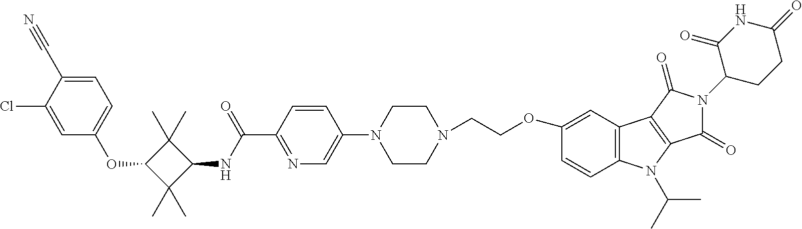 [SMILES: CC(C)n1c2c(c3cc(OCCN4CCN(c5ccc(C(=O)N[C@H]6C(C)(C)[C@H](Oc7ccc(C#N)c(Cl)c7)C6(C)C)nc5)CC4)ccc31)C(=O)N(C1CCC(=O)NC1=O)C2=O] | Com- pound 13 |
| 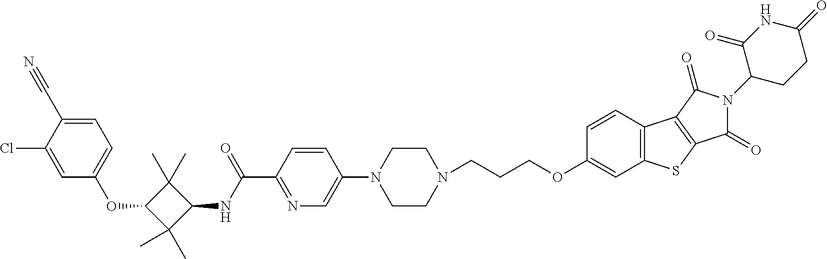 [SMILES: CC1(C)[C@H](NC(=O)c2ccc(N3CCN(CCCOc4ccc5c6c(sc5c4)C(=O)N(C4CCC(=O)NC4=O)C6=O)CC3)cn2)C(C)(C)[C@H]1Oc1ccc(C#N)c(Cl)c1] | Com- pound 14 |
| 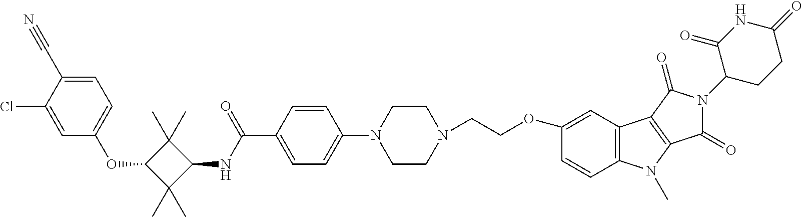 [SMILES: Cn1c2c(c3cc(OCCN4CCN(c5ccc(C(=O)N[C@H]6C(C)(C)[C@H](Oc7ccc(C#N)c(Cl)c7)C6(C)C)cc5)CC4)ccc31)C(=O)N(C1CCC(=O)NC1=O)C2=O] | Com- pound 15 |
| 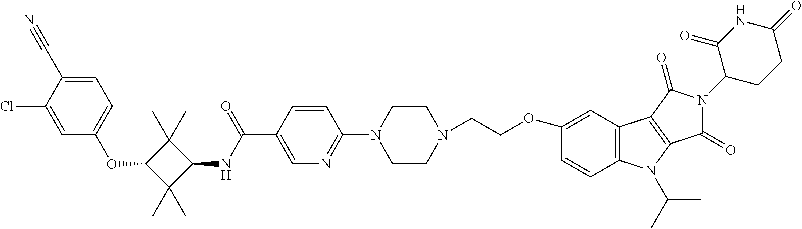 [SMILES: CC(C)n1c2c(c3cc(OCCN4CCN(c5ccc(C(=O)N[C@H]6C(C)(C)[C@H](Oc7ccc(C#N)c(Cl)c7)C6(C)C)cn5)CC4)ccc31)C(=O)N(C1CCC(=O)NC1=O)C2=O] | Com- pound 16 |
| 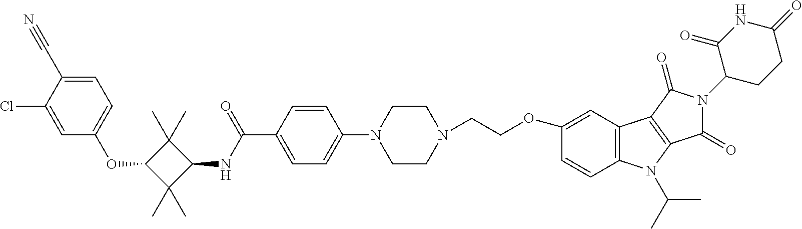 [SMILES: CC(C)n1c2c(c3cc(OCCN4CCN(c5ccc(C(=O)N[C@H]6C(C)(C)[C@H](Oc7ccc(C#N)c(Cl)c7)C6(C)C)cc5)CC4)ccc31)C(=O)N(C1CCC(=O)NC1=O)C2=O] | Com- pound 17 |
| 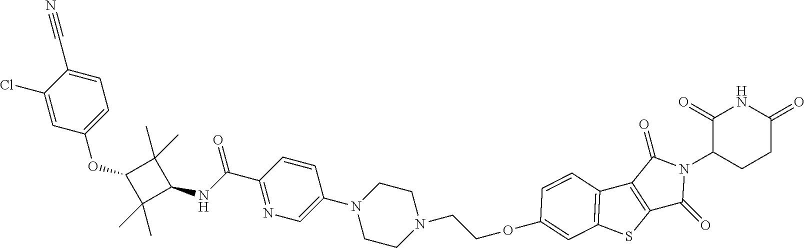 [SMILES: CC1(C)[C@H](NC(=O)c2ccc(N3CCN(CCOc4ccc5c6c(sc5c4)C(=O)N(C4CCC(=O)NC4=O)C6=O)CC3)cn2)C(C)(C)[C@H]1Oc1ccc(C#N)c(Cl)c1] | Com- pound 18 |
| 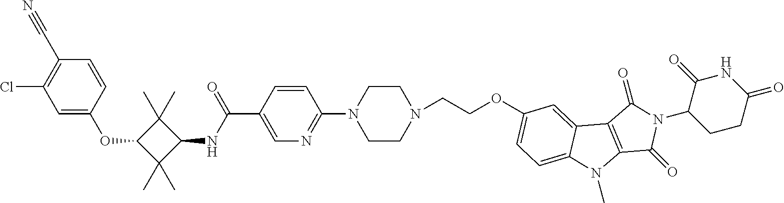 [SMILES: Cn1c2c(c3cc(OCCN4CCN(c5ccc(C(=O)N[C@H]6C(C)(C)[C@H](Oc7ccc(C#N)c(Cl)c7)C6(C)C)cn5)CC4)ccc31)C(=O)N(C1CCC(=O)NC1=O)C2=O] | Com- pound 19 |
| 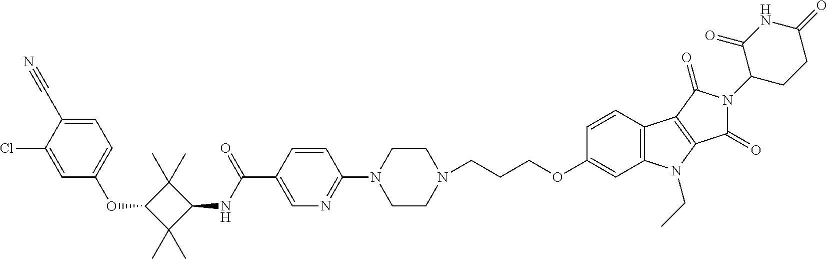 [SMILES: CCn1c2c(c3ccc(OCCCN4CCN(c5ccc(C(=O)N[C@H]6C(C)(C)[C@H](Oc7ccc(C#N)c(Cl)c7)C6(C)C)cn5)CC4)cc31)C(=O)N(C1CCC(=O)NC1=O)C2=O] | Com- pound 20 |
| 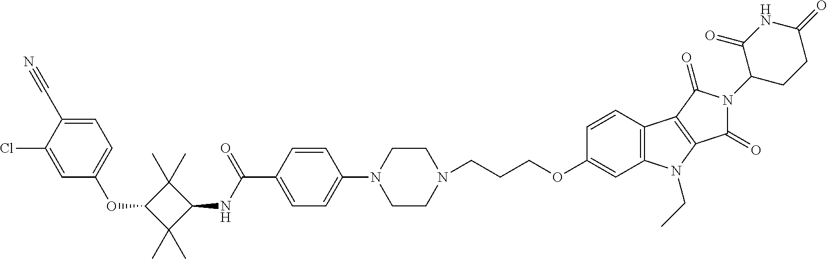 [SMILES: CCn1c2c(c3ccc(OCCCN4CCN(c5ccc(C(=O)N[C@H]6C(C)(C)[C@H](Oc7ccc(C#N)c(Cl)c7)C6(C)C)cc5)CC4)cc31)C(=O)N(C1CCC(=O)NC1=O)C2=O] | Com- pound 21 |
| 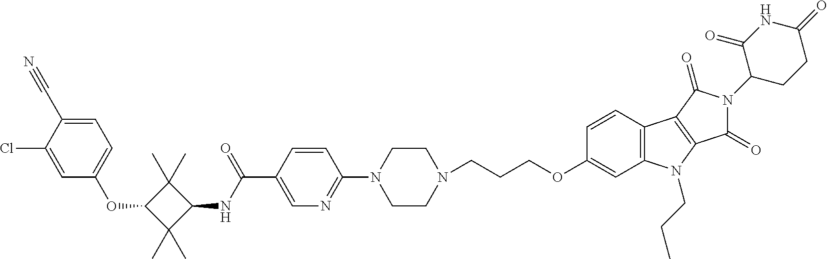 [SMILES: CCCn1c2c(c3ccc(OCCCN4CCN(c5ccc(C(=O)N[C@H]6C(C)(C)[C@H](Oc7ccc(C#N)c(Cl)c7)C6(C)C)cn5)CC4)cc31)C(=O)N(C1CCC(=O)NC1=O)C2=O] | Com- pound 22 |
| 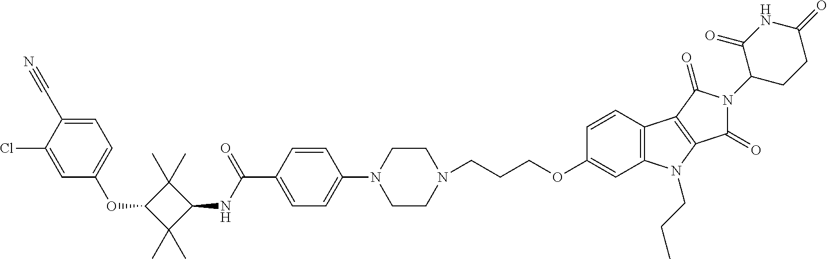 [SMILES: CCCn1c2c(c3ccc(OCCCN4CCN(c5ccc(C(=O)N[C@H]6C(C)(C)[C@H](Oc7ccc(C#N)c(Cl)c7)C6(C)C)cc5)CC4)cc31)C(=O)N(C1CCC(=O)NC1=O)C2=O] | Com- pound 23 |
| 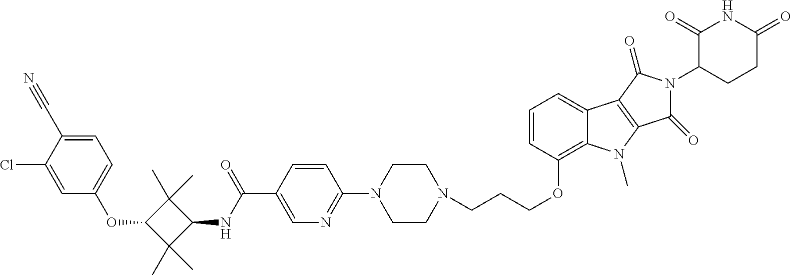 [SMILES: Cn1c2c(c3cccc(OCCCN4CCN(c5ccc(C(=O)N[C@H]6C(C)(C)[C@H](Oc7ccc(C#N)c(Cl)c7)C6(C)C)cn5)CC4)c31)C(=O)N(C1CCC(=O)NC1=O)C2=O] | Com- pound 24 |
| 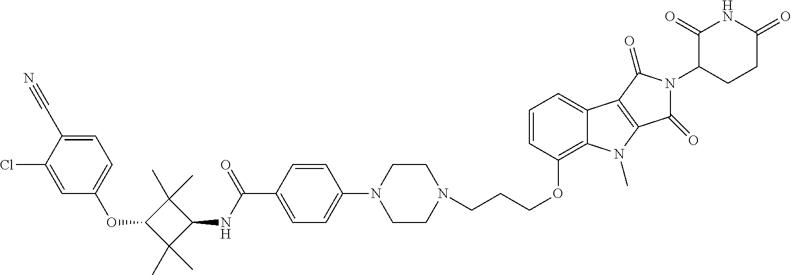 [SMILES: Cn1c2c(c3cccc(OCCCN4CCN(c5ccc(C(=O)N[C@H]6C(C)(C)[C@H](Oc7ccc(C#N)c(Cl)c7)C6(C)C)cc5)CC4)c31)C(=O)N(C1CCC(=O)NC1=O)C2=O] | Com- pound 25 |
| 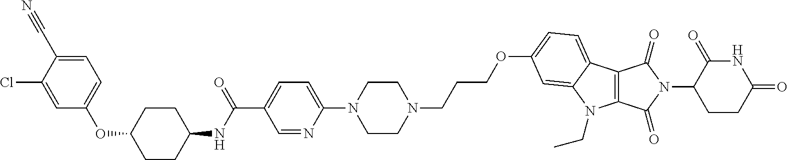 [SMILES: CCn1c2c(c3ccc(OCCCN4CCN(c5ccc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 26 |
| 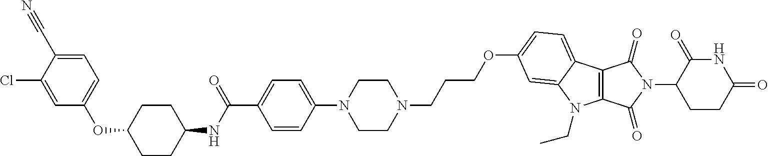 [SMILES: CCn1c2c(c3ccc(OCCCN4CCN(c5ccc(C(=O)N[C@H]6CC[C@H](Oc7ccc(C#N)c(Cl)c7)CC6)cc5)CC4)cc31)C(=O)N(C1CCC(=O)NC1=O)C2=O] | Com- pound 27 |
| 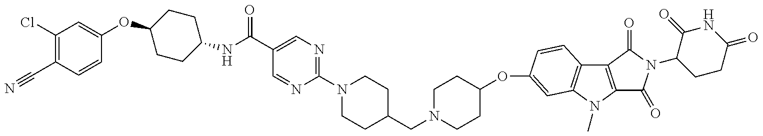 [SMILES: Cn1c2c(c3ccc(OC4CCN(CC5CCN(c6ncc(C(=O)N[C@H]7CC[C@H](Oc8ccc(C#N)c(Cl)c8)CC7)cn6)CC5)CC4)cc31)C(=O)N(C1CCC(=O)NC1=O)C2=O] | Com- pound 28 |
| 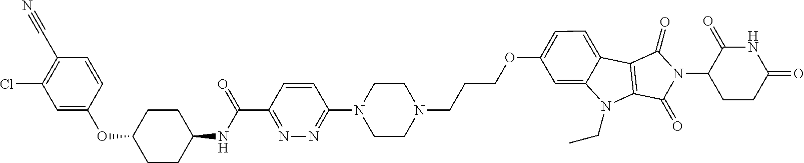 [SMILES: CCn1c2c(c3ccc(OCCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 29 |
| 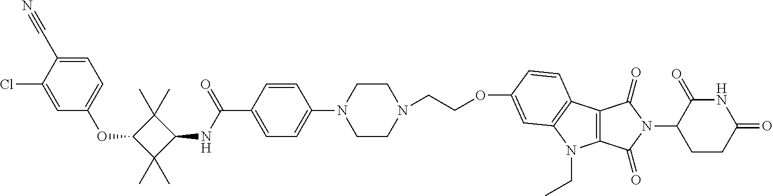 [SMILES: CCn1c2c(c3ccc(OCCN4CCN(c5ccc(C(=O)N[C@H]6C(C)(C)[C@H](Oc7ccc(C#N)c(Cl)c7)C6(C)C)cc5)CC4)cc31)C(=O)N(C1CCC(=O)NC1=O)C2=O] | Com- pound 30 |
| 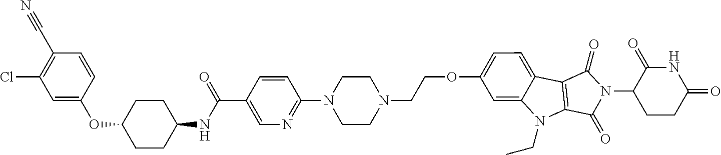 [SMILES: CCn1c2c(c3ccc(OCCN4CCN(c5ccc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 31 |
| 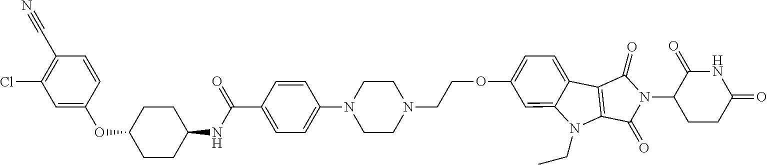 [SMILES: CCn1c2c(c3ccc(OCCN4CCN(c5ccc(C(=O)N[C@H]6CC[C@H](Oc7ccc(C#N)c(Cl)c7)CC6)cc5)CC4)cc31)C(=O)N(C1CCC(=O)NC1=O)C2=O] | Com- pound 32 |
| 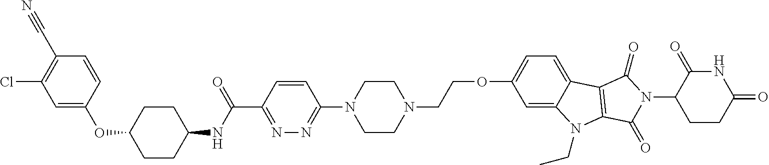 [SMILES: CCn1c2c(c3ccc(OCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 33 |
| 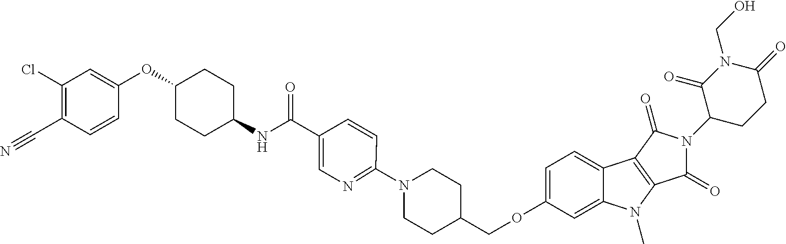 [SMILES: Cn1c2c(c3ccc(OCC4CCN(c5ccc(C(=O)N[C@H]6CC[C@H](Oc7ccc(C#N)c(Cl)c7)CC6)cn5)CC4)cc31)C(=O)N(C1CCC(=O)N(CO)C1=O)C2=O] | Com- pound 34 |
| 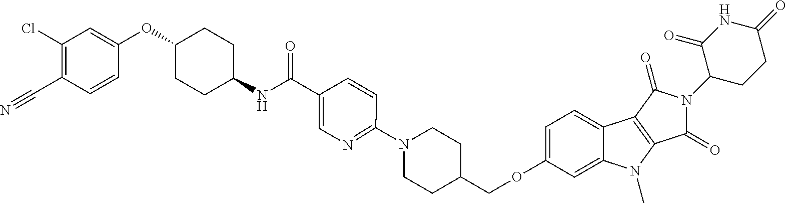 [SMILES: Cn1c2c(c3ccc(OCC4CCN(c5ccc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 35 |
| 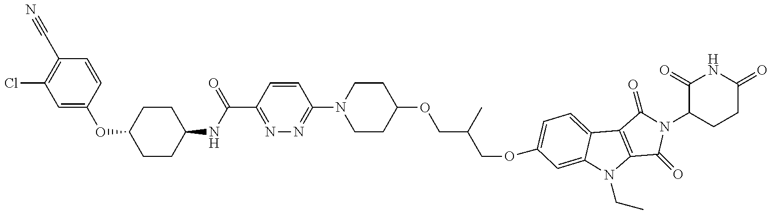 [SMILES: CCn1c2c(c3ccc(OCC(C)COC4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 36 |
| 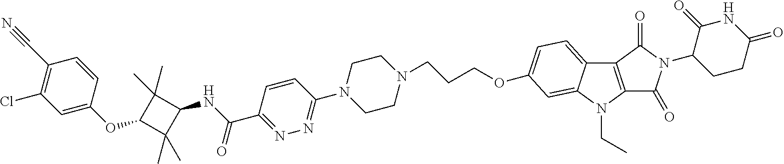 [SMILES: CCn1c2c(c3ccc(OCCCN4CCN(c5ccc(C(=O)N[C@H]6C(C)(C)[C@H](Oc7ccc(C#N)c(Cl)c7)C6(C)C)nn5)CC4)cc31)C(=O)N(C1CCC(=O)NC1=O)C2=O] | Com- pound 37 |
| 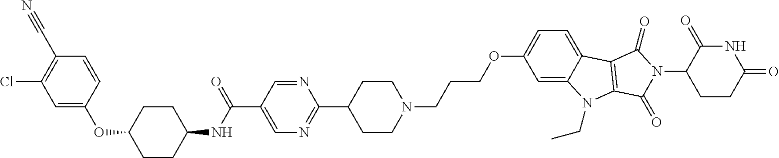 [SMILES: CCn1c2c(c3ccc(OCCCN4CCC(c5ncc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 38 |
| 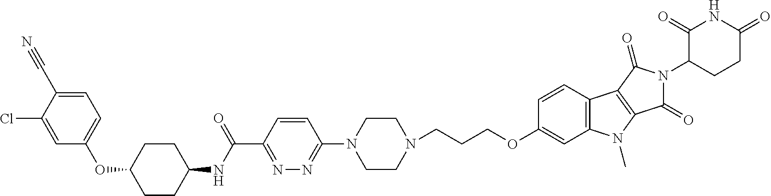 [SMILES: Cn1c2c(c3ccc(OCCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 39 |
| 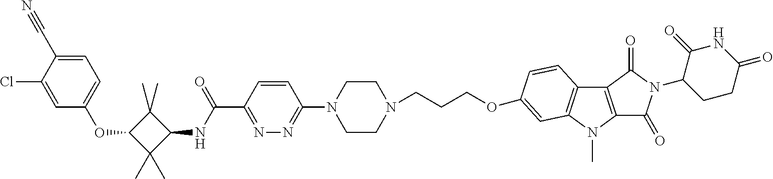 [SMILES: Cn1c2c(c3ccc(OCCCN4CCN(c5ccc(C(=O)N[C@H]6C(C)(C)[C@H](Oc7ccc(C#N)c(Cl)c7)C6(C)C)nn5)CC4)cc31)C(=O)N(C1CCC(=O)NC1=O)C2=O] | Com- pound 40 |
| 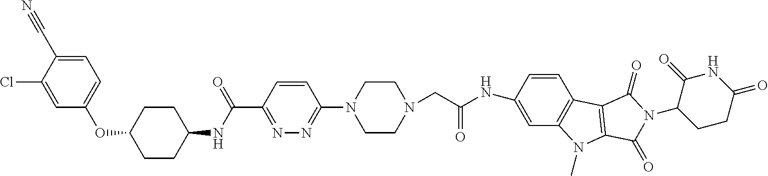 [SMILES: Cn1c2c(c3ccc(NC(=O)CN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 41 |
| 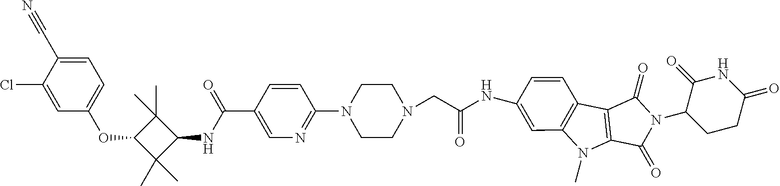 [SMILES: Cn1c2c(c3ccc(NC(=O)CN4CCN(c5ccc(C(=O)N[C@H]6C(C)(C)[C@H](Oc7ccc(C#N)c(Cl)c7)C6(C)C)cn5)CC4)cc31)C(=O)N(C1CCC(=O)NC1=O)C2=O] | Com- pound 42 |
| 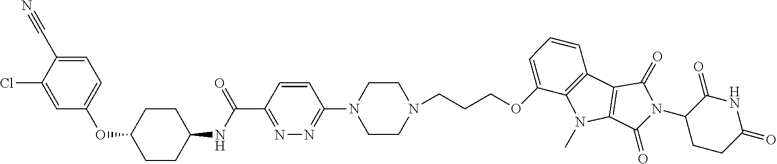 [SMILES: Cn1c2c(c3cccc(OCCCN4CCN(c5ccc(C(=O)N[C@H]6CC[C@H](Oc7ccc(C#N)c(Cl)c7)CC6)nn5)CC4)c31)C(=O)N(C1CCC(=O)NC1=O)C2=O] | Com- pound 43 |
| 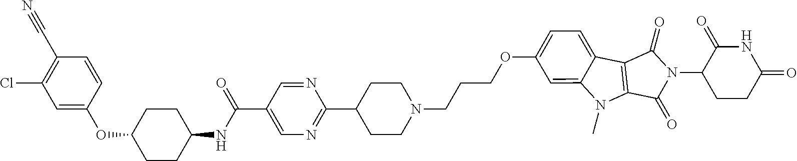 [SMILES: Cn1c2c(c3ccc(OCCCN4CCC(c5ncc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 44 |
| 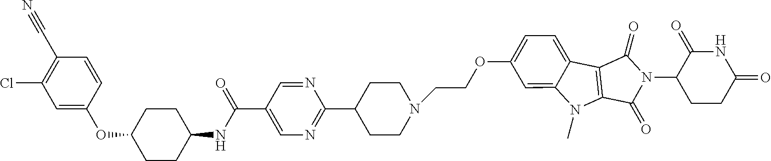 [SMILES: Cn1c2c(c3ccc(OCCN4CCC(c5ncc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 45 |
| 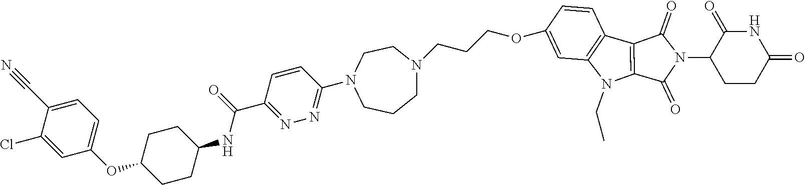 [SMILES: CCn1c2c(c3ccc(OCCCN4CCCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 46 |
| 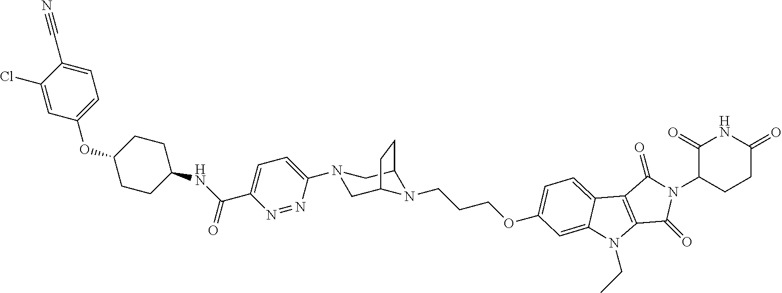 [SMILES: CCn1c2c(c3ccc(OCCCN4C5CCC4CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 47 |
| 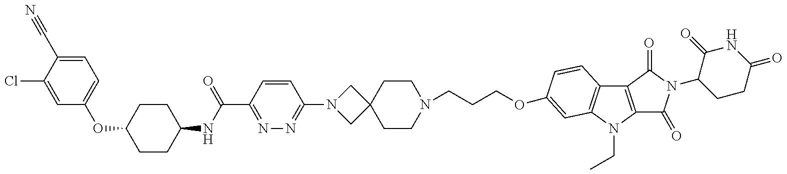 [SMILES: CCn1c2c(c3ccc(OCCCN4CCC5(CC4)CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 48 |
| 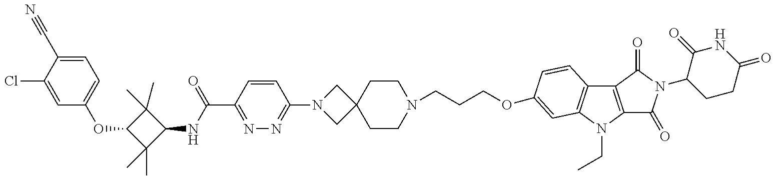 [SMILES: CCn1c2c(c3ccc(OCCCN4CCC5(CC4)CN(c4ccc(C(=O)N[C@H]6C(C)(C)[C@H](Oc7ccc(C#N)c(Cl)c7)C6(C)C)nn4)C5)cc31)C(=O)N(C1CCC(=O)NC1=O)C2=O] | Com- pound 49 |
| 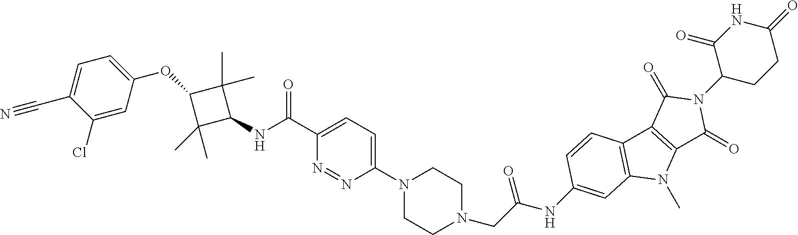 [SMILES: Cn1c2c(c3ccc(NC(=O)CN4CCN(c5ccc(C(=O)N[C@H]6C(C)(C)[C@H](Oc7ccc(C#N)c(Cl)c7)C6(C)C)nn5)CC4)cc31)C(=O)N(C1CCC(=O)NC1=O)C2=O] | Com- pound 50 |
| 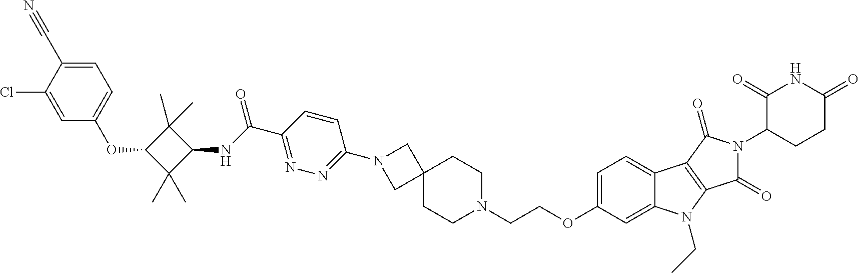 [SMILES: CCn1c2c(c3ccc(OCCN4CCC5(CC4)CN(c4ccc(C(=O)N[C@H]6C(C)(C)[C@H](Oc7ccc(C#N)c(Cl)c7)C6(C)C)nn4)C5)cc31)C(=O)N(C1CCC(=O)NC1=O)C2=O] | Com- pound 51 |
| 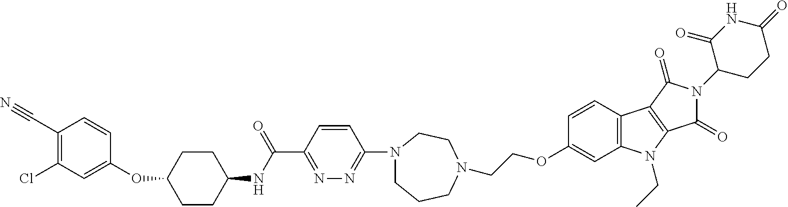 [SMILES: CCn1c2c(c3ccc(OCCN4CCCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 52 |
| 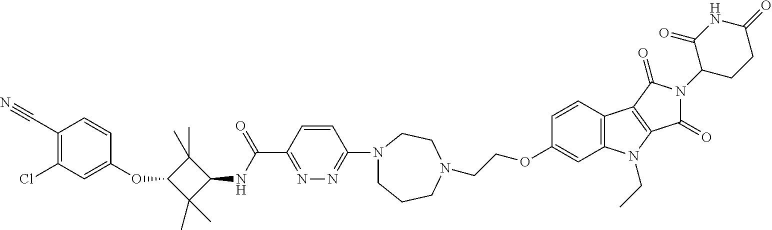 [SMILES: CCn1c2c(c3ccc(OCCN4CCCN(c5ccc(C(=O)N[C@H]6C(C)(C)[C@H](Oc7ccc(C#N)c(Cl)c7)C6(C)C)nn5)CC4)cc31)C(=O)N(C1CCC(=O)NC1=O)C2=O] | Com- pound 53 |
| 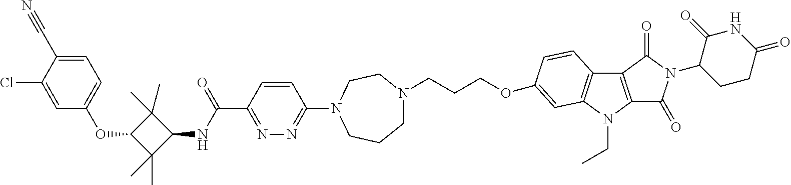 [SMILES: CCn1c2c(c3ccc(OCCCN4CCCN(c5ccc(C(=O)N[C@H]6C(C)(C)[C@H](Oc7ccc(C#N)c(Cl)c7)C6(C)C)nn5)CC4)cc31)C(=O)N(C1CCC(=O)NC1=O)C2=O] | Com- pound 54 |
| 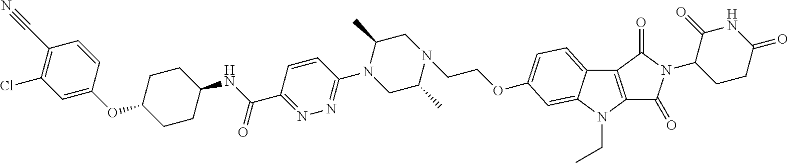 [SMILES: CCn1c2c(c3ccc(OCCN4C[C@H](C)N(c5ccc(C(=O)N[C@H]6CC[C@H](Oc7ccc(C#N)c(Cl)c7)CC6)nn5)C[C@H]4C)cc31)C(=O)N(C1CCC(=O)NC1=O)C2=O] | Com- pound 55 |
| 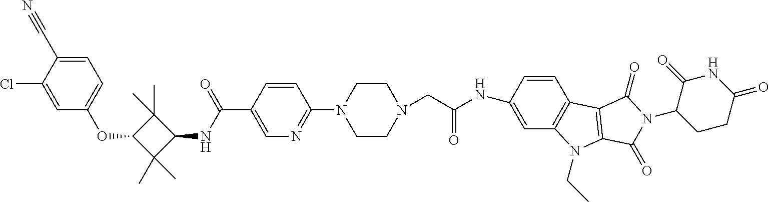 [SMILES: CCn1c2c(c3ccc(NC(=O)CN4CCN(c5ccc(C(=O)N[C@H]6C(C)(C)[C@H](Oc7ccc(C#N)c(Cl)c7)C6(C)C)cn5)CC4)cc31)C(=O)N(C1CCC(=O)NC1=O)C2=O] | Com- pound 56 |
| 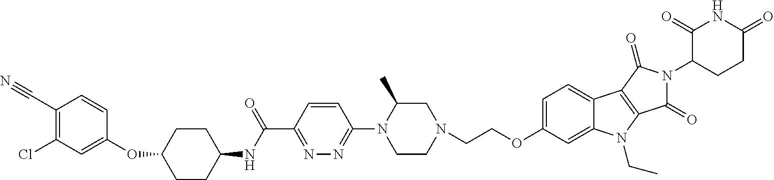 [SMILES: CCn1c2c(c3ccc(OCCN4CCN(c5ccc(C(=O)N[C@H]6CC[C@H](Oc7ccc(C#N)c(Cl)c7)CC6)nn5)[C@@H](C)C4)cc31)C(=O)N(C1CCC(=O)NC1=O)C2=O] | Com- pound 57 |
| 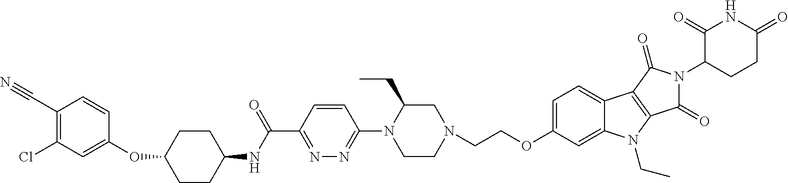 [SMILES: CC[C@H]1CN(CCOc2ccc3c4c(n(CC)c3c2)C(=O)N(C2CCC(=O)NC2=O)C4=O)CCN1c1ccc(C(=O)N[C@H]2CC[C@H](Oc3ccc(C#N)c(Cl)c3)CC2)nn1] | Com- pound 58 |
| 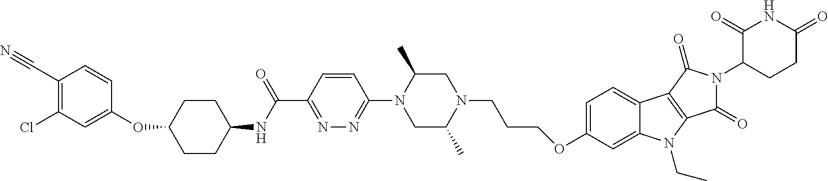 [SMILES: CCn1c2c(c3ccc(OCCCN4C[C@H](C)N(c5ccc(C(=O)N[C@H]6CC[C@H](Oc7ccc(C#N)c(Cl)c7)CC6)nn5)C[C@H]4C)cc31)C(=O)N(C1CCC(=O)NC1=O)C2=O] | Com- pound 59 |
| 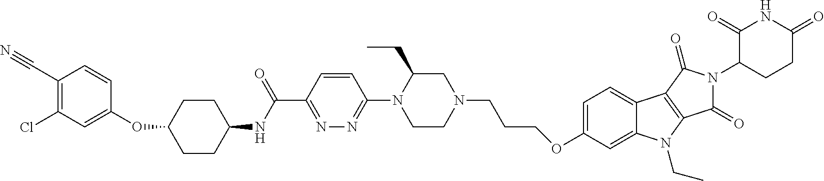 [SMILES: CC[C@H]1CN(CCCOc2ccc3c4c(n(CC)c3c2)C(=O)N(C2CCC(=O)NC2=O)C4=O)CCN1c1ccc(C(=O)N[C@H]2CC[C@H](Oc3ccc(C#N)c(Cl)c3)CC2)nn1] | Com- pound 60 |
| 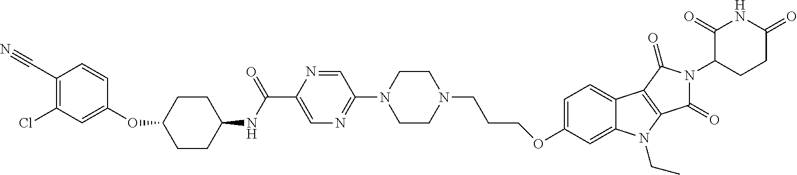 [SMILES: CCn1c2c(c3ccc(OCCCN4CCN(c5cnc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 61 |
| 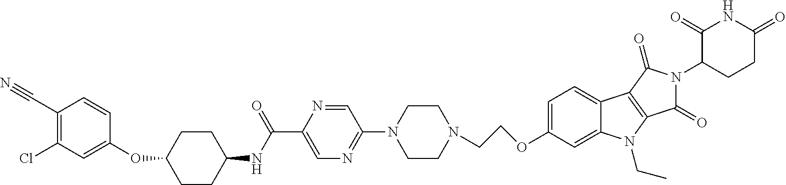 [SMILES: CCn1c2c(c3ccc(OCCN4CCN(c5cnc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 62 |
| 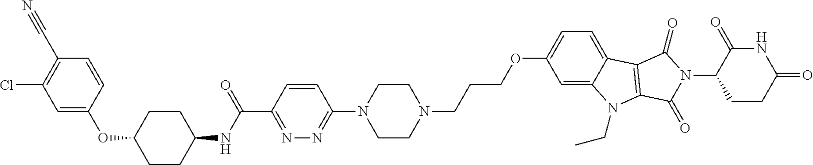 [SMILES: CCn1c2c(c3ccc(OCCCN4CCN(c5ccc(C(=O)N[C@H]6CC[C@H](Oc7ccc(C#N)c(Cl)c7)CC6)nn5)CC4)cc31)C(=O)N([C@H]1CCC(=O)NC1=O)C2=O] | Com- pound 63 |
| 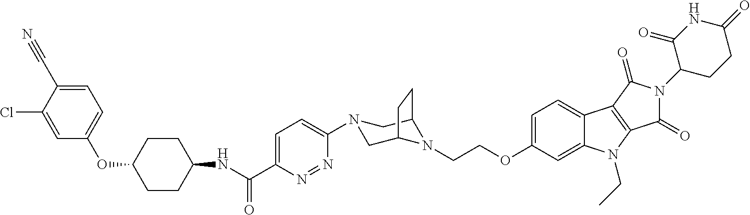 [SMILES: CCn1c2c(c3ccc(OCCN4C5CCC4CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 64 |
| 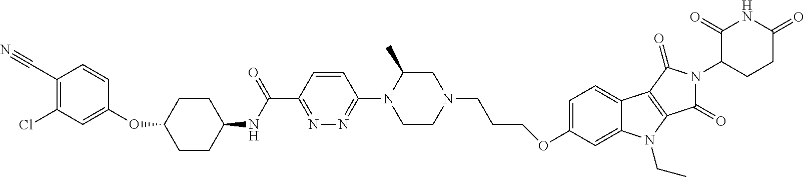 [SMILES: CCn1c2c(c3ccc(OCCCN4CCN(c5ccc(C(=O)N[C@H]6CC[C@H](Oc7ccc(C#N)c(Cl)c7)CC6)nn5)[C@@H](C)C4)cc31)C(=O)N(C1CCC(=O)NC1=O)C2=O] | Com- pound 65 |
| 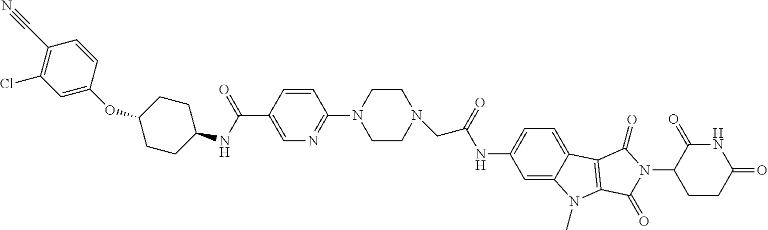 [SMILES: Cn1c2c(c3ccc(NC(=O)CN4CCN(c5ccc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 66 |
| 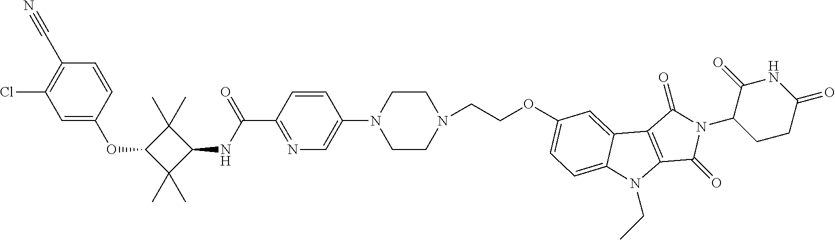 [SMILES: CCn1c2c(c3cc(OCCN4CCN(c5ccc(C(=O)N[C@H]6C(C)(C)[C@H](Oc7ccc(C#N)c(Cl)c7)C6(C)C)nc5)CC4)ccc31)C(=O)N(C1CCC(=O)NC1=O)C2=O] | Com- pound 67 |
| 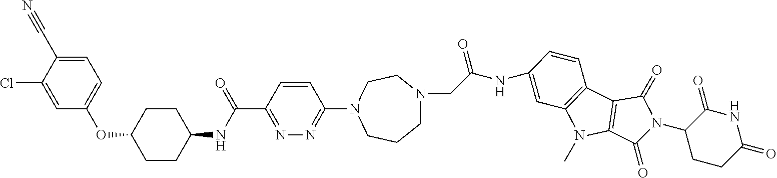 [SMILES: Cn1c2c(c3ccc(NC(=O)CN4CCCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 68 |
| 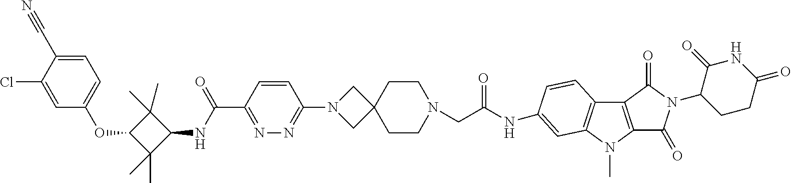 [SMILES: Cn1c2c(c3ccc(NC(=O)CN4CCC5(CC4)CN(c4ccc(C(=O)N[C@H]6C(C)(C)[C@H](Oc7ccc(C#N)c(Cl)c7)C6(C)C)nn4)C5)cc31)C(=O)N(C1CCC(=O)NC1=O)C2=O] | Com- pound 69 |
| 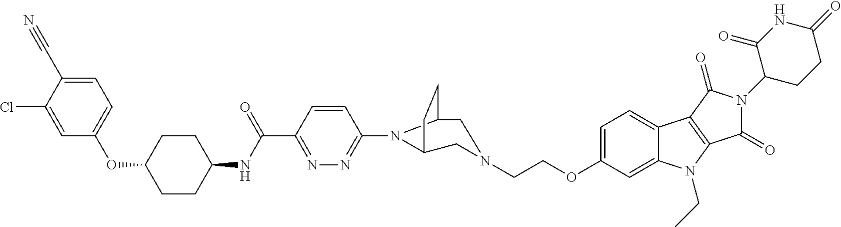 [SMILES: CCn1c2c(c3ccc(OCCN4CC5CCC(C4)N5c4ccc(C(=O)N[C@H]5CC[C@H](Oc6ccc(C#N)c(Cl)c6)CC5)nn4)cc31)C(=O)N(C1CCC(=O)NC1=O)C2=O] | Com- pound 70 |
| 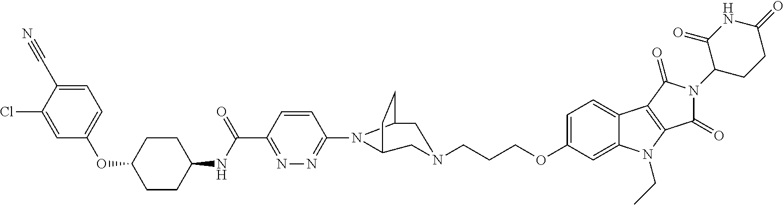 [SMILES: CCn1c2c(c3ccc(OCCCN4CC5CCC(C4)N5c4ccc(C(=O)N[C@H]5CC[C@H](Oc6ccc(C#N)c(Cl)c6)CC5)nn4)cc31)C(=O)N(C1CCC(=O)NC1=O)C2=O] | Com- pound 71 |
| 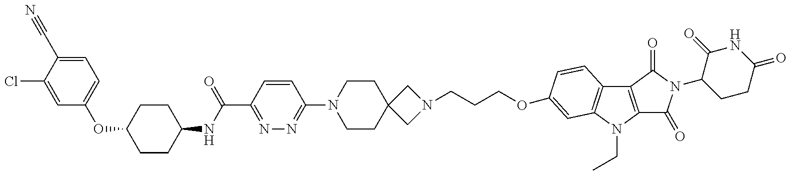 [SMILES: CCn1c2c(c3ccc(OCCCN4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)NC1=O)C2=O] | Com- pound 72 |
| 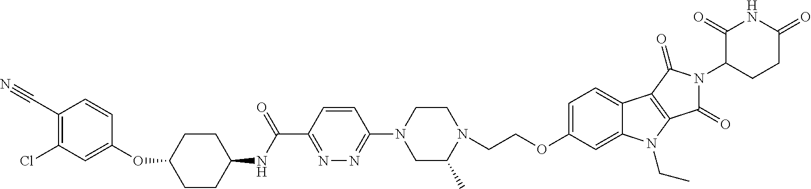 [SMILES: CCn1c2c(c3ccc(OCCN4CCN(c5ccc(C(=O)N[C@H]6CC[C@H](Oc7ccc(C#N)c(Cl)c7)CC6)nn5)C[C@H]4C)cc31)C(=O)N(C1CCC(=O)NC1=O)C2=O] | Com- pound 73 |
| 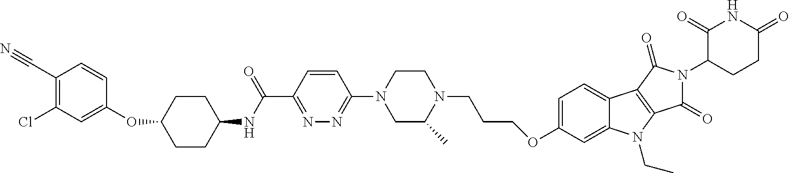 [SMILES: CCn1c2c(c3ccc(OCCCN4CCN(c5ccc(C(=O)N[C@H]6CC[C@H](Oc7ccc(C#N)c(Cl)c7)CC6)nn5)C[C@H]4C)cc31)C(=O)N(C1CCC(=O)NC1=O)C2=O] | Com- pound 74 |
| 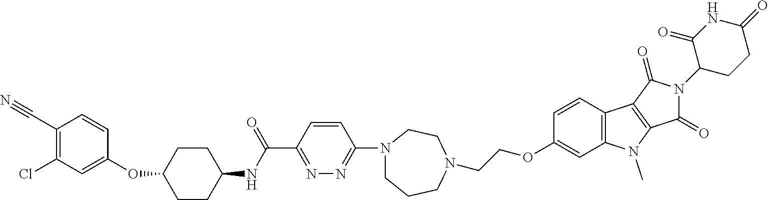 [SMILES: Cn1c2c(c3ccc(OCCN4CCCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 75 |
| 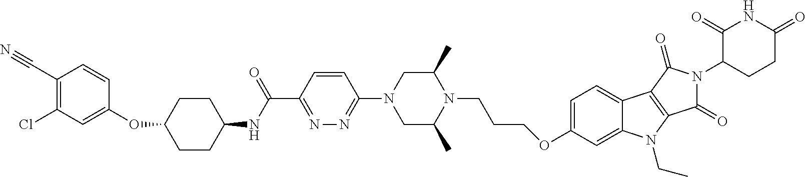 [SMILES: CCn1c2c(c3ccc(OCCCN4[C@H](C)CN(c5ccc(C(=O)N[C@H]6CC[C@H](Oc7ccc(C#N)c(Cl)c7)CC6)nn5)C[C@@H]4C)cc31)C(=O)N(C1CCC(=O)NC1=O)C2=O] | Com- pound 76 |
| 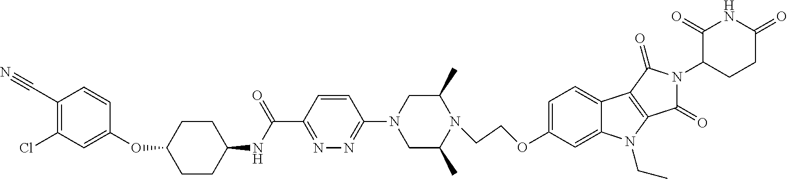 [SMILES: CCn1c2c(c3ccc(OCCN4[C@H](C)CN(c5ccc(C(=O)N[C@H]6CC[C@H](Oc7ccc(C#N)c(Cl)c7)CC6)nn5)C[C@@H]4C)cc31)C(=O)N(C1CCC(=O)NC1=O)C2=O] | Com- pound 77 |
| 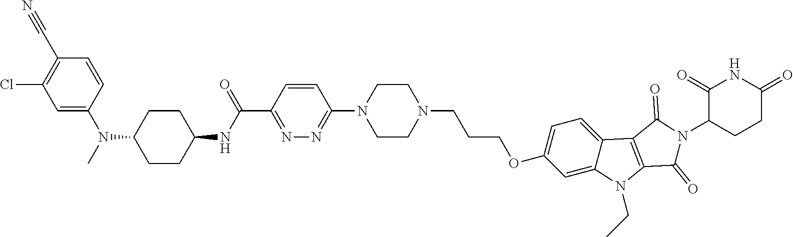 [SMILES: CCn1c2c(c3ccc(OCCCN4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 78 |
| 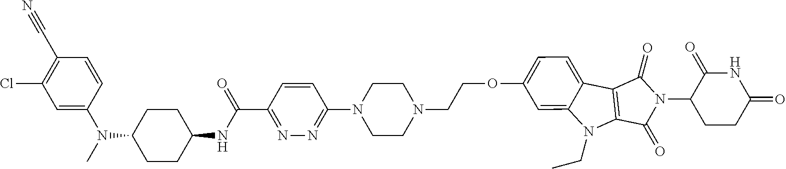 [SMILES: CCn1c2c(c3ccc(OCCN4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 79 |
| 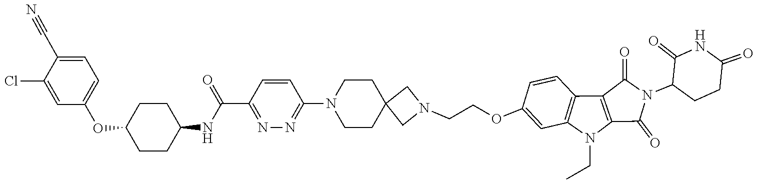 [SMILES: CCn1c2c(c3ccc(OCCN4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)NC1=O)C2=O] | Com- pound 80 |
| 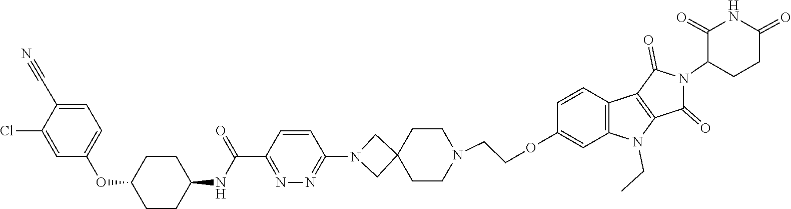 [SMILES: CCn1c2c(c3ccc(OCCN4CCC5(CC4)CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 81 |
| 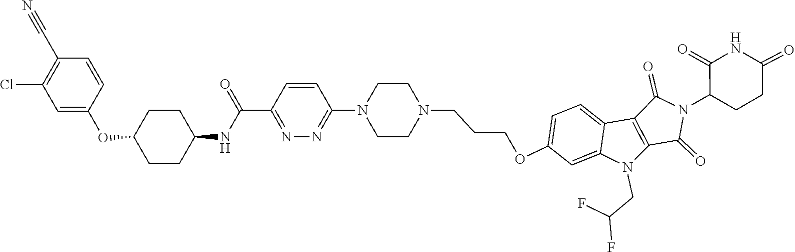 [SMILES: N#Cc1ccc(O[C@H]2CC[C@H](NC(=O)c3ccc(N4CCN(CCCOc5ccc6c7c(n(CC(F)F)c6c5)C(=O)N(C5CCC(=O)NC5=O)C7=O)CC4)nn3)CC2)cc1Cl] | Com- pound 82 |
| 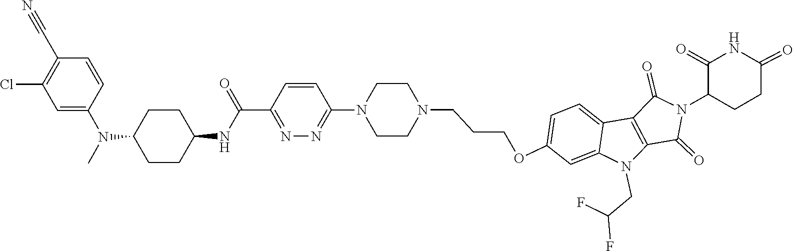 [SMILES: CN(c1ccc(C#N)c(Cl)c1)[C@H]1CC[C@H](NC(=O)c2ccc(N3CCN(CCCOc4ccc5c6c(n(CC(F)F)c5c4)C(=O)N(C4CCC(=O)NC4=O)C6=O)CC3)nn2)CC1] | Com- pound 83 |
| 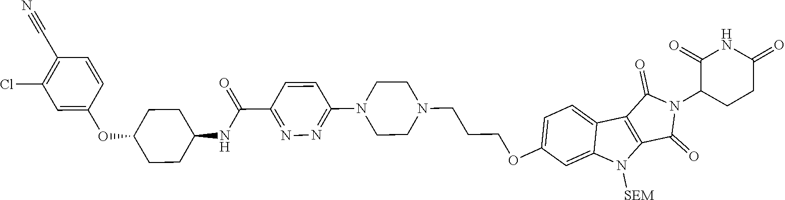 [SMILES: C[Si](C)(C)CCOCn1c2c(c3ccc(OCCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 84 |
| 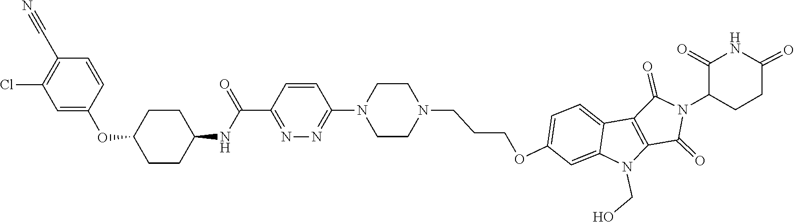 [SMILES: N#Cc1ccc(O[C@H]2CC[C@H](NC(=O)c3ccc(N4CCN(CCCOc5ccc6c7c(n(CO)c6c5)C(=O)N(C5CCC(=O)NC5=O)C7=O)CC4)nn3)CC2)cc1Cl] | Com- pound 85 |
| 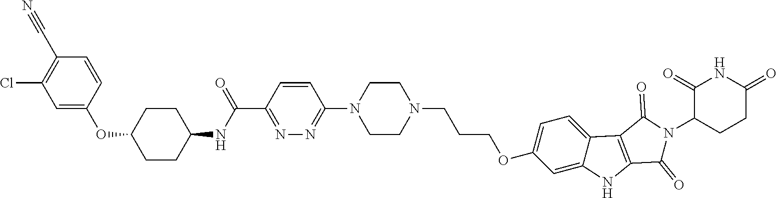 [SMILES: N#Cc1ccc(O[C@H]2CC[C@H](NC(=O)c3ccc(N4CCN(CCCOc5ccc6c7c([nH]c6c5)C(=O)N(C5CCC(=O)NC5=O)C7=O)CC4)nn3)CC2)cc1Cl] | Com- pound 86 |
| 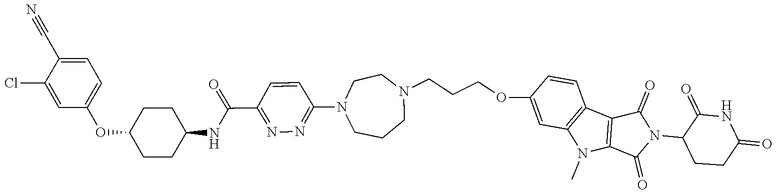 [SMILES: Cn1c2c(c3ccc(OCCCN4CCCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 87 |
| 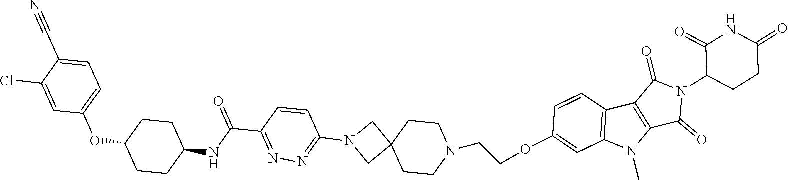 [SMILES: Cn1c2c(c3ccc(OCCN4CCC5(CC4)CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 88 |
| 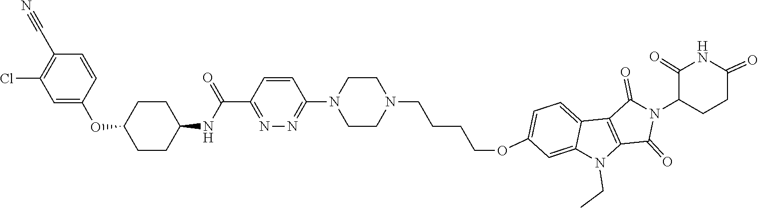 [SMILES: CCn1c2c(c3ccc(OCCCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 89 |
| 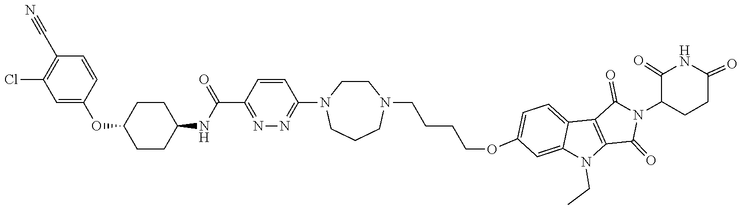 [SMILES: CCn1c2c(c3ccc(OCCCCN4CCCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 90 |
| 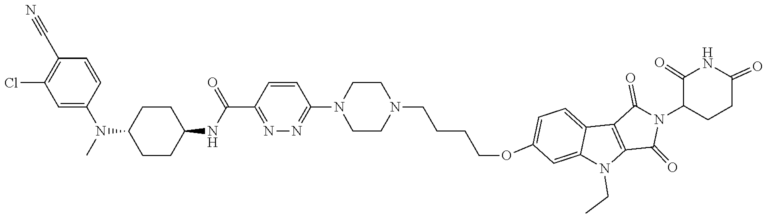 [SMILES: CCn1c2c(c3ccc(OCCCCN4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 91 |
| 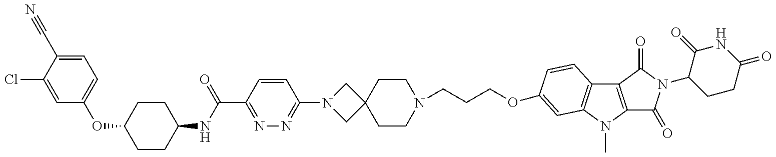 [SMILES: Cn1c2c(c3ccc(OCCCN4CCC5(CC4)CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 92 |
| 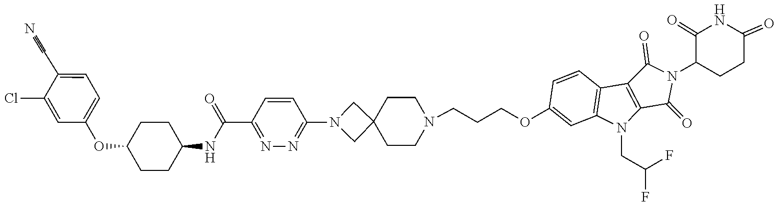 [SMILES: C=Nc1ccc(O[C@H]2CC[C@H](NC(=O)c3ccc(N4CC5(CCN(CCCOc6ccc7c8c(n(CC(F)F)c7c6)C(=O)N(C6CCC(=O)NC6=O)C8=O)CC5)C4)nn3)CC2)cc1Cl] | Com- pound 93 |
| 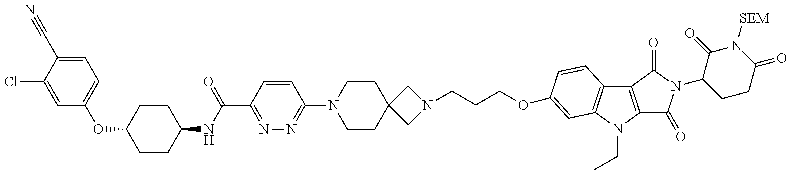 [SMILES: CCn1c2c(c3ccc(OCCCN4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)N(COCC[Si](C)(C)C)C1=O)C2=O] | Com- pound 94 |
| 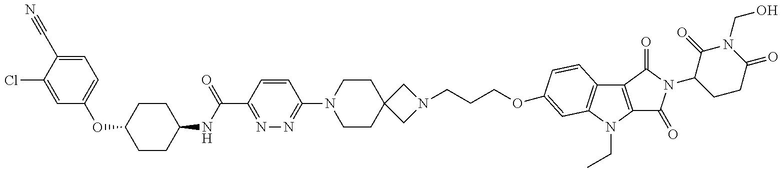 [SMILES: CCn1c2c(c3ccc(OCCCN4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)N(CO)C1=O)C2=O] | Com- pound 95 |
| 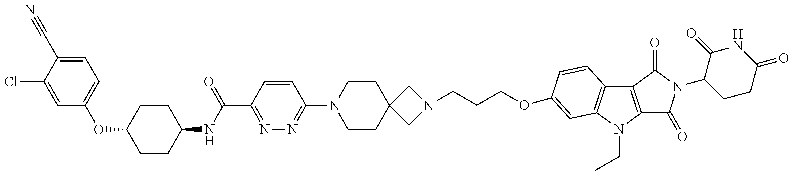 [SMILES: CCn1c2c(c3ccc(OCCCN4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)NC1=O)C2=O] | Com- pound 96 |
| 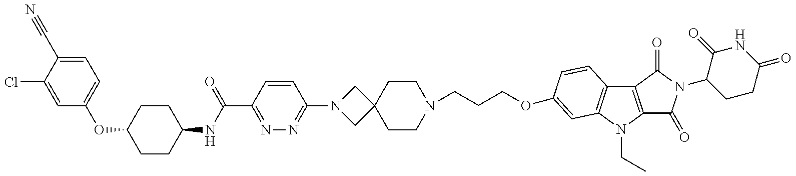 [SMILES: CCn1c2c(c3ccc(OCCCN4CCC5(CC4)CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 97 |
| 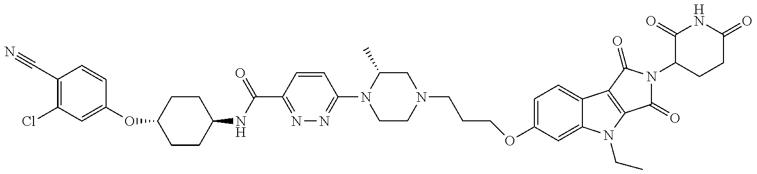 [SMILES: CCn1c2c(c3ccc(OCCCN4CCN(c5ccc(C(=O)N[C@H]6CC[C@H](Oc7ccc(C#N)c(Cl)c7)CC6)nn5)[C@H](C)C4)cc31)C(=O)N(C1CCC(=O)NC1=O)C2=O] | Com- pound 98 |
| 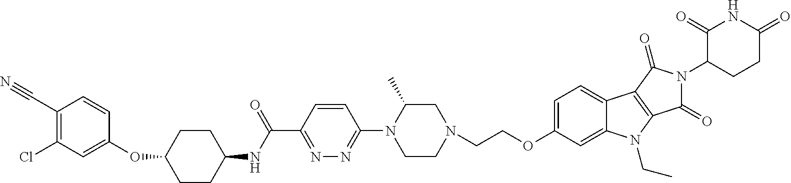 [SMILES: CCn1c2c(c3ccc(OCCN4CCN(c5ccc(C(=O)N[C@H]6CC[C@H](Oc7ccc(C#N)c(Cl)c7)CC6)nn5)[C@H](C)C4)cc31)C(=O)N(C1CCC(=O)NC1=O)C2=O] | Com- pound 99 |
| 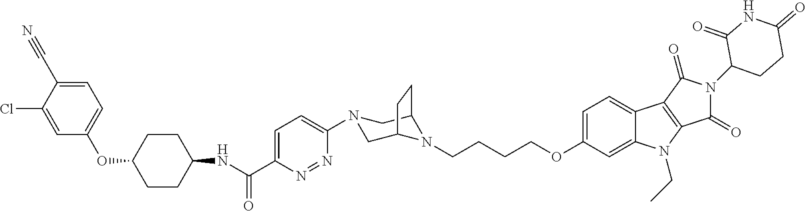 [SMILES: CCn1c2c(c3ccc(OCCCCN4C5CCC4CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 100 |
| 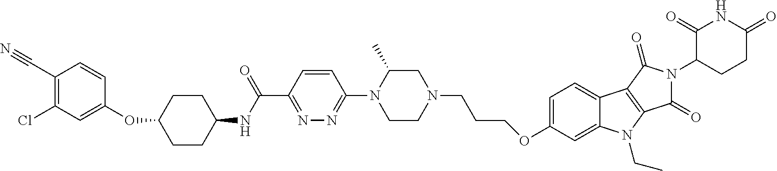 [SMILES: CCn1c2c(c3ccc(OCCCN4CCN(c5ccc(C(=O)N[C@H]6CC[C@H](Oc7ccc(C#N)c(Cl)c7)CC6)nn5)[C@H](C)C4)cc31)C(=O)N(C1CCC(=O)NC1=O)C2=O] | Com- pound 101 |
| 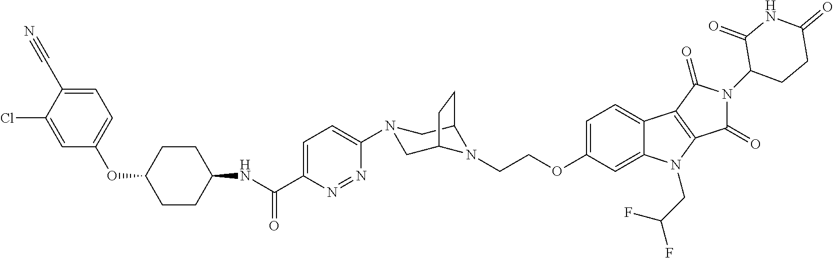 [SMILES: N#Cc1ccc(O[C@H]2CC[C@H](NC(=O)c3ccc(N4CC5CCC(C4)N5CCOc4ccc5c6c(n(CC(F)F)c5c4)C(=O)N(C4CCC(=O)NC4=O)C6=O)nn3)CC2)cc1Cl] | Com- pound 102 |
| 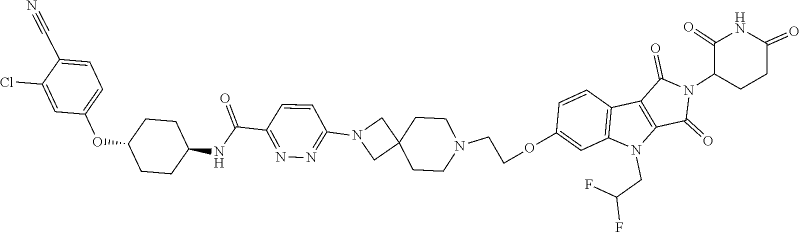 [SMILES: N#Cc1ccc(O[C@H]2CC[C@H](NC(=O)c3ccc(N4CC5(CCN(CCOc6ccc7c8c(n(CC(F)F)c7c6)C(=O)N(C6CCC(=O)NC6=O)C8=O)CC5)C4)nn3)CC2)cc1Cl] | Com- pound 103 |
| 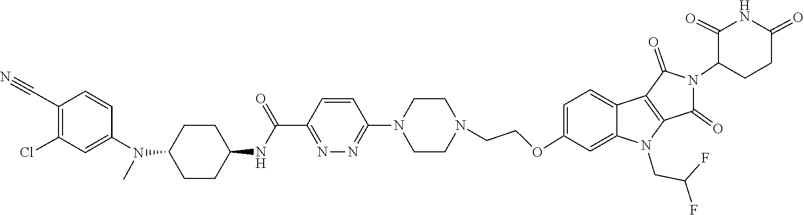 [SMILES: CN(c1ccc(C#N)c(Cl)c1)[C@H]1CC[C@H](NC(=O)c2ccc(N3CCN(CCOc4ccc5c6c(n(CC(F)F)c5c4)C(=O)N(C4CCC(=O)NC4=O)C6=O)CC3)nn2)CC1] | Com- pound 104 |
| 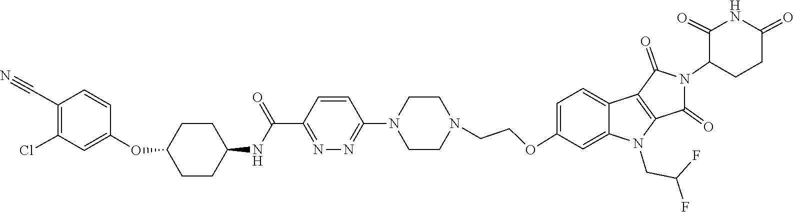 [SMILES: N#Cc1ccc(O[C@H]2CC[C@H](NC(=O)c3ccc(N4CCN(CCOc5ccc6c7c(n(CC(F)F)c6c5)C(=O)N(C5CCC(=O)NC5=O)C7=O)CC4)nn3)CC2)cc1Cl] | Com- pound 105 |
| 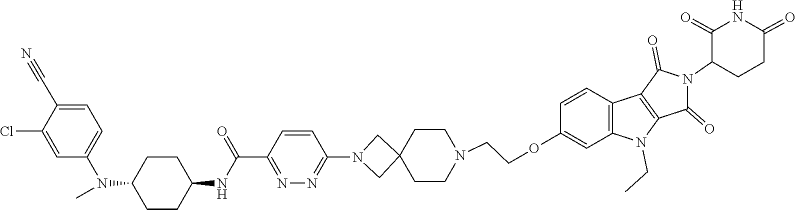 [SMILES: CCn1c2c(c3ccc(OCCN4CCC5(CC4)CN(c4ccc(C(=O)N[C@H]6CC[C@H](N(C)c7ccc(C#N)c(Cl)c7)CC6)nn4)C5)cc31)C(=O)N(C1CCC(=O)NC1=O)C2=O] | Com- pound 106 |
| 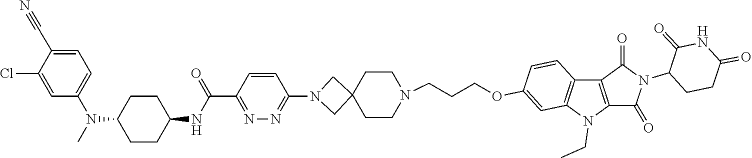 [SMILES: CCn1c2c(c3ccc(OCCCN4CCC5(CC4)CN(c4ccc(C(=O)N[C@H]6CC[C@H](N(C)c7ccc(C#N)c(Cl)c7)CC6)nn4)C5)cc31)C(=O)N(C1CCC(=O)NC1=O)C2=O] | Com- pound 107 |
| 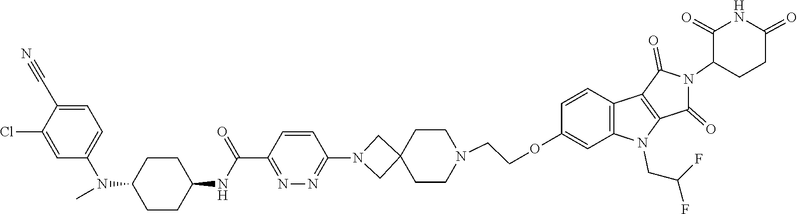 [SMILES: CN(c1ccc(C#N)c(Cl)c1)[C@H]1CC[C@H](NC(=O)c2ccc(N3CC4(CCN(CCOc5ccc6c7c(n(CC(F)F)c6c5)C(=O)N(C5CCC(=O)NC5=O)C7=O)CC4)C3)nn2)CC1] | Com- pound 108 |
| 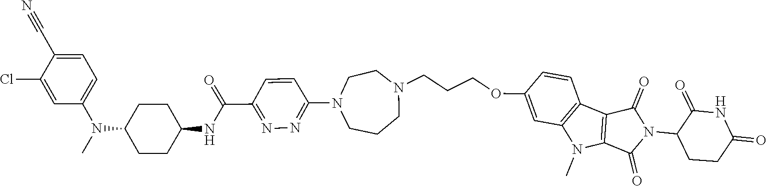 [SMILES: Cn1c2c(c3ccc(OCCCN4CCCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 109 |
| 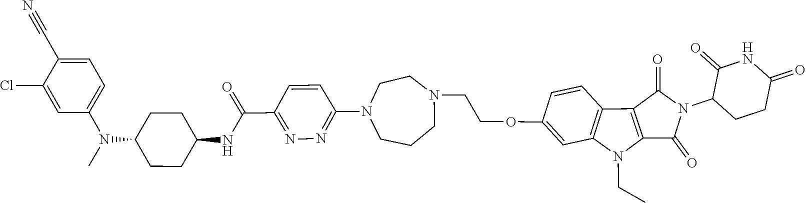 [SMILES: CCn1c2c(c3ccc(OCCN4CCCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 110 |
| 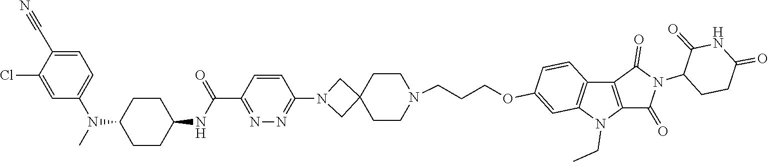 [SMILES: CCn1c2c(c3ccc(OCCCN4CCC5(CC4)CN(c4ccc(C(=O)N[C@H]6CC[C@H](N(C)c7ccc(C#N)c(Cl)c7)CC6)nn4)C5)cc31)C(=O)N(C1CCC(=O)NC1=O)C2=O] | Com- pound 111 |
| 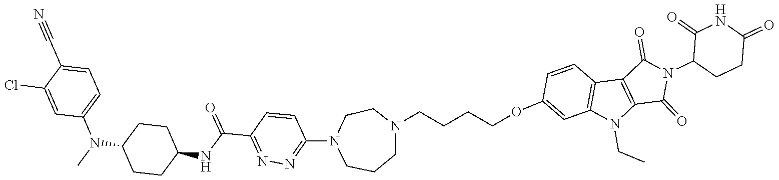 [SMILES: CCn1c2c(c3ccc(OCCCCN4CCCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 112 |
| 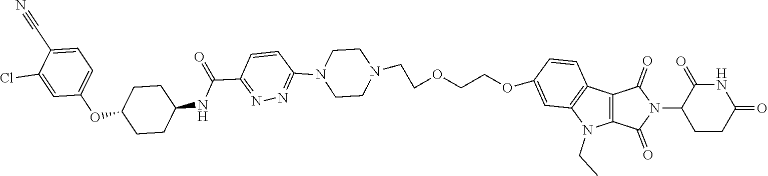 [SMILES: CCn1c2c(c3ccc(OCCOCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 113 |
| 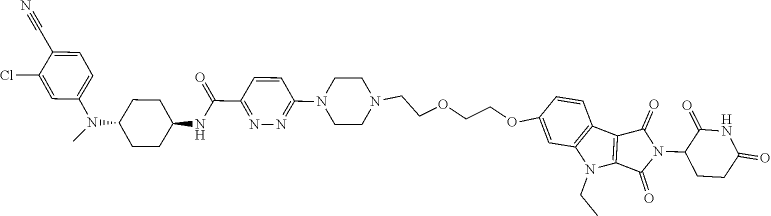 [SMILES: CCn1c2c(c3ccc(OCCOCCN4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 114 |
| 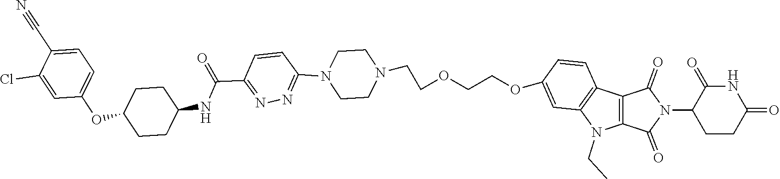 [SMILES: CCn1c2c(c3ccc(OCCOCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 115 |
| 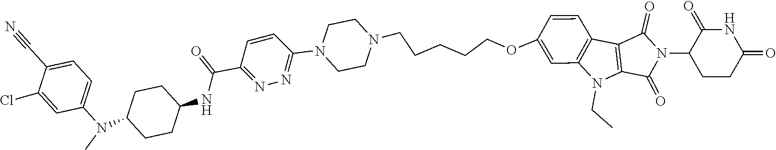 [SMILES: CCn1c2c(c3ccc(OCCCCCN4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 116 |
| 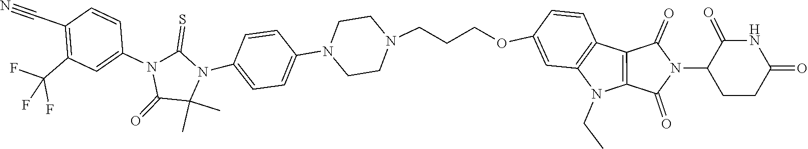 [SMILES: CCn1c2c(c3ccc(OCCCN4CCN(c5ccc(N6C(=S)N(c7ccc(C#N)c(C(F)(F)F)c7)C(=O)C6(C)C)cc5)CC4)cc31)C(=O)N(C1CCC(=O)NC1=O)C2=O] | Com- pound 117 |
| 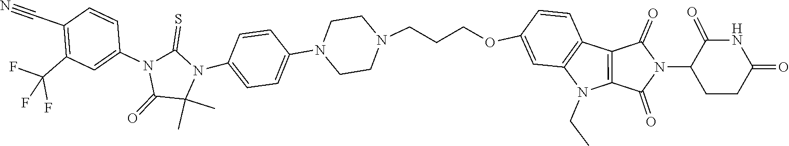 [SMILES: CCn1c2c(c3ccc(OCCCN4CCN(c5ccc(N6C(=S)N(c7ccc(C#N)c(C(F)(F)F)c7)C(=O)C6(C)C)cc5)CC4)cc31)C(=O)N(C1CCC(=O)NC1=O)C2=O] | Com- pound 118 |
| 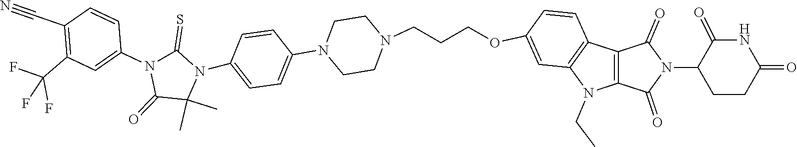 [SMILES: CCn1c2c(c3ccc(OCCCN4CCN(c5ccc(N6C(=S)N(c7ccc(C#N)c(C(F)(F)F)c7)C(=O)C6(C)C)cc5)CC4)cc31)C(=O)N(C1CCC(=O)NC1=O)C2=O] | Com- pound 119 |
| 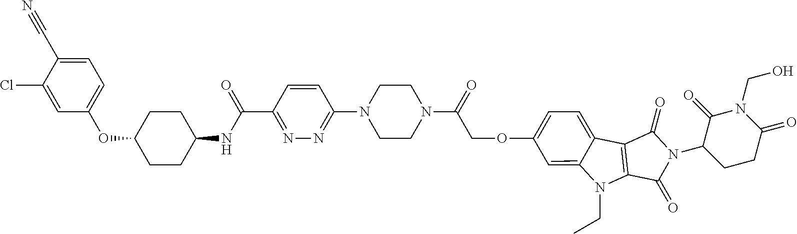 [SMILES: CCn1c2c(c3ccc(OCC(=O)N4CCN(c5ccc(C(=O)N[C@H]6CC[C@H](Oc7ccc(C#N)c(Cl)c7)CC6)nn5)CC4)cc31)C(=O)N(C1CCC(=O)N(CO)C1=O)C2=O] | Com- pound 120 |
| 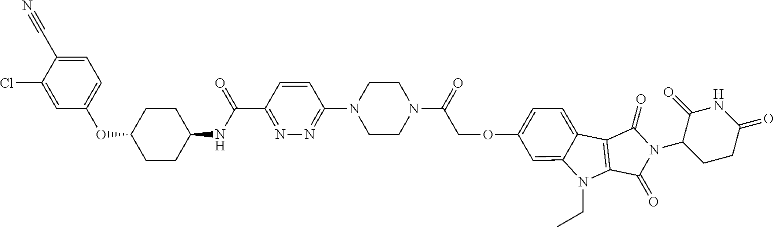 [SMILES: CCn1c2c(c3ccc(OCC(=O)N4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 121 |
| 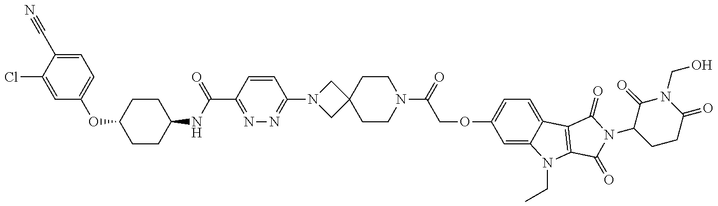 [SMILES: CCn1c2c(c3ccc(OCC(=O)N4CCC5(CC4)CN(c4ccc(C(=O)N[C@H]6CC[C@H](Oc7ccc(C#N)c(Cl)c7)CC6)nn4)C5)cc31)C(=O)N(C1CCC(=O)N(CO)C1=O)C2=O] | Com- pound 122 |
| 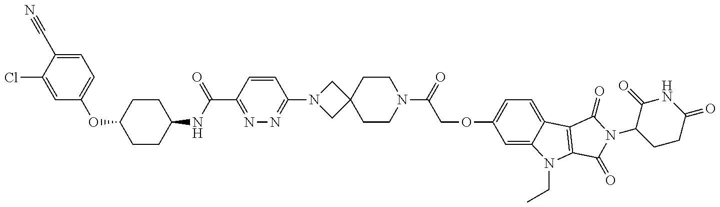 [SMILES: CCn1c2c(c3ccc(OCC(=O)N4CCC5(CC4)CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 123 |
| 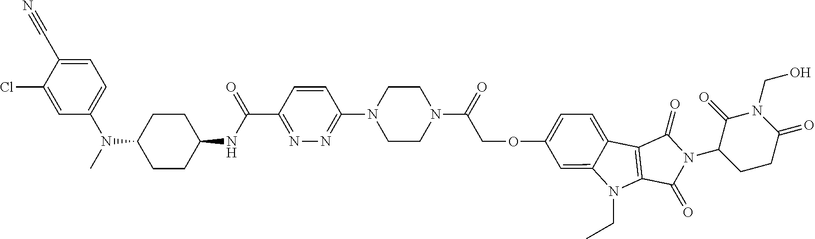 [SMILES: CCn1c2c(c3ccc(OCC(=O)N4CCN(c5ccc(C(=O)N[C@H]6CC[C@H](N(C)c7ccc(C#N)c(Cl)c7)CC6)nn5)CC4)cc31)C(=O)N(C1CCC(=O)N(CO)C1=O)C2=O] | Com- pound 124 |
| 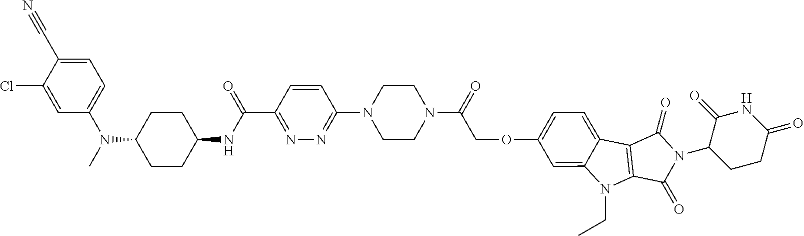 [SMILES: CCn1c2c(c3ccc(OCC(=O)N4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 125 |
| 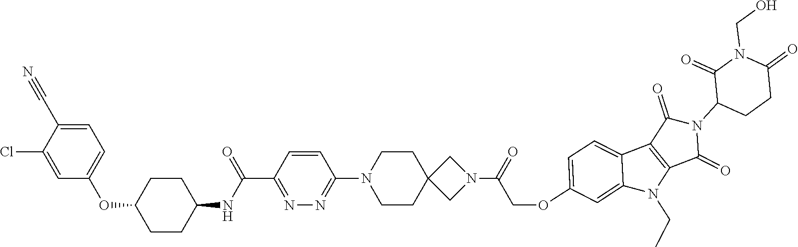 [SMILES: CCn1c2c(c3ccc(OCC(=O)N4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)N(CO)C1=O)C2=O] | Com- pound 126 |
| 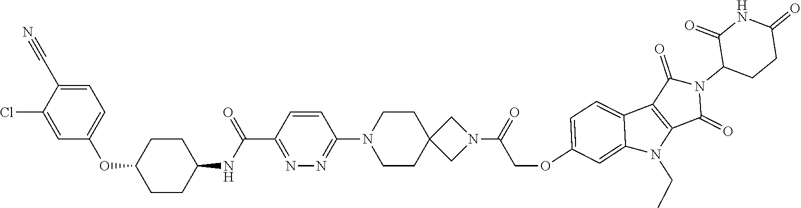 [SMILES: CCn1c2c(c3ccc(OCC(=O)N4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)NC1=O)C2=O] | Com- pound 127 |
| 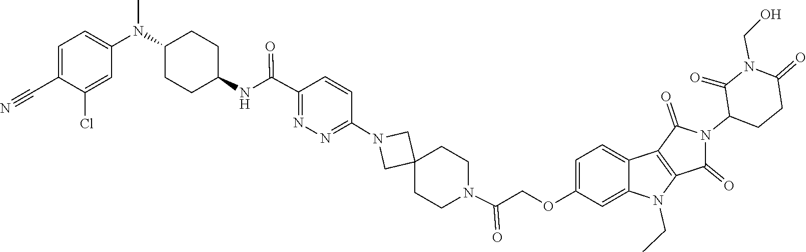 [SMILES: CCn1c2c(c3ccc(OCC(=O)N4CCC5(CC4)CN(c4ccc(C(=O)N[C@H]6CC[C@H](N(C)c7ccc(C#N)c(Cl)c7)CC6)nn4)C5)cc31)C(=O)N(C1CCC(=O)N(CO)C1=O)C2=O] | Com- pound 128 |
| 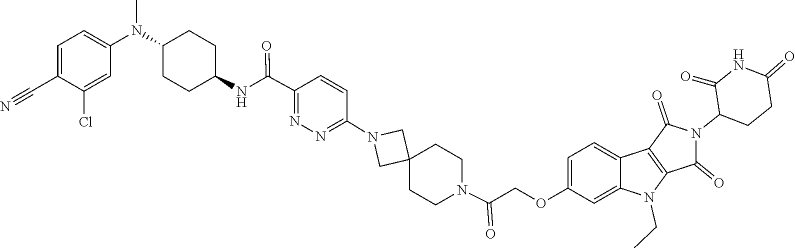 [SMILES: CCn1c2c(c3ccc(OCC(=O)N4CCC5(CC4)CN(c4ccc(C(=O)N[C@H]6CC[C@H](N(C)c7ccc(C#N)c(Cl)c7)CC6)nn4)C5)cc31)C(=O)N(C1CCC(=O)NC1=O)C2=O] | Com- pound 129 |
| 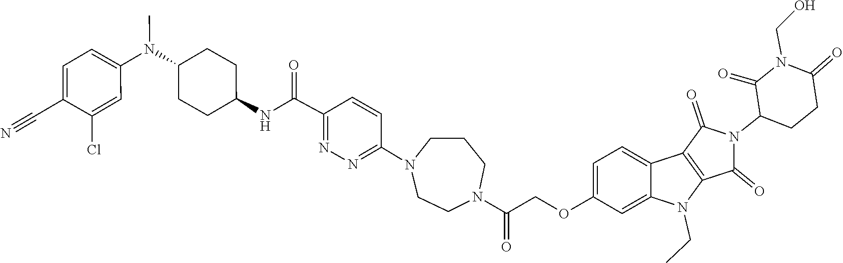 [SMILES: CCn1c2c(c3ccc(OCC(=O)N4CCCN(c5ccc(C(=O)N[C@H]6CC[C@H](N(C)c7ccc(C#N)c(Cl)c7)CC6)nn5)CC4)cc31)C(=O)N(C1CCC(=O)N(CO)C1=O)C2=O] | Com- pound 130 |
| 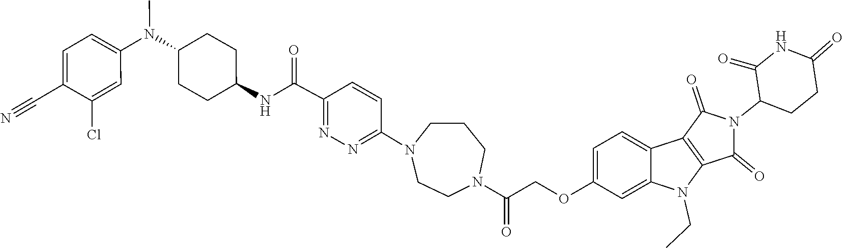 [SMILES: CCn1c2c(c3ccc(OCC(=O)N4CCCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 131 |
| 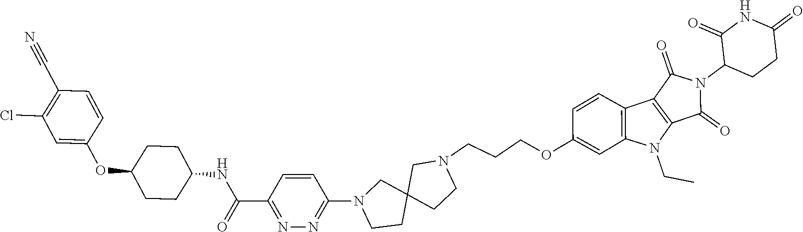 [SMILES: CCn1c2c(c3ccc(OCCCN4CCC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O] | Com- pound 132 |
| 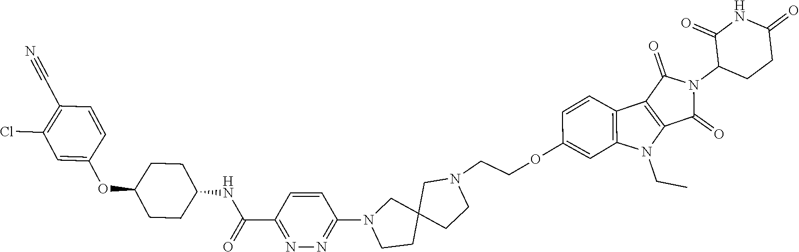 [SMILES: CCn1c2c(c3ccc(OCCN4CCC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O] | Com- pound 133 |
| 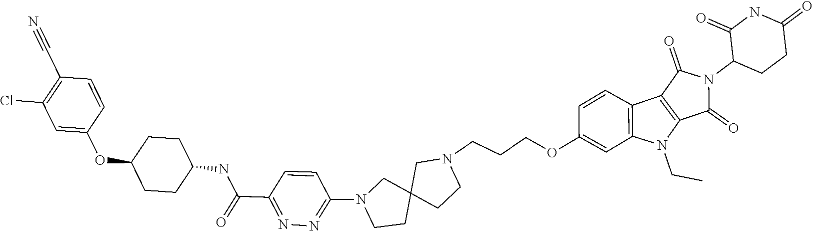 [SMILES: CCn1c2c(c3ccc(OCCCN4CCC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O] | Com- pound 134 |
| 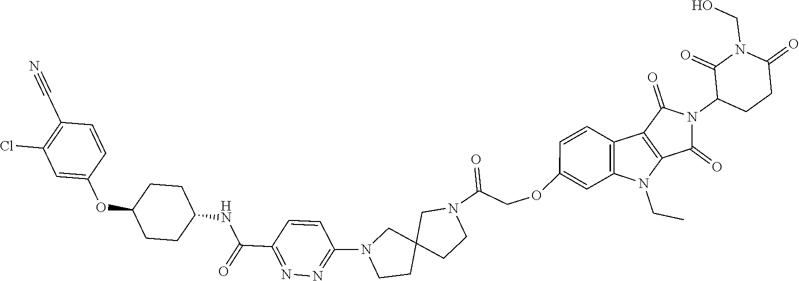 [SMILES: CCn1c2c(c3ccc(OCC(=O)N4CCC5(CCN(c6ccc(C(=O)N[C@H]7CC[C@H](Oc8ccc(C#N)c(Cl)c8)CC7)nn6)C5)C4)cc31)C(=O)N(C1CCC(=O)N(CO)C1=O)C2=O] | Com- pound 135 |
| 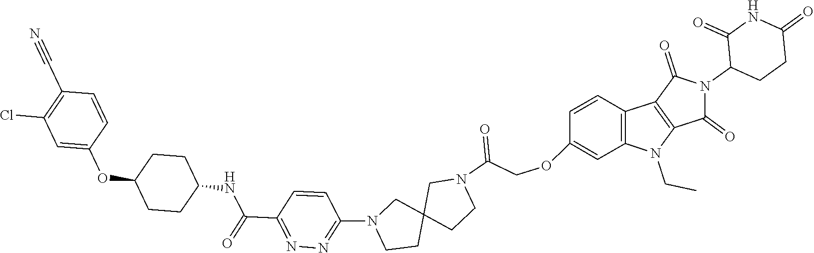 [SMILES: CCn1c2c(c3ccc(OCC(=O)N4CCC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O] | Com- pound 136 |
| 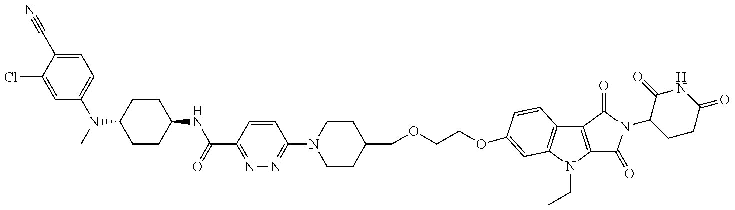 [SMILES: CCn1c2c(c3ccc(OCCOCC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 137 |
| 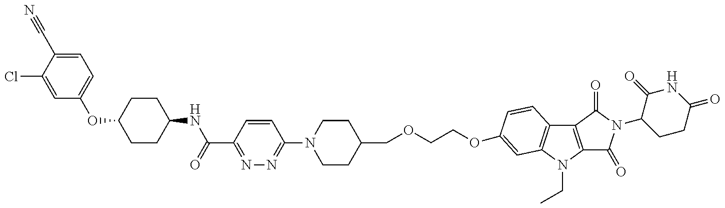 [SMILES: CCn1c2c(c3ccc(OCCOCC4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 138 |
| 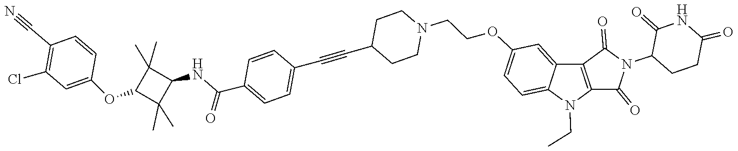 | Com- pound 139 |
| 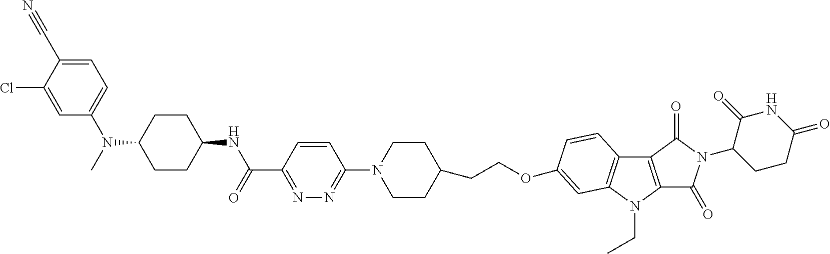 [SMILES: CCn1c2c(c3ccc(OCCC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 140 |
| 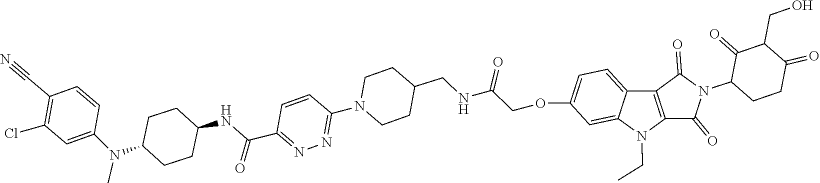 [SMILES: CCn1c2c(c3ccc(OCC(=O)NCC4CCN(c5ccc(C(=O)N[C@H]6CC[C@H](N(C)c7ccc(C#N)c(Cl)c7)CC6)nn5)CC4)cc31)C(=O)N(C1CCC(=O)C(CO)C1=O)C2=O] | Com- pound 141 |
| 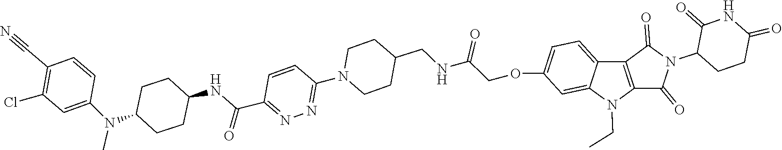 [SMILES: CCn1c2c(c3ccc(OCC(=O)NCC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 142 |
| 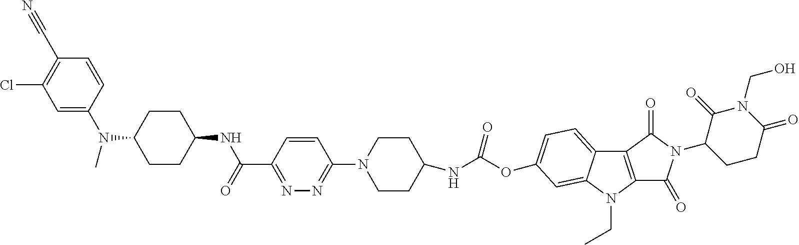 [SMILES: CCn1c2c(c3ccc(OC(=O)NC4CCN(c5ccc(C(=O)N[C@H]6CC[C@H](N(C)c7ccc(C#N)c(Cl)c7)CC6)nn5)CC4)cc31)C(=O)N(C1CCC(=O)N(CO)C1=O)C2=O] | Com- pound 143 |
| 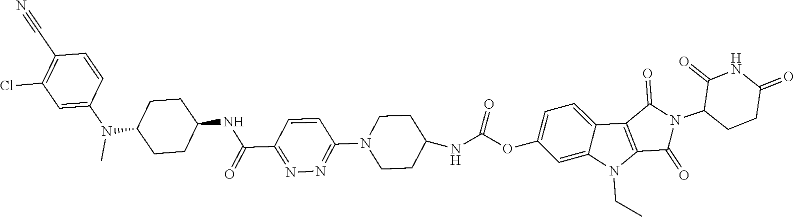 [SMILES: CCn1c2c(c3ccc(OC(=O)NC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 144 |
| 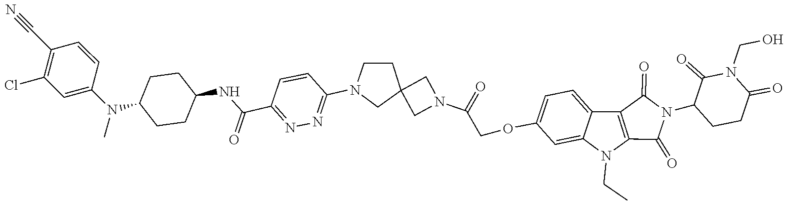 [SMILES: CCn1c2c(c3ccc(OCC(=O)N4CC5(CCN(c6ccc(C(=O)N[C@H]7CC[C@H](N(C)c8ccc(C#N)c(Cl)c8)CC7)nn6)C5)C4)cc31)C(=O)N(C1CCC(=O)N(CO)C1=O)C2=O] | Com- pound 145 |
| 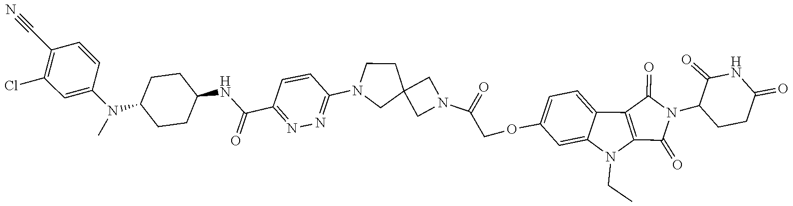 [SMILES: CCn1c2c(c3ccc(OCC(=O)N4CC5(CCN(c6ccc(C(=O)N[C@H]7CC[C@H](N(C)c8ccc(C#N)c(Cl)c8)CC7)nn6)C5)C4)cc31)C(=O)N(C1CCC(=O)NC1=O)C2=O] | Com- pound 146 |
| 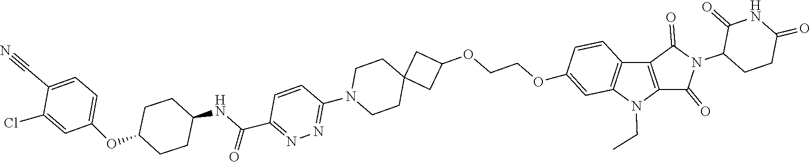 [SMILES: CCn1c2c(c3ccc(OCCOC4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)NC1=O)C2=O] | Com- pound 147 |
| 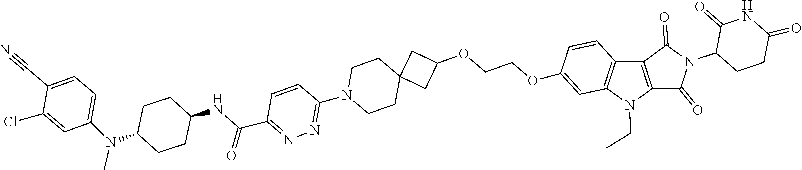 [SMILES: CCn1c2c(c3ccc(OCCOC4CC5(CCN(c6ccc(C(=O)N[C@H]7CC[C@H](N(C)c8ccc(C#N)c(Cl)c8)CC7)nn6)CC5)C4)cc31)C(=O)N(C1CCC(=O)NC1=O)C2=O] | Com- pound 148 |
| 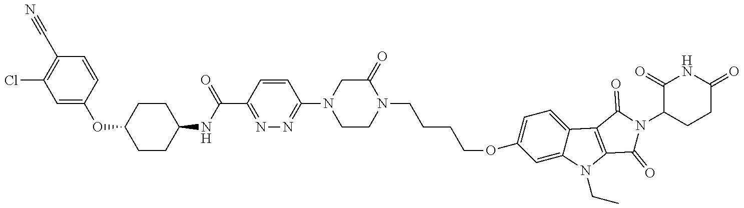 [SMILES: CCn1c2c(c3ccc(OCCCCN4CCN(c5ccc(C(=O)N[C@H]6CC[C@H](Oc7ccc(C#N)c(Cl)c7)CC6)nn5)CC4=O)cc31)C(=O)N(C1CCC(=O)NC1=O)C2=O] | Com- pound 149 |
| 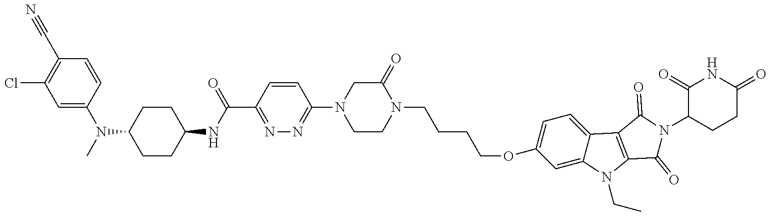 [SMILES: CCn1c2c(c3ccc(OCCCCN4CCN(c5ccc(C(=O)N[C@H]6CC[C@H](N(C)c7ccc(C#N)c(Cl)c7)CC6)nn5)CC4=O)cc31)C(=O)N(C1CCC(=O)NC1=O)C2=O] | Com- pound 150 |
| 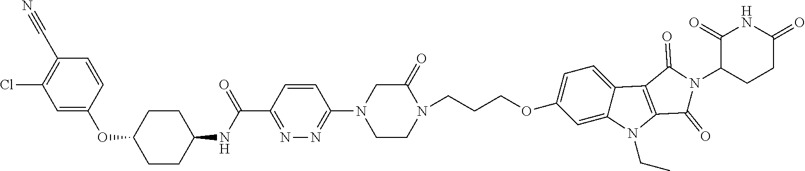 [SMILES: CCn1c2c(c3ccc(OCCCN4CCN(c5ccc(C(=O)N[C@H]6CC[C@H](Oc7ccc(C#N)c(Cl)c7)CC6)nn5)CC4=O)cc31)C(=O)N(C1CCC(=O)NC1=O)C2=O] | Com- pound 151 |
| 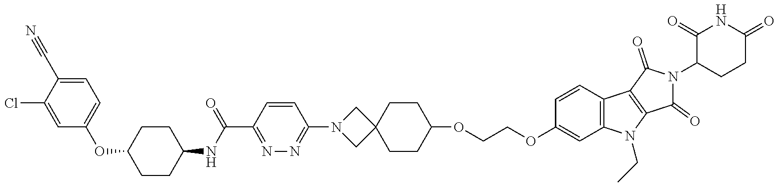 [SMILES: CCn1c2c(c3ccc(OCCOC4CCC5(CC4)CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 152 |
| 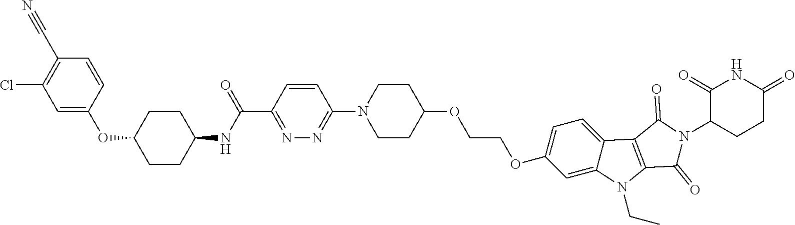 [SMILES: CCn1c2c(c3ccc(OCCOC4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 153 |
| 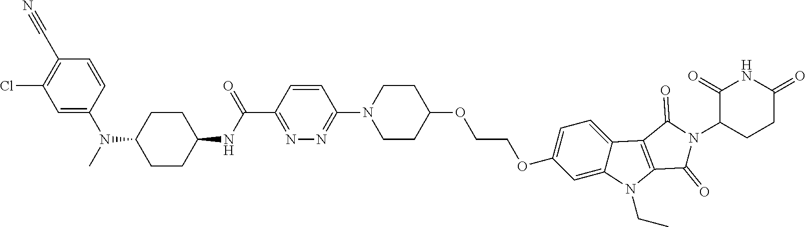 [SMILES: CCn1c2c(c3ccc(OCCOC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 154 |
| 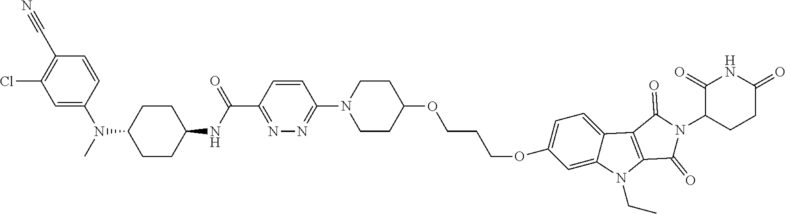 [SMILES: CCn1c2c(c3ccc(OCCCOC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 155 |
| 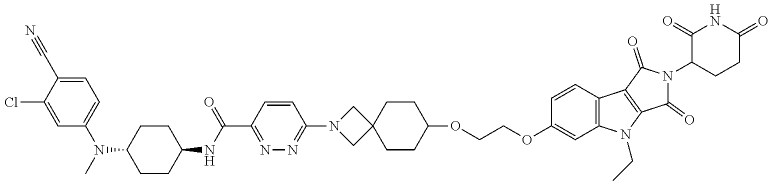 [SMILES: CCn1c2c(c3ccc(OCCOC4CCC5(CC4)CN(c4ccc(C(=O)N[C@H]6CC[C@H](N(C)c7ccc(C#N)c(Cl)c7)CC6)nn4)C5)cc31)C(=O)N(C1CCC(=O)NC1=O)C2=O] | Com- pound 156 |
| 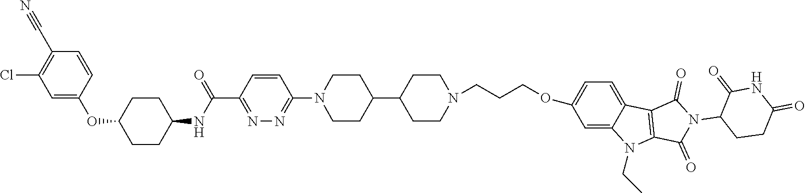 [SMILES: CCn1c2c(c3ccc(OCCCN4CCC(C5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O] | Com- pound 157 |
| 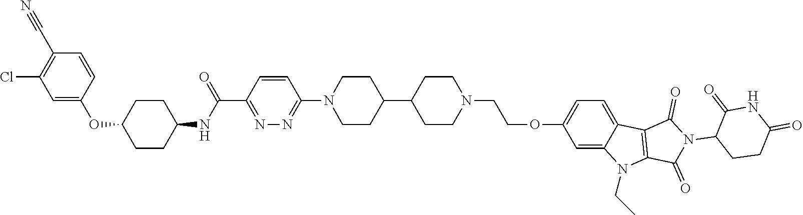 [SMILES: CCn1c2c(c3ccc(OCCN4CCC(C5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O] | Com- pound 158 |
| 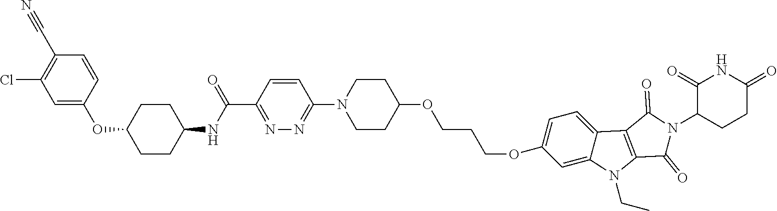 [SMILES: CCn1c2c(c3ccc(OCCCOC4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 159 |
| 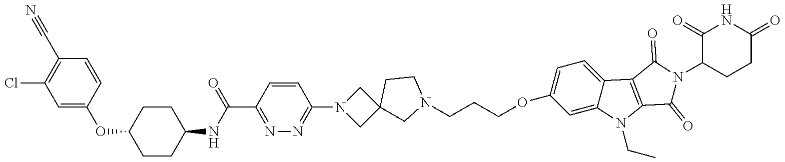 [SMILES: CCn1c2c(c3ccc(OCCCN4CCC5(C4)CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 160 |
| 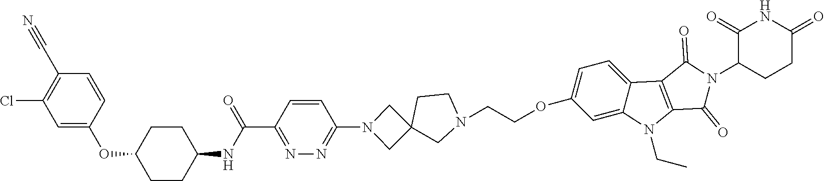 [SMILES: CCn1c2c(c3ccc(OCCN4CCC5(C4)CN(c4ccc(C(=O)N[C@H]6CC[C@H](Oc7ccc(C#N)c(Cl)c7)CC6)nn4)C5)cc31)C(=O)N(C1CCC(=O)NC1=O)C2=O] | Com- pound 161 |
| 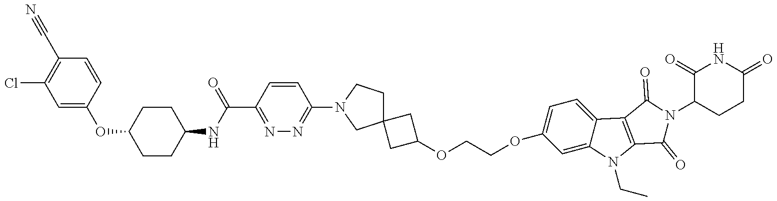 [SMILES: CCn1c2c(c3ccc(OCCOC4CC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O] | Com- pound 162 |
| 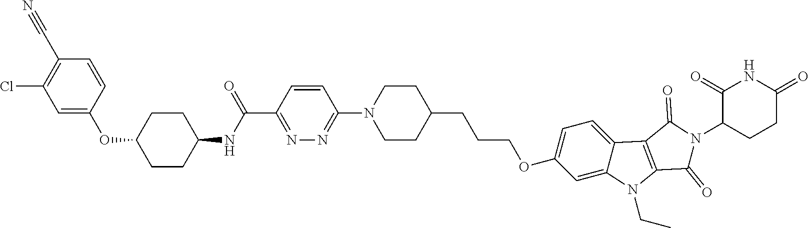 [SMILES: CCn1c2c(c3ccc(OCCCC4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 163 |
| 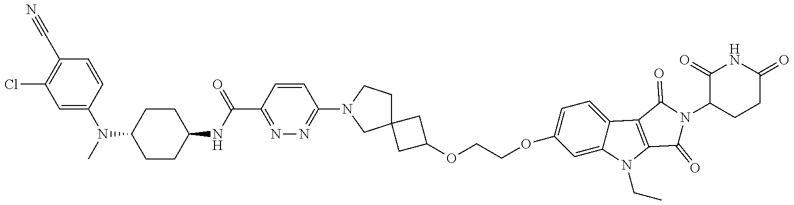 [SMILES: CCn1c2c(c3ccc(OCCOC4CC5(CCN(c6ccc(C(=O)N[C@H]7CC[C@H](N(C)c8ccc(C#N)c(Cl)c8)CC7)nn6)C5)C4)cc31)C(=O)N(C1CCC(=O)NC1=O)C2=O] | Com- pound 164 |
| 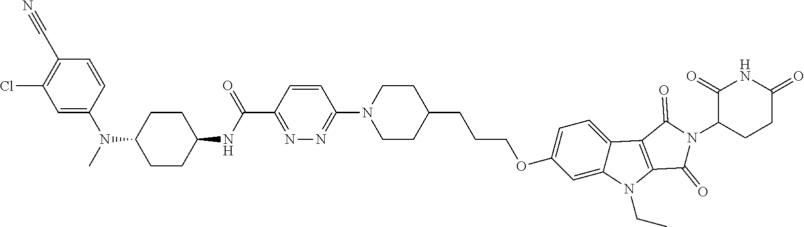 [SMILES: CCn1c2c(c3ccc(OCCCC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 165 |
| 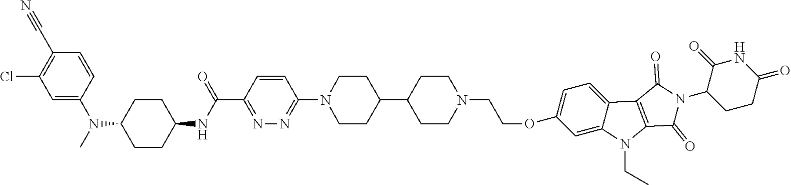 [SMILES: CCn1c2c(c3ccc(OCCN4CCC(C5CCN(c6ccc(C(=O)N[C@H]7CC[C@H](N(C)c8ccc(C#N)c(Cl)c8)CC7)nn6)CC5)CC4)cc31)C(=O)N(C1CCC(=O)NC1=O)C2=O] | Com- pound 166 |
| 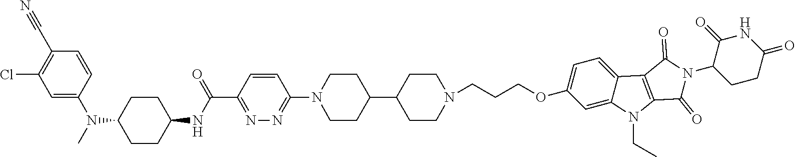 [SMILES: CCn1c2c(c3ccc(OCCCN4CCC(C5CCN(c6ccc(C(=O)N[C@H]7CC[C@H](N(C)c8ccc(C#N)c(Cl)c8)CC7)nn6)CC5)CC4)cc31)C(=O)N(C1CCC(=O)NC1=O)C2=O] | Com- pound 167 |
| 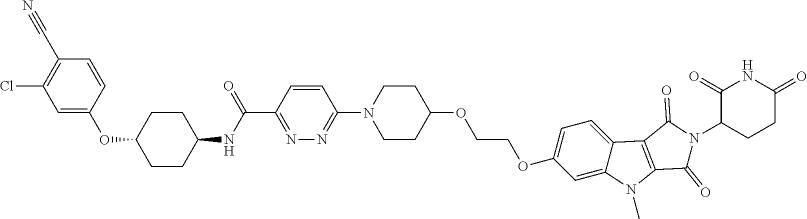 [SMILES: Cn1c2c(c3ccc(OCCOC4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 168 |
| 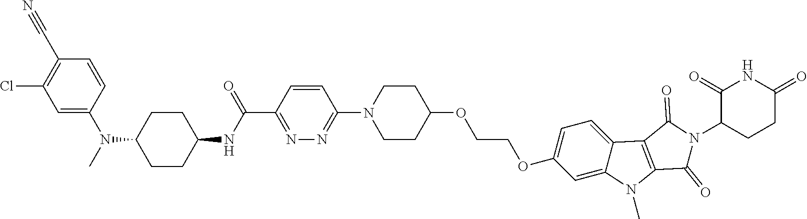 [SMILES: Cn1c2c(c3ccc(OCCOC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 169 |
| 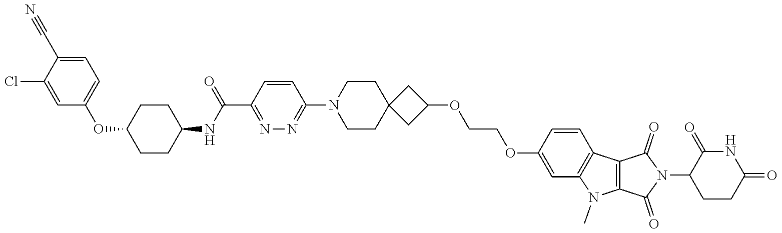 [SMILES: Cn1c2c(c3ccc(OCCOC4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)NC1=O)C2=O] | Com- pound 170 |
| 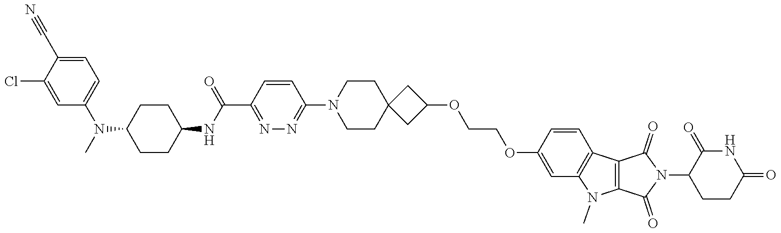 [SMILES: Cn1c2c(c3ccc(OCCOC4CC5(CCN(c6ccc(C(=O)N[C@H]7CC[C@H](N(C)c8ccc(C#N)c(Cl)c8)CC7)nn6)CC5)C4)cc31)C(=O)N(C1CCC(=O)NC1=O)C2=O] | Com- pound 171 |
| 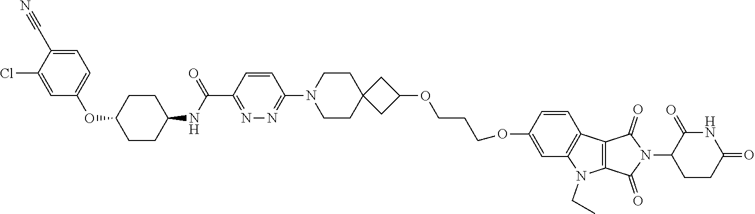 [SMILES: CCn1c2c(c3ccc(OCCCOC4CC5(CCN(c6ccc(C(=O)N[C@H]7CC[C@H](Oc8ccc(C#N)c(Cl)c8)CC7)nn6)CC5)C4)cc31)C(=O)N(C1CCC(=O)NC1=O)C2=O] | Com- pound 172 |
| 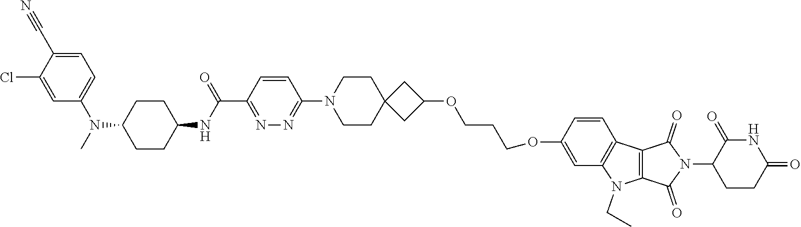 [SMILES: CCn1c2c(c3ccc(OCCCOC4CC5(CCN(c6ccc(C(=O)N[C@H]7CC[C@H](N(C)c8ccc(C#N)c(Cl)c8)CC7)nn6)CC5)C4)cc31)C(=O)N(C1CCC(=O)NC1=O)C2=O] | Com- pound 173 |
| 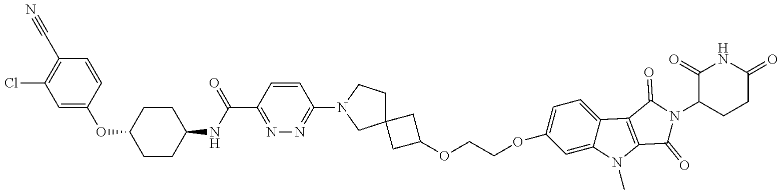 [SMILES: Cn1c2c(c3ccc(OCCOC4CC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O] | Com- pound 174 |
| 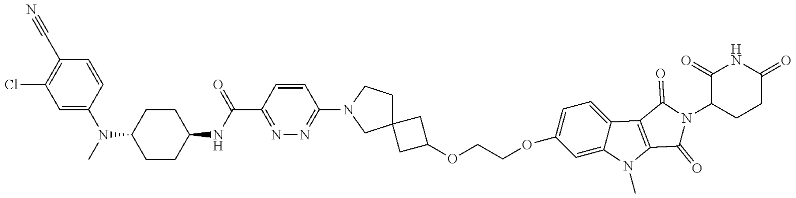 [SMILES: Cn1c2c(c3ccc(OCCOC4CC5(CCN(c6ccc(C(=O)N[C@H]7CC[C@H](N(C)c8ccc(C#N)c(Cl)c8)CC7)nn6)C5)C4)cc31)C(=O)N(C1CCC(=O)NC1=O)C2=O] | Com- pound 175 |
| 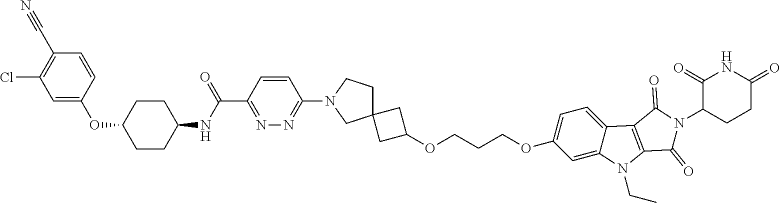 [SMILES: CCn1c2c(c3ccc(OCCCOC4CC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O] | Com- pound 176 |
| 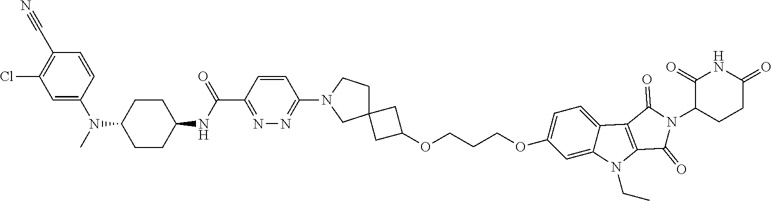 [SMILES: CCn1c2c(c3ccc(OCCCOC4CC5(CCN(c6ccc(C(=O)N[C@H]7CC[C@H](N(C)c8ccc(C#N)c(Cl)c8)CC7)nn6)C5)C4)cc31)C(=O)N(C1CCC(=O)NC1=O)C2=O] | Com- pound 177 |
| 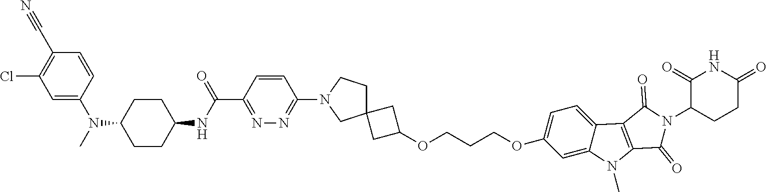 [SMILES: Cn1c2c(c3ccc(OCCCOC4CC5(CCN(c6ccc(C(=O)N[C@H]7CC[C@H](N(C)c8ccc(C#N)c(Cl)c8)CC7)nn6)C5)C4)cc31)C(=O)N(C1CCC(=O)NC1=O)C2=O] | Com- pound 178 |
| 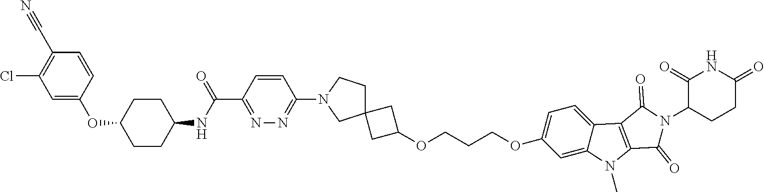 [SMILES: Cn1c2c(c3ccc(OCCCOC4CC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O] | Com- pound 179 |
| 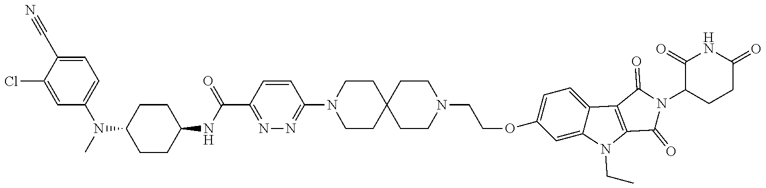 [SMILES: CCn1c2c(c3ccc(OCCN4CCC5(CC4)CCN(c4ccc(C(=O)N[C@H]6CC[C@H](N(C)c7ccc(C#N)c(Cl)c7)CC6)nn4)CC5)cc31)C(=O)N(C1CCC(=O)NC1=O)C2=O] | Com- pound 180 |
| 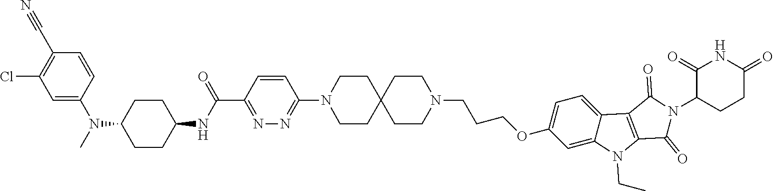 [SMILES: CCn1c2c(c3ccc(OCCCN4CCC5(CC4)CCN(c4ccc(C(=O)N[C@H]6CC[C@H](N(C)c7ccc(C#N)c(Cl)c7)CC6)nn4)CC5)cc31)C(=O)N(C1CCC(=O)NC1=O)C2=O] | Com- pound 181 |
| 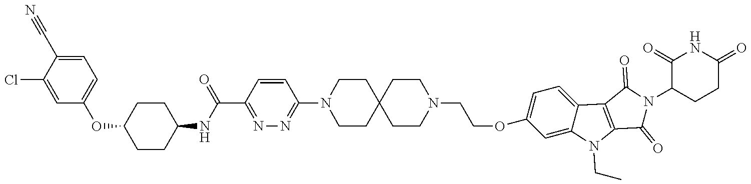 [SMILES: CCn1c2c(c3ccc(OCCN4CCC5(CC4)CCN(c4ccc(C(=O)N[C@H]6CC[C@H](Oc7ccc(C#N)c(Cl)c7)CC6)nn4)CC5)cc31)C(=O)N(C1CCC(=O)NC1=O)C2=O] | Com- pound 182 |
| 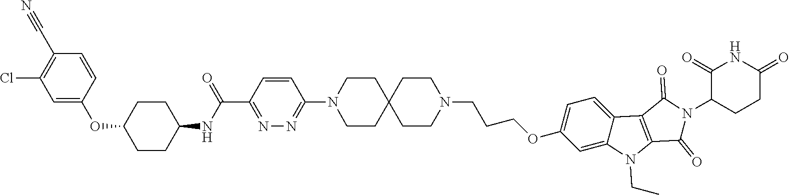 [SMILES: CCn1c2c(c3ccc(OCCCN4CCC5(CC4)CCN(c4ccc(C(=O)N[C@H]6CC[C@H](Oc7ccc(C#N)c(Cl)c7)CC6)nn4)CC5)cc31)C(=O)N(C1CCC(=O)NC1=O)C2=O] | Com- pound 183 |
| 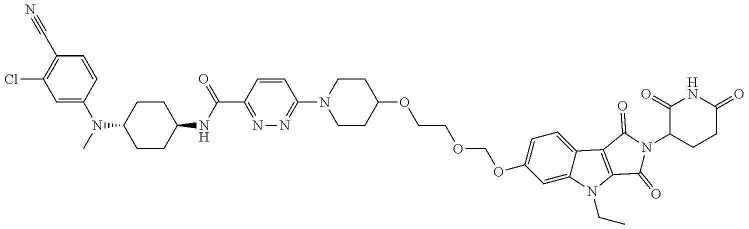 [SMILES: CCn1c2c(c3ccc(OCOCCOC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 184 |
| 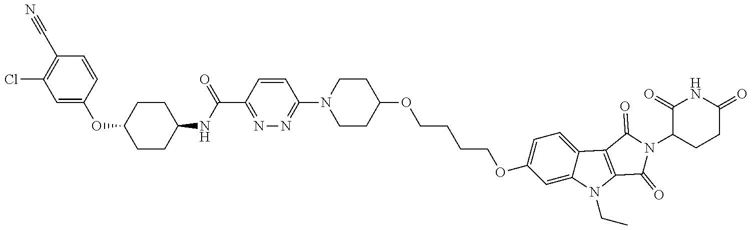 [SMILES: CCn1c2c(c3ccc(OCCCCOC4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 185 |
| 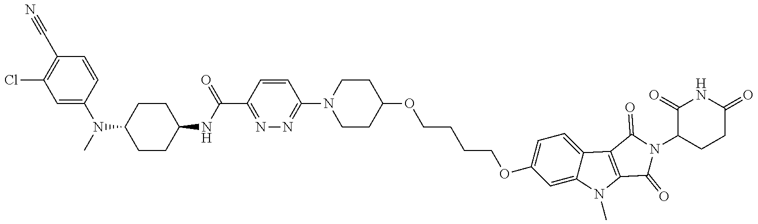 [SMILES: Cn1c2c(c3ccc(OCCCCOC4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 186 |
| 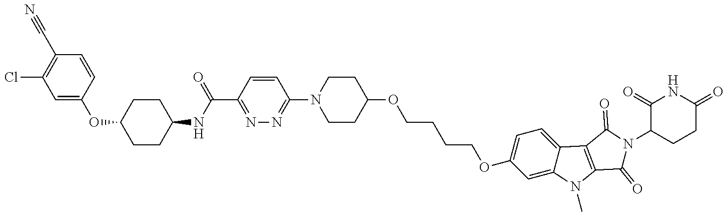 [SMILES: Cn1c2c(c3ccc(OCCCCOC4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 187 |
| 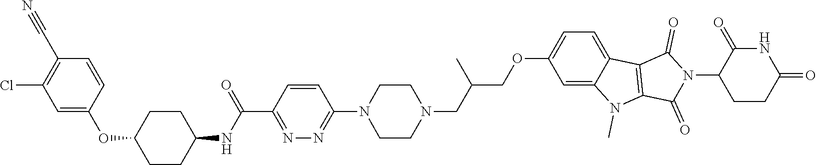 [SMILES: CC(COc1ccc2c3c(n(C)c2c1)C(=O)N(C1CCC(=O)NC1=O)C3=O)CN1CCN(c2ccc(C(=O)N[C@H]3CC[C@H](Oc4ccc(C#N)c(Cl)c4)CC3)nn2)CC1] | Com- pound 188 |
| 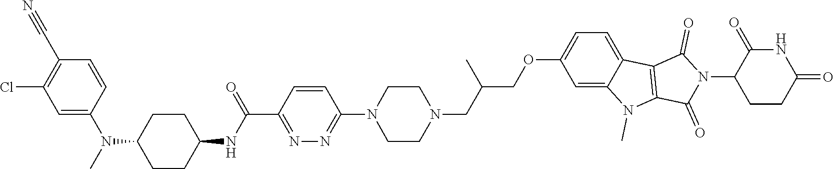 [SMILES: CC(COc1ccc2c3c(n(C)c2c1)C(=O)N(C1CCC(=O)NC1=O)C3=O)CN1CCN(c2ccc(C(=O)N[C@H]3CC[C@H](N(C)c4ccc(C#N)c(Cl)c4)CC3)nn2)CC1] | Com- pound 189 |
| 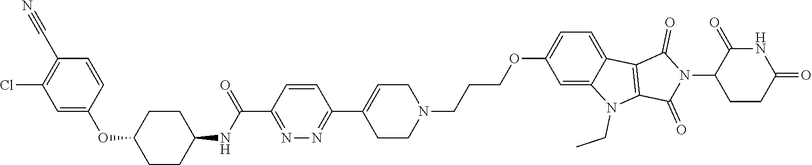 [SMILES: CCn1c2c(c3ccc(OCCCN4CC=C(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 190 |
| 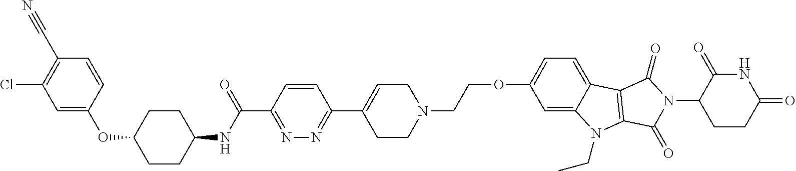 [SMILES: CCn1c2c(c3ccc(OCCN4CC=C(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 191 |
| 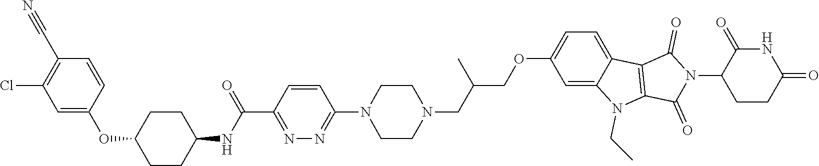 [SMILES: CCn1c2c(c3ccc(OCC(C)CN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 192 |
| 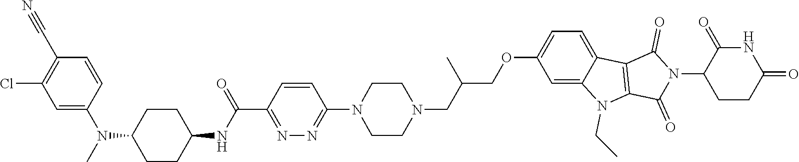 [SMILES: CCn1c2c(c3ccc(OCC(C)CN4CCN(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 193 |
| 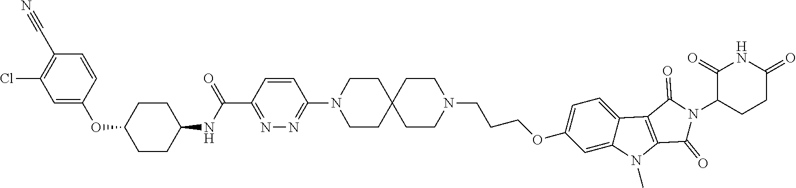 [SMILES: Cn1c2c(c3ccc(OCCCN4CCC5(CC4)CCN(c4ccc(C(=O)N[C@H]6CC[C@H](Oc7ccc(C#N)c(Cl)c7)CC6)nn4)CC5)cc31)C(=O)N(C1CCC(=O)NC1=O)C2=O] | Com- pound 194 |
| 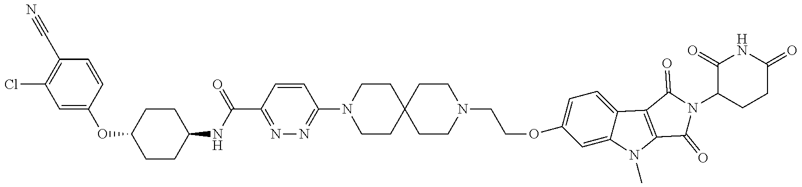 [SMILES: Cn1c2c(c3ccc(OCCN4CCC5(CC4)CCN(c4ccc(C(=O)N[C@H]6CC[C@H](Oc7ccc(C#N)c(Cl)c7)CC6)nn4)CC5)cc31)C(=O)N(C1CCC(=O)NC1=O)C2=O] | Com- pound 195 |
| 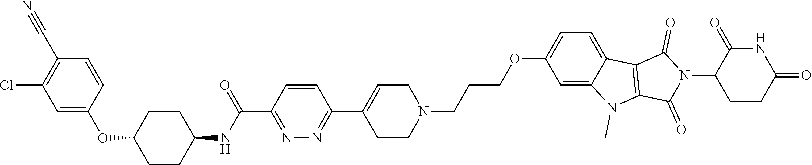 [SMILES: Cn1c2c(c3ccc(OCCCN4CC=C(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 196 |
| 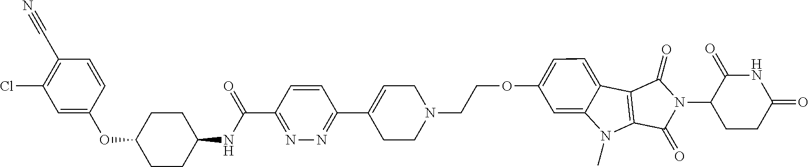 [SMILES: Cn1c2c(c3ccc(OCCN4CC=C(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 197 |
| 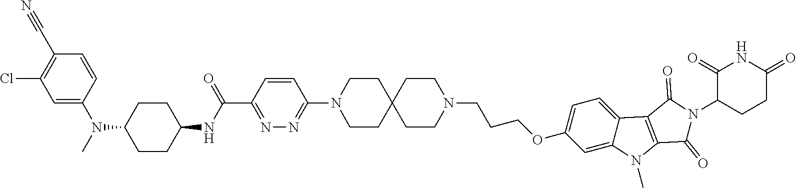 [SMILES: Cn1c2c(c3ccc(OCCCN4CCC5(CC4)CCN(c4ccc(C(=O)N[C@H]6CC[C@H](N(C)c7ccc(C#N)c(Cl)c7)CC6)nn4)CC5)cc31)C(=O)N(C1CCC(=O)NC1=O)C2=O] | Com- pound 198 |
| 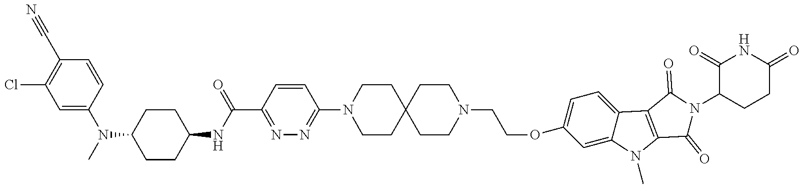 [SMILES: Cn1c2c(c3ccc(OCCN4CCC5(CC4)CCN(c4ccc(C(=O)N[C@H]6CC[C@H](N(C)c7ccc(C#N)c(Cl)c7)CC6)nn4)CC5)cc31)C(=O)N(C1CCC(=O)NC1=O)C2=O] | Com- pound 199 |
| 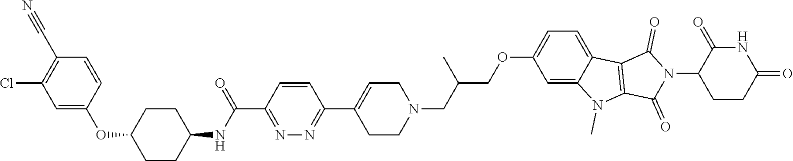 [SMILES: CC(COc1ccc2c3c(n(C)c2c1)C(=O)N(C1CCC(=O)NC1=O)C3=O)CN1CC=C(c2ccc(C(=O)N[C@H]3CC[C@H](Oc4ccc(C#N)c(Cl)c4)CC3)nn2)CC1] | Com- pound 200 |
| 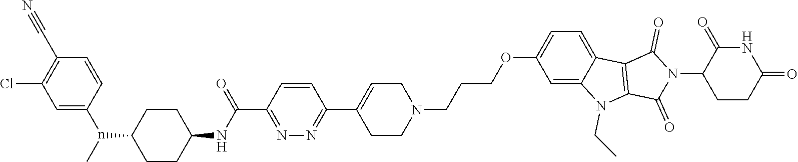 | Com- pound 201 |
| 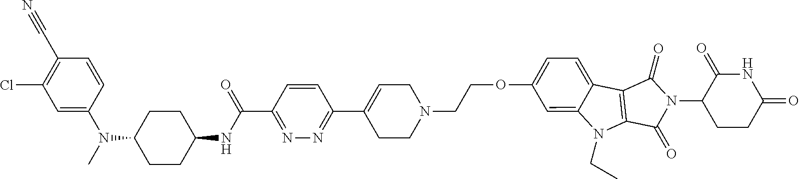 [SMILES: CCn1c2c(c3ccc(OCCN4CC=C(c5ccc(C(=O)N[C@H]6CC[C@H](N(C)c7ccc(C#N)c(Cl)c7)CC6)nn5)CC4)cc31)C(=O)N(C1CCC(=O)NC1=O)C2=O] | Com- pound 202 |
| 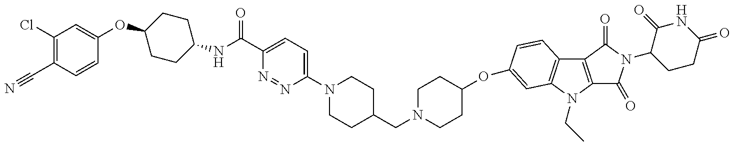 [SMILES: CCn1c2c(c3ccc(OC4CCN(CC5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O] | Com- pound 203 |
| 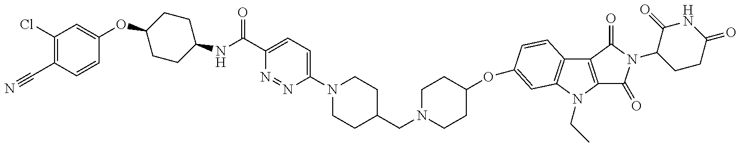 [SMILES: CCn1c2c(c3ccc(OC4CCN(CC5CCN(c6ccc(C(=O)N[C@H]7CC[C@@H](Oc8ccc(C#N)c(Cl)c8)CC7)nn6)CC5)CC4)cc31)C(=O)N(C1CCC(=O)NC1=O)C2=O] | Com- pound 204 |
| 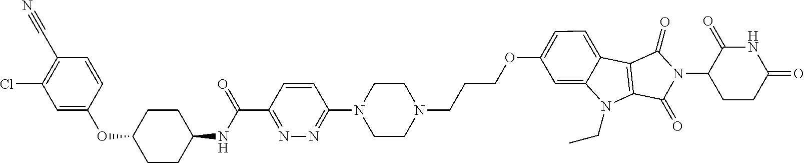 [SMILES: CCn1c2c(c3ccc(OCCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 205 |
| 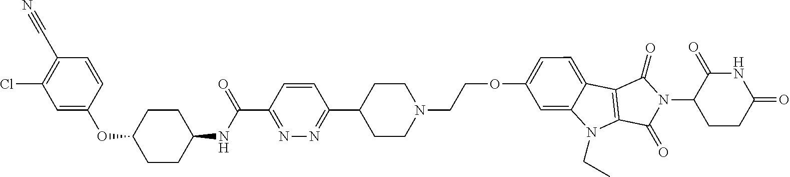 [SMILES: CCn1c2c(c3ccc(OCCN4CCC(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 206 |
| 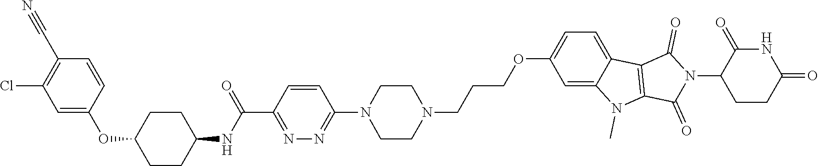 [SMILES: Cn1c2c(c3ccc(OCCCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 207 |
| 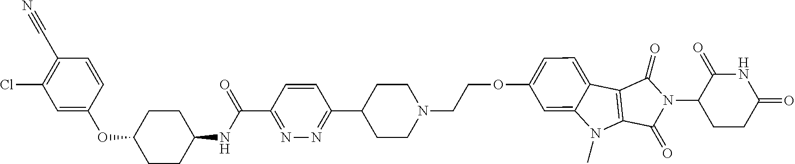 [SMILES: Cn1c2c(c3ccc(OCCN4CCC(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 208 |
| 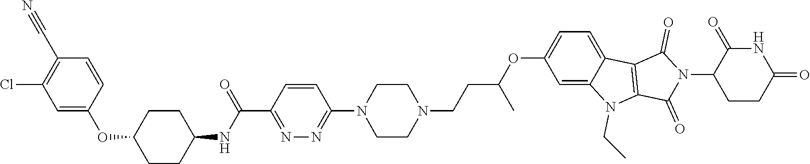 [SMILES: CCn1c2c(c3ccc(OC(C)CCN4CCN(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 209 |
| 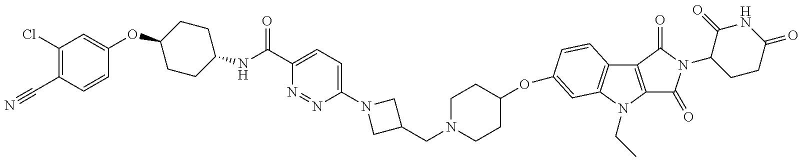 [SMILES: CCn1c2c(c3ccc(OC4CCN(CC5CN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O] | Com- pound 210 |
| 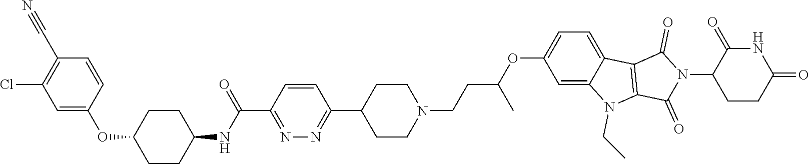 [SMILES: CCn1c2c(c3ccc(OC(C)CCN4CCC(c5ccc(C(=O)N[C@H]6CC[C@H](Oc7ccc(C#N)c(Cl)c7)CC6)nn5)CC4)cc31)C(=O)N(C1CCC(=O)NC1=O)C2=O] | Com- pound 211 |
| 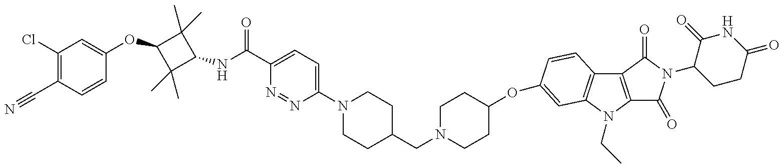 [SMILES: CCn1c2c(c3ccc(OC4CCN(CC5CCN(c6ccc(C(=O)N[C@H]7C(C)(C)[C@H](Oc8ccc(C#N)c(Cl)c8)C7(C)C)nn6)CC5)CC4)cc31)C(=O)N(C1CCC(=O)NC1=O)C2=O] | Com- pound 212 |
| 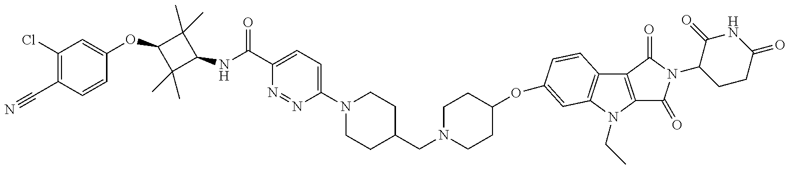 [SMILES: CCn1c2c(c3ccc(OC4CCN(CC5CCN(c6ccc(C(=O)N[C@H]7C(C)(C)[C@@H](Oc8ccc(C#N)c(Cl)c8)C7(C)C)nn6)CC5)CC4)cc31)C(=O)N(C1CCC(=O)NC1=O)C2=O] | Com- pound 213 |
| 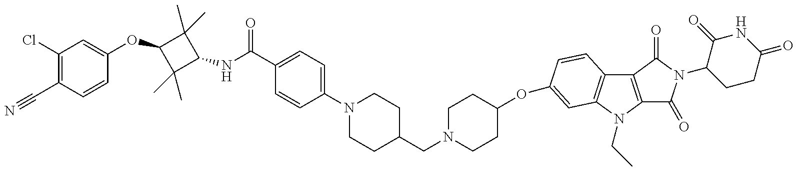 [SMILES: CCn1c2c(c3ccc(OC4CCN(CC5CCN(c6ccc(C(=O)N[C@H]7C(C)(C)[C@H](Oc8ccc(C#N)c(Cl)c8)C7(C)C)cc6)CC5)CC4)cc31)C(=O)N(C1CCC(=O)NC1=O)C2=O] | Com- pound 214 |
| 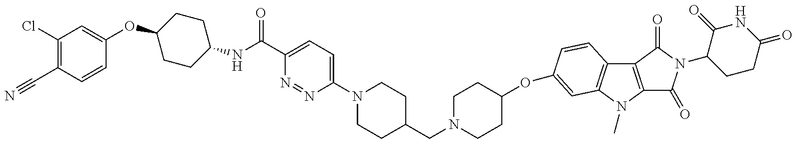 [SMILES: Cn1c2c(c3ccc(OC4CCN(CC5CCN(c6ccc(C(=O)N[C@H]7CC[C@H](Oc8ccc(C#N)c(Cl)c8)CC7)nn6)CC5)CC4)cc31)C(=O)N(C1CCC(=O)NC1=O)C2=O] | Com- pound 215 |
| 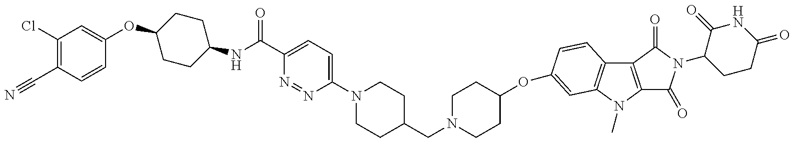 [SMILES: Cn1c2c(c3ccc(OC4CCN(CC5CCN(c6ccc(C(=O)N[C@H]7CC[C@@H](Oc8ccc(C#N)c(Cl)c8)CC7)nn6)CC5)CC4)cc31)C(=O)N(C1CCC(=O)NC1=O)C2=O] | Com- pound 216 |
| 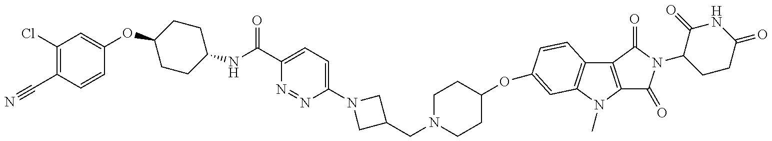 [SMILES: Cn1c2c(c3ccc(OC4CCN(CC5CN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O] | Com- pound 217 |
| 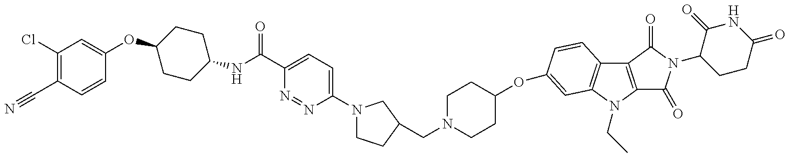 [SMILES: CCn1c2c(c3ccc(OC4CCN(CC5CCN(c6ccc(C(=O)N[C@H]7CC[C@H](Oc8ccc(C#N)c(Cl)c8)CC7)nn6)C5)CC4)cc31)C(=O)N(C1CCC(=O)NC1=O)C2=O] | Com- pound 218 |
| 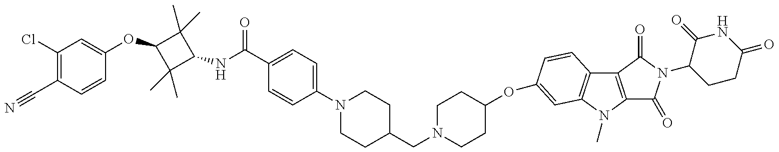 [SMILES: Cn1c2c(c3ccc(OC4CCN(CC5CCN(c6ccc(C(=O)N[C@H]7C(C)(C)[C@H](Oc8ccc(C#N)c(Cl)c8)C7(C)C)cc6)CC5)CC4)cc31)C(=O)N(C1CCC(=O)NC1=O)C2=O] | Com- pound 219 |
| 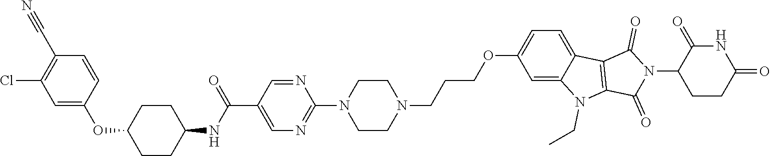 [SMILES: CCn1c2c(c3ccc(OCCCN4CCN(c5ncc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 220 |
| 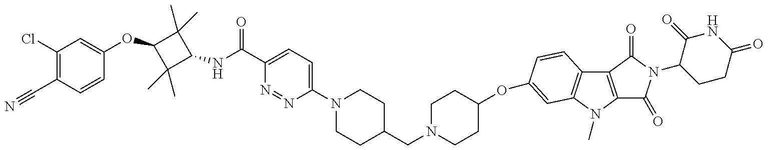 [SMILES: Cn1c2c(c3ccc(OC4CCN(CC5CCN(c6ccc(C(=O)N[C@H]7C(C)(C)[C@H](Oc8ccc(C#N)c(Cl)c8)C7(C)C)nn6)CC5)CC4)cc31)C(=O)N(C1CCC(=O)NC1=O)C2=O] | Com- pound 221 |
| 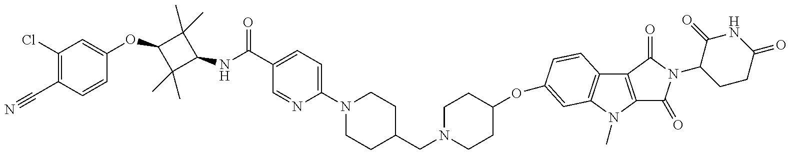 [SMILES: Cn1c2c(c3ccc(OC4CCN(CC5CCN(c6ccc(C(=O)N[C@H]7C(C)(C)[C@@H](Oc8ccc(C#N)c(Cl)c8)C7(C)C)cn6)CC5)CC4)cc31)C(=O)N(C1CCC(=O)NC1=O)C2=O] | Com- pound 222 |
| 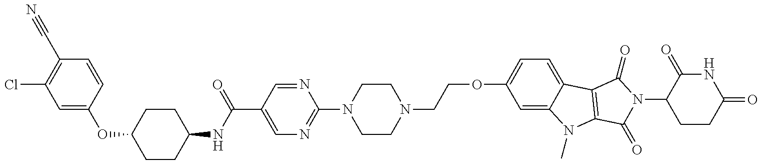 [SMILES: Cn1c2c(c3ccc(OCCN4CCN(c5ncc(C(=O)N[C@H]6CC[C@H](Oc7ccc(C#N)c(Cl)c7)CC6)cn5)CC4)cc31)C(=O)N(C1CCC(=O)NC1=O)C2=O] | Com- pound 223 |

### Method for Producing Compound of the Present Invention

A general method for producing the compounds of the present invention will be exemplified below. And, as extraction and purification, treatment which is performed in a normal experiment of organic chemistry may be conducted.

Synthesis of the compound of the present invention can be carried out referring to the procedures known in the art.

As a raw material compound, commercially available compounds, compounds described in the present description, compounds described in the references cited in the present description, and other known compounds can be utilized.

Among the compounds of the present invention, there are compounds in which a tautomer can be present, and the present invention includes all possible isomers and a mixture thereof, including them.

When one wants to obtain a salt of the compound of the present invention, in the case where the compound of the present invention is obtained in a form of a salt, it may be purified as it is and, in the case where the compound of the present invention is obtained in a free form, a salt may be formed by a normal method by dissolving or suspending the compound in a suitable organic solvent, and adding an acid or a base.

In addition, the compound of the present invention and a pharmaceutically acceptable salt thereof are present in a form of adducts with water or various solvents (hydrate or solvate) in some cases, and these adducts are included in the present invention.

The details of one or more embodiments of the invention are set forth in the description below. Other features, objects, and advantages of the invention will be apparent from the description and from the claims.

The compounds of this invention can be synthesized from commercially available starting materials by methods well known in the art. For example, one can prepare the compounds of this invention via the route shown below:

### General Procedure for the Compound Preparation

### Procedure 1

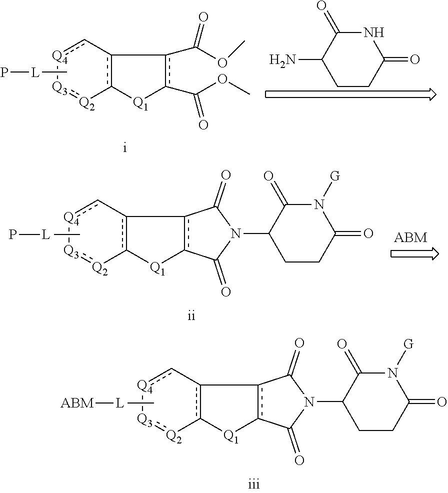

The Procedure 1 illustrates a general process for the preparation of the bifunctional compound of the present disclosure. In the first stage, a di-ester compound (I) was condensed with 3-aminopiperidine-2,6-dione to form an intermediate (ii). In the second stage, the intermediate (ii) was further condensed with a specific ABM compound to afford the desired compound (iii). The letter “P”, shown in the above Procedure 1, represents a protecting group.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Synthetic Method A—Synthesis of Compound 1

### (1) Preparation of Compound I-A

The compound I-A was first prepared from commercially available 2-benzyloxyethanol via the route shown below:

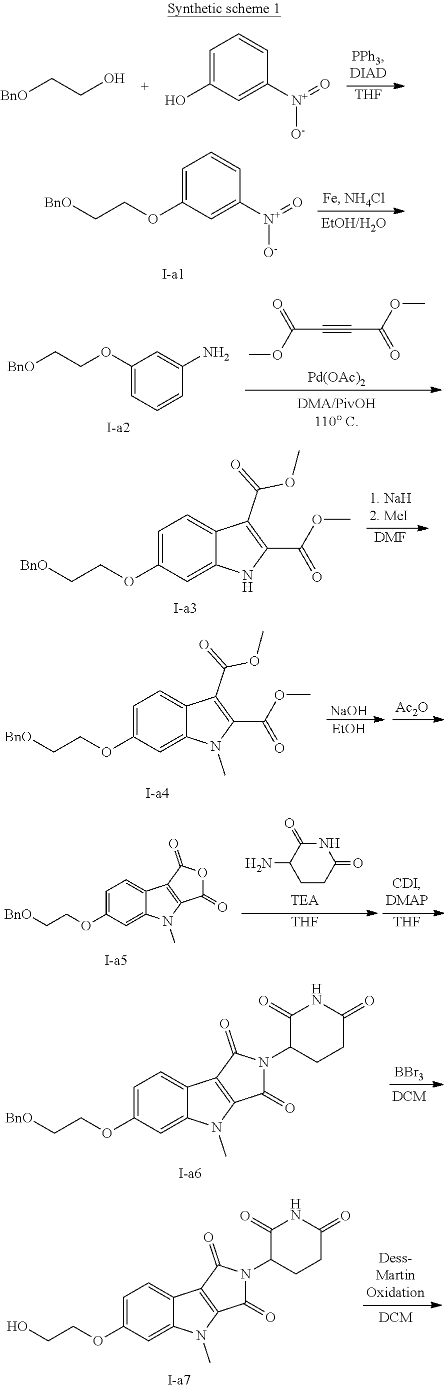

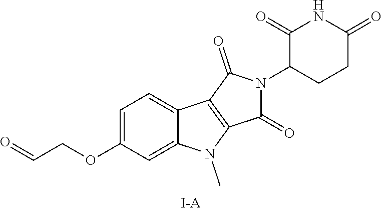

To a solution of 3-nitrophenol (6.67 g, 48.0 mmol), triphenylphosphine (PPh₃) (15.11 g, 57.6 mmol), and 2-benzyloxyethanol (6.89 ml, 48.5 mmol) in tetrahydrofuran (THF) (80 ml), diisopropyl azodicarboxylate (DIAD) (11.3 ml, 57.6 mmol) was added dropwise. After addition of the DIAD, the mixture was stirred under nitrogen for 18 hours (h) to give a crude mixture. After the solvent was evaporated, ether was added to re-suspend the crude mixture, and the triphenylphosphine oxide was filtered off. The solid was washed with ether (10 mL), and the filtrate was evaporated to give a yellow oil. The yellow oil was purified by column chromatography on silica gel (KM3 SCIENTIFIC CORP., Particle size 45-75 um) with an eluent (hexane:ethyl acetate=4:1) to give a compound I-a1 (13 g, 47.6 mmol).

To a solution of compound I-a1 (21.0 g, 0.077 mol) and NH₄Cl (9.96 g, 0.18 mol) in EtOH/H₂ (263 mL, v/v=4:1), iron powder (10.29 g, 0.18 mol) was added at room temperature. The reaction mixture was heated to reflux for 2 h. then cooled down to room temperature. Celite (10 g) was then added and stirred for 30 minutes (min.). The mixture was filtered and the solid was washed with MeOH (20 mL) and dichloromethane (DCM) (20 mL) for three times. The filtrate was washed with 30 mL of saturated NaHCO₃ aqueous solution. The aqueous phase was extracted with DCM (20 mL×3). The combined organic phases were washed with brine (20 mL×3), dried over Na₂SO₄, and concentrated in vacuum to give a compound I-a2 (15.7 g, yield: 84%).

Pd(OAc)₂ (262 mg, 20 mol %) was added to a mixture of compound I-a2 (1.7 g, 6.99 mmol), dimethyl acetylenedicarboxylate (0.71 mL, 5.8 mmol), and dimethylacetamide (DMA)/pivalic acid (PivOH) (4:1 v/v; 28 mL) in a 100 mL two-necks bottle, and the bottle then purged with air. The reaction mixture was gradually heated from room temperature to 110° C. under purged with air and stirred for 8 h during which it was monitored by LC-MASS. After reaction was completed, the solution was then cooled to room temperature, diluted with ethyl acetate (30 mL), washed with H₂O (3×10 mL), dried over MgSO₄, filtered, and evaporated under vacuum to give a crude product. The crude product was purified by column chromatography on silica gel (KM3 SCIENTIFIC CORP., Particle size 45-75 um) with an eluent (hexane:ethyl acetate=5:1) to afford a brown solid of 5-(2-benzyloxy-ethoxy)-1H-indole-2,3-dicarboxylic acid dimethyl ester I-a3 (1.14 g, yield: 50%)

An ice cold solution of 5-(2-benzyloxy-ethoxy)-1H-indole-2,3-dicarboxylic acid dimethyl ester I-a3 (3.0 g, 8.84 mmol) in N,N-dimethylformamide (DMF) (15 ml) was treated with 60% NaH (0.53 g, 13.3 mmol) and methyl iodide (0.82 ml, 13.3 mmol) and then warmed to room temperature. After 1 h, the reaction mixture was partitioned between DCM and saturated aqueous NH₄Cl. The combined organic layers were collected and washed with water (3×) and brine, then dried (with MgSO₄), filtered and concentrated to give a crude product. The crude product was purified by column chromatography on silica gel (KM3 SCIENTIFIC CORP., Particle size 45-75 um) with an eluent (hexane:ethyl acetate=3:1) to afford a brown solid compound I-a4 (2.7 g, yield: 87%).

The compound I-a4 (0.6 g, 1.51 mmol) was treated with a solution of NaOH (0.6 g, 15.1 mmol) in EtOH (30 mL) and heated to reflux (85° C. bath temp). After 4 h, the reaction mixture was cooled to ambient temperature, removed excess EtOH, diluted with EtOAc (30 mL) and acidified with 1 M HCl to pH=3 and the layers were separated. The aqueous layer was then extracted with EtOAc (30 mL) and the combined organic layers were washed with brine, dried (with MgSO₄), filtered and concentrated to afford a white solid. Then the white solid and Ac₂O (5 mL) were combined and heated to 140° C. After 2 h, a mono-ester was found by LC-MASS, and the reaction mixture was cooled to ambient temperature. Then excess Ac₂O was removed, and then dried toluene was added and concentrated. The residue I-a5 was thus produced and then used without any further purification.

The above residue I-a5 was dissolved in THF (20 mL) followed by the addition of 3-aminopiperidine-2,6-dione hydrochloride (0.25 g, 3.0 mmol) and trimethylamine (TEA) (1.52 g, 15.1 mmol). The mixture was stirred at 50° C. for 4 h. After reaction completed, the reaction mixture was cooled to ambient temperature, diluted with EtOAc and acidified with 1 M HCl to pH=3. The organic layer was washed with brine, dried (with MgSO₄), filtered and concentrated to afford a residue. Then the residue was dissolved in THF (20 mL) followed by the addition of N,N′-carbonyldiimidazole (CDI) (0.49 g, 3.0 mmol) and DMAP (18 mg, 0.15 mmol) to give a mixture. The mixture was stirred at 50° C. for 4 h. After reaction completed, the reaction mixture was cooled to ambient temperature, diluted with EtOAc, and acidified with 1 M HCl to pH=3. The organic layer was washed with brine, dried (with MgSO₄), filtered and concentrated to afford a crude product. The crude product was purified by column chromatography on silica gel (KM3 SCIENTIFIC CORP., Particle size 45-75 um) with an eluent (hexane:ethyl acetate=1:4) to afford a compound I-a6 (0.41 g, yield: 60%).

To a solution of compound I-a6 (100 mg, 0.22 mmol) in DCM (6 mL), 1M BBr₃ in DCM (0.87 mL) was added slowly at −78° C. The mixture was stirred at −60° C. for 3 h. After reaction completed, water was added slowly and the resulting mixture was warmed to ambient temperature, and diluted with THF. The organics were washed with brine, dried (with Na₂SO₄), filtered and concentrated to afford a compound I-a7. Then compound I-a7 was dissolved in DCM (10 mL) followed by the addition of Dess-Martin periodinane (186 mg, 0.44 mmol) and NaHCO₃ (37 mg, 0.44 mmol). The mixture was stirred at room temperature for 17 h. After reaction was completed, water was added to give a mixture, and the mixture was diluted with DCM. The organics were washed with brine, dried (with Na₂SO₄), filtered and concentrated to afford a compound I-A. The compound I-A was used for the next synthetic step without further purification.

### (2) Preparation of Secondary Amine Compound I-B

The secondary amine compound I-B was prepared from commercially available benzonitrile, 4-[(trans-3-amino-2,2,4,4-tetramethylcyclobutyl)oxy]-2-chloro-, hydrochloride via the route shown below:

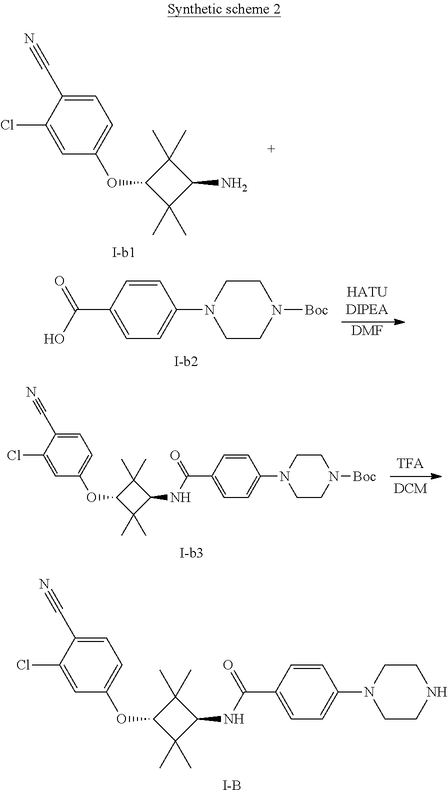

To a mixture of benzonitrile, 4-[(trans-3-amino-2,2,4,4-tetramethylcyclobutyl)oxy]-2-chloro-, hydrochloride (1 g, 3.59 mmol) I-b1 and 4-(4-carboxy-phenyl)-piperazine-1-carboxylic acid tert-butyl ester (1.2 g, 4.00 mmol) I-b2 in DMF (8 mL), N,N-Diisopropylethylamine (DIPEA) (3.0 mL, 17.20 mmol) and 1-[Bis(dimethylamino)methylene]-1H-1,2,3-triazolo[4,5-b]pyridinium 3-oxide hexafluorophosphate (HATU) (1.6 g, 4.30 mmol) were added at room temperature then stirred at room temperature for 24 h. The reaction was concentrated to remove the solvent, then water was added to give a solution and extracted the solution with DCM (30 mL×2). The organics were washed with brine, dried (with MgSO₄), filtered and concentrated to afford compound I-b3 (1.6 g, 76%). Then the compound I-b3 and CF₃COOH (2.5 ml) were combined and stirred in CH₂Cl₂(10 mL) at room temperature for 4 h. After reaction completed, the excess CF₃COOH was removed under vacuum to give a residue. Then the residue was diluted with DCM (10 mL) basified with Na₂CO₃ to pH=10 and the organic layer was washed with brine (3 mL), dried (with Na₂SO₄), filtered and concentrated to afford a compound I-B, which was directly used without any purification.

### (3) Preparation of Compound 1 from Compound I-A and Secondary Amine Compound I-B

Compound 1 was prepared via the route shown below:

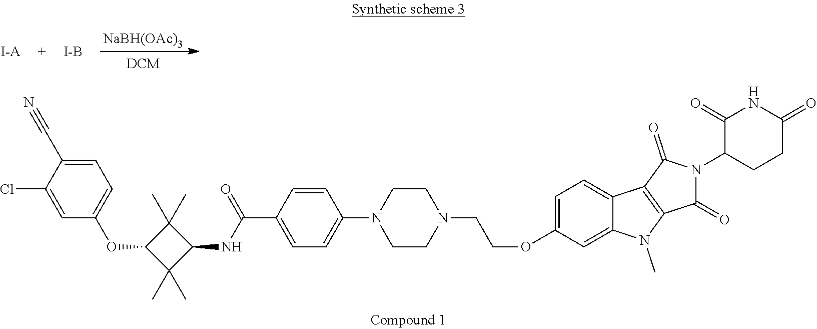 [SMILES: "I-A" ; "I-B" ; Cn1c2c(c3ccc(OCCN4CCN(c5ccc(C(=O)N[C@H]6C(C)(C)[C@H](Oc7ccc(C#N)c(Cl)c7)C6(C)C)cc5)CC4)cc31)C(=O)N(C1CCC(=O)NC1=O)C2=O ; "NaBH(OAc)3"]

To a mixture of the compound I-A (46 mg, 0.125 mmol) and compound I-B (23 mg, 0.05 mmol) in DCM (1 mL), NaBH(OAc)₃ (36 mg, 0.17 mmol) was added. The resulting solution was stirred for 17 h at room temperature. After reaction was completed, the solution was diluted with water (3 mL). The resulting solution was extracted with DCM (5 mL×3) and the organic layers combined. The organic layer was washed with brine (5 mL), dried (with Na₂SO₄), filtered and concentrated to afford a crude product. The crude product was purified by Preparative thin-layer chromatography (PLC) (DCM/Acetone=9/1) to give Compound 1 (7 mg, yield: 17%). MS: m/z 820.3 (M⁺+1); ¹H NMR (DMSO-d₆) δ 11.06 (s, 1H), 7.90 (d, 1H), 7.75 (d, 2H), 7.62 (d, 1H), 7.51 (d, 1H), 7.33 (s, 1H), 7.20 (s, 1H), 7.02-6.98 (m, 4H), 4.97 (dd, 11H), 4.32 (s, 1H), 4.16 (t, 2H), 4.05 (d, 1H), 3.96 (s, 3H), 3.28 (m, 4H), 2.92-2.86 (m, 1H), 2.84 (t, 2H), 2.68 (m, 4H), 2.61-2.50 (m, 2H), 2.04-2.02 (m, 1H), 1.21 (s, 6H). 1.12 (s, 6H).

### Synthetic Method B—Synthesis of Compound 2

### (1) Preparation of Compound I-C

The compound I-C was first prepared from commercially available 4-(2-hydroxy-ethyl)-piperidine-1-carboxylic acid tert-butyl ester via the route shown below:

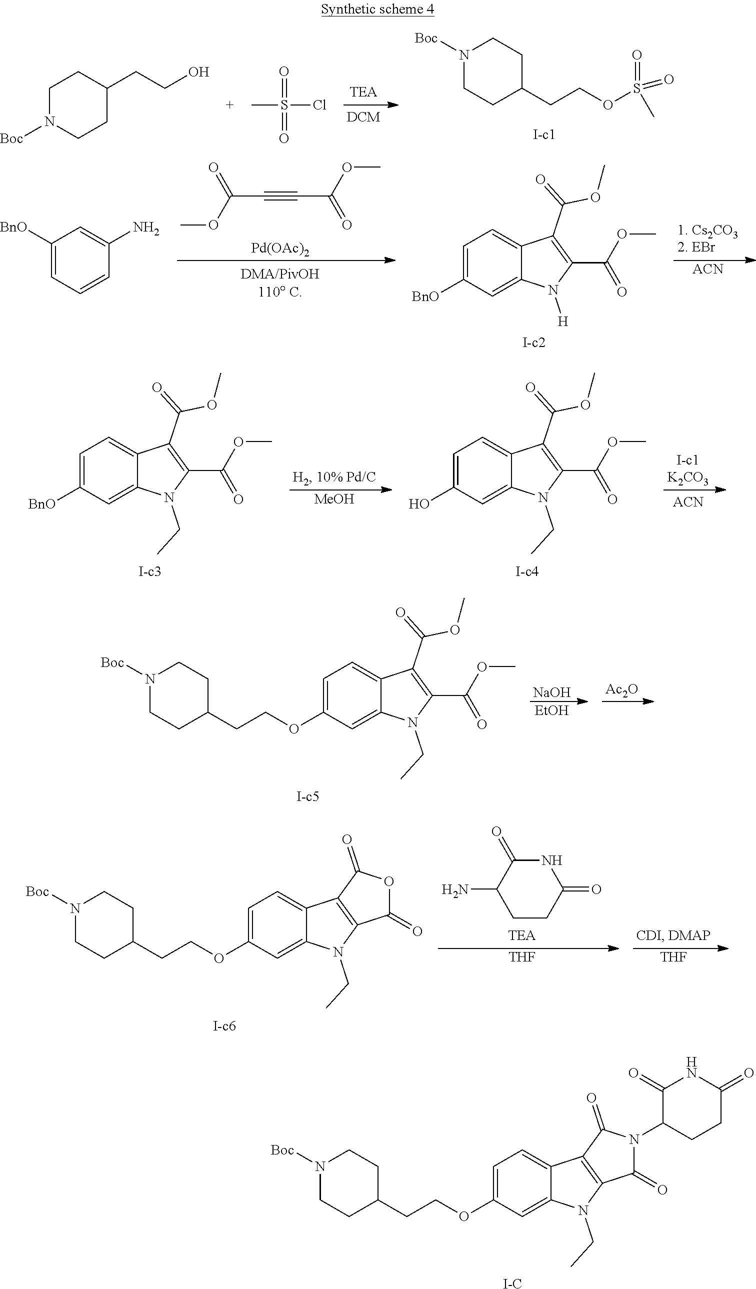

To a solution of 4-(2-hydroxy-ethyl)-piperidine-1-carboxylic acid tert-butyl ester (3.5 g, 15.5 mmol) in DCM (50 mL), triethylamine (6.3 mL, 46.5 mmol) was added, and then methanesulfonyl chloride (MsCl) (1.8 mL, 23.25 mmol) was added under ice bath and stirred at room temperature for 1 h. The reaction mixture was washed with saturated NaHCO₃ (10 mL) and saturated NH₄Cl_{(aq)} (10 mL), and dried over MgSO₄. The solvent was removed to give a crude product, and the crude product was purified by flash column with an eluent (Hexane/EtOAc=3/1 to 1/1) to afford a compound I-c1 (4.67 g, 98%).

Pd(OAc)₂ (1.13 g, 10 mol %) was added to a solution of 3-benzyloxy-phenylamine (10 g, 50.1 mmol), dimethyl acetylenedicarboxylate (5.86 mL, 47.6 mmol), and dimethylacetamide (DMA)/pivalic acid (PivOH) (4:1 v/v; 100 mL) in a container purged with air. The reaction mixture was gradually heated to 120° C. for 8 h. After reaction was completed, the solution was then cooled to room temperature, diluted with ethyl acetate (200 mL), washed with water (50 mL×3) and brine (50 mL), dried over MgSO₄, filtered, and evaporated under vacuum to give a crude product. The crude product was purified by column chromatography on silica gel (KM3 SCIENTIFIC CORP., Particle size 45-75 um) with an eluent (hexane:ethyl acetate=5:1) to afford a brown solid of compound I-c2 (8.4 g, 54%)

To a solution of compound I-c2 (5.0 g, 14.73 mmol, 1 eq.) in CH₃CN (50 mL), Cs₂CO₃ (9.6 g, 29.47 mmol, 2.0 eq) and bromoethane (1.65 mL, 22.10 mmol, 1.5 eq.) were sequentially added at 25° C. under N₂ atmosphere. The mixture was heated at 80° C. for 4 hours until no I-c2 was left and the reaction mixture was cooled to room temperature. Solvent was reduced to half of the original volume, diluted with ethyl acetate (50 mL), and washed with water (100 mL). Layers were separated and the aqueous layer was extracted with ethyl acetate (30 mL×2). The combined organic layers were washed with brine (100 mL), dried over MgSO₄, filtered, and concentrated under vacuum. The crude material was purified by flash column chromatography on silica gel (KM3 SCIENTIFIC CORP., Particle size 45-75 um) eluting with 15-20% ethyl acetate/hexane to afford the compound I-c3 (4.8 g, 88%).

A solution of compound I-c3 (2.0 g) in MeOH (60 mL) was degassed, and Pd/C (0.2 g, 10 wt %) was added, then the reaction mixture was bubbled with a balloon of hydrogen gas and stirred at room temperature for 2 h. While the reaction was completed, the reaction mixture was filtered through a plug of Celite, and the filter cake was washed with ethyl acetate (EA). The filtrate was concentrated by rotary evaporation to get compound I-c4 (1.49 g, 99% yield).

To a solution of the compound I-c4 (1.75 g, 6.31 mmol) in acetonitrile (15 mL), K₂CO₃ (2.65 g, 3.0 eq) was added and stirred at 60° C. to give a suspension. After 30 min, the above suspension was cooled down, and added a solution of the compound I-c1 (2.33 g, 1.2 eq) in acetonitrile (5 mL) to give a mixture. The mixture was heated to 80° C. and maintained at this temperature for 16 h. After that, the reaction mixture was cooled down, and EtOAc (20 mL) and water (20 mL) was added for extraction. The mixture stood still for a moment to separate water and organic layers, and water layer was extracted with EtOAc (20 mL) again. All organic layers were combined, washed with brine (20 mL), dried with MgSO₄, and purified by flash column to afford a compound I-c5 (2.26 g, 87%).

To a solution of the compound I-c5 (0.38 g, 1 eq) in EtOH (20 mL), NaOH (0.56 g, 20 eq) was added and stirred at 80° C. for 17 h. After cooling down, EtOH was removed by a rotor, and the residue was diluted with water (10 mL). 1N HCl_{(aq)} was then added under ice bath until pH value of the diluted residue was less than 2 (<2). The aqueous layer was extracted with EtOAc (20 mL×2), and the organic layers were combined, washed with brine (10 mL×1), dried over MgSO₄, and concentrated to afford a crude residue. Then the crude residue in Ac₂O (6 mL) was heated to 80° C. and stirred for 4 h. After cooling down, Ac₂O was evaporated under vacuum, and toluene (2-3 mL) was added. The solvent was evaporated under vacuum to afford a compound I-c6 (0.30 g, 87%).

A solution of 3-Aminopiperidine-2,6-dione hydrochloride (0.11 g, 2 eq) and TEA (0.19 mL, 4 eq) in THF (2 mL) was stirred at room temperature for 30 min, then a solution of the compound I-c6 (0.15 g, 1 eq) in THF (2 mL) was added. The reaction was stirred at room temperature for 50 min, then the mixture was diluted with EtOAc (10 mL×1) and water (5 mL×1). 1N HCl_{(aq)} was then added until the pH value of aqueous layer was less than 2 (<2). The aqueous layer was extracted with EtOAc (10 mL×2), and the organic layers were combined, washed with brine (5 mL×1), and dried with MgSO₄. The solvent was removed to give a crude material, and the crude material was used in the next step without purification. The above crude material was dissolved in THF (4 mL), and CDI (0.11 g, 2 eq) and DMAP (4 mg, 0.1 eq) were added. The reaction mixture was stirred at 50° C. for 2 h. After cooling down, the reaction mixture was diluted with EtOAc (10 mL) and water (5 mL). 1N HCl_{(aq)} was then added until the pH value of aqueous layer was less than 2 (<2), and the aqueous layer was then extracted with EtOAc (10 mL×2). The organic layers were collected, washed with brine (10 mL), and dried over Na₂SO₄. The solvent was removed to give a crude product, and the crude product was purified by flash column chromatography with an eluent (Hexane/EtOAc=1/1 to 1/2) to afford a compound I-C (0.16 g, 85%).

### (2) Preparation of Compound I-D

The compound I-D was prepared from commercially available 4-(4-Amino-cyclohexyloxy)-2-chloro-benzonitrile via the route shown below:

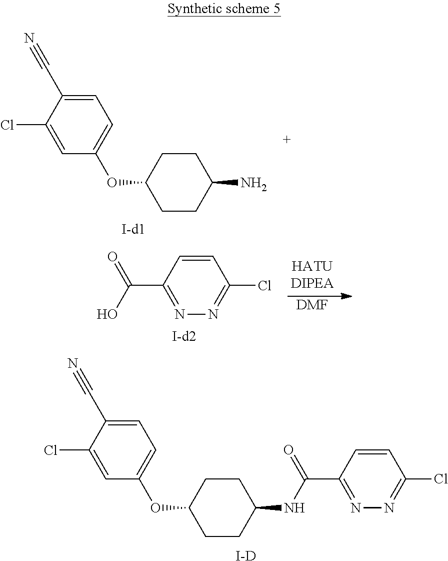

To a solution of 4-(4-amino-cyclohexyloxy)-2-chloro-benzonitrile I-d1 (2.06 g, 1 eq), compound I-d2 (1.31 g, 1 eq), and HATU (4.99 g, 1.6 eq) in DCM (30 mL), DIPEA (4.3 mL, 3 eq) was added under ice bath and stirred at room temperature for 21 h. After that, water (10 mL) was added and mixed, and the organic layer was separated, dried with Na₂SO₄, concentrated, purified by flash column with an eluent (Hexane/EtOAc=1/1) and then washed with DCM and Hexane to afford a compound I-D (1.8 g, 56%). MS: m/z 391.2 (M⁺+1); ¹H NMR (DMSO-d₆) δ 9.14 (d, 1H), 8.22 (d, 1H), 8.10 (d, 1H), 7.85 (d, 1H), 7.39 (d, 1H), 7.14 (dd, 1H), 4.55-4.51 (m, 1H), 3.94-3.88 (m, 1H), 2.13-2.11 (m, 2H), 1.91-1.89 (m, 2H), 1.73-1.66 (m, 2H), 1.55-1.48 (m, 2H).

### (3) Preparation of Compound 2 from Boc-Protected Amine Compound I-C and Compound I-D

Compound 2 was prepared via the route shown below:

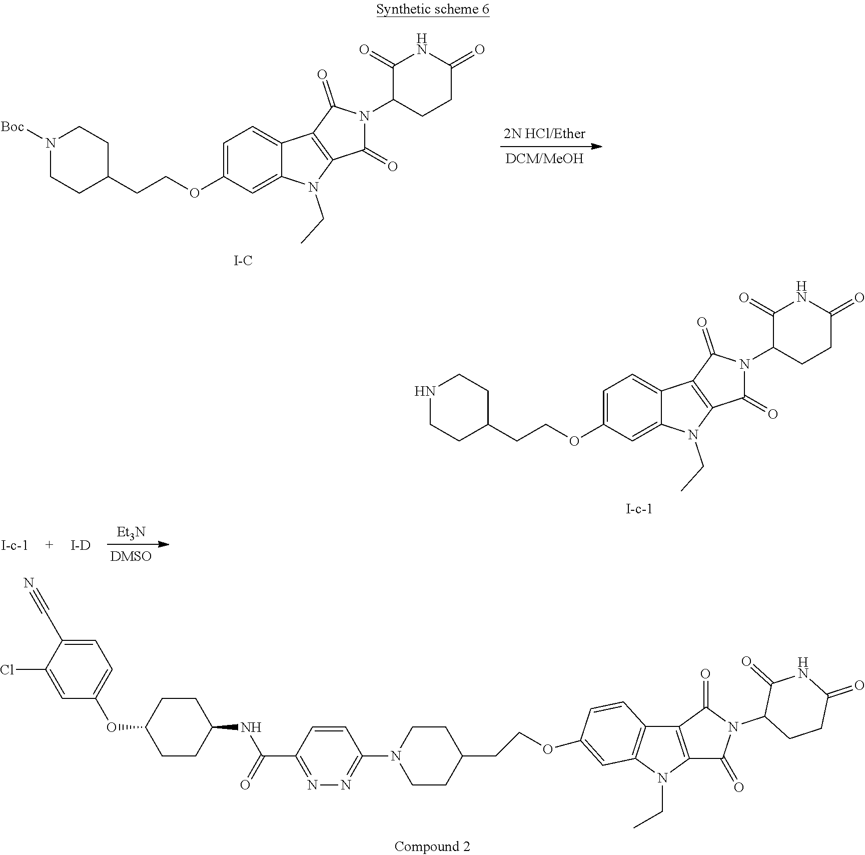

To a solution of compound I-C (100 mg, 1 eq) in DCM (2 mL) and MeOH (0.4 mL), 2N HCl in ether (1.6 mL, 20 eq) was added. The reaction was stirred at room temperature for 40 min, and the solvent was removed to afford a compound I-c1 (80 mg, 98%).

Then, to a solution of the compound I-c1 (51 mg, 1 eq) and compound I-D (44 mg, 1.2 eq) in DMSO (0.5 mL), TEA (0.13 mL, 10 eq) was added and stirred at 80° C. for 17 h. After cooling down, the reaction mixture was diluted with DCM (10 mL) and water (5 mL). After that, the organic layer was washed with water (5 mL), saturated NH₄Cl (3 mL×3) and dried over Na₂SO₄. The solvent was removed to give a crude material, and the crude material was purified by preparative TLC with a mobile phase (DCM/Acetone=9/1) to afford a compound 2 (38 mg, 47%). MS: m/z 807.4 (M++1); ¹H NMR (CDCl₃) δ 7.97 (d, 1H), 7.96-7.90 (m, 1H), 7.87 (d, 1H), 7.74 (d, 1H), 7.56 (d, 1H), 7.00-6.97 (m, 3H), 6.85 (dd, 1H), 6.83 (d, 1H), 4.85 (dd, 1H), 4.55-4.52 (m, 2H), 4.38 (q, 2H), 4.35-4.29 (m, 1H), 4.21 (t, 2H), 4.08-4.02 (m, 1H), 3.10-3.06 (m, 2H), 2.91-2.88 (m, 1H), 2.84-2.70 (m, 2H), 2.22-2.15 (m, 5H), 2.00-1.94 (m, 3H), 1.85-1.82 (m, 2H), 1.71-1.65 (m, 2H), 1.53 (t, 3H), 1.50-1.42 (m, 2H), 1.38-1.33 (m, 2H).

### Synthetic Method C—Synthesis of Compound 3

### (1) Preparation of Compound I-E

The compound I-E was prepared from commercially available piperazine-1-carboxylic acid tert-butyl ester via the route shown below:

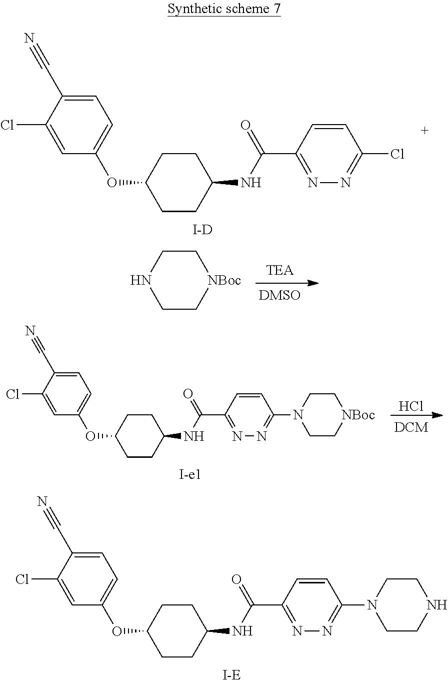

A mixture of compound I-D (800 mg, 1 eq), piperazine-1-carboxylic acid tert-butyl ester (399 mg, 1.1 eq), and TEA (0.57 mL, 2 eq) in DMSO (6 mL) was stirred at 100° C. for 24 h. After cooling down, the mixture was added slowly into water to give a suspension. The suspension was filtered to afford compound I-e1 (1.03 g, 94%).

HCl gas was slowly bubbled into a mixture of I-e1 (500 mg, 1 eq) in CH₂Cl₂ (6 mL) to give a mixture. The mixture was stirred at room temperature for 2 h, and then concentrated and washed with methyl tert-butyl ether (MTBE) to afford compound I-E (388 mg, 97%).

### (2) Preparation of Compound I-F

The compound I-F was prepared via the route shown below:

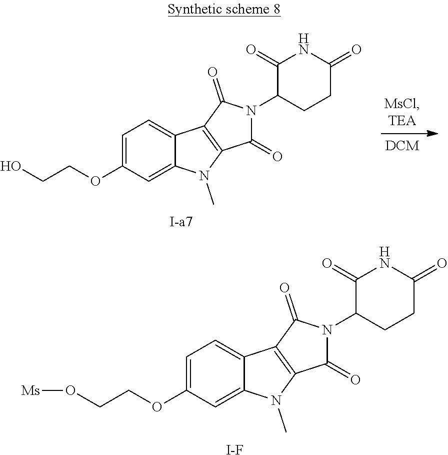

To a solution of I-a7 (0.1 g, 0.250 mmol) in DCM (2 mL) was added TEA (0.139 mL, 4.0 eq) at room temperature. Then methane sulfonyl chloride (MsCl) (0.0387 mL, 2.0 eq) was added dropwise. The mixture reaction was stirred at room temperature for 2 h. After the reaction completed, the mixture was diluted with DCM (10 mL) and washed with saturated NaHCO₃ (5 mL), saturated NH₄Cl (5 mL). After extraction, the organic layer was washed with bine (5 mL), dried over Na₂SO₄(s), concentrated, and purified by TLC (DCM/Acetone=10/1), to afford a compound I-F (0.1 g, yield-83.85%).

### (3) Preparation of Compound 3 from Compound I-E and Compound I-F

Compound 3 was prepared via the route shown below:

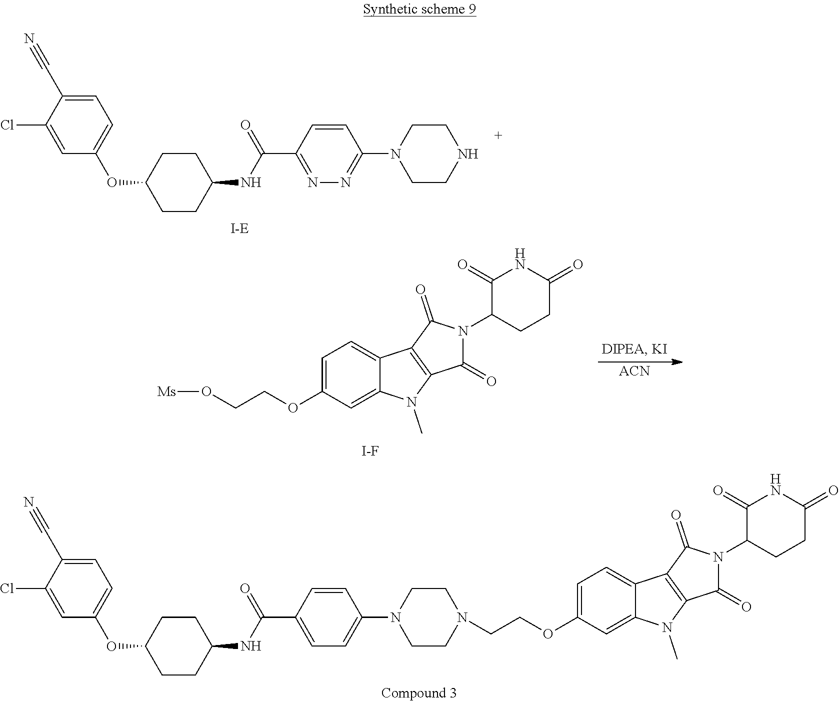

To a solution of I-E (100 mg, 0.227 mmol), I-F (102 mg, 0.227 mmol), KI (113 mg, 3.0 eq) and DIPEA (0.2 mL, 5.0 eq) in dry CH₃CN (1 mL) was heated to 75° C. for 17 h. After the reaction completed, diluted with EA (10 mL) and alkalized by Na₂CO_{3(aq)}, the organic layer was washed with brine, dried over Na₂SO₄(s), concentrated and purified by preparative TLC with a mobile phase (DCM/Acetone=9/1) to get Compound 3 (104 mg, 58%). MS: m/z 794.4 (M⁺+1); ¹H NMR (DMSO-d6) δ 11.06 (s, 1H), 8.61 (d, 1H), 7.86-7.82 (m, 2H), 7.62 (d, 1H), 7.39 (d, 1H), 7.37 (d, 1H), 7.33 (d, 1H), 7.13 (dd, 1H), 7.02 (dd, 1H), 6.95 (d, 1H), 4.98 (dd, 1H), 4.57-4.49 (m, 1H), 4.44 (q, 2H), 4.26 (t, 2H), 3.96 (s, 3H), 3.90-3.82 (m, 1H), 3.74 (bs, 4H), 2.93-2.85 (m, 1H), 2.84 (t, 2H), 2.66 (bs, 4H), 2.62-2.49 (m, 2H), 2.14-2.07 (m, 2H), 2.07-2.00 (m, 1H), 1.93-1.86 (m, 2H), 1.69-1.59 (m, 2H), 1.56-1.45 (m, 2H).

The preparation of Compounds 1, 2 and 3 of the present invention is exemplified above. Other compounds in the present invention can be synthesized by similar methods shown in Synthetic Method A (Synthetic schemes 1 to 3), Synthetic Method B (Synthetic schemes 4 to 6) or Synthetic Method C (Synthetic schemes 7 to 9), or by any known synthesis methods based on the general knowledge of organic chemistry.

Following Synthetic Method A (Synthetic schemes 1 to 3), Synthetic Method B (Synthetic schemes 4 to 6), or Synthetic Method C (Synthetic schemes 7 to 9), compounds 4-223 were prepared by a method similar to Synthetic Method A or Synthetic Method B with changing one or more starting materials to obtain the desired products.

Compound 4: MS: m/z 834.9 (M⁺+1).

Compound 5: MS: m/z 835.9 (M⁺+1).

Compound 6: MS: m/z 821.6 (M⁺+1).

Compound 7: MS: m/z 824.1 (M⁺+1).

Compound 8: MS: m/z 835.5 (M⁺+1); ¹H NMR (CD₃CN) δ 8.86 (bs, 1H), 8.58 (d, 1H), 7.90 (dd, 1H), 7.69 (d, 1H), 7.63 (d, 1H), 7.10 (d, 1H), 7.05 (d, 1H), 7.00 (dd, 1H), 6.93 (dd, 1H), 6.73 (d, 1H), 6.49 (d, 1H), 4.86 (q, 1H), 4.22 (s, 1H), 4.17 (t, 2H), 4.08-4.04 (m, 1H), 3.93 (s, 3H), 3.63-3.62 (m, 4H), 2.79-2.63 (m, 3H), 2.57-2.55 (m, 2H), 2.54-2.53 (m, 4H), 2.02-2.00 (m, 2H), 1.97 (m, 1H), 1.23 (s, 6H), 1.15 (s, 6H).

Compound 9: MS: m/z 821.3 (M⁺+1); ¹H NMR (CD₃CN) δ 8.86 (bs, 1H), 8.24 (d, 1H), 8.08 (d, 1H), 7.91 (d, 1H), 7.69 (d, 1H), 7.64 (d, 1H), 7.33 (dd, 1H), 7.11 (d, 1H), 7.09 (d, 1H), 7.00 (dd, 1H), 6.94 (dd, 1H), 4.87-4.84 (m, 1H), 4.26 (s, 1H), 4.24 (t, 2H), 4.01 (d, 1H), 3.93 (s, 3H), 3.37-3.36 (m, 4H), 2.87 (t, 2H), 2.79-2.63 (m, 7H), 2.14 (m, 1H), 1.22 (s, 6H), 1.16 (s, 6H).

Compound 10: MS: m/z 821.5 (M⁺+1); ¹H NMR (DMSO-d₆) δ 11.06 (s, 1H), 8.35 (d, 1H), 8.10 (d, 1H), 7.90 (d, 1H), 7.84 (d, 1H), 7.69 (d, 1H), 7.44 (dd, 1H), 7.25 (d, 1H), 7.21 (s, 1H), 7.10 (dd, 1H), 7.03 (dd, 1H), 5.00 (dd, 1H), 4.43 (s, 1H), 4.21 (t, 2H), 3.97 (s, 3H), 3.94 (d, 1H), 3.37 (m, 4H), 2.92-2.86 (m, 1H), 2.81 (t, 2H), 2.68 (m, 4H), 2.60-2.55 (m, 2H), 2.05-2.03 (m, 1H), 1.19 (s, 6H), 1.12 (s, 6H).

Compound 11: MS: m/z 835.4 (M⁺+1).

Compound 12: MS: m/z 835.0 (M⁺+1); ¹H NMR (DMSO-d₆) δ 11.06 (s, 1H), 7.90 (d, 1H), 7.75 (d, 2H), 7.62 (d, 1H), 7.51 (d, 1H), 7.27 (d, 1H), 7.20 (d, 1H), 7.02-6.99 (m, 2H), 6.97 (d, 2H), 4.97 (dd, 1H), 4.32 (s, 1H), 4.16 (t, 2H), 4.05 (d, 1H), 3.96 (s, 3H), 3.27 (m, 4H), 2.92-2.86 (m, 1H), 2.61-2.50 (m, 8H), 2.04-1.96 (m, 3H), 1.21 (s, 6H), 1.12 (s, 6H).

Compound 13: MS: m/z 850.5 (M⁺+1); ¹H NMR (DMSO-d₆) δ 11.06 (s, 1H), 8.36 (d, 1H), 8.10 (d, 1H), 7.90 (d, 1H), 7.85 (d, 1H), 7.82 (d, 1H), 7.44 (dd, 1H), 7.25 (d, 1H), 7.20 (d, 1H), 7.09 (dd, 1H), 7.03 (dd, 1H), 5.10-5.05 (m, 1H), 5.01 (dd, 1H), 4.43 (s, 1H), 4.21 (t, 2H), 3.94 (d, 1H), 3.37 (m, 4H), 2.92-2.86 (m, 1H), 2.81 (t, 2H), 2.68 (m, 4H), 2.61-2.50 (m, 2H), 2.07-2.05 (m, 1H), 1.59-1.57 (m, 6H), 1.19 (s, 6H), 1.12 (s, 6H).

Compound 14: MS: m/z 839.0 (M⁺+1).

Compound 15: MS: m/z 820.3 (M⁺+1); ¹H NMR (DMSO-d₆) δ 11.04 (bs, 1H), 7.88 (d, 1H), 7.73 (d, 2H), 7.67 (d, 1H), 7.49 (d, 1H), 7.18 (s, 2H), 7.09 (dd, 1H), 6.99-6.95 (m, 3H), 4.98 (dd, 1H), 4.30 (s, 1H), 4.19 (t, 2H), 4.04 (d, 1H), 3.96 (s, 3H), 3.26 (m, 4H), 2.90-2.85 (m, 1H), 2.78 (t, 2H), 2.65 (m, 4H), 2.58-2.55 (m, 2H), 2.03-2.01 (m, 1H), 1.19 (s, 6H), 1.11 (s, 6H).

Compound 16: MS: m/z 849.3 (M⁺+1); ¹H NMR (DMSO-d₆) δ 11.06 (s, 1H), 8.62 (s, 1H), 7.96 (d, 1H), 7.90 (d, 1H), 7.82 (d, 1H), 7.61 (d, 1H), 7.21-7.20 (m, 2H), 7.09 (d, 1H), 7.00 (d, 1H), 6.88 (d, 1H), 5.10-5.05 (m, 1H), 5.01 (dd, 1H), 4.30 (s, 1H), 4.21 (t, 2H), 4.05 (d, 1H), 3.63 (m, 4H), 2.92-2.86 (m, 1H), 2.80 (t, 2H), 2.61 (m, 4H), 2.58-2.50 (m, 2H), 2.07-2.05 (m, 1H), 1.59-1.57 (m, 6H), 1.21 (s, 6H), 1.12 (s, 6H).

Compound 17: MS: m/z 848.5 (M⁺+1); ¹H NMR (DMSO-d₆) δ 11.06 (s, 1H), 7.90 (d, 1H), 7.82 (d, 1H), 7.75 (d, 2H), 7.51 (d, 1H), 7.21-7.20 (m, 2H), 7.09 (d, 1H), 7.00-6.97 (m, 3H), 5.10-5.05 (m, 1H), 5.01 (dd, 1H), 4.32 (s, 1H), 4.21 (t, 2H), 4.05 (d, 1H), 3.28 (m, 4H), 2.92-2.86 (m, 1H), 2.80 (t, 2H), 2.66 (m, 4H), 2.58-2.50 (m, 2H), 2.07-2.05 (m, 1H), 1.59-1.57 (m, 6H), 1.21 (s, 6H), 1.12 (s, 6H).

Compound 18: MS: m/z 848.5 (M⁺+1)

Compound 19: MS: m/z 821.6 (M⁺+1);

Compound 20: MS: m/z 849.5 (M⁺+1); ¹H NMR (DMSO-d₆) δ 11.06 (s, 1H), 8.63 (s, 1H), 7.97 (d, 1H), 7.90 (d, 1H), 7.61 (dd, 2H), 7.34 (d, 1H), 7.20 (d, 1H), 7.02-6.99 (m, 2H), 6.87 (d, 1H), 4.97 (dd, 1H), 4.45-4.41 (m, 2H), 4.29 (s, 1H), 4.17 (t, 2H), 4.06 (d, 1H), 3.62 (m, 4H), 2.91-2.85 (m, 1H), 2.61-2.57 (m, 1H), 2.52-2.49 (m, 7H), 2.06-2.02 (m, 1H), 2.00-1.95 (m, 2H), 1.42 (t, 3H), 1.21 (s, 6H), 1.11 (s, 6H).

Compound 21: MS: m/z 848.6 (M⁺+1); ¹H NMR (DMSO-d₆) δ 11.03 (s, 1H), 7.90 (d, 1H), 7.76 (dd, 2H), 7.62 (d, 1H), 7.51 (d, 1H), 7.33 (s, 1H), 7.19 (s, 1H), 7.02-6.97 (m, 4H), 4.98 (dd, 1H), 4.43(q, 2H), 4.32 (s, 1H), 4.17 (t, 2H), 4.04 (d, 1H), 3.27 (m, 4H), 2.91-2.85 (m, 1H), 2.61-2.57 (m, 1H), 2.56-2.50 (m, 4H), 2.50-2.49 (m, 2H), 2.07-2.02 (m, 1H), 2.00-1.96 (m, 2H), 1.41 (t, 3H), 1.22 (s, 6H), 1.12 (s, 6H).

Compound 22: MS: m/z 863.5 (M⁺+1); ¹H NMR (DMSO-d₆) δ 11.04 (s, 1H), 8.62 (s, 1H), 7.96 (d, 1H), 7.90 (d, 1H), 7.62 (dd, 2H), 7.34 (d, 1H), 7.21 (d, 1H), 7.04-6.97 (m, 2H), 6.85 (d, 1H), 4.97 (dd, 1H), 4.42-4.33 (m, 2H), 4.30 (s, 1H), 4.16 (t, 2H), 4.06 (d, 1H), 3.62 (m, 4H), 2.93-2.83 (m, 1H), 2.63-2.55 (m, 1H), 2.55-2.49 (m, 7H), 2.08-2.02 (m, 1H), 2.00-1.95 (m, 2H), 1.88-1.81 (m, 2H), 1.42 (t, 3H), 1.21 (s, 6H), 1.11 (s, 6H), 0.86 (t, 3H).

Compound 23: MS: m/z 862.5 (M⁺+1); ¹H NMR (DMSO-d₆) δ 11.05 (s, 1H), 7.91 (d, 1H), 7.75 (d, 2H), 7.63 (d, 1H), 7.51 (d, 1H), 7.34 (s, 1H), 7.20 (s, 1H), 7.04-6.95 (m, 4H), 4.98 (dd, 1H), 4.37 (q, 2H), 4.32 (s, 1H), 4.16 (t, 2H), 4.05 (d, 1H), 3.27 (m, 4H), 2.93-2.84 (m, 1H), 2.61-2.58 (m, 1H), 2.56-2.50 (m, 4H), 2.50-2.49 (m, 2H), 2.07-2.02 (m, 1H), 2.01-1.94 (m, 2H), 1.89-1.81 (m, 2H), 1.21 (s, 6H), 1.12 (s, 6H), 0.86 (t, 3H).

Compound 24: MS: m/z 835.3 (M⁺+1); ¹H NMR (DMSO-d₆) δ 11.08 (s, 1H), 8.62 (s, 1H), 7.96 (d, 1H), 7.89 (d, 1H), 7.61 (d, 1H), 7.30-7.22 (m, 2H), 7.20 (s, 1H), 7.02 (dd, 1H), 6.96 (d, 1H), 6.87 (d, 1H), 5.00 (dd, 1H), 4.31 (s, 1H), 4.26-4.17 (m, 5H), 4.05 (d, 1H), 3.62 (m, 4H), 2.93-2.83 (m, 1H), 2.63-2.55 (m, 1H), 2.55-2.49 (m, 7H), 2.07-2.03 (m, 3H), 1.21 (s, 6H), 1.12 (s, 6H).

Compound 25: MS: m/z 834.3 (M⁺+1); ¹H NMR (DMSO-d₆) δ 11.07 (s, 1H), 7.90 (d, 1H), 7.75 (d, 2H), 7.50 (d, 1H), 7.31-7.21 (m, 2H), 7.19 (s, 1H), 7.01 (dd, 1H), 6.99-6.94 (m, 3H), 5.00 (dd, 1H), 4.32 (s, 1H), 4.26-4.20 (m, 5H), 4.05 (d, 1H), 3.26 (m, 4H), 2.94-2.85 (m, 1H), 2.63-2.59 (m, 1H), 2.59-2.53 (m, 7H), 2.07-2.02 (m, 3H), 1.21 (s, 6H), 1.12 (s, 6H).

Compound 26: MS: m/z 821.4 (M⁺+1); ¹H NMR (DMSO-d₆) δ 11.05 (s, 1H), 8.60 (s, 1H), 8.05 (d, 1H), 7.97 (d, 1H), 7.85 (d, 1H), 7.61 (d, 1H), 7.40 (s, 1H), 7.32 (s, 1H), 7.15 (d, 1H), 7.01 (d, 1H), 6.84 (d, 1H), 4.97 (dd, 1H), 4.60-4.48 (m, 1H), 4.42 (q, 2H), 4.16 (t, 2H), 3.87-3.74 (m, 1H), 3.60 (m, 4H), 2.95-2.82 (m, 1H), 2.65-2.55 (m, 1H), 2.54-2.49 (m, 7H), 2.15-2.08 (m, 2H), 2.08-2.01 (m, 1H), 2.00-1.95 (m, 2H), 1.95-1.88 (m, 2H), 1.56-1.44 (m, 4H), 1.41 (t, 3H)

Compound 27: MS: m/z 820.4 (M⁺+1); ¹H NMR (DMSO-d₆) δ 11.07 (s, 1H), 8.01 (d, 1H), 7.86 (d, 1H), 7.74 (d, 2H), 7.63 (d, 1H), 7.40 (s, 1H), 7.33 (s, 1H), 7.14 (d, 1H), 7.02 (d, 1H), 6.93 (d, 2H), 4.98 (dd, 1H), 4.58-4.49 (m, 1H), 4.43 (q, 2H), 4.14 (t, 2H), 3.86-3.74 (m, 1H), 3.24 (m, 4H), 2.95-2.80 (m, 1H), 2.63-2.57 (m, 1H), 2.57-2.49 (m, 7H), 2.13-2.07 (m, 2H), 2.07-2.01 (m, 1H), 2.00-1.94 (m, 2H), 1.93-1.84 (m, 2H), 1.58-1.44 (m, 4H), 1.41 (t, 3H)

Compound 28: MS: m/z 863.0 (M⁺+1); ¹H NMR (DMSO-d₆) δ 11.06 (s, 1H), 8.75 (s, 2H), 8.12 (d, 1H), 7.87 (d, 1H), 7.62 (bs, 1H), 7.39 (d, 1H), 7.32 (bs, 1H), 7.15 (dd, 1H), 7.03 (bs, 1H), 4.98 (dd, 1H), 4.74-4.72 (m, 2H), 4.57-4.53 (m, 2H), 3.96 (s, 3H), 3.83-3.76 (m, 1H), 3.03-2.93 (m, 2H), 2.93-2.86 (m, 1H), 2.78-2.67 (m, 1H), 2.64-2.56 (m, 1H), 2.52-2.49 (m, 1H), 2.34-2.15 (m, 4H), 2.14-2.07 (m, 3H), 2.07-1.97 (m, 3H), 1.92-1.76 (m, 5H), 1.75-1.61 (m, 2H), 1.55-1.46 (m, 4H), 1.11-0.98 (m, 2H).

Compound 29: MS: m/z 822.3 (M⁺+1); ¹H NMR (DMSO-d₆) δ 11.05 (s, 1H), 8.61 (d, 1H), 7.85 (d, 1H), 7.84 (d, 1H), 7.62 (d, 1H), 7.39 (d, 1H), 7.36-7.32 (m, 2H), 7.13 (dd, 1H), 7.01 (dd, 1H), 4.97 (dd, 1H), 4.58-4.49 (m, 1H), 4.42 (q, 2H), 4.16 (t, 2H), 3.91-3.79 (m, 1H), 3.71 (bs, 4H), 2.93-2.83 (m, 1H), 2.64-2.56 (m, 1H), 2.56-2.49 (m, 7H), 2.14-2.07 (m, 2H), 2.07-2.02 (m, 1H), 2.02-1.95 (m, 2H), 1.94-1.86 (m, 2H), 1.70-1.59 (m, 2H), 1.56-1.47 (m, 2H), 1.41 (t, 3H).

Compound 30: MS: m/z 835.5 (M⁺+1); ¹H NMR (DMSO-d₆) δ 11.06 (s, 1H), 8.63 (d, 1H), 7.96 (dd, 1H), 7.90 (d, 1H), 7.61 (dd, 2H), 7.39 (d, 1H), 7.20 (d, 1H), 7.02-6.99 (m, 2H), 6.88 (dd, 1H), 4.97 (dd, 1H), 4.48-4.40 (m, 2H), 4.30 (s, 1H), 4.25 (t, 2H), 4.06 (d, 1H), 3.63 (m, 4H), 2.92-2.85 (m, 1H), 2.82 (t, 2H), 2.62 (m, 4H), 2.60-2.54 (m, 1H), 2.50 (m, 1H), 2.08-2.03 (m, 1H), 1.43 (t, 3H), 1.21 (s, 6H), 1.11 (s, 6H).

Compound 31: MS: m/z 807.4 (M⁺+1); ¹H NMR (DMSO-d₆) δ 11.05 (s, 1H), 8.60 (d, 1H), 8.05 (d, 1H), 7.96 (d, 1H), 7.86 (d, 1H), 7.61 (d, 1H), 7.39 (m, 2H), 7.14 (dd, 1H), 7.01 (dd, 1H), 6.86 (d, 1H), 4.97 (dd, 1H), 4.59-4.49 (m, 1H), 4.44 (q, 2H), 4.25 (t, 2H), 3.84-3.74 (m, 1H), 3.61 (m, 4H), 2.94-2.85 (m, 1H), 2.82 (t, 2H), 2.62 (m, 4H), 2.59-2.53 (m, 1H), 2.49 (m, 1H), 2.10-2.08 (m, 2H), 2.08-2.02 (m, 1H), 1.94-1.86 (m, 2H), 1.57-1.45 (m, 4H), 1.42 (t, 3H).

Compound 32: MS: m/z 828.5 (M⁺+23); ¹H NMR (DMSO-d₆) δ 11.06 (s, 1H), 7.96 (d, 1H), 7.85 (d, 1H), 7.74 (d, 2H), 7.61 (d, 1H), 7.39 (m, 2H), 7.14 (dd, 1H), 7.01 (dd, 1H), 6.95 (d, 1H), 4.97 (dd, 1H), 4.59-4.49 (m, 1H), 4.44 (q, 2H), 4.25 (t, 2H), 3.83-3.75 (m, 1H), 3.27 (m, 4H), 2.92-2.85 (m, 1H), 2.82 (t, 2H), 2.67 (m, 4H), 2.62-2.55 (m, 1H), 2.49 (m, 1H), 2.12-2.07 (m, 2H), 2.07-2.01 (m, 1H), 1.92-1.86 (m, 2H), 1.58-1.45 (m, 4H), 1.42 (t, 3H).

Compound 33: MS: m/z 808.4 (M⁺+1); ¹H NMR (DMSO-d₆) δ 11.06 (s, 1H), 8.61 (d, 1H), 7.87-7.81 (m, 2H), 7.62 (d, 1H), 7.39 (m, 2H), 7.37 (d, 1H), 7.13 (d, 1H), 7.01 (d, 1H), 6.95 (d, 1H), 4.98 (dd, 1H), 4.58-4.49 (m, 1H), 4.44 (q, 2H), 4.26 (t, 2H), 3.91-3.81 (m, 1H), 3.73 (m, 4H), 2.93-2.85 (m, 1H), 2.66 (m, 4H), 2.63-2.55 (m, 1H), 2.49 (m, 1H), 2.12-2.07 (m, 2H), 2.07-2.00 (m, 1H), 1.93-1.86 (m, 2H), 1.68-1.59 (m, 2H), 1.56-1.46 (m, 2H), 1.42 (t, 3H).

Compound 34: MS: m/z 808.3 (M⁺+1).

Compound 35: MS: m/z 778.3 (M⁺+1).

Compound 36: MS: m/z 873.6 (M⁺+23); ¹H NMR (DMSO-d₆) δ 11.06 (s, 1H), 8.59 (d, 1H), 7.86 (d, 1H), 7.79 (d, 1H), 7.62 (d, 1H), 7.39 (d, 1H), 7.34-7.33 (m, 2H), 7.14 (dd, 1H), 7.02 (dd, 1H), 4.98 (dd, 1H), 4.56-4.51 (m, 1H), 4.42 (q, 2H), 4.08-4.00 (m, 4H), 3.89-3.82 (m, 1H), 3.65-3.61 (m, 1H), 3.57-3.55 (m, 1H), 3.51-3.48 (m, 1H), 3.47-3.43 (m, 2H), 2.92-2.86 (m, 1H), 2.62-2.57 (m, 2H), 2.23-2.18 (m, 1H), 2.12-2.09 (m, 2H), 2.08-2.03 (m, 1H), 1.93-1.88 (m, 4H), 1.67-1.60 (m, 2H), 1.54-1.48 (m, 4H), 1.40 (t, 3H), 1.06 (d, 3H).

Compound 37: MS: m/z 850.3 (M⁺+1).

Compound 38: MS: m/z 821.6 (M⁺+1). ¹H NMR (DMSO-d₆) δ 11.06 (s, 1H), 9.10 (bs, 2H), 8.60 (d, 1H), 7.87 (d, 1H), 7.63 (d, 1H), 7.40 (d, 1H), 7.35 (bs, 1H), 7.15 (dd, 1H), 7.02 (dd, 1H), 4.98 (dd, 1H), 4.60-4.55 (m, 1H), 4.44 (q, 2H), 4.17 (bs, 2H), 3.87-3.82 (m, 1H), 3.08-2.95 (m, 1H), 2.93-2.86 (m, 1H), 2.64-2.56 (m, 2H), 2.56-2.48 (m, 6H), 2.15-2.09 (m, 4H), 2.08-2.02 (m, 3H), 2.00-1.88 (m, 4H), 1.58-1.49 (m, 4H), 1.43 (t, 3H).

Compound 39: MS: m/z 808.5 (M⁺+1); ¹H NMR (DMSO-d₆) δ 11.06 (s, 1H), 8.61 (d, 1H), 7.88-7.81 (m, 2H), 7.62 (d, 1H), 7.38 (d, 1H), 7.35 (d, 1H), 7.28 (d, 1H) 7.13 (dd, 1H), 7.01 (dd, 1H), 6.84 (d, 1H), 4.97 (dd, 1H), 4.57-4.49 (m, 1H), 4.16 (t, 2H), 3.95 (s, 3H), 3.90-3.80 (m, 1H), 3.71 (m, 4H), 2.93-2.83 (m, 1H), 2.63-2.58 (m, 1H), 2.56-2.49 (m, 7H), 2.14-2.07 (m, 2H), 2.07-1.96 (m, 3H), 1.94-1.85 (m, 2H), 1.70-1.59 (m, 2H), 1.56-1.45 (m, 2H).

Compound 40: MS: m/z 836.3 (M⁺+1); ¹H NMR (DMSO-d₆) δ 11.06 (s, 1H), 8.25 (d, 1H), 7.91 (d, 1H), 7.86 (d, 1H), 7.62 (d, 2H), 7.39 (d, 1H), 7.28 (d, 1H), 7.25 (d, 1H), 7.02-6.99 (m, 2H), 4.98 (dd, 1H), 4.46 (s, 1H), 4.17 (t, 2H), 4.00 (d, 1H), 3.96 (s, 3H), 3.74 (m, 4H), 2.92-2.85 (m, 1H), 2.62-2.58 (m, 1H), 2.56-2.49 (m, 7H), 2.06-2.02 (m, 1H), 2.02-1.96 (m, 2H), 1.22 (s, 6H), 1.14 (s, 6H).

Compound 41: MS: m/z 807.5 (M⁺+1); ¹H NMR (DMSO-d₆) δ 11.06 (s, 1H), 8.62 (d, 1H), 8.23(s, 1H), 7.86-7.85 (m, 2H), 7.70 (d, 1H), 7.54 (dd, 1H), 7.41-7.37 (m, 2H), 7.37 (d, 1H), 7.14 (dd, 1H), 5.00 (dd, 1H), 4.50 (m, 1H), 3.94 (s, 3H), 3.90-3.83 (m, 1H), 3.81 (m, 4H), 3.29 (s, 2H), 2.93-2.85 (m, 1H), 2.70 (m, 4H), 2.62-2.59 (m, 1H), 2.58-2.56 (m, 1H), 2.13-2.07 (m, 2H), 2.07-2.00 (m, 1H), 1.94-1.86 (m, 2H), 1.68-1.59 (m, 2H), 1.56-1.46 (m, 2H).

Compound 42: MS: m/z 834.4 (M⁺+1); ¹H NMR (DMSO-d₆) δ 11.06 (s, 1H), 10.08 1H), 8.63 (s, 1H), 8.22 (s, 1H), 7.96 (d, 1H), 7.89 (d, 1H), 7.69 (d, 1H), 7.64 (d, 1H), 7.53 (d, 1H), 7.20 (d, 1H), 7.00 (dd, 1H), 6.89 (d, 1H), 4.99 (dd, 1H), 4.30 (s, 1H), 4.05 (d, 1H), 3.93 (s, 3H), 3.71 (m, 4H), 3.26 (s, 2H), 2.92-2.83 (m, 1H), 2.65 (m, 4H), 2.62-2.59 (m, 1H), 2.59-2.56 (m, 1H), 2.08-2.00 (m, 1H), 1.21 (s, 6H), 1.12 (s, 6H).

Compound 43: MS: m/z 808.5 (M⁺+1); ¹H NMR (DMSO-d₆) δ 11.06 (s, 1H), 8.61 (d, 1H), 7.88-7.80 (m, 2H), 7.39 (d, 1H), 7.36 (d, 1H), 7.30-7.21 (m, 2H) 7.13 (dd, 1H), 6.96 (d, 1H), 5.00 (dd, 1H), 4.58-4.50 (m, 1H), 4.26-4.20 (m, 2H), 4.22 (s, 3H), 3.90-3.81 (m, 1H), 3.71 (m, 4H), 2.94-2.85 (m, 1H), 2.63-2.58 (m, 1H), 2.56-2.49 (m, 7H), 2.15-2.08 (m, 2H), 2.08-2.00 (m, 3H), 1.94-1.86 (m, 2H), 1.70-1.59 (m, 2H), 1.56-1.46 (m, 2H).

Compound 44: MS: m/z 807.2 (M⁺+1); ¹H NMR (DMSO-d₆) δ 11.06 (s, 1H), 9.10 (bs, 2H), 8.62 (bs, 1H), 7.87 (d, 1H), 7.63 (d, 1H), 7.41 (d, 1H), 7.29 (bs, 1H), 7.15 (dd, 1H), 7.02 (d, 1H), 4.98 (dd, 1H), 4.60-4.56 (m, 1H), 4.17 (bs, 2H), 3.97 (s, 3H), 3.87-3.82 (m, 1H), 3.08-2.95 (m, 1H), 2.93-2.86 (m, 1H), 2.63-2.60 (m, 1H), 2.59-2.50 (m, 7H), 2.15-2.08 (m, 4H), 2.06-2.01 (m, 3H), 1.97-1.89 (m, 4H), 1.58-1.48 (m, 4H).

Compound 45: MS: m/z 793.4 (M⁺+1); ¹H NMR (DMSO-d₆) δ 11.06 (s, 1H), 9.09 (s, 2H), 8.62 (d, 1H), 7.87 (d, 1H), 7.62 (d, 1H), 7.40 (d, 1H), 7.35 (bs, 1H), 7.15 (dd, 1H), 7.03 (d, 1H), 4.98 (dd, 1H), 4.60-4.55 (m, 1H), 4.24 (bs, 2H), 3.97 (s, 3H), 3.87-3.81 (m, 1H), 3.08 (bs, 2H), 2.93-2.86 (m, 2H), 2.81 (bs, 2H), 2.62-2.56 (m, 1H), 2.52-2.49 (m, 3H), 2.27-2.19 (m, 1H), 2.12-2.08 (m, 2H), 2.06-2.02 (m, 1H), 1.98-1.90 (m, 3H), 1.89-1.81 (m, 1H), 1.58-1.48 (m, 4H).

Compound 46: MS: m/z 836.4 (M⁺+1); ¹H NMR (DMSO-d₆) δ 11.05 (s, 1H), 8.53 (d, 1H), 7.85 (d, 1H), 7.80 (d, 1H), 7.60 (d, 1H), 7.38 (d, 1H), 7.26 (s, 1H), 7.17 (d, 1H), 7.13 (dd, 1H), 6.94 (dd, 1H), 4.97 (dd, 1H), 4.56-4.49 (m, 1H), 4.41 (q, 2H), 4.06 (t, 2H), 3.90-3.80 (m, 3H), 3.76 (bs, 2H), 2.92-2.84 (m, 1H), 2.80 (bs, 2H), 2.66-2.57 (m, 4H), 2.56-2.49 (m, 2H), 2.12-2.06 (m, 2H), 2.06-2.00 (m, 1H), 1.92-1.82 (m, 6H), 1.66-1.57 (m, 2H), 1.55-1.46 (m, 2H), 1.40 (t, 3H).

Compound 47: MS: m/z 848.4 (M⁺+1); ¹H NMR (DMSO-d₆) δ 11.05 (s, 1H), 8.58 (d, 1H), 7.86 (d, 1H), 7.82 (d, 1H), 7.62 (d, 1H), 7.38 (d, 1H), 7.33 (s, 1H), 7.20 (d, 1H), 7.13 (dd, 1H), 7.02 (d, 1H), 4.97 (dd, 1H), 4.58-4.49 (m, 1H), 4.42 (q, 2H), 4.20 (t, 2H), 4.05-3.94 (m, 2H), 3.90-3.80 (m, 1H), 3.42 (bs, 2H), 3.13 (d, 2H), 2.93-2.84 (m, 1H), 2.63-2.54 (m, 4H), 2.14-2.06 (m, 2H), 2.06-2.01 (m, 1H), 2.00-1.95 (m, 2H), 1.95-1.86 (m, 4H), 1.68-1.58 (m, 2H), 1.58-1.46 (m, 4H), 1.41 (t, 3H).

Compound 48: MS: m/z 862.4 (M⁺+1); ¹H NMR (DMSO-d₆) δ 11.05 (s, 1H), 8.54 (d, 1H), 7.85 (d, 1H), 7.82 (d, 1H), 7.62 (d, 1H), 7.38 (d, 1H), 7.33 (d, 1H), 7.13 (dd, 1H), 7.00 (dd, 1H), 6.85 (d, 1H), 4.97 (dd, 1H), 4.58-4.50 (m, 1H), 4.44 (q, 2H), 4.13 (t, 2H), 3.84 (bs, 5H), 2.93-2.84 (m, 1H), 2.64-2.56 (m, 3H), 2.47-2.43 (m, 3H), 2.14-2.07 (m, 2H), 2.07-2.00 (m, 1H), 1.97-1.86 (m, 4H), 1.79 (bs, 4H), 1.70-1.59 (m, 2H), 1.69-1.58 (m 2H), 1.56-1.46 (m, 2H), 1.42 (t, 3H).

Compound 49: MS: m/z 890.4 (M⁺+1); ¹H NMR (DMSO-d₆) δ 11.06 (s, 1H), 8.22 (d, 1H), 7.90 (d, 1H), 7.85 (d, 1H), 7.63 (d, 1H), 7.33 (d, 1H), 7.25 (d, 1H), 7.03 (dd, 1H), 7.00 (dd, 1H), 6.88 (d, 1H), 4.98 (dd, 1H), 4.46 (s, 1H), 4.44 (q, 2H), 4.14 (bs, 2H), 4.00 (d, 1H), 3.88 (bs, 4H), 2.93-2.85 (m, 1H), 2.75 (bs, 2H), 2.62-2.58 (m, 1H), 2.58-2.55 (m, 1H), 2.54-2.49 (m, 4H), 2.08-2.01 (m, 1H), 2.01-1.93 (m, 2H), 1.83 (bs, 4H), 1.42 (t, 3H), 1.21 (s, 6H), 1.14 (s, 6H).

Compound 50: MS: m/z 835.5 (M⁺+1); ¹H NMR (DMSO-d₆) δ 11.06 (s, 1H), 10.10 (s, 1H), 8.26 (d, 1H), 8.23 (s, 1H), 7.90 (d, 1H), 7.87 (d, 1H), 7.70 (d, 1H), 7.54 (d, 1H), 7.41 (d, 1H), 7.25 (d, 1H), 7.03 (dd, 1H), 4.99 (dd, 1H), 4.46 (s, 1H), 4.00 (d, 1H), 3.94 (s, 3H), 3.83 (bs, 4H), 3.29 (s, 2H), 2.91-2.86 (m, 1H), 2.70 (bs, 4H), 2.60-2.57 (m, 1H), 2.56-2.49 (m, 1H), 2.07-2.03 (m, 1H), 1.22 (s, 6H), 1.14 (s, 6H).

Compound 51: MS: m/z 876.5 (M⁺+1); ¹H NMR (DMSO-d₆) δ 11.06 (s, 1H), 8.22 (d, 1H), 7.90 (d, 1H), 7.84 (d, 1H), 7.62 (d, 1H), 7.38 (d, 1H), 7.25 (d, 1H), 7.04 (dd, 1H), 7.01 (dd, 1H), 6.88 (d, 1H), 4.98 (dd, 1H), 4.46 (s, 1H), 4.44 (q, 2H), 4.21 (bs, 2H), 4.00 (d, 1H), 3.87 (s, 4H), 2.93-2.85 (m, 1H), 2.75 (bs, 2H), 2.62-2.58 (m, 1H), 2.58-2.54 (m, 1H), 2.54-2.49 (m, 4H), 2.08-2.01 (m, 1H), 1.81 (bs, 4H), 1.43 (t, 3H), 1.21 (s, 6H), 1.14 (s, 6H).

Compound 52: MS: m/z 822.3 (M⁺+1); ¹H NMR (DMSO-d₆) δ 11.05 (s, 1H), 8.56 (d, 1H), 7.85 (d, 1H), 7.79 (d, 1H), 7.59 (d, 1H), 7.38 (s, 1H), 7.33 (s, 1H), 7.18 (d, 1H), 7.14 (d, 1H), 6.96 (d, 1H), 4.97 (dd, 1H), 4.57-4.49 (m, 1H), 4.42 (q, 2H), 4.16 (t, 2H), 3.92-3.80 (m, 3H), 3.76 (bs, 2H), 2.96-2.89 (m, 4H), 2.89-2.84 (m, 1H), 2.75-2.69 (m, 2H), 2.62-2.55 (m, 1H), 2.55-2.49 (m, 1H), 2.14-2.07 (m, 2H), 2.07-2.00 (m, 1H), 1.93-1.84 (m, 4H), 1.67-1.57 (m, 2H), 1.55-1.46 (m, 2H), 1.40 (t, 3H).

Compound 53: MS: m/z 850.4 (M⁺+1); ¹H NMR (DMSO-d₆) δ 11.05 (s, 1H), 8.22 (d, 1H), 7.90 (d, 1H), 7.82 (d, 1H), 7.60 (d, 1H), 7.34 (d, 1H), 7.25 (d, 1H), 7.20 (d, 1H), 7.03 (dd, 1H), 6.97 (dd, 1H), 4.97 (dd, 1H), 4.45 (s, 1H), 4.42 (q, 2H), 4.17 (t, 2H), 4.00 (d, 1H), 3.88 (bs, 2H), 3.78 (bs, 2H), 2.97-2.91 (m, 4H), 2.91-2.84 (m, 1H), 2.76-2.71 (m, 2H), 2.63-2.55 (m, 1H), 2.55-2.49 (m, 1H), 2.07-2.00 (m, 1H), 1.94-1.87 (m, 2H), 1.41 (t, 3H), 1.22 (s, 6H), 1.14 (s, 6H).

Compound 54: MS: m/z 864.5 (M⁺+1); ¹H NMR (DMSO-d) δ 11.05 (s, 1H), 8.21 (d, 1H), 7.90 (d, 1H), 7.82 (d, 1H), 7.60 (d, 1H), 7.28 (s, 1H), 7.25 (d, 1H), 7.20 (d, 1H), 7.03 (dd, 1H), 6.96 (dd, 1H), 4.97 (dd, 1H), 4.45 (s, 1H), 4.42 (q, 2H), 4.08 (bs, 2H), 3.99 (d, 1H), 3.86 (bs, 2H), 3.77 (bs, 2H), 2.93-2.84 (m, 1H), 2.81 (bs, 2H), 2.68-2.55 (m, 5H), 2.55-2.49 (m, 1H), 2.07-2.00 (m, 1H), 1.94-1.82 (m, 4H), 1.40 (t, 3H), 1.22 (s, 6H), 1.13 (s, 6H).

Compound 55: MS: m/z 836.5 (M⁺+1); ¹H NMR (DMSO-d₆) δ 11.06 (s, 1H), 8.59 (d, 1H), 7.86 (d, 1H), 7.81 (d, 1H), 7.63 (d, 1H), 7.40-7.37 (m, 2H), 7.30 (d, 1H), 7.14 (dd, 1H), 7.02 (dd, 1H), 4.98 (dd, 1H), 4.62 (bs, 1H), 4.57-4.49 (m, 1H), 4.45 (q, 2H), 4.28-4.16 (m, 3H), 3.90-3.81 (m, 1H), 3.42-3.36 (m, 1H), 3.25-3.10 (m, 1H), 2.96-2.84 (m, 3H), 2.82-2.75 (m, 1H), 2.66-2.56 (m, 2H), 2.54-2.49 (m, 1H), 2.14-2.07 (m, 2H), 2.07-2.01 (m, 1H), 1.94-1.86 (m, 2H), 1.68-1.58 (m, 2H), 1.56-1.46 (m, 2H), 1.43 (t, 3H), 1.23 (d, 3H), 0.98 (d, 3H).

Compound 56: MS: m/z 848.5 (M⁺+1); ¹H NMR (DMSO-d₆) δ 11.07 (s, 1H), 10.06 (s, 1H), 8.64 (d, 1H), 8.26 (s, 1H), 7.97 (dd, 1H), 7.90 (d, 1H), 7.70 (d, 1H), 7.62 (d, 1H), 7.55 (dd, 1H), 7.21 (d, 1H), 7.00 (dd, 1H), 6.89 (d, 1H), 5.00 (dd, 1H), 4.41-4.36 (m, 2H), 4.30 (s, 1H), 4.06 (d, 1H), 3.72 (bs, 4H), 3.26 (s, 2H), 2.92-2.86 (m, 1H), 2.67-2.65 (m, 4H), 2.61-2.57 (m, 2H), 2.07-2.03 (m, 1H), 1.44 (t, 3H), 1.22 (s, 6H), 1.12 (s, 6H).

Compound 57: MS: m/z 822.4 (M⁺+1); ¹H NMR (DMSO-d₆) δ 11.06 (s, 1H), 8.61 (d, 1H), 7.87-7.81 (m, 2H), 7.62 (d, 1H), 7.40-7.37 (m, 2H), 7.31 (d, 1H), 7.13 (dd, 1H), 7.03 (dd, 1H), 4.98 (dd, 1H), 4.67 (bs, 1H), 4.57-4.49 (m, 1H), 4.44 (q, 2H), 4.28 (t, 2H), 4.28-4.20 (m, 1H), 3.91-3.81 (m, 1H), 3.22-3.09 (m, 2H), 2.96 (d, 1H), 2.93-2.85 (m, 1H), 2.82 (t, 2H), 2.63-2.49 (m, 2H), 2.43-2.37 (m, 1H), 2.26-2.18 (m, 1H), 2.14-2.07 (m, 2H), 2.07-2.00 (m, 1H), 1.93-1.86 (m, 2H), 1.68-1.59 (m, 2H), 1.56-1.46 (m, 2H), 1.43 (t, 3H), 1.21 (d, 3H).

Compound 58: MS: m/z 836.4 (M⁺+1); ¹H NMR (DMSO-d₆) δ 11.06 (s, 1H), 8.61 (d, 1H), 7.85 (d, 1H), 7.81 (d, 1H), 7.62 (d, 1H), 7.40-7.37 (m, 2H), 7.31 (d, 1H), 7.13 (dd, 1H), 7.02 (dd, 1H), 4.98 (dd, 1H), 4.57-4.49 (m, 1H), 4.50-4.40 (m, 3H), 4.34-4.22 (m, 3H), 3.90-3.82 (m, 1H), 3.20-3.13 (m, 1H), 3.09 (t, 2H), 2.93-2.84 (m, 1H), 2.84-2.78 (m, 2H), 2.63-2.49 (m, 2H), 2.32-2.25 (m, 1H), 2.25-2.18 (m, 1H), 2.14-2.08 (m, 2H), 2.08-2.01 (m, 1H), 1.93-1.86 (m, 3H), 1.69-1.57 (m, 3H), 1.56-1.46 (m, 2H), 1.43 (t, 3H), 0.81 (d, 3H).

Compound 59: MS: m/z 850.4 (M⁺+1); ¹H NMR (DMSO-d₆) δ 11.05 (s, 1H), 8.57 (d, 1H), 7.85 (d, 1H), 7.80 (d, 1H), 7.62 (d, 1H), 7.39 (d, 1H), 7.33 (d, 1H), 7.29 (d, 1H), 7.13 (dd, 1H), 7.02 (dd, 1H), 4.97 (dd, 1H), 4.62 (bs, 1H), 4.57-4.50 (m, 1H), 4.43 (q, 2H), 4.22-4.13 (m, 3H), 3.90-3.80 (m, 1H), 3.40-3.34 (m, 1H), 3.15-3.10 (m, 1H), 2.93-2.85 (m, 1H), 2.82-2.76 (m, 1H), 2.66-2.58 (m, 2H), 2.58-2.49 (m, 3H), 2.13-2.07 (m, 2H), 2.07-2.01 (m, 1H), 1.98-1.85 (m, 4H), 1.68-1.58 (m, 2H), 1.56-1.46 (m, 2H), 1.41 (t, 3H), 1.22 (d, 3H), 0.92 (d, 3H).

Compound 60: MS: m/z 850.5 (M⁺+1); ¹H NMR (DMSO-d₆) δ 11.06 (s, 1H), 8.61 (d, 1H), 7.85 (d, 1H), 7.80 (d, 1H), 7.62 (d, 1H), 7.38 (d, 1H), 7.33 (s, 1H), 7.30 (d, 1H), 7.14 (dd, 1H), 7.02 (d, 1H), 4.97 (dd, 1H), 4.57-4.49 (m, 1H), 4.49-4.40 (m, 3H), 4.33-4.24 (m, 1H), 4.21-4.14 (m, 2H), 3.90-3.81 (m, 1H), 3.19-3.11 (m, 1H), 2.99 (t, 2H), 2.94-2.84 (m, 1H), 2.63-2.55 (m, 2H), 2.54-2.49 (m, 1H), 2.14-2.02 (m, 6H), 2.00-1.93 (m, 2H), 1.93-1.86 (m, 2H), 1.87-1.80 (m, 1H), 1.69-1.56 (m, 3H), 1.56-1.46 (m, 2H), 1.42 (t, 3H), 0.80 (d, 3H).

Compound 61: MS: m/z 822.3 (M⁺+1); ¹H NMR (DMSO-d₆) 11.06 (bs, 1H), 8.61 (s, 1H), 8.26 (s, 1H), 8.12 (d, 1H), 7.86 (d, 1H), 7.62 (d, 1H), 7.37 (d, 1H), 7.34 (s, 1H), 7.12 (dd, 1H), 7.01 (dd, 1H), 4.97 (dd, 1H), 4.53-4.50 (m, 1H), 4.43 (q, 2H), 4.16 (t, 2H), 3.86-3.80 (m, 1H), 3.70 (bs, 4H), 2.92-2.85 (m, 1H), 2.64-2.56 (m, 1H), 2.56-2.49 (m, 6H), 2.11-2.07 (m, 2H), 2.06-2.02 (m, 1H), 2.00-1.96 (m, 2H), 1.90-1.87 (m, 2H), 1.64-1.58 (m, 2H), 1.53-1.47 (m, 2H), 1.42 (t, 3H).

Compound 62: MS: m/z 808.3 (M⁺+1); ¹H NMR (DMSO-d₆) δ 11.06 (s, 1H), 8.61 (s, 1H), 8.27 (s, 1H), 8.11 (d, 1H), 7.85 (d, 1H), 7.62 (d, 1H), 7.38 (d, 1H), 7.37 (d, 1H), 7.12 (dd, 1H), 7.01 (dd, 1H), 4.97 (dd, 1H), 4.54-4.49 (m, 1H), 4.43 (q, 2H), 4.25 (t, 2H), 3.86-3.81 (m, 1H), 3.72 (bs, 4H), 2.93-2.82 (m, 3H), 2.65 (bs, 4H), 2.62-2.57 (m, 1H), 2.49 (m, 1H), 2.12-2.02 (m, 3H), 1.88-1.86 (m, 2H), 1.63-1.57 (m, 2H), 1.53-1.47 (m, 2H), 1.42 (t, 3H).

Compound 63: MS: m/z 822.6 (M⁺+1); ¹H NMR (DMSO-d₆) δ 11.06 (s, 1H), 8.62 (d, 1H), 7.87-7.83 (m, 2H), 7.62 (d, 1H), 7.39-7.34 (m, 3H), 7.14 (dd, 1H), 7.02 (dd, 1H), 4.98 (dd, 1H), 4.56-4.51 (m, 1H), 4.44 (q, 2H), 4.18 (t, 2H), 3.89-3.83 (m, 1H), 3.72 (bs, 4H), 2.93-2.86 (m, 1H), 2.63-2.58 (m, 1H), 2.58-2.53 (m, 7H), 2.14-2.08 (m, 2H), 2.07-2.03 (m, 1H), 2.02-1.97 (m, 2H), 1.93-1.88 (m, 2H), 1.68-1.62 (m, 2H), 1.55-1.49 (m, 2H), 671.42 (t, 3H).

Compound 64: MS: m/z 834.4 (M⁺+1); ¹H NMR (DMSO-d₆) δ 11.06 (s, 1H), 8.59 (d, 1H), 7.85 (d, 1H), 7.82 (d, 1H), 7.62 (d, 1H), 7.39 (m, 2H), 7.22 (d, 1H), 7.13 (d, 1H), 7.02 (d, 1H), 4.98 (dd, 1H), 4.57-4.50 (m, 1H), 4.44 (q, 2H), 4.25 (t, 2H), 4.07-3.97 (m, 2H), 3.91-3.81 (m, 1H), 3.54 (m, 2H), 3.15 (d, 2H), 2.92-2.86 (m, 1H), 2.86-2.82 (m, 2H), 2.62-2.49 (m, 2H), 2.12-2.07 (m, 2H), 2.07-2.01 (m, 1H), 1.99-1.93 (m, 2H), 1.93-1.86 (m, 2H), 1.67-1.60 (m, 2H), 1.60-1.55 (m, 2H), 1.55-1.47 (m, 2H), 1.42 (t, 3H).

Compound 65: MS: m/z 836.4 (M⁺+1); ¹H NMR (DMSO-d₆) δ 11.05 (s, 1H), 8.61 (d, 1H), 7.86 (d, 1H), 7.83 (d, 1H), 7.62 (d, 1H), 7.39 (d, 1H), 7.34 (d, 1H), 7.30 (d, 1H), 7.13 (dd, 1H), 7.02 (dd, 1H), 4.98 (dd, 1H), 4.66 (bs, 1H), 4.57-4.49 (m, 1H), 4.43 (q, 2H), 4.26-4.14 (m, 3H), 3.91-3.81 (m, 1H), 3.20-3.12 (m, 1H), 3.02 (d, 1H), 2.93-2.83 (m, 2H), 2.63-2.53 (m, 2H), 2.53-2.49 (m, 2H), 2.25-2.18 (m, 1H), 2.14-2.08 (m, 2H), 2.07-2.02 (m, 2H), 2.02-1.94 (m, 2H), 1.93-1.86 (m, 2H), 1.69-1.59 (m, 2H), 1.56-1.46 (m, 2H), 1.41 (t, 3H), 1.20 (d, 3H).

Compound 66: MS: m/z 806.4 (M⁺+1)

Compound 67: MS: m/z 794.4 (M⁺+1); ¹H NMR (DMSO-d₆) δ 11.06 (s, 1H), 8.61 (d, 1H), 7.86-7.82 (m, 2H), 7.62 (d, 1H), 7.39 (d, 1H), 7.37 (d, 1H), 7.33 (d, 1H), 7.13 (dd, 1H), 7.02 (dd, 1H), 6.95 (d, 1H), 4.98 (dd, 1H), 4.57-4.49 (m, 1H), 4.44 (q, 2H), 4.26 (t, 2H), 3.96 (s, 3H), 3.90-3.82 (m, 1H), 3.74 (bs, 4H), 2.93-2.85 (m, 1H), 2.84 (t, 2H), 2.66 (bs, 4H), 2.62-2.49 (m, 2H), 2.14-2.07 (m, 2H), 2.07-2.00 (m, 1H), 1.93-1.86 (m, 2H), 1.69-1.59 (m, 2H), 1.56-1.45 (m, 2H).

Compound 68: MS: m/z 821.4 (M⁺+1); ¹H NMR (DMSO-d₆) δ 11.07 (s, 1H), 9.86 (s, 1H), 8.57 (d, 1H), 8.17 (s, 1H), 7.86 (d, 1H), 7.82 (d, 1H), 7.65 (d, 1H), 7.38 (d, 1H), 7.30 (d, 1H), 7.23 (d, 1H), 7.14 (dd, 1H), 4.99 (dd, 1H), 4.56-4.49 (m, 1H), 4.00-3.88 (m, 2H), 3.92 (s, 3H), 3.88-3.78 (m, 3H), 3.36 (s, 2H), 2.99-2.93 (m, 2H), 2.92-2.84 (m, 1H), 2.81-2.75 (m, 2H), 2.62-2.54 (m, 1H), 2.55-2.49 (m, 1H), 2.14-2.06 (m, 2H), 2.06-1.98 (m, 1H), 1.98-1.92 (m, 2H), 1.92-1.84 (m, 2H), 1.67-1.57 (m, 2H), 1.55-1.46 (m, 2H).

Compound 69: MS: m/z 875.3 (M⁺+1); ¹H NMR (DMSO-d₆) δ 11.07 (s, 1H), 10.00 (s, 1H), 8.23 (d, 1H), 8.22 (s, 1H), 7.90 (d, 1H), 7.84 (d, 1H), 7.69 (d, 1H), 7.54 (d, 1H), 7.25 (d, 1H), 7.03 (dd, 1H), 6.90 (d, 1H), 5.00 (dd, 1H), 4.46 (s, 1H), 4.00 (d, 1H), 3.95 (s, 3H), 3.89 (s, 4H), 3.19 (s, 2H), 2.94-2.84 (m, 1H), 2.63-2.56 (m, 2H), 2.56-2.54 (m, 2H), 2.54-2.49 (m, 2H), 2.07-2.00 (m, 1H), 1.89 (bs, 4H), 1.21 (s, 6H), 1.14 (s, 6H).

Compound 70: MS: m/z 834.3 (M⁺+1); ¹H NMR (DMSO-d₆) δ 11.05 (s, 1H), 8.58 (d, 1H), 7.86 (d, 1H), 7.82 (d, 1H), 7.59 (d, 1H), 7.39 (d, 1H), 7.34 (s, 1H), 7.26 (d, 1H), 7.14 (dd, 1H), 6.96 (d, 1H), 4.97 (dd, 1H), 4.73 (bs, 2H) 4.57-4.50 (m, 1H), 4.42 (q, 2H), 4.19 (t, 2H), 3.90-3.81 (m, 1H), 2.93-2.84 (m, 1H), 2.81-2.75 (m, 2H), 2.75-2.70 (m, 2H), 2.63-2.55 (m, 1H), 2.50-2.49 (m, 1H), 2.47-2.43 (m, 2H), 2.14-2.06 (m, 2H), 2.06-2.00 (m, 1H), 1.99-1.96 (m, 2H), 1.94-1.85 (m, 4H), 1.68-1.58 (m, 2H), 1.56-1.47 (m, 2H), 1.42 (t, 3H).

Compound 71: MS: m/z 848.4 (M⁺+1); ¹H NMR (DMSO-d₆) δ 11.05 (s, 1H), 8.58 (d, 1H), 7.86 (d, 1H), 7.82 (d, 1H), 7.61 (d, 1H), 7.39 (d, 1H), 7.32 (s, 1H), 7.25 (d, 1H), 7.14 (dd, 1H), 7.00 (d, 1H), 4.97 (dd, 1H), 4.72 (bs, 2H), 4.57-4.50 (m, 1H), 4.43 (q, 2H), 4.14 (t, 2H), 3.90-3.82 (m, 1H), 2.93-2.84 (m, 1H), 2.75-2.69 (m, 2H), 2.62-2.55 (m, 1H), 2.50-2.49 (m, 1H), 2.44-2.39 (m, 2H), 2.29-2.24 (m, 2H), 2.14-2.07 (m, 2H), 2.07-2.01 (m, 1H), 1.98-1.93 (m, 2H), 1.93-1.85 (m, 6H), 1.68-1.59 (m, 2H), 1.56-1.47 (m, 2H), 1.41 (t, 3H).

Compound 72: MS: m/z 862.4 (M⁺+1); ¹H NMR (DMSO-d₆) δ 11.06 (s, 1H), 8.58 (d, 1H), 7.86 (d, 1H), 7.80 (d, 1H), 7.62 (d, 1H), 7.38-7.33 (m, 3H), 7.13 (d, 1H), 7.00 (dd, 1H), 4.97 (dd, 1H), 4.56-4.51 (m, 1H), 4.44 (q, 2H), 4.12 (t, 2H), 3.86-3.85 (m, 1H), 3.68 (s, 4H), 3.03 (bs, 4H), 2.92-2.85 (m, 1H), 2.61-2.57 (m, 2H), 2.11-2.09 (m, 2H), 2.05-2.03 (m, 1H), 1.90-1.88 (m, 2H), 1.80 (bs, 2H), 1.75 (bs, 4H), 1.66-1.60 (m, 2H), 1.54-1.48 (m, 2H), 1.42 (t, 3H).

Compound 73: MS: m/z 822.3 (M⁺+1); ¹H NMR (DMSO-d₆) δ 11.05 (s, 1H), 8.57 (d, 1H), 7.85 (d, 1H), 7.82 (d, 1H), 7.61 (d, 1H), 7.40-7.35 (m, 3H), 7.13 (d, 1H), 7.00 (d, 1H), 4.97 (dd, 1H), 4.57-4.49 (m, 1H), 4.43 (q, 2H), 4.22 (t, 2H), 4.19-4.08 (m, 2H), 3.90-3.81 (m, 1H), 3.17-3.09 (m, 1H), 3.07-3.00 (m, 2H), 2.93-2.83 (m, 1H), 2.83-2.75 (m, 1H), 2.68-2.58 (m, 2H), 2.58-2.49 (m, 3H), 2.14-2.07 (m, 2H), 2.07-2.00 (m, 1H), 1.94-1.86 (m, 2H), 1.69-1.58 (m, 2H), 1.56-1.46 (m, 2H), 1.42 (t, 3H). 1.12 (d, 3H).

Compound 74: MS: m/z 836.4 (M⁺+1); ¹H NMR (DMSO-d₆) δ 11.05 (s, 1H), 8.59 (d, 1H), 7.86 (d, 1H), 7.82 (d, 1H), 7.62 (d, 1H), 7.39 (s, 1H), 7.36 (d, 1H), 7.34 (s, 1H), 7.13 (d, 1H), 7.01 (d, 1H), 4.98 (dd, 1H), 4.57-4.49 (m, 1H), 4.43 (q, 2H), 4.18-4.10 (m, 3H), 4.10-4.03 (m, 1H), 3.90-3.81 (m, 1H), 3.07-2.99 (m, 1H), 2.97-2.83 (m, 3H), 2.63-2.55 (m, 1H), 2.55-2.49 (m, 3H), 2.46-2.40 (m, 1H), 2.37-2.31 (m, 1H), 2.14-2.07 (m, 2H), 2.07-2.00 (m, 1H), 1.98-1.87 (m, 4H), 1.69-1.59 (m, 2H), 1.56-1.46 (m, 2H), 1.41 (t, 3H), 1.05 (d, 3H).

Compound 75: MS: m/z 808.5 (M⁺+1); ¹H NMR (DMSO-d₆) δ 11.06 (s, 1H), 8.56 (d, 1H), 7.86 (d, 1H), 7.79 (d, 1H), 7.59 (d, 1H), 7.39 (s, 1H), 7.27 (s, 1H), 7.18 (d, 1H), 7.13 (d, 1H), 6.97 (d, 1H), 4.97 (dd, 1H), 4.57-4.50 (m, 1H), 4.20-4.12 (m, 2H), 3.94 (s, 3H), 3.91-3.81 (m, 3H), 3.76 (bs, 2H), 2.99-2.90 (m, 4H), 2.90-2.84 (m, 1H), 2.77-2.69 (m, 2H), 2.62-2.55 (m, 1H), 2.55-2.49 (m, 1H), 2.14-2.06 (m, 2H), 2.06-2.00 (m, 1H), 1.94-1.85 (m, 4H), 1.67-1.58 (m, 2H), 1.56-1.46 (m, 2H).

Compound 76: MS: m/z 850.4 (M⁺+1); ¹H NMR (DMSO-d₆) δ 11.05 (s, 1H), 8.56 (d, 1H), 7.85 (d, 1H), 7.81 (d, 1H), 7.62 (d, 1H), 7.41-7.35 (m, 2H), 7.32 (s, 1H), 7.14 (dd, 1H), 6.98 (d, 1H), 4.97 (dd, 1H), 4.57-4.50 (m, 1H), 4.42 (q, 2H), 4.33 (d, 2H), 4.11 (t, 2H), 3.91-3.81 (m, 1H), 3.71 (m, 4H), 2.94-2.84 (m, 3H), 2.78-2.70 (m, 2H), 2.66-2.55 (m, 3H), 2.54-2.49 (m, 1H), 2.13-2.07 (m, 2H), 2.07-2.00 (m, 1H), 1.93-1.87 (m, 2H), 1.87-80 (m, 2H), 1.67-1.59 (m, 2H), 1.56-1.46 (m, 2H), 1.41 (t, 3H), 1.12 (d, 6H).

Compound 77: MS: m/z 836.4 (M⁺+1); ¹H NMR (DMSO-d₆) δ 11.05 (s, 1H), 8.56 (d, 1H), 7.85 (d, 1H), 7.82 (d, 1H), 7.60 (d, 1H), 7.41-7.37 (m, 2H), 7.35 (s, 1H), 7.13 (d, 1H), 6.96 (d, 1H), 4.97 (dd, 1H), 4.57-4.50 (m, 1H), 4.43 (q, 2H), 4.40-4.33 (m, 2H), 4.13 (t, 2H), 3.90-3.81 (m, 1H), 3.12 (t, 2H), 2.94-2.84 (m, 1H), 2.82-2.70 (m, 4H), 2.64-2.54 (m, 1H), 2.54-2.49 (m, 1H), 2.15-2.07 (m, 2H), 2.07-2.01 (m, 1H), 1.94-1.87 (m, 2H), 1.68-1.57 (m, 2H), 1.56-1.46 (m, 2H), 1.41 (t, 3H), 1.18 (d, 6H).

Compound 78: MS: m/z 835.7 (M⁺+1); ¹H NMR (DMSO-d₆) δ 11.06 (s, 1H), 8.51 (d, 1H), 7.84 (d, 1H), 7.62 (d, 1H), 7.61 (d, 1H), 7.37-7.34 (m, 2H), 7.02 (d, 1H), 6.94 (s, 1H), 6.82 (d, 1H), 4.97 (dd, 1H), 4.44 (q, 2H), 4.26 (t, 2H), 3.86-3.84 (m, 1H), 3.80-3.76 (m, 1H), 3.74 (bs, 4H), 2.91-2.85 (m, 1H), 2.85 (s, 3H), 2.60-2.54 (m, 8H), 2.05-2.03 (m, 1H), 2.00-1.98 (m, 2H), 1.93-1.91 (m, 2H), 1.78-1.72 (m, 2H), 1.68-1.63 (m, 4H), 1.42 (t, 3H).

Compound 79: MS: m/z 821.6 (M⁺+1); ¹H NMR (DMSO-d₆) δ 11.06 (s, 1H), 8.51 (d, 1H), 7.84 (d, 1H), 7.62 (d, 1H), 7.61 (d, 1H), 7.39 (s, 1H), 7.37 (d, 1H), 7.01 (d, 1H), 6.94 (s, 1H), 6.82 (d, 1H), 4.97 (dd, 1H), 4.43 (q, 2H), 4.16 (t, 2H), 3.85-3.84 (m, 1H), 3.80-3.76 (m, 1H), 3.72 (bs, 4H), 2.89-2.85 (m, 3H), 2.85 (s, 3H), 2.67 (bs, 4H), 2.60-2.54 (m, 1H), 2.54-2.49 (m, 1H), 2.06-2.04 (m, 1H), 1.92-1.91 (m, 2H), 1.78-1.72 (m, 2H), 1.68-1.63 (m, 4H), 1.43 (t, 3H).

Compound 80: MS: m/z 848.2 (M⁺+1)

Compound 81: MS: m/z 848.4 (M⁺+1); ¹H NMR (DMSO-d₆) δ 11.06 (s, 1H), 8.55 (d, 1H), 7.86 (d, 1H), 7.83 (d, 1H), 7.61 (d, 1H), 7.40-7.33 (m, 2H), 7.13 (dd, 1H), 7.00 (dd, 1H), 6.85 (d, 1H), 4.97 (dd, 1H), 4.58-4.50 (m, 1H), 4.44 (q, 2H), 4.21 (t, 2H), 3.85 (bs, 5H), 2.93-2.84 (m, 1H), 2.74 (t, 2H), 2.64-2.56 (m, 1H), 2.56-2.49 (m, 5H), 2.14-2.07 (m, 2H), 2.07-2.01 (m, 1H), 1.94-1.86 (m, 2H), 1.81 (bs, 4H), 1.68-1.58 (m, 2H), 1.56-1.46 (m 2H), 1.42 (t, 3H).

Compound 82: MS: m/z 858.4 (M⁺+1); ¹H NMR (DMSO-d₆) δ 11.07 (s, 1H), 8.61 (d, 1H), 7.85 (d, 1H), 7.84 (d, 1H), 7.64 (d, 1H), 7.43 (s, 1H), 7.39 (s, 1H), 7.36 (d, 1H), 7.13 (d, 1H), 7.04 (d, 1H), 6.52 (t, 1H), 4.98 (dd, 1H), 4.91 (t, 2H), 4.58-4.49 (m, 1H), 4.15 (t, 2H), 3.91-3.81 (m, 1H), 3.71 (bs, 4H), 2.93-2.84 (m, 1H), 2.63-2.56 (m, 1H), 2.56-2.49 (m, 7H), 2.14-2.07 (m, 2H), 2.07-2.02 (m, 1H), 2.02-1.95 (m, 2H), 1.94-1.86 (m, 2H), 1.70-1.59 (m, 2H), 1.57-1.46 (m, 2H).

Compound 83: MS: m/z 871.4 (M⁺+1); ¹H NMR (DMSO-d₆) δ 11.07 (s, 1H), 8.51 (d, 1H), 7.84 (d, 1H), 7.64 (d, 1H), 7.61 (d, 1H), 7.44 (s, 1H), 7.36 (d, 1H), 7.04 (d, 1H), 6.94 (s, 1H), 6.83 (d, 1H), 6.52 (t, 1H), 4.99 (dd, 1H), 4.92 (t, 2H), 4.15 (t, 2H), 3.86-3.84 (m, 1H), 3.80-3.76 (m, 1H), 3.71 (bs, 4H), 2.92-2.85 (m, 1H), 2.85 (s, 3H), 2.60-2.57 (m, 1H), 2.57-2.50 (m, 3H), 2.54 (bs, 4H), 2.06-2.04 (m, 1H), 2.00-1.98 (m, 2H), 1.93-1.91 (m, 2H), 1.78-1.72 (m, 2H), 1.68-1.63 (m, 4H).

Compound 84: MS: m/z 924.5 (M⁺+1); ¹H NMR (DMSO-d₆) δ 11.06 (s, 1H), 8.61 (d, 1H), 7.85 (d, 1H), 7.83 (d, 1H), 7.65 (d, 1H), 7.39 (d, 1H), 7.38-7.33 (m, 2H), 7.14 (dd, 1H), 7.05 (dd, 1H), 5.75 (dd, 2H), 5.00 (dd, 1H), 4.57-4.50 (m, 1H), 4.15 (t, 2H), 3.90-3.80 (m, 1H), 3.71 (bs, 4H), 3.57 (t, 2H), 2.94-2.85 (m, 1H), 2.63-2.56 (m, 1H), 2.56-2.49 (m, 7H), 2.14-2.07 (m, 2H), 2.07-2.01 (m, 1H), 2.01-1.95 (m, 2H), 1.94-1.85 (m, 2H), 1.69-1.59 (m, 2H), 1.56-1.46 (m, 2H), 0.89-0.80 (m, 2H), −0.11 (s, 9H).

Compound 85: MS: m/z 824.4 (M⁺+1); ¹H NMR (DMSO-d₆) δ 11.07 (s, 1H), 9.81(bs, 1H), 8.69 (d, 1H), 7.96 (d, 1H), 7.86 (d, 1H), 7.67 (d, 1H), 7.51 (d, 1H), 7.39 (s, 1H), 7.36 (s, 1H), 7.14 (d, 1H), 7.04 (d, 1H), 5.71 (s, 2H), 5.00 (dd, 1H), 4.70-4.58 (m, 2H), 4.57-4.49 (m, 1H), 4.19 (t, 2H), 3.90-3.80 (m, 1H), 3.43-3.32 (m, 4H), 3.25-3.14 (m, 2H), 2.94-2.84 (m, 1H), 2.64-2.56 (m, 1H), 2.56-2.49 (m, 3H), 2.29-2.20 (m, 2H), 2.15-2.07 (m, 2H), 2.07-2.00 (m, 1H), 1.94-1.86 (m, 2H), 1.71-1.60 (m, 2H), 1.56-1.46 (m, 2H).

Compound 86: MS: m/z 794.4 (M⁺+1)

Compound 87: MS: m/z 822.4 (M⁺+1); ¹H NMR (DMSO-d₆) δ 11.05 (s, 1H), 8.53 (d, 1H), 7.86 (d, 1H), 7.79 (d, 1H), 7.60 (d, 1H), 7.38 (d, 1H), 7.18 (s, 1H), 7.17 (d, 1H), 7.13 (dd, 1H), 6.94 (dd, 1H), 4.97 (dd, 1H), 4.56-4.49 (m, 1H), 4.09-4.01 (m, 2H), 3.94 (s, 3H), 3.90-3.79 (m, 3H), 3.76 (bs, 2H), 2.92-2.84 (m, 1H), 2.80 (bs, 2H), 2.66-2.57 (m, 4H), 2.57-2.49 (m, 2H), 2.14-2.06 (m, 2H), 2.05-1.99 (m, 1H), 1.94-1.79 (m, 6H), 1.66-1.56 (m, 2H), 1.55-1.45 (m, 2H).

Compound 88: MS: m/z 834.4 (M⁺+1); ¹H NMR (DMSO-d₆) δ 11.06 (s, 1H), 8.55 (d, 1H), 7.86 (d, 1H), 7.82 (d, 1H), 7.61 (d, 1H), 7.38 (d, 1H), 7.32 (d, 1H), 7.14 (dd, 1H), 7.01 (dd, 1H). 6.85 (d, 1H), 4.98 (dd, 1H), 4.57-4.50 (m, 1H), 4.21 (t, 2H), 3.96 (s, 3H), 3.85 (bs, 5H), 2.94-2.84 (m, 1H), 2.75 (t, 2H), 2.64-2.56 (m, 2H), 2.56-2.49 (m, 4H), 2.14-2.07 (m, 2H), 2.07-2.00 (m, 1H), 1.94-1.86 (m, 2H), 1.81 (bs, 4H), 1.69-1.58 (m, 2H), 1.56-1.46 (m 2H).

Compound 89: MS: m/z 836.4 (M⁺+1); ¹H NMR (DMSO-d₆) δ 11.05 (s, 1H), 8.61 (d, 1H), 7.86 (d, 1H), 7.83 (d, 1H), 7.62 (d, 1H), 7.39 (dd, 1H), 7.36-7.32 (m, 2H), 7.13 (dd, 1H), 7.00 (dd, 1H), 4.97 (dd, 1H), 4.57-4.50 (m, 1H), 4.43 (q, 2H), 4.13 (t, 2H), 3.90-3.81 (m, 1H), 3.69 (m, 4H), 2.93-2.83 (m, 1H), 2.62-2.55 (m, 1H), 2.55-2.49 (m, 5H), 2.42 (t, 2H), 2.13-2.07 (m, 2H), 2.07-2.00 (m, 1H), 1.93-1.86 (m, 2H), 1.86-1.78 (m, 2H), 1.70-1.59 (m, 4H), 1.55-1.47 (m, 2H), 1.41 (t, 3H).

Compound 90: MS: m/z 850.4 (M⁺+1); ¹H NMR (DMSO-d₆) δ 11.05 (s, 1H), 8.55 (d, 1H), 7.85 (d, 1H), 7.79 (d, 1H), 7.60 (d, 1H), 7.38 (d, 1H), 7.30 (d, 1H), 7.16 (d, 1H), 7.13 (dd, 1H), 6.98 (dd, 1H), 4.98 (dd, 1H), 4.57-4.49 (m, 1H), 4.43 (q, 2H), 4.06 (t, 2H), 3.90-3.79 (m, 3H), 3.76 (bs, 2H), 2.93-2.84 (m, 1H), 2.79-2.73 (m, 2H), 2.63-2.58 (m, 1H), 2.58-2.54 (m, 2H), 2.54-2.49 (m, 3H), 2.13-2.07 (m, 2H), 2.07-2.00 (m, 1H), 1.92-1.81 (m, 4H), 1.76-1.69 (m, 2H), 1.67-1.54 (m, 4H), 1.54-1.46 (m, 2H), 1.41 (t, 3H).

Compound 91: MS: m/z 849.4 (M⁺+1); ¹H NMR (DMSO-d₆) δ 11.05 (s, 1H), 8.50 (d, 1H), 7.84 (d, 1H), 7.62 (d, 1H), 7.60 (d, 1H), 7.36-7.33 (m, 2H), 7.02 (d, 1H), 6.94 (d, 1H), 6.82 (dd, 1H), 4.97 (dd, 1H), 4.43 (q, 2H), 4.13 (t, 2H), 3.86-3.84 (m, 1H), 3.80-3.76 (m, 1H), 3.70 (bs, 4H), 2.91-2.85 (m, 1H), 2.85 (s, 3H), 2.62-2.56 (m, 2H), 2.54-2.49 (m, 4H), 2.42 (t, 2H), 2.06-2.03 (m, 1H), 1.92-1.91 (m, 2H), 1.85-1.80 (m, 2H), 1.78-1.72 (m, 2H), 1.68-1.62 (m, 6H), 1.41 (t, 3H).

Compound 92: MS: m/z 848.3 (M⁺+1); ¹H NMR (DMSO-d₆) δ 11.06 (s, 1H), 8.54 (d, 1H), 7.85 (d, 1H), 7.82 (d, 1H), 7.62 (d, 1H), 7.38 (d, 1H), 7.26 (d, 1H), 7.13 (dd, 1H), 7.00 (dd, 1H), 6.85 (d, 1H), 4.98 (dd, 1H), 4.57-4.49 (m, 1H), 4.13 (t, 2H), 3.96 (s, 3H), 3.84 (bs, 5H), 2.93-2.84 (m, 1H), 2.63-2.54 (m, 2H), 2.54-2.43 (m, 6H), 2.14-2.07 (m, 2H), 2.07-2.00 (m, 1H), 1.99-1.92 (m, 2H), 1.92-1.85 (m 2H), 1.80 (bs, 4H), 1.68-1.58 (m, 2H), 1.56-1.46 (m 2H).

Compound 93: MS: m/z 898.8 (M⁺+1); ¹H NMR (DMSO-d₆) δ 11.07 (s, 1H), 8.55 (d, 1H), 7.86 (d, 1H), 7.83 (d, 1H), 7.65 (d, 1H), 7.43 (s, 1H), 7.39 (d, 1H), 7.13 (dd, 1H), 7.03 (dd, 1H), 6.85 (d, 1H), 6.52 (t, 1H), 4.99 (dd, 1H), 4.91 (t, 2H), 4.58-4.49 (m, 1H), 4.12 (bs, 2H), 3.84 (bs, 5H), 2.94-2.84 (m, 1H), 2.63-2.54 (m, 2H), 2.54-2.31 (m, 6H), 2.14-2.08 (m, 2H), 2.08-2.01 (m, 1H), 2.01-1.86 (m, 4H), 1.80 (bs, 4H), 1.69-1.58 (m, 2H), 1.56-1.46 (m 2H).

Compound 94: MS: m/z 992.4 (M⁺+1); ¹H NMR (DMSO-d₆) δ 8.58 (d, 1H), 7.86 (d, 1H), 7.81 (d, 1H), 7.62 (d, 1H), 7.38-7.34 (m, 3H), 7.13 (dd, 1H), 7.01 (dd, 1H), 5.10 (dd, 1H), 5.08 (s, 2H), 4.56-4.51 (m, 1H), 4.44 (q, 2H), 4.14 (bs, 2H), 3.88-3.83 (m, 1H), 3.69 (bs, 4H), 3.58-3.54 (m, 1H), 3.50-3.46 (m, 1H), 3.32-3.31 (m, 4H), 3.05-2.99 (m, 1H), 2.80-2.77 (m, 1H), 2.58-2.49 (m, 3H), 2.11-2.08 (m, 3H), 1.91-1.79 (m, 8H), 1.66-1.60 (m 2H), 1.53-1.48 (m, 2H), 1.42 (t, 3H), 0.89-0.81 (m, 2H), −0.01 (s, 9H).

Compound 95: MS: m/z 892.4 (M⁺+1); ¹H NMR (DMSO-d₆) δ 9.88 (bs, 1H), 8.58 (d, 1H), 7.86 (d, 1H), 7.84 (d, 1H), 7.67 (d, 1H), 7.41 (d, 1H), 7.39 (d, 1H), 7.35 (d, 1H), 7.13 (dd, 1H), 7.03 (dd, 1H), 5.08-5.03 (m, 3H), 4.56-4.51 (m, 1H), 4.44 (q, 2H), 4.18 (t, 2H), 4.10-4.07 (m, 2H), 3.97-3.94 (m, 2H), 3.88-3.83 (m, 1H), 3.46-3.41 (m, 4H), 3.05-2.99 (m, 1H), 2.78-2.75 (m, 1H), 2.61-2.49 (m, 3H), 2.11-2.06 (m, 3H), 2.04-2.00 (m, 2H), 1.91-1.87 (m, 6H), 1.66-1.60 (m 2H), 1.55-1.49 (m, 2H), 1.43 (t, 3H).

Compound 96: MS: m/z 862.4 (M⁺+1)

Compound 97: MS: m/z 876.4 (M⁺+1); ¹H NMR (DMSO-d₆) δ 11.06 (s, 1H), 8.55 (d, 1H), 7.86 (d, 1H), 7.82 (d, 1H), 7.62 (d, 1H), 7.39 (d, 1H), 7.32 (d, 1H), 7.13 (dd, 1H), 7.00 (dd, 1H), 6.85 (d, 1H), 4.97 (dd, 1H), 4.57-4.50 (m, 1H), 4.43 (q, 2H), 4.11 (t, 2H), 3.83 (bs, 5H), 2.93-2.84 (m, 1H), 2.74 (t, 2H), 2.64-2.54 (m, 2H), 2.33 (bs, 4H), 2.13-2.07 (m, 2H), 2.07-2.00 (m, 1H), 1.94-1.85 (m, 2H), 1.85-1.71 (m, 6H), 1.69-1.57 (m, 4H), 1.56-1.46 (m 2H), 1.42 (t, 3H).

Compound 98: MS: m/z 836.3 (M⁺+1); ¹H NMR (DMSO-d₆) δ 11.05 (s, 1H), 8.61 (d, 1H), 7.85 (d, 1H), 7.83 (d, 1H), 7.62 (d, 1H), 7.39 (d, 1H), 7.34 (d, 1H), 7.30 (d, 1H), 7.13 (dd, 1H), 7.02 (dd, 1H), 4.97 (dd, 1H), 4.65 (bs, 1H), 4.57-4.49 (m, 1H), 4.43 (q, 2H), 4.25-4.15 (m, 3H), 3.90-3.81 (m, 1H), 3.20-3.12 (m, 1H), 3.02 (d, 1H), 2.93-2.84 (m, 2H), 2.63-2.53 (m, 2H), 2.53-2.49 (m, 2H), 2.25-2.19 (m, 1H), 2.14-2.08 (m, 2H), 2.07-2.02 (m, 2H), 2.02-1.95 (m, 2H), 1.93-1.86 (m, 2H), 1.69-1.59 (m, 2H), 1.56-1.46 (m, 2H), 1.41 (t, 3H), 1.20 (d, 3H).

Compound 99: MS: m/z 822.5 (M⁺+1); ¹H NMR (DMSO-d₆) δ 11.06 (s, 1H), 8.61 (d, 1H), 7.85 (d, 1H), 7.83 (d, 1H), 7.63 (d, 1H), 7.39-7.38 (m, 2H), 7.31 (d, 1H), 7.13 (dd, 1H), 7.03 (dd, 1H), 4.97 (dd, 1H), 4.67 (bs, 1H), 4.55-4.51 (m, 1H), 4.44 (q, 2H), 4.28-4.22 (m, 3H), 3.90-3.81 (m, 1H), 3.20-3.10 (m, 2H), 2.97 (d, 1H), 2.93-2.84 (m, 1H), 2.81 (t, 2H), 2.63-2.55 (m, 1H), 2.53-2.49 (m, 1H), 2.42-2.38 (m, 1H), 2.25-2.19 (m, 1H), 2.14-2.08 (m, 2H), 2.07-2.02 (m, 1H), 1.93-1.86 (m, 2H), 1.69-1.59 (m, 2H), 1.56-1.46 (m, 2H), 1.43 (t, 3H), 1.21 (d, 3H).

Compound 100: MS: m/z 862.6 (M⁺+1); ¹H NMR (DMSO-d₆) δ 11.05 (s, 1H), 8.58 (d, 1H), 7.86 (d, 1H), 7.81 (d, 1H), 7.61 (d, 1H), 7.39 (d, 1H), 7.32 (s, 1H), 7.18 (d, 1H), 7.13 (dd, 1H), 7.01 (dd, 1H), 4.97 (dd, 1H), 4.55-4.52 (m, 1H), 4.42 (q, 2H), 4.15 (t, 2H), 3.98-3.97 (m, 2H), 3.87-3.85 (m, 1H), 3.39 (bs, 2H), 3.10 (d, 2H), 2.91-2.85 (m, 1H), 2.59-2.57 (m, 2H), 2.45 (bs, 2H), 2.11-2.09 (m, 2H), 2.05-2.04 (m 1H), 1.91-1.84 (m, 6H), 1.66-1.60 (m, 4H), 1.53-1.48 (m, 4H), 1.40 (t, 3H).

Compound 101: MS: m/z 850.5 (M⁺+1); ¹H NMR (DMSO-d₆) δ 11.05 (s, 1H), 8.61 (d, 1H), 7.86 (d, 1H), 7.83 (d, 1H), 7.62 (d, 1H), 7.39 (d, 1H), 7.33 (d, 1H), 7.29 (d, 1H), 7.13 (dd, 1H), 7.01 (dd, 1H), 4.97 (dd, 1H), 4.65 (bs, 1H), 4.55-4.50 (m, 1H), 4.43 (q, 2H), 4.21 (d, 1H), 4.14 (t, 2H), 3.88-3.81 (m, 1H), 3.18-3.10 (m, 1H), 3.00 (d, 1H), 2.93-2.84 (m, 2H), 2.63-2.55 (m, 1H), 2.53-2.49 (m, 1H), 2.46-2.40 (m, 1H), 2.40-2.33 (m, 1H), 2.20-2.14 (m, 1H), 2.13-2.08 (m, 2H), 2.07-2.00 (m, 2H), 1.93-1.86 (m, 2H), 1.86-1.82 (m, 2H), 1.69-1.59 (m, 4H), 1.56-1.46 (m, 2H), 1.41 (t, 3H), 1.19 (d, 3H).

Compound 102: MS: m/z 884.3 (M⁺+1); ¹H NMR (DMSO-d₆) δ 11.06 (s, 1H), 8.59 (d, 1H), 7.86 (d, 1H), 7.81 (d, 1H), 7.64 (d, 1H), 7.43 (d, 1H), 7.39 (d, 1H), 7.20 (d, 1H), 7.14 (dd, 1H), 7.05 (d, 1H), 6.51 (t, 1H), 4.98 (dd. 1H), 4.94-4.88 (m, 2H), 4.19 (t, 2H), 4.00-3.98 (m, 2H), 3.88-3.84 (m, 1H), 3.42 (bs, 2H), 3.12 (d, 2H), 2.92-2.85 (m, 1H), 2.60-2.57 (m, 4H), 2.11-2.09 (m, 2H), 2.08-2.02 (m, 1H), 2.00-1.94 (m, 2H), 1.93-1.89 (m, 4H), 1.67-1.60 (m, 2H), 1.56-1.48 (m, 4H).

Compound 103: MS: m/z 884.5 (M⁺+1); ¹H NMR (DMSO-d₆) δ 11.07 (s, 1H), 8.55 (d, 1H), 7.86 (d, 1H), 7.83 (d, 1H), 7.64 (d, 1H), 7.47 (d, 1H), 7.39 (d, 1H), 7.13 (dd, 1H), 7.04 (dd, 1H), 6.85 (d, 1H), 6.52 (t, 1H), 4.99 (dd, 1H), 4.91 (t, 2H), 4.57-4.50 (m, 1H), 4.19 (t, 2H), 3.84 (bs, 5H), 2.94-2.84 (m, 1H), 2.75 (t, 2H), 2.63-2.54 (m, 2H), 2.54-2.49 (m, 4H), 2.13-2.07 (m, 2H), 2.07-2.01 (m, 1H), 1.94-1.85 (m, 2H), 1.81 (bs, 4H), 1.69-1.58 (m, 2H), 1.56-1.46 (m 2H).

Compound 104: MS: m/z 857.3 (M⁺+1); ¹H NMR (DMSO-d₆) δ 11.07 (s, 1H), 8.51 (d, 1H), 7.84 (d, 1H), 7.65 (d, 1H), 7.61 (d, 1H), 7.49 (dd, 1H), 7.37 (d, 1H), 7.06 (dd, 1H), 6.94 (d, 1H), 6.83 (dd, 1H), 6.53 (t, 1H), 4.99 (dd, 1H), 4.92 (t, 2H), 4.24 (t, 2H), 3.87-3.82 (m, 1H), 3.80-3.76 (m, 1H), 3.73 (bs, 4H), 2.92-2.87 (m, 1H), 2.85-2.83 (m, 2H), 2.85 (s, 3H), 2.66 (bs, 4H), 2.60-2.57 (m, 2H), 2.07-2.03 (m, 1H), 1.92-1.91 (m, 2H), 1.78-1.72 (m, 2H), 1.68-1.63 (m, 4H).

Compound 105: MS: m/z 844.3 (M⁺+1); ¹H NMR (DMSO-d₆) δ 11.07 (s, 1H), 8.61 (d, 1H), 7.86 (d, 1H), 7.84 (d, 1H), 7.65 (d, 1H), 7.49 (s, 1H), 7.39 (d, 1H), 7.37 (d, 1H), 7.14 (dd, 1H), 7.05 (dd, 1H), 6.53 (t, 1H), 4.99 (dd, 1H), 4.92 (t, 2H), 4.56-4.52 (m, 1H), 4.25 (t, 2H), 3.91-3.81 (m, 1H), 3.73 (bs, 4H), 2.92-2.87 (m, 1H), 2.84 (t, 2H), 2.67-2.66 (m, 4H), 2.63-2.56 (m, 1H), 2.56-2.49 (m, 1H), 2.14-2.07 (m, 2H), 2.07-2.02 (m, 1H), 1.93-1.87 (m, 2H), 1.67-1.61 (m, 2H), 1.54-1.49 (m, 2H).

Compound 106: MS: m/z 861.4 (M⁺+1); ¹H NMR (DMSO-d₆) δ 11.06 (s, 1H), 8.44 (d, 1H), 7.83 (d, 1H), 7.64-7.58 (m, 2H), 7.38 (s, 1H), 7.00 (d, 1H), 6.95 (d, 1H), 6.85 (d, 1H), 6.82 (dd, 1H), 4.98 (dd, 1H), 4.44 (q, 2H), 4.21 (bs, 2H), 3.85 (bs, 5H), 3.81-3.74 (m, 1H), 2.93-2.84 (m, 1H), 2.85 (s, 3H), 2.75 (bs, 2H), 2.63-2.54 (m, 2H), 2.54-2.49 (m, 4H), 2.08-2.01 (m, 1H), 1.96-1.87 (m, 2H), 1.81 (bs, 4H), 1.78-1.70 (m, 2H), 1.70-1.61 (m 4H), 1.43 (t, 3H).

Compound 107: MS: m/z 875.5 (M⁺+1); ¹H NMR (DMSO-d₆) δ 11.06 (s, 1H), 8.44 (d, 1H), 7.83 (d, 1H), 7.64-7.58 (m, 2H), 7.33 (s, 1H), 7.00 (dd, 1H), 6.94 (d, 1H), 6.85 (d, 1H), 6.83 (dd, 1H), 4.98 (dd, 1H), 4.44 (q, 2H), 4.13 (bs, 2H), 3.84 (bs, 5H), 3.81-3.74 (m, 1H), 2.93-2.82 (m, 1H), 2.85 (s, 3H), 2.63-2.54 (m, 2H), 2.54-2.42 (m, 4H), 2.42-2.31 (m, 2H), 2.08-2.01 (m, 1H), 1.98-1.88 (m, 4H), 1.80 (bs, 4H), 1.78-1.70 (m, 2H), 1.70-1.60 (m 4H), 1.42 (t, 3H).

Compound 108: MS: m/z 897.4 (M⁺+1); ¹H NMR (DMSO-d₆) δ 11.07 (s, 1H), 8.45 (d, 1H), 7.83 (d, 1H), 7.64 (d, 1H), 7.61 (d, 1H), 7.48 (s, 1H), 7.04 (d, 1H), 6.94 (d, 1H), 6.86 (d, 1H), 6.82 (dd, 1H), 6.52 (t, 1H), 4.99 (dd, 1H), 4.92 (t, 2H), 4.19 (bs, 2H), 3.85 (bs, 5H), 3.82-3.75 (m, 1H), 2.94-2.83 (m, 1H), 2.85 (s, 3H), 2.75 (bs, 2H), 2.63-2.54 (m, 2H), 2.54-2.49 (m, 4H), 2.09-2.01 (m, 1H), 1.95-1.87 (m, 2H), 1.81 (bs, 4H), 1.78-1.71 (m, 2H), 1.70-1.61 (m 4H).

Compound 109: MS: m/z 849.5 (M⁺+1); ¹H NMR (DMSO-d₆) δ 11.05 (s, 1H), 8.43 (d, 1H), 7.80 (d, 1H), 7.63-7.57 (m, 2H), 7.26 (s, 1H), 7.17 (d, 1H), 6.97-6.91 (m, 2H), 6.82 (dd, 1H), 4.97 (dd, 1H), 4.41 (q, 2H), 4.13-4.00 (m, 2H), 3.95-3.67 (m, 6H), 2.92-2.84 (m, 1H), 2.84 (s, 3H), 2.80 (bs, 2H), 2.66-2.58 (m, 4H), 2.58-2.49 (m, 2H), 2.07-2.00 (m, 1H), 1.94-1.82 (m, 6H), 1.78-1.69 (m, 2H), 1.69-1.58 (m, 4H), 1.40 (t, 3H).

Compound 110: MS: m/z 837.4 (M⁺+1); ¹H NMR (DMSO-d₆) δ 11.05 (s, 1H), 8.45 (d, 1H), 7.80 (d, 1H), 7.62-7.58 (m, 2H), 7.33 (d, 1H), 7.18 (d, 1H), 6.96 (dd, 1H), 6.94 (d, 1H), 6.83 (dd, 1H), 4.97 (dd, 1H), 4.42 (q, 2H), 4.16 (t, 2H), 3.95-3.69 (m, 6H), 2.97-2.91 (m, 4H), 2.91-2.84 (m, 1H), 2.85 (s, 3H), 2.69-2.70 (m, 2H), 2.63-2.54 (m, 1H), 2.58-2.49 (m, 1H), 2.07-2.00 (m, 1H), 1.94-1.85 (m, 4H), 1.80-1.70 (m, 2H), 1.70-1.60 (m, 4H), 1.40 (t, 3H).

Compound 111: MS: m/z 889.2 (M⁺+1); ¹H NMR (DMSO-d₆) δ 11.06 (s, 1H), 8.44 (d, 1H), 7.84 (d, 1H), 7.64-7.58 (m, 2H), 7.33 (d, 1H), 7.00 (dd, 1H), 6.94 (d, 1H), 6.85 (d, 1H), 6.83 (dd, 1H), 4.97 (dd, 1H), 4.43 (q, 2H), 4.12 (bs, 2H), 3.84 (bs, 5H), 3.81-3.74 (m, 1H), 2.93-2.82 (m, 1H), 2.85 (s, 3H), 2.63-2.53 (m, 2H), 2.53-2.49 (m, 4H), 2.34 (bs, 4H), 2.08-2.00 (m, 1H), 1.96-1.87 (m, 2H), 1.80 (bs, 4H), 1.80-1.70 (m, 4H), 1.70-1.59 (m, 6H), 1.42 (t, 3H).

Compound 112: MS: m/z 863.5 (M⁺+1); ¹H NMR (DMSO-d) δ 11.05 (s, 1H), 8.45 (bs, 1H), 7.80 (bs, 1H), 7.60 (d, 2H), 7.30 (bs, 1H), 7.16 (bs, 1H), 6.98 (d, 1H), 6.94 (bs, 1H), 6.82 (d, 1H), 4.97 (dd, 1H), 4.42 (q, 2H), 4.07 (bs, 2H), 3.91-3.68 (m, 6H), 2.93-2.82 (m, 1H), 2.85 (s, 3H), 2.76 (bs, 2H), 2.63-2.53 (m, 3H), 2.58-2.49 (m, 3H), 2.07-2.00 (m, 1H), 1.94-1.80 (m, 4H), 1.80-1.70 (m, 4H), 1.70-1.60 (m, 4H), 1.60-1.52 (m, 2H), 1.41 (t, 3H).

Compound 113: MS: m/z 852.5 (M⁺+1); ¹H NMR (DMSO-d₆) δ 11.06 (s, 1H), 8.60 (d, 1H), 7.85 (d, 1H), 7.82 (d, 1H), 7.62 (d, 1H), 7.39-7.36 (m, 2H), 7.33 (d, 1H), 7.13 (dd, 1H), 7.01 (dd, 1H), 4.97 (dd, 1H), 4.55-4.51 (m, 1H), 4.42 (q, 2H), 4.24 (t, 2H), 3.88-3.82 (m, 1H), 3.80 (t, 2H), 3.69-3.65 (m, 6H), 2.91-2.85 (m, 1H), 2.62-2.53 (m, 7H), 2.53-2.49 (m, 1H), 2.11-2.09 (m, 2H), 2.06-2.02 (m, 1H), 1.91-1.89 (m, 2H), 1.67-1.61 (m, 2H), 1.54-1.48 (m, 2H), 1.41 (t, 3H).

Compound 114: MS: m/z 865.4 (M⁺+1); ¹H NMR (DMSO-d₆) δ 11.06 (s, 1H), 8.50 (d, 1H), 7.83 (d, 1H), 7.62 (d, 1H), 7.60 (d, 1H), 7.38 (d, 1H), 7.33 (d, 1H), 7.01 (d, 1H), 6.94 (d, 1H), 6.83 (dd, 1H), 4.98 (dd, 1H), 4.43 (q, 2H), 4.24 (t, 2H), 3.85-3.76 (m, 2H), 3.80 (t, 2H), 3.67-3.66 (m, 6H), 2.91-2.85 (m, 1H), 2.85 (s, 3H), 2.59-2.50 (m, 7H), 2.50-2.49 (m, 1H), 2.06-2.03 (m, 1H), 1.92-1.91 (m, 2H), 1.78-1.72 (m, 2H), 1.68-1.62 (m, 4H), 1.42 (t, 3H).

Compound 115: MS: m/z 850.5 (M⁺+1); ¹H NMR (DMSO-d₆) δ 11.06 (s, 1H), 8.61 (d, 1H), 7.86 (d, 1H), 7.83 (d, 1H), 7.61 (d, 1H), 7.39 (d, 1H), 7.36-7.33 (m, 2H), 7.14 (dd, 1H), 6.99 (dd, 1H), 4.97 (dd, 1H), 4.56-4.50 (m, 1H), 4.43 (q, 2H), 4.10 (t, 2H), 3.90-3.81 (m, 1H), 3.69 (m, 4H), 2.92-2.85 (m, 1H), 2.62-2.55 (m, 1H), 2.55-2.49 (m, 5H), 2.36 (m, 2H), 2.11-2.07 (m, 2H), 2.07-2.02 (m, 1H), 1.91-1.89 (m, 2H), 1.83-1.78 (m, 2H), 1.67-1.61 (m, 2H), 1.60-1.53 (m, 2H), 1.53-1.47 (m, 4H), 1.41 (t, 3H).

Compound 116: MS: m/z 863.4 (M⁺+1); ¹H NMR (DMSO-d₆) δ 11.06 (s, 1H), 8.51 (d, 1H), 7.83 (d, 1H), 7.62-7.60 (m, 2H), 7.35-7.33 (m, 2H), 7.00 (dd, 1H), 6.94 (d, 1H), 6.82 (dd, 1H), 4.97 (dd, 1H), 4.43 (q, 2H), 4.10 (t, 2H), 3.87-3.82 (m, 1H), 3.80-3.76 (m, 1H), 3.69 (bs, 4H), 2.92-2.88 (m, 1H), 2.85 (s, 3H), 2.60-2.54 (m, 1H), 2.54-2.49 (m, 5H), 2.36 (t, 2H), 2.06-2.03 (m, 1H), 1.93-1.91 (m, 2H), 1.83-1.78 (m, 2H), 1.76-1.72 (m, 2H), 1.68-1.62 (m, 4H), 1.58-1.54 (m, 2H), 1.52-1.48 (m, 2H), 1.41 (t, 3H).

Compound 117: MS: m/z 855.6 (M⁺+1); ¹H NMR (DMSO-d₆) δ 11.06 (s, 1H), 8.38 (d, 1H), 8.29 (d, 1H), 8.01 (dd, 1H). 7.62 (d, 1H), 7.35 (d, 1H), 7.18 (d, 2H), 7.06 (d, 2H), 7.02 (dd, 1H), 4.98 (dd, 1H), 4.44 (q, 2H), 4.16 (t, 2H), 3.25 (bs, 4H), 2.92-2.86 (m, 1H), 2.60-2.49 (m, 8H), 2.07-2.02 (m, 1H), 2.00-1.99 (m, 2H), 1.48 (s, 6H), 1.42 (t, 3H).

Compound 118: MS: m/z 841.1 (M⁺+1); ¹H NMR (DMSO-d₆) δ 11.06 (s, 1H), 8.38 (d, 1H), 8.29 (d, 1H), 8.07 (dd, 1H), 7.62 (d, 1H), 7.40 (d, 1H), 7.18 (d, 2H), 7.07 (d, 2H), 7.02 (dd, 1H), 4.98 (dd, 1H), 4.44 (q, 2H), 4.27 (t, 2H), 3.26 (bs, 4H), 2.92-2.84 (m, 3H), 2.69 (bs, 4H), 2.62-2.57 (m, 1H), 2.50-2.49 (m, 1H), 2.07-2.02 (m, 1H), 1.48 (s, 6H), 1.42 (t, 3H).

Compound 119: MS: m/z 869.2 (M⁺+1); ¹H NMR (DMSO-d₆) δ 11.06 (s, 1H), 8.38 (d, 1H), 8.29 (d, 1H), 8.07 (dd, 1H), 7.62 (d, 1H), 7.33 (d, 1H), 7.18 (d, 2H), 7.05 (d, 2H), 7.01 (dd, 1H), 4.98 (dd, 1H), 4.43 (q, 2H), 4.14 (t, 2H), 3.22 (bs, 4H), 2.91-2.85 (m, 1H), 2.60-2.57 (m, 1H), 2.53-2.52 (m, 5H), 2.43 (t, 2H), 2.08-2.03 (m, 1H), 1.85-1.80 (m, 2H), 1.68-1.66 (m, 2H), 1.48 (s, 6H), 1.41 (t, 3H).

Compound 120: MS: m/z 852.4 (M⁺+1); ¹H NMR (DMSO-d₆) δ 8.65 (d, 1H), 7.88 (d, 1H), 7.86 (d, 1H), 7.65 (d, 1H), 7.41 (d, 1H), 7.39 (d, 1H), 7.37 (d, 1H), 7.14 (d, 1H), 7.07 (d, 1H), 5.09-5.02 (m, 5H), 4.57-4.51 (m, 1H), 4.42 (q, 2H), 3.86 (bs, 4H), 3.75 (bs, 4H), 3.05-2.99 (m, 1H), 2.77-2.75 (m, 1H), 2.61-2.55 (m, 1H), 2.55-2.49 (m, 1H), 2.12-2.07 (m, 3H), 1.91-1.89 (m, 2H), 1.69-1.62 (m, 2H), 1.54-1.49 (m, 2H), 1.43 (t, 3H).

Compound 121: MS: m/z 822.3 (M⁺+1).

Compound 122: MS: m/z 914.1 (M⁺+23); ¹H NMR (DMSO-d₆) δ 8.56 (d, 1H), 7.88 (d, 1H), 7.86 (d, 1H), 7.64 (d, 1H), 7.39 (d, 1H), 7.34 (d, 1H), 7.14 (dd, 1H), 7.04 (dd, 1H), 6.93 (d, 1H), 5.10-5.02 (m, 3H), 4.95 (s, 2H), 4.57-4.50 (m, 1H), 4.42 (q, 2H), 3.98-3.91 (m, 5H), 3.50 (bs, 4H), 3.06-2.98 (m, 1H), 2.80-2.74 (m, 1H), 2.63-2.54 (m, 1H), 2.14-2.04 (m, 3H), 1.89 (bs, 4H), 1.77 (bs, 2H), 1.68-1.59 (m, 2H), 1.56-1.46 (m 2H), 1.43 (t, 3H).

Compound 123: MS: m/z 862.4 (M⁺+1).

Compound 124: MS: m/z 865.3 (M⁺+1).

Compound 125: MS: m/z 835.3 (M⁺+1).

Compound 126: MS: m/z 892.3 (M⁺+1); ¹H NMR (DMSO-d₆) δ 8.60 (d, 1H), 7.85 (d, 1H), 7.82 (d, 1H), 7.64 (d, 1H), 7.41-7.38 (m, 2H), 7.35 (d, 1H), 7.14 (dd, 1H), 7.06 (dd, 1H), 5.10-5.03 (m, 3H), 4.76 (s, 2H), 4.55-4.52 (m, 1H), 4.43 (q, 2H), 4.09 (s, 2H), 3.89-3.82 (m, 1H), 3.73 (bs, 6H), 3.06-2.99 (m, 1H), 2.78-2.75 (m, 1H), 2.63-2.55 (m, 1H), 2.11-2.06 (m, 3H), 1.91-1.88 (m, 2H), 1.79 (bs, 4H), 1.67-1.61 (m, 2H), 1.54-1.48 (m 2H), 1.43 (t, 3H).

Compound 127: MS: m/z 862.3 (M⁺+1).

Compound 128: MS: m/z 905.4 (M⁺+1); ¹H NMR (DMSO-d₆) δ 8.45 (d, 1H), 7.88 (d, 1H), 7.64 (d, 1H), 7.61 (d, 1H), 7.34 (d, 1H), 7.04 (dd, 1H), 6.94 (d, 1H), 6.92 (d, 1H), 6.82 (dd, 1H), 5.10-5.02 (m, 3H), 4.95 (s, 2H), 4.42 (q, 2H), 3.98-3.90 (m, 4H), 3.88-3.82 (m, 1H), 3.82-3.74 (m, 1H), 3.06-2.98 (m, 1H), 2.85 (s, 3H), 2.80-2.72 (m, 1H), 2.63-2.53 (m, 1H), 2.10-2.04 (m, 3H), 1.95-1.84 (m, 4H), 1.80-1.71 (m, 4H), 1.70-1.60 (m, 4H), 1.43 (t, 3H).

Compound 129: MS: m/z 875.3 (M⁺+1).

Compound 130: MS: m/z 879.5 (M⁺+1).

Compound 131: MS: m/z 849.3 (M⁺+1).

Compound 132: MS: m/z 862.3 (M⁺+1); ¹H NMR (DMSO-d₆) δ 11.06 (s, 1H), 8.53 (d, 1H), 7.86-7.82 (m, 2H), 7.62 (d, 1H), 7.39 (d, 1H), 7.34 (s, 1H), 7.13 (dd, 1H), 7.00 (d, 1H), 6.94 (d, 1H), 4.98 (dd, 1H), 4.55-4.52 (m, 1H), 4.43 (q, 2H), 4.16 (bs, 2H), 3.88-3.84 (m, 1H), 3.58 (bs, 4H), 2.92-2.85 (m, 1H), 2.60-2.57 (m, 2H), 2.50-2.49 (m, 6H), 2.11-2.02 (m, 7H), 1.91-1.81 (m, 4H), 1.66-1.60 (m, 2H), 1.54-1.48 (m, 2H), 1.41 (t, 3H).

Compound 133: MS: m/z 848.3 (M⁺+1); ¹H NMR (DMSO-d₆) δ 11.05 (s, 1H), 8.53 (d, 1H), 7.85 (d, 1H), 7.81 (d, 1H), 7.60 (d, 1H), 7.39 (d, 1H), 7.36 (d, 1H), 7.13 (dd, 1H), 7.00 (dd, 1H), 6.94 (d, 1H), 4.97 (dd, 1H), 4.55-4.52 (m, 1H), 4.42 (q, 2H), 4.20 (t, 2H), 3.88-3.82 (m, 1H), 3.57-3.42 (m, 4H), 2.92-2.86 (m, 3H), 2.79-2.69 (m, 1H), 2.70-2.68 (m, 2H), 2.60-2.57 (m, 2H), 2.11-2.09 (m, 2H), 2.07-2.01 (m, 2H), 2.00-1.97 (m, 1H), 1.90-1.89 (m, 2H), 1.83-1.81 (m, 2H), 1.67-1.60 (m, 2H), 1.54-1.48 (m, 2H), 1.42 (t, 3H).

Compound 134: MS: m/z 876.4 (M⁺+1); ¹H NMR (DMSO-d₆) δ 11.05 (s, 1H), 8.53 (d, 1H), 7.86-7.82 (m, 2H), 7.61 (d, 1H), 7.38 (d, 1H), 7.33 (s, 1H), 7.13 (dd, 1H), 6.99 (d, 1H), 6.93 (d, 1H), 4.97 (dd, 1H), 4.55-4.52 (m, 1H), 4.43 (q, 2H), 4.12 (bs, 2H), 3.88-3.83 (m, 1H), 3.58 (bs, 4H), 2.92-2.85 (m, 1H), 2.60-2.57 (m, 2H), 2.50-2.49 (m, 6H), 2.11-2.02 (m, 7H), 1.91-1.81 (m, 6H), 1.66-1.60 (m, 2H), 1.54-1.48 (m, 2H), 1.41 (t, 3H).

Compound 135: MS: m/z 892.3 (M⁺+1); ¹H NMR (DMSO-d₆) δ 8.54-8.51 (m, 1H), 7.88-7.85 (m, 2H), 7.65-7.62 (m, 1H), 7.39-7.38 (m, 1H), 7.33-7.31 (m, 1H), 7.14-7.12 (m, 1H), 7.07-7.00 (m, 2H), 5.09-5.03 (m, 3H), 4.90 (s, 1H), 4.84 (s, 1H), 4.55-4.52 (m, 1H), 4.44-4.39 (m, 2H), 3.88-3.82 (m, 5H), 3.53-3.38 (m, 4H), 3.00-2.99 (m, 1H), 2.78-2.74 (m, 1H), 2.60-2.57 (m, 1H), 2.11-2.04 (m, 6H), 1.97-1.90 (m, 3H), 1.67-1.61 (m, 2H), 1.54-1.49 (m, 2H), 1.44-1.40 (m, 3H).

Compound 136: MS: m/z 862.3 (M⁺+1).

Compound 137: MS: m/z 850.5 (M⁺+1); ¹H NMR (CDCl₃) δ 7.98 (s, 1H), 7.96 (d, 1H), 7.84 (d, 1H), 7.73 (d, 1H), 7.43 (d, 1H), 7.01 (dd, 1H), 6.96 (d, 1H), 6.87 (d, 1H), 6.73 (d, 1H), 6.61 (dd, 1H), 4.85 (dd, 1H), 4.53-4.51 (m, 2H), 4.37 (q, 2H), 4.21 (t, 2H), 4.00-3.92 (m, 1H), 3.85 (t, 2H), 3.69-3.64 (m, 1H), 3.45 (d, 2H), 3.07-3.02 (m, 2H), 2.91-2.88 (m, 1H), 2.85 (s, 3H), 2.82-2.70 (m, 2H), 2.22-2.21 (m, 2H), 2.18-2.15 (m, 1H), 2.04-1.98 (m, 1H), 1.93-1.91 (m, 2H), 1.86-1.84 (m, 2H), 1.78-1.71 (m, 2H), 1.52 (t, 3H), 1.48-1.43 (m, 2H), 1.38-1.31 (m, 2H).

Compound 138: MS: m/z 837.5 (M⁺+1); ¹H NMR (CDCl₃) δ 7.97-7.95 (m, 2H), 7.86 (d, 1H), 7.73 (d, 1H), 7.56 (d, 1H), 7.02-7.00 (m, 2H), 6.97 (d, 1H), 6.87-6.84 (m, 2H), 4.85 (dd, 1H), 4.53-4.51 (m, 2H), 4.37 (q, 2H), 4.35-4.29 (m, 1H), 4.21 (t, 2H), 4.08-4.02 (m, 1H), 3.85 (t, 2H), 3.45 (d, 2H), 3.07-3.02 (m, 2H), 2.91-2.88 (m, 1H), 2.84-2.70 (m, 2H), 2.22-2.13 (m, 5H), 2.04-1.97 (m, 1H), 1.93-1.91 (m, 2H), 1.71-1.65 (m, 2H), 1.52 (t, 3H), 1.50-1.42 (m, 2H), 1.38-1.31 (m, 2H).

Compound 139: MS: m/z 857.8 (M⁺+1).

Compound 140: MS: m/z 820.1 (M⁺+1); ¹H NMR (CDCl₃) δ 7.98-7.96 (m, 2H), 7.85 (d, 1H), 7.74 (d, 1H), 7.43 (d, 1H), 7.00-6.97 (m, 2H), 6.83 (d, 1H), 6.73 (d, 1H), 6.61 (dd, 1H), 4.85 (dd, 1H), 4.55-4.52 (m, 2H), 4.38 (q, 2H), 4.12 (t, 2H), 4.00-3.93 (m, 1H), 3.69-3.64 (m, 1H), 3.10-3.05 (m, 2H), 2.91-2.88 (m, 1H), 2.85 (s, 3H), 2.82-2.70 (m, 2H), 2.24-2.22 (m, 2H), 2.19-2.15 (m, 1H), 2.00-1.95 (m, 3H), 1.86-1.83 (m, 4H), 1.78-1.72 (m, 2H), 1.53 (t, 3H), 1.48-1.42 (m, 2H), 1.40-1.34 (m, 2H).

Compound 141: MS: m/z 893.3 (M⁺+1); ¹H NMR (DMSO-d₆) δ 8.48 (d, 1H), 8.23 (t, 1H), 7.82 (d, 1H), 7.67 (d, 1H), 7.61 (d, 1H), 7.37-7.36 (m, 2H), 7.10 (dd, 1H), 6.94 (d, 1H), 6.83 (dd, 1H), 5.09-5.03 (m, 3H), 4.64 (s, 2H), 4.45-4.40 (m, 4H), 3.85-3.75 (m, 2H), 3.10-3.08 (m, 2H), 3.06-2.95 (m, 3H), 2.85 (s, 3H), 2.78-2.73 (m, 1H), 2.61-2.53 (m, 1H), 2.08-2.06 (m, 1H), 1.92-1.91 (m, 2H), 1.85-1.82 (m, 1H), 1.78-1.62 (m, 1H), 1.42 (t, 3H).

Compound 142: MS: m/z 863.4 (M⁺+1).

Compound 143: MS: m/z 879.4 (M⁺+1); ¹H NMR (DMSO-d₆) δ 8.52 (d, 1H), 8.09 (d, 1H), 7.84 (d, 1H), 7.65 (d, 1H), 7.61 (d, 1H), 7.40 (d, 1H), 7.34 (d, 1H), 7.10 (dd, 1H), 6.95 (d, 1H), 6.83 (dd, 1H), 5.08-5.03 (m, 3H), 4.62 (s, 2H), 4.45-4.40 (m, 4H), 4.10-4.04 (m, 1H), 3.85-3.75 (m, 2H), 3.19-3.13 (m, 2H), 3.05-2.99 (m, 1H), 2.85 (s, 3H), 2.78-2.73 (m, 1H), 2.61-2.53 (m, 1H), 2.08-2.06 (m, 1H), 1.92-1.91 (m, 2H), 1.84-1.83 (m, 2H), 1.78-1.72 (m, 2H), 1.68-1.62 (m, 4H), 1.56-1.50 (m, 2H), 1.41 (t, 3H).

Compound 144: MS: m/z 849.3 (M⁺+1).

Compound 145: MS: m/z 891.4 (M⁺+1); ¹H NMR (DMSO-d₆) δ 8.45 (d, 1H), 7.88 (d, 1H), 7.88 (d, 1H), 7.66 (d, 1H), 7.61 (d, 1H), 7.36 (d, 1H), 7.06 (dd, 1H), 7.02 (d, 1H), 6.94 (d, 1H), 6.83 (dd, 1H), 5.08-5.02 (m, 3H), 4.76 (s, 2H), 4.43 (q, 2H), 4.37 (d, 1H), 4.32 (d, 1H), 4.00-4.94 (m, 2H), 3.86-3.75 (m, 6H), 3.05-2.99 (m, 1H), 2.85 (s, 3H), 2.77-2.75 (m, 1H), 2.61-2.55 (m, 1H), 2.29-2.25 (m, 2H), 2.08-2.06 (m, 1H), 1.93-1.91 (m, 2H), 1.78-1.72 (m, 2H), 1.68-1.62 (m, 4H), 1.43 (t, 3H).

Compound 146: MS: m/z 861.3 (M⁺+1).

Compound 147: MS: m/z 863.6 (M⁺+1); ¹H NMR (CDCl₃) δ 7.97 (s, 1H), 7.96 (d, 1H), 7.86 (d, 1H), 7.74 (d, 1H), 7.56 (d, 1H), 7.02 (dd, 1H), 7.00 (d, 1H), 6.97 (d, 1H), 6.88 (d, 1H), 6.85 (dd, 1H), 4.85 (dd, 1H), 4.38 (q, 2H), 4.35-4.29 (m, 1H), 4.21 (t, 2H), 4.18-4.14 (m, 1H), 4.08-4.02 (m, 1H), 3.77 (t, 2H), 3.75-3.73 (m, 2H), 3.67-3.65 (m, 2H), 2.91-2.88 (m, 1H), 2.84-2.70 (m, 2H), 2.33-2.29 (m, 2H), 2.20-2.15 (m, 5H), 1.89-1.86 (m, 2H), 1.72-1.67 (m, 6H), 1.52 (t, 3H), 1.49-1.42 (m, 2H).

Compound 148: MS: m/z 876.4 (M⁺+1); ¹H NMR (CDCl₃) δ 7.97 (s, 1H), 7.96 (d, 1H), 7.84 (d, 1H), 7.74 (d, 1H), 7.43 (d, 1H), 7.02 (dd, 1H), 6.96 (d, 1H), 6.88 (d, 1H), 6.73 (d, 1H), 6.61 (dd, 1H), 4.85 (dd, 1H), 4.38 (q, 2H), 4.21 (t, 2H), 4.18-4.14 (m, 1H), 4.00-3.92 (m, 1H), 3.77 (t, 2H), 3.75-3.74 (m, 2H), 3.66-3.64 (m, 3H), 2.91-2.88 (m, 1H), 2.85 (s, 3H), 2.83-2.70 (m, 2H), 2.33-2.30 (m, 2H), 2.23-2.21 (m, 2H), 2.19-2.15 (m, 1H), 1.89-1.84 (m, 4H), 1.78-1.69 (m, 6H), 1.52 (t, 3H), 1.48-1.42 (m, 2H).

Compound 149: MS: m/z 872.4(M⁺+23); ¹H NMR (DMSO-d₆) δ 11.05 (s, 1H), 8.66 (d, 1H), 7.87 (d, 1H), 7.85 (d, 1H), 7.60 (d, 1H), 7.39 (d, 1H), 7.34 (d, 1H), 7.32 (d, 1H), 7.14 (dd, 1H), 6.99 (dd, 1H), 4.97 (dd, 1H), 4.55-4.51 (m, 1H), 4.42 (q, 2H), 4.27 (s, 2H), 4.11 (t, 2H), 3.94 (t, 2H), 3.89-3.83 (m, 1H), 3.54-3.52 (m, 2H), 3.49-3.45 (m, 2H), 2.91-2.86 (m, 1H), 2.62-2.53 (m, 2H), 2.11-2.09 (m, 2H), 2.07-2.03 (m, 1H), 1.90-1.88 (m, 2H), 178-1.72 (m, 4H), 1.68-1.62 (m, 2H), 1.53-1.48 (m, 2H), 1.40 (t, 3H).

Compound 150: MS: m/z 863.3 (M⁺+1).

Compound 151: MS: m/z 836.5 (M⁺+1); ¹H NMR (DMSO-d₆) δ 11.06 (s, 1H), 8.67 (d, 1H), 7.88 (d, 1H), 7.86 (d, 1H), 7.60 (d, 1H), 7.39 (d, 1H), 7.36 (d, 1H), 7.32 (d, 1H), 7.14 (dd, 1H), 6.97 (dd, 1H), 4.98 (dd, 1H), 4.56-4.51 (m, 1H), 4.41 (q, 2H), 4.27 (s, 2H), 4.14 (t, 2H), 3.96 (t, 2H), 3.89-3.84 (m, 1H), 3.59-3.55 (m, 4H), 2.91-2.86 (m, 1H), 2.62-2.51 (m, 2H), 2.11-2.09 (m, 2H), 2.06-2.02 (m, 3H), 1.90-1.88 (m, 2H), 1.68-1.62 (m, 2H), 1.54-1.48 (m, 2H), 1.40 (t, 3H).

Compound 152: MS: m/z 863.8 (M⁺+1); ¹H NMR (DMSO-d₆) δ 11.06 (s, 1H), 8.53 (d, 1H), 7.86 (d, 1H), 7.82 (d, 1H), 7.62 (d, 1H), 7.39 (d, 1H), 7.37 (d, 1H), 7.13 (dd, 1H), 7.01 (dd, 1H), 6.84 (d, 1H), 4.98 (dd, 1H), 4.58-4.50 (m, 1H), 4.43 (q, 2H), 4.21 (t, 2H), 3.88-3.84 (m, 1H), 3.82-3.80 (m, 6H), 3.44-3.42 (m, 1H), 2.92-2.85 (m, 1H), 2.61-2.56 (m, 1H), 2.55-2.49 (m, 1H), 2.10-2.07 (m, 2H), 2.06-2.03 (m, 1H), 1.92-1.86 (m, 4H), 1.81 (bs, 2H), 1.66-1.57 (m, 4H), 1.53-1.48 (m 2H), 1.42 (t, 3H), 1.43-1.36 (m, 2H).

Compound 153: MS: m/z 823.6 (M⁺+1); ¹H NMR (DMSO-d₆) δ 11.06 (s, 1H), 8.60 (d, 1H), 7.86 (d, 1H), 7.81 (d, 1H), 7.62 (d, 1H), 7.39-7.36 (m, 3H), 7.13 (dd, 1H), 7.02 (dd, 1H), 4.97 (dd, 1H), 4.56-4.51 (m, 1H), 4.42 (q, 2H), 4.25 (t, 2H), 4.11-4.09 (m, 2H), 3.88-3.82 (m, 3H), 3.76-3.72 (m, 1H), 3.44-3.41 (m, 2H), 2.92-2.85 (m, 1H), 2.61-2.56 (m, 1H), 2.54-2.49 (m, 1H), 2.12-2.09 (m, 2H), 2.06-2.02 (m, 1H), 1.97-1.94 (m, 2H), 1.90-1.88 (m, 2H), 1.66-1.60 (m, 2H), 1.55-1.48 (m 4H), 1.41 (t, 3H).

Compound 154: MS: m/z 836.9 (M⁺+1); ¹H NMR (DMSO-d₆) δ 11.06 (s, 1H), 8.49 (d, 1H), 7.82 (d, 1H), 7.62 (d, 1H), 7.60 (d, 1H), 7.39-7.36 (m, 2H), 7.02 (dd, 1H), 6.94 (d, 1H), 6.82 (dd, 1H), 4.97 (dd, 1H), 4.42 (q, 2H), 4.25 (t, 2H), 4.12-4.08 (m, 2H), 3.88-3.82 (m, 3H), 3.82-3.72 (m, 2H), 3.44-3.41 (m, 2H), 2.92-2.85 (m, 1H), 2.84 s, 3H), 2.61-2.56 (m, 1H), 2.54-2.49 (m, 1H), 2.07-2.02 (m, 1H), 1.98-1.95 (m, 2H), 1.92-1.90 (m, 2H), 1.78-1.72 (m, 2H), 1.67-1.62 (m, 4H), 1.55-1.49 (m 2H), 1.41 (t, 3H).

Compound 155: MS: m/z 850.7 (M⁺+1); ¹H NMR (DMSO-d₆) δ 11.06 (s, 1H), 8.48 (d, 1H), 7.80 (d, 1H), 7.62 (d, 1H), 7.61 (d, 1H), 7.35-7.33 (m, 2H), 7.00 (dd, 1H), 6.94 (d, 1H), 6.82 (dd, 1H), 4.97 (dd, 1H), 4.42 (q, 2H), 4.18 (t, 2H), 4.07-4.04 (m, 2H), 3.85-3.82 (m, 1H), 3.80-3.75 (m, 1H), 3.67-3.62 (m, 3H), 3.43-3.39 (m, 2H), 2.92-2.84 (m, 1H), 2.84 (s, 3H), 2.61-2.56 (m, 1H), 2.54-2.49 (m, 1H), 2.07-1.99 (m, 3H), 1.92-1.90 (m, 4H), 1.78-1.72 (m, 2H), 1.68-1.62 (m, 4H), 1.52-1.47 (m, 2H), 1.40 (t, 3H).

Compound 156: MS: m/z 877.0 (M⁺+1); ¹H NMR (DMSO-d₆) δ 11.06 (s, 1H), 8.42 (d, 1H), 7.82 (d, 1H), 7.63-7.60 (m, 2H), 7.37 (d, 1H), 7.01 (dd, 1H), 6.94 (d, 1H), 6.84-6.81 (m, 2H), 4.98 (dd, 1H), 4.43 (q, 2H), 4.22 (t, 2H), 3.86-3.78 (m, 8H), 3.44-3.42 (m, 1H), 2.92-2.85 (m, 1H), 2.84 (s, 3H), 2.61-2.57 (m, 1H), 2.55-2.49 (m, 1H), 2.06-2.03 (m, 1H), 1.92-1.91 (m, 4H), 1.81 (bs, 2H), 1.78-1.71 (m, 2H), 1.68-1.64 (m, 4H), 1.60-1.56 (m 2H), 1.42 (t, 3H), 1.41-1.36 (m, 2H).

Compound 157: MS: m/z 904.8 (M⁺+1); ¹H NMR (DMSO-d₆) δ 11.06 (s, 1H), 8.57 (d, 1H), 7.86 (d, 1H), 7.80 (d, 1H), 7.62 (d, 1H), 7.39 (d, 1H), 7.34-7.32 (m, 2H), 7.13 (dd, 1H), 6.99 (dd, 1H), 4.97 (dd, 1H), 4.56-4.49 (m, 3H), 4.43 (q, 2H), 4.12 (bs, 2H), 3.89-3.82 (m, 1H), 2.95-2.85 (m, 3H), 2.61-2.56 (m, 1H), 2.55-2.48 (m, 7H), 2.14-2.08 (m, 2H), 2.07-2.02 (m, 1H), 1.97-1.86 (m, 4H), 1.85-1.76 (m, 3H), 1.72-1.60 (m, 3H), 1.54-1.48 (m, 2H), 1.41 (t, 3H), 1.29-1.00 (6H).

Compound 158: MS: m/z 912.7 (M⁺+23); ¹H NMR (DMSO-d₆) 11.06 (s, 1H), 8.58 (d, 1H), 7.86 (d, 1H), 7.80 (d, 1H), 7.62 (bs, 1H), 7.39-7.36 (m, 2H), 7.33 (d, 1H), 7.13 (dd, 1H), 7.00 (bs, 1H), 4.98 (dd, 1H), 4.56-4.49 (m, 3H), 4.43 (q, 2H), 4.21 (bs, 2H), 3.89-3.82 (M, 1H), 3.00 (bs, 1H), 2.95-2.86 (m, 3H), 2.71 (bs, 1H), 2.61-2.56 (m, 1H), 2.55-2.48 (m, 5H), 2.14-2.08 (m, 2H), 2.07-2.02 (m, 1H), 2.00-1.93 (m, 1H), 1.92-1.86 (m, 2H), 1.82-1.76 (m, 2H), 1.69-1.60 (m, 3H), 1.54-1.48 (m, 2H), 1.42 (t, 3H), 1.29-1.13 (m, 6H).

Compound 159: MS: m/z 837.5 (M⁺+1); ¹H NMR (DMSO-d₆) δ 11.05 (s, 1H), 8.59 (d, 1H), 7.85 (d, 1H), 7.79 (d, 1H), 7.61 (d, 1H), 7.39 (d, 1H), 7.34-7.33 (m, 2H), 7.13 (dd, 1H), 7.00 (dd, 1H), 4.97 (dd, 1H), 4.55-4.51 (m, 1H), 4.42 (q, 2H), 4.18 (t, 2H), 4.07-4.04 (m, 2H), 3.88-3.82 (m, 1H), 3.67-3.62 (m, 3H), 3.43-3.39 (m, 2H), 2.91-2.85 (m, 1H), 2.62-2.57 (m, 1H), 2.54-2.49 (m, 1H), 2.11-2.08 (m, 2H), 2.06-1.99 (m, 3H), 1.94-1.88 (m, 4H), 1.67-1.60 (m, 2H), 1.53-1.46 (m, 4H), 1.40 (t, 3H).

Compound 160: MS: m/z 848.6 (M⁺+1); ¹H NMR (DMSO-d₆) δ 11.05 (s, 1H), 8.55 (d, 1H), 7.85 (d, 1H), 7.84 (d, 1H), 7.62 (d, 1H), 7.38 (d, 1H), 7.34 (d, 1H), 7.13 (dd, 1H), 7.00 (dd, 1H), 6.86 (d, 1H), 4.97 (dd, 1H), 4.56-4.51 (m, 1H), 4.43 (q, 2H), 4.15 (bs, 2H), 4.08-4.06 (m, 4H), 3.88-3.82 (m, 1H), 2.91-2.86 (m, 1H), 2.80 (bs, 2H), 2.61-2.57 (m, 3H), 2.54-2.49 (m, 3H), 2.11-2.09 (m, 4H), 2.07-2.02 (m, 1H), 1.96 (bs, 2H), 1.91-1.88 (m, 2H), 1.66-1.60 (m, 2H), 1.54-1.48 (m, 2H), 1.42 (t, 3H).

Compound 161: MS: m/z 834.1 (M⁺+1); ¹H NMR (DMSO-d₆) δ 11.06 (s, 1H), 8.54 (d, 1H), 7.85 (d, 1H), 7.83 (d, 1H), 7.61 (d, 1H), 7.38 (d, 1H), 7.37 (d, 1H), 7.13 (dd, 1H), 7.01 (dd, 1H), 6.86 (d, 1H), 4.97 (dd, 1H), 4.56-4.51 (m, 1H), 4.43 (q, 2H), 4.21 (t, 2H), 4.09-4.04 (m, 4H), 3.88-3.82 (m, 1H), 2.90-2.87 (m, 5H), 2.70-2.67 (m, 2H), 2.61-2.57 (m, 1H), 2.54-2.49 (m, 1H), 2.12-2.09 (m, 4H), 2.07-2.02 (m, 1H), 1.91-1.89 (m, 2H), 1.65-1.60 (m, 2H), 1.54-1.48 (m, 2H), 1.42 (t, 3H).

Compound 162: MS: m/z 849.6 (M⁺+1); ¹H NMR (DMSO-d₆) δ 11.06 (s, 1H), 8.52 (d, 1H), 7.86 (dd, 1H), 7.83-7.80 (m, 1H), 7.62 (dd, 1H), 7.39-7.36 (m, 2H), 7.14 (dd, 1H), 7.02 (dd, 1H), 6.94-6.90 (m, 1H), 4.97 (dd, 1H), 4.56-4.51 (m, 1H), 4.43 (q, 2H), 4.21 (t, 2H), 4.18-4.13 (m, 1H), 3.88-3.82 (m, 1H), 3.73-3.68 (m, 2H), 3.62-3.43 (m, 4H), 2.92-2.85 (m, 1H), 2.62-2.55 (m, 1H), 2.54-2.49 (m, 1H), 2.36-2.31 (m, 2H), 2.12-2.09 (m, 2H), 2.07-1.96 (m, 5H), 1.91-1.89 (m, 2H), 1.66-1.60 (m, 2H), 1.54-1.48 (m, 2H), 1.42 (t, 3H).

Compound 163: MS: m/z 843.5 (M⁺+23); ¹H NMR (DMSO-d₆) δ 11.05 (s, 1H), 8.58 (d, 1H), 7.85 (d, 1H), 7.80 (d, 1H), 7.61 (d, 1H), 7.39 (d, 1H), 7.35-7.32 (m, 2H), 7.13 (dd, 1H), 7.00 (dd, 1H), 4.97 (dd, 1H), 4.56-4.47 (m, 3H), 4.42 (q, 2H), 4.10 (t, 2H), 3.89-3.82 (m, 1H), 3.00 (t, 2H), 2.91-2.85 (m, 1H), 2.61-2.56 (m, 1H), 2.52-2.49 (m, 1H), 2.12-2.09 (m, 2H), 2.07-2.02 (m, 1H), 1.91-1.88 (m, 2H), 1.83-1.81 (m, 4H), 1.69-1.60 (m, 3H), 1.54-1.48 (m, 4H), 1.41 (t, 3H), 1.19-1.12 (m, 2H).

Compound 164: MS: m/z 884.9 (M⁺+23); ¹H NMR (DMSO-d₆) δ 11.05 (s, 1H), 8.42 (dd, 1H), 7.83-7.80 (m, 1H), 7.63-7.60 (m, 2H), 7.36 (d, 1H), 7.02 (dd, 1H), 6.94-6.89 (m, 3H), 6.82 (dd, 1H), 4.97 (dd, 1H), 4.43 (q, 2H), 4.23-4.22 (m, 2H), 4.18-4.13 (m, 1H), 3.88-3.75 (m, 2H), 3.72-3.70 (m, 2H), 3.60-3.43 (m, 4H), 2.91-2.85 (m, 1H), 2.85 (s, 3H), 2.62-2.55 (m, 1H), 2.54-2.49 (m, 1H), 2.36-2.31 (m, 2H), 2.06-1.95 (m, 5H), 1.95-1.89 (m, 2H), 1.78-1.71 (m, 2H), 1.69-1.62 (m, 4H), 1.41 (t, 3H).

Compound 165: MS: m/z 856.8 (M⁺+23); ¹H NMR (DMSO-d₆) δ 11.05 (s, 1H), 8.48 (d, 1H), 7.81 (d, 1H), 7.60 (dd, 1H), 7.35-7.32 (m, 2H), 6.99 (dd, 1H), 6.94 (d, 1H), 6.82 (dd, 1H), 4.97 (dd, 1H), 4.51-4.48 (m, 2H), 4.43 (q, 2H), 4.10 (t, 2H), 3.88-3.75 (m, 2H), 3.02-2.98 (m, 2H), 2.91-2.85 (m, 1H), 2.85 (s, 3H), 2.61-2.56 (m, 1H), 2.52-2.49 (m, 1H), 2.07-2.02 (m, 1H), 1.93-1.90 (m, 2H), 1.83-1.81 (m, 4H), 1.78-1.72 (m, 2H), 1.69-1.62 (m, 5H), 1.44-1.40 (m, 2H), 1.41 (t, 3H), 1.19-1.12 (m, 2H).

Compound 166: MS: m/z 925.7 (M⁺+23); ¹H NMR (DMSO-d₆) δ 11.06 (s, 1H), 8.47 (d, 1H), 7.81 (d, 1H), 7.64-7.59 (m, 2H), 7.38 (bs, 1H), 7.33 (d, 1H), 7.00 (bs, 1H), 6.94 (d, 1H), 6.82 (dd, 1H), 4.98 (dd, 1H), 4.54-4.52 (m, 2H), 4.43 (q, 2H), 4.19 (bs, 1H), 3.89-3.75 (m, 2H), 3.52-3.33 (m, 1H), 3.00 (bs, 1H), 2.95-2.86 (m, 3H), 2.85 (s, 3H), 2.71 (bs, 1H), 2.61-2.56 (m, 2H), 2.55-2.48 (m, 4H), 2.07-2.02 (m, 1H), 2.01-1.94 (m, 1H), 1.94-1.89 (m, 2H), 1.82-1.71 (m, 4H), 1.70-1.62 (m, 5H), 1.42 (t, 3H), 1.29-1.14 (6H).

Compound 167: MS: m/z 939.8 (M⁺+23); ¹H NMR (DMSO-d₆) δ 11.06 (s, 1H), 8.47 (d, 1H), 7.80 (d, 1H), 7.62-7.60 (m, 2H), 7.34-7.32 (m, 2H), 6.99 (dd, 1H), 6.94 (d, 1H), 6.82 (dd, 1H), 4.97 (dd, 1H), 4.53-4.52 (m, 2H), 4.43 (q, 2H), 4.13 (bs, 2H), 3.88-3.76 (m, 2H), 2.96-2.87 (m, 3H), 2.85 (s, 3H), 2.61-2.56 (m, 1H), 2.55-2.48 (m, 5H), 2.07-2.02 (m, 1H), 1.95-1.89 (m, 3H), 1.82-1.70 (m, 4H), 1.70-1.62 (m, 5H), 1.42 (t, 3H), 1.29-1.04 (m, 6H).

Compound 168: MS: m/z 831.6 (M⁺+23); ¹H NMR (DMSO-d₆) δ 11.05 (s, 1H), 8.59 (d, 1H), 7.85 (d, 1H), 7.82 (d, 1H), 7.61 (d, 1H), 7.39-7.36 (m, 2H), 7.31 (d, 1H), 7.13 (dd, 1H), 7.02 (dd, 1H), 4.97 (dd, 1H), 4.56-4.51 (m, 1H), 4.25-4.24 (m, 2H), 4.12-4.07 (m, 2H), 3.95 (s, 3H), 3.88-3.82 (m, 3H), 3.76-3.72 (m, 1H), 3.45-3.41 (m, 2H), 2.92-2.85 (m, 1H), 2.62-2.52 (m, 2H), 2.12-2.08 (m, 2H), 2.06-2.00 (m, 1H), 1.98-1.93 (m, 2H), 1.93-1.87 (m, 2H), 1.67-1.61 (m, 2H), 1.56-1.48 (m 4H).

Compound 169: MS: m/z 844.6 (M⁺+23); ¹H NMR (DMSO-d₆) δ 11.05 (s, 1H), 8.49 (d, 1H), 7.82 (d, 1H), 7.62-7.59 (m, 2H), 7.37 (d, 1H), 7.31 (d, 1H), 7.02 (dd, 1H), 6.94 (d, 1H), 6.82 (dd, 1H), 4.97 (dd, 1H), 4.24 (t, 2H), 4.12-4.08 (m, 2H), 3.95 (s, 3H), 3.88-3.82 (m, 3H), 3.81-3.72 (m, 2H), 3.45-3.41 (m, 2H), 2.92-2.85 (m, 1H), 2.85 (s, 3H), 2.62-2.55 (m, 1H), 2.54-2.49 (m, 1H), 2.05-2.00 (m, 1H), 1.98-1.93 (m, 2H), 1.93-1.89 (m, 2H), 1.78-1.72 (m, 2H), 1.70-1.62 (m, 4H), 1.55-1.50 (m 2H).

Compound 170: MS: m/z 871.7 (M⁺+23); H NMR (DMSO-d₆) δ 11.04 (s, 1H), 8.56 (d, 1H), 7.84 (d, 1H), 7.78 (d, 1H), 7.60 (d, 1H), 7.37 (d, 1H), 7.32 (d, 1H), 7.28 (d, 1H), 7.11 (dd, 1H), 7.00 (dd, 1H), 4.96 (dd, 1H), 4.54-4.49 (m, 1H), 4.21-4.19 (m, 2H), 4.13-4.08 (m, 1H), 3.94 (s, 3H), 3.87-3.80 (m, 1H), 3.68-3.65 (m, 4H), 3.63-3.60 (m, 2H), 2.94-2.84 (m, 1H), 2.60-2.53 (m, 1H), 2.53-2.47 (m, 1H), 2.24-2.21 (m, 2H), 2.11-2.06 (m, 2H), 2.04-1.99 (m, 1H), 1.90-1.86 (m, 2H), 1.72-1.68 (m, 2H), 1.65-1.55 (m, 6H), 1.52-1.46 (m, 2H).

Compound 171: MS: m/z 884.7 (M⁺+23); ¹H NMR (DMSO-d₆) δ 11.06 (s, 1H), 8.48 (d, 1H), 7.81 (d, 1H), 7.64-7.60 (m, 2H), 7.35 (d, 1H), 7.31 (d, 1H), 7.03 (dd, 1H), 6.95 (d, 1H), 6.83 (dd, 1H), 4.98 (dd, 1H), 4.23-4.22 (m, 2H), 4.15-4.11 (m, 1H), 3.96 (s, 3H), 3.88-3.76 (m, 2H), 3.70-3.68 (m, 4H), 3.65-3.63 (m, 2H), 2.92-2.85 (m, 1H), 2.85 (s, 3H), 2.62-2.56 (m, 1H), 2.55-2.48 (m, 1H), 2.26-2.23 (m, 2H), 2.06-2.02 (m, 1H), 1.94-1.90 (m, 2H), 1.78-1.65 (m, 8H), 1.63-1.58 (m, 4H).

Compound 172: MS: m/z 899.7 (M⁺+23); ¹H NMR (DMSO-d₆) δ 11.06 (s, 1H), 8.56 (d, 1H), 7.86 (d, 1H), 7.78 (d, 1H), 7.62 (d, 1H), 7.39 (d, 1H), 7.35 (d, 1H), 7.31 (d, 1H), 7.13 (dd, 1H), 7.00 (dd, 1H), 4.97 (dd, 1H), 4.55-4.51 (m, 1H), 4.44 (q, 2H), 4.16 (t, 2H), 4.05-4.00 (m, 1H), 3.88-3.82 (m, 1H), 3.67-3.65 (m, 2H), 3.59-3.57 (m, 2H), 3.47 (t, 2H), 2.92-2.85 (m, 1H), 2.62-2.56 (m, 1H), 2.55-2.48 (m, 1H), 2.22-2.19 (m, 2H), 2.11-2.08 (m, 2H), 2.07-2.02 (m, 1H), 2.02-1.98 (m, 2H), 1.92-1.86 (m, 2H), 1.68-1.60 (m, 4H), 1.59-1.48 (m, 6H), 1.42 (t, 3H).

Compound 173: MS: m/z 913.0 (M⁺+23); ¹H NMR (DMSO-d₆) δ 11.06 (s, 1H), 8.47 (d, 1H), 7.79 (d, 1H), 7.62-7.59 (m, 2H), 7.35 (d, 1H), 7.31 (d, 1H), 7.00 (dd, 1H), 6.94 (d, 1H), 6.82 (dd, 1H), 4.98 (dd, 1H), 4.44 (q, 2H), 4.16 (t, 2H), 4.05-4.00 (m, 1H), 3.87-3.74 (m, 2H), 3.67-3.65 (m, 2H), 3.59-3.57 (m, 2H), 3.47 (t, 2H), 2.92-2.85 (m, 1H), 2.84 (s, 3H), 2.61-2.55 (m, 1H), 2.54-2.48 (m, 1H), 2.22-2.18 (m, 2H), 2.07-2.02 (m, 1H), 2.02-1.98 (m, 2H), 1.92-1.89 (m, 2H), 1.78-1.71 (m, 2H), 1.70-1.62 (m, 6H), 1.59-1.51 (m, 4H), 1.42 (t, 3H).

Compound 174: MS: m/z 857.6 (M⁺+23); ¹H NMR (DMSO-d₆) δ 11.05 (s, 1H), 8.52 (d, 1H), 7.85 (d, 1H), 7.83-7.80 (m, 1H), 7.62 (dd, 1H), 7.39 (d, 1H), 7.30 (d, 1H), 7.13 (dd, 1H), 7.02 (d, 1H), 6.93-6.89 (m, 1H), 4.97 (dd, 1H), 4.56-4.51 (m, 1H), 4.23-4.21 (m, 2H), 4.18-4.13 (m, 1H), 3.95 (s, 3H), 3.89-3.82 (m, 1H), 3.73-3.69 (m, 2H), 3.62-3.42 (m, 4H), 2.92-2.86 (m, 1H), 2.62-2.55 (m, 1H), 2.54-2.49 (m, 1H), 2.36-2.31 (m, 2H), 2.12-2.09 (m, 2H), 2.06-1.96 (m, 5H), 1.92-1.89 (m, 2H), 1.66-1.60 (m, 2H), 1.54-1.48 (m, 2H).

Compound 175: MS: m/z 870.2 (M⁺+23); ¹H NMR (DMSO-d₆) δ 11.06 (s, 1H), 8.42 (d, 1H), 7.83-7.80 (m, 1H), 7.63-7.60 (m, 2H), 7.30 (bs, 1H), 7.03 (d, 1H), 6.94-6.90 (m, 2H), 6.82 (d, 1H), 4.98 (dd, 1H), 4.23-4.21 (m, 2H), 4.18-4.13 (m, 1H), 3.95 (s, 3H), 3.87-3.77 (m, 2H), 3.71 (bs, 2H), 3.60-3.44 (m, 4H), 2.92-2.85 (m, 1H), 2.85 (s, 3H), 2.62-2.55 (m, 1H), 2.54-2.49 (m, 1H), 2.36-2.31 (m, 2H), 2.06-1.96 (m, 5H), 1.92-1.90 (m, 2H), 1.78-1.72 (m, 2H), 1.70-1.62 (m, 4H).

Compound 176: MS: m/z 863.8 (M⁺+1); ¹H NMR (DMSO-d₆) δ 11.05 (s, 1H), 8.51 (d, 1H), 7.85 (d, 1H), 7.82-7.78 (m, 1H), 7.61 (dd, 1H), 7.39 (d, 1H), 7.35 (bs, 1H), 7.13 (dd, 1H), 7.00 (d, 1H), 6.91-6.86 (m, 1H), 4.97 (dd, 1H). 4.56-4.51 (m, 1H), 4.43 (q. 2H), 4.16 (t, 2H), 3.88-3.81 (m. 1H), 3.58-3.40 (m, 6H), 2.92-2.85 (m, 1H), 2.61-2.56 (m, 1H), 2.55-2.49 (m, 1H), 2.32-2.28 (m, 2H), 2.12-2.07 (m, 2H), 2.06-1.97 (m, 5H), 1.97-1.87 (m, 4H), 1.66-1.60 (m, 2H), 1.54-1.48 (m, 2H), 4.41 (t, 3H).

Compound 177: MS: m/z 899.0 (M⁺+23); ¹H NMR (DMSO-d₆) δ 11.05 (s, 1H), 8.41 (d, 1H), 7.82-7.78 (m, 1H), 7.62-7.60 (m, 2H), 7.35 (bs, 1H), 7.00 (d, 1H), 6.94 (d, 1H), 6.91-6.87 (m, 1H), 6.82 (dd, 1H), 4.97 (dd, 1H), 4.43 (q, 2H), 4.17-4.15 (m, 2H), 4.08-4.04 (m, 1H), 3.87-3.76 (m, 2H), 3.57-3.43 (m, 6H), 2.92-2.85 (m, 1H), 2.85 (s, 3H), 2.62-2.55 (m, 1H), 2.54-2.49 (m, 1H), 2.32-2.28 (m, 2H), 2.06-1.89 (m, 9H), 1.78-1.72 (m, 2H), 1.70-1.61 (m, 4H), 1.41 (t, 3H).

Compound 178: MS: m/z 884.6 (M⁺+23); ¹H NMR (DMSO-d₆) δ 11.05 (s, 1H), 8.41 (d, 1H), 7.82-7.81 (m, 1H), 7.62-7.60 (m, 2H), 7.29 (bs, 1H), 7.00 (d, 1H), 6.94 (d, 1H), 6.91-6.87 (m, 1H), 6.83 (dd, 1H), 4.97 (dd, 1H), 4.17-4.15 (m, 2H), 4.06 (s, 3H), 3.87-3.76 (m, 2H), 3.57-3.43 (m, 6H), 2.92-2.85 (m, 1H), 2.85 (s, 3H), 2.62-2.55 (m, 1H), 2.54-2.49 (m, 1H), 2.32-2.28 (m, 2H), 2.00-1.89 (m, 9H), 1.78-1.72 (m, 2H), 1.70-1.60 (m, 4H).

Compound 179: MS: m/z 872.0 (M⁺+23); ¹H NMR (DMSO-d₆) δ 11.05 (s, 1H), 8.51 (dd, 1H), 7.85 (d, 1H), 7.82-7.77 (m, 1H), 7.61 (dd, 1H), 7.39 (d, 1H), 7.29 (bs, 1H), 7.13 (dd, 1H), 7.02-7.00 (m, 1H), 6.92-6.87 (m, 1H), 4.97 (dd, 1H), 4.56-4.51 (m, 1H), 4.16 (t, 2H), 4.08-4.03 (m, 1H), 3.96 (s, 3H), 3.88-3.81 (m, 1H), 3.56-3.42 (m, 6H), 2.92-2.85 (m, 1H), 2.62-2.56 (m, 1H), 2.55-2.49 (m, 1H), 2.32-2.27 (m, 2H), 2.12-2.08 (m, 2H), 2.06-1.97 (m, 5H), 1.97-1.87 (m, 4H), 1.66-1.60 (m, 2H), 1.54-1.48 (m, 2H).

Compound 180: MS: m/z 889.8 (M⁺+1); ¹H NMR (DMSO-d₆) δ 11.06 (s, 1H), 8.48 (d, 1H), 7.81 (d, 1H), 7.65-7.60 (m, 2H), 7.41-7.31 (m, 2H), 7.01 (bs, 1H), 6.94 (d, 1H), 6.83 (dd, 1H), 4.98 (dd, 1H), 4.44 (q, 2H), 4.20 (bs, 2H), 3.87-3.75 (m, 2H), 3.71 (bs, 4H), 2.92-2.85 (m, 1H), 2.85 (s, 3H), 2.77 (bs, 2H), 2.63-2.56 (m, 2H), 2.56-2.50 (m, 4H), 2.07-2.02 (m, 1H), 1.93-1.89 (m, 2H), 1.78-1.72 (m, 2H), 1.71-1.62 (m, 4H), 1.62-1.46 (m, 8H), 1.42 (t, 3H).

Compound 181: MS: m/z 925.8 (M⁺+23); ¹H NMR (DMSO-d₆) δ 11.06 (s, 1H), 8.48 (d, 1H), 7.81 (d, 1H), 7.68-7.60 (m, 2H), 7.35 (bs, 2H), 7.00 (d, 1H), 6.94 (bs, 1H), 6.82 (d, 1H), 4.98 (dd, 1H), 4.48-4.42 (m, 2H), 4.16 (bs, 2H), 3.89-3.74 (m, 2H), 3.72 (bs, 4H), 2.92-2.85 (m, 1H), 2.85 (s, 3H), 2.63-2.55 (m, 2H), 2.56-2.50 (m, 4H), 2.45-2.33 (m, 2H), 2.09-2.02 (m, 1H), 1.97-1.89 (m, 4H), 1.78-1.62 (m, 8H), 1.62-1.46 (m, 6H), 1.42 (t, 3H).

Compound 182: MS: m/z 890.0 (M⁺+23); ¹H NMR (DMSO-d₆) δ 11.06 (s, 1H), 8.58 (d, 1H), 7.86 (d, 1H), 7.80 (d, 1H), 7.62 (bs, 1H), 7.42-7.36 (m, 2H), 7.33 (d, 1H), 7.13 (dd, 1H), 7.01 (bs, 1H), 4.98 (dd, 1H), 4.56-4.51 (m, 1H), 4.44 (q, 2H), 4.21 (bs, 2H), 3.89-3.82 (m, 1H), 3.71 (bs, 4H), 2.92-2.85 (m, 1H), 2.77 (bs, 2H), 2.63-2.56 (m, 2H), 2.55-2.48 (m, 4H), 2.14-2.08 (m, 2H), 2.07-2.02 (m, 1H), 1.93-1.86 (m, 2H), 1.67-1.47 (m, 12H), 1.42 (t, 3H).

Compound 183: MS: m/z 891.0 (M⁺+1); ¹H NMR (DMSO-d₆) δ 11.06 (s, 1H), 8.58 (d, 1H), 7.85 (d, 1H), 7.81 (d, 1H), 7.63 (d, 1H), 7.39 (d, 1H), 7.37-7.31 (m, 2H), 7.14 (dd, 1H), 7.00 (dd, 1H), 4.98 (dd, 1H), 4.56-4.51 (m, 1H), 4.44 (q, 2H), 4.15 (bs, 2H), 3.89-3.82 (m, 1H), 3.71 (bs, 4H), 2.92-2.85 (m, 1H), 2.63-2.55 (m, 2H), 2.55-2.46 (m, 4H), 2.46-2.31 (m, 2H), 2.14-2.07 (m, 2H), 2.06-2.01 (m, 1H), 1.98-1.87 (m, 4H), 1.67-1.45 (m, 12H), 1.42 (t, 3H).

Compound 184: MS: m/z 886.9 (M⁺+23); ¹H NMR (DMSO-d₆) δ 11.05 (s, 1H), 8.49 (d, 1H), 7.81 (d, 1H), 7.61-7.60 (m, 2H), 7.36 (d, 1H), 7.32 (d, 1H), 7.00 (dd, 1H), 6.94 (d, 1H), 6.82 (dd, 1H), 4.97 (dd, 1H), 4.20 (q, 2H), 4.12 (t, 2H), 4.09-4.04 (m, 2H), 3.88-3.82 (m, 1H), 3.80-3.76 (m, 1H), 3.63-3.59 (m, 1H), 3.54 (t, 2H), 3.45-3.40 (m, 2H), 2.92-2.85 (m, 1H), 2.85 (s, 3H), 2.61-2.55 (m, 1H), 2.54-2.50 (m, 1H), 2.07-2.02 (m, 1H), 1.94-1.89 (m, 4H), 1.86-1.82 (m, 2H), 1.78-1.62 (m, 6H), 1.52-1.46 (m, 2H), 1.41 (t, 3H).

Compound 185: MS: m/z 873.9 (M⁺+23); ¹H NMR (DMSO-d₆) δ 11.05 (s, 1H), 8.59 (d, 1H), 7.85 (d, 1H), 7.81 (d, 1H), 7.61 (d, 1H), 7.39 (d, 1H), 7.36 (d, 1H), 7.32 (d, 1H), 7.13 (dd, 1H). 6.99 (dd, 1H), 4.97 (dd, 1H), 4.56-4.51 (m, 1H), 4.42 (q, 2H), 4.12 (t, 2H), 4.08-4.04 (m, 2H), 3.89-3.82 (m, 1H), 3.63-3.59 (m, 1H), 3.54 (t, 2H), 3.45-3.40 (m, 2H), 2.91-2.85 (m, 1H), 2.62-2.52 (m, 2H), 2.11-2.08 (m, 2H), 2.07-2.07 (m, 1H), 1.93-1.87 (m, 4H), 1.86-1.81 (m, 2H), 1.73-1.67 (m, 2H), 1.67-1.61 (m, 2H), 1.54-1.48 (m, 4H), 1.41 (t, 3H).

Compound 186: MS: m/z 851.0 (M⁺+1); ¹H NMR (DMSO-d₆) δ 11.05 (s, 1H), 8.49 (d, 1H), 7.81 (d, 1H), 7.61-7.59 (m, 2H), 7.36 (d, 1H), 7.26 (d, 1H), 7.00 (dd, 1H), 6.94 (d, 1H), 6.82 (dd, 1H), 4.97 (dd, 1H), 4.12 (t, 2H), 4.09-4.04 (m, 2H), 3.94 (s, 3H), 3.87-3.82 (m, 1H), 3.80-3.76 (m, 1H), 3.63-3.60 (m, 1H), 3.55 (t, 2H), 3.45-3.41 (m, 2H), 2.92-2.84 (m, 1H), 2.85 (s, 3H), 2.61-2.55 (m, 1H), 2.54-2.50 (m, 1H), 2.06-2.00 (m, 1H), 1.95-1.89 (m, 4H), 1.87-1.82 (m, 2H), 1.78-1.62 (m, 6H), 1.52-1.46 (m, 2H).

Compound 187: MS: m/z 837.6 (M⁺+1); ¹H NMR (DMSO-d₆) δ 11.05 (s, 1H), 8.59 (d, 1H), 7.85 (d, 1H), 7.80 (d, 1H), 7.61 (d, 1H), 7.39 (d, 1H), 7.36 (d, 1H), 7.26 (d, 1H), 7.13 (dd, 1H), 7.00 (dd, 1H), 4.97 (dd, 1H), 4.56-4.51 (m, 1H), 4.12 (t, 2H), 4.08-4.04 (m, 2H), 3.94 (s, 3H), 3.89-3.82 (m, 1H), 3.63-3.59 (m, 1H), 3.54 (t, 2H), 3.45-3.40 (m, 2H), 2.92-2.85 (m, 1H), 2.61-2.53 (m, 2H), 2.11-2.08 (m, 2H), 2.05-2.00 (m, 1H), 1.95-1.87 (m, 4H), 1.87-1.82 (m, 2H), 1.73-1.67 (m, 2H), 1.66-1.61 (m, 2H), 1.54-1.46 (m, 4H).

Compound 188: MS: m/z 844.9 (M⁺+23); ¹H NMR (DMSO-d₆) δ 11.06 (s, 1H), 8.61 (d, 1H), 7.86-7.83 (m, 2H), 7.62 (d, 1H), 7.39 (d, 1H), 7.35 (d, 1H), 7.27 (d, 1H), 7.13 (dd, 1H), 7.02 (d, 1H), 4.97 (dd, 1H), 4.55-4.51 (m, 1H), 4.11-4.08 (m, 1H), 4.00-3.96 (m, 1H), 3.96 (s, 3H), 3.89-3.83 (m, 1H), 3.72 (bs, 4H), 2.92-2.86 (m, 1H), 2.62-2.56 (m, 1H), 2.54-2.46 (m, 6H), 2.30-2.24 (m, 2H), 2.12-2.08 (m, 2H), 2.05-2.01 (m, 1H), 1.91-1.87 (m, 2H), 1.67-1.61 (m, 2H), 1.54-1.48 (m, 2H), 1.07 (d, 3H).

Compound 189: MS: m/z 857.6 (M⁺+23); ¹H NMR (DMSO-d₆) δ 11.06 (s, 1H), 8.50 (d, 1H), 7.84 (d, 1H), 7.63-7.60 (m, 2H), 7.35 (d, 1H), 7.27 (d, 1H), 7.03 (dd, 1H), 6.94 (d, 1H), 6.82 (dd, 1H), 4.97 (dd, 1H), 4.11-4.08 (m, 1H), 3.99-3.96 (m, 1H), 3.96 (s, 3H), 3.88-3.81 (m, 1H), 3.80-3.75 (m, 1H), 3.71 (bs, 4H), 2.92-2.86 (m, 1H), 2.85 (s, 3H), 2.62-2.58 (m, 1H), 2.58-2.46 (m, 6H), 2.29-2.24 (m, 2H), 2.05-2.01 (m, 1H), 1.94-1.89 (m, 2H), 1.78-1.71 (m, 2H), 1.70-1.62 (m, 4H), 1.07 (d, 3H).

Compound 190: MS: m/z 819.5 (M⁺+1); ¹H NMR (DMSO-d₆) δ 11.05 (s, 1H), 9.03 (d, 1H), 8.11-8.07 (m, 2H), 7.86 (d, 1H), 7.62 (d, 1H), 7.40 (d, 1H), 7.34 (d, 1H), 7.14 (dd, 1H), 7.01 (dd, 1H), 6.95-6.93 (m, 1H), 4.97 (dd, 1H), 4.57-4.52 (m, 1H), 4.43 (q, 2H), 4.17 (t, 2H), 3.95-3.88 (m, 1H), 3.26 (bs, 2H), 2.92-2.85 (m, 1H), 2.74 (bs, 4H), 2.66-2.63 (m, 2H), 2.62-2.55 (m, 1H), 2.54-2.48 (m, 1H), 2.15-2.10 (m, 2H), 2.06-2.00 (m, 3H), 1.94-1.89 (m, 2H), 1.72-1.66 (m, 2H), 1.56-1.50 (m, 2H), 1.41 (t, 3H).

Compound 191: MS: m/z 827.6 (M⁺+23); ¹H NMR (DMSO-d₆) δ 11.06 (s, 1H), 9.04 (d, 1H), 8.12-8.09 (m, 2H), 7.86 (d, 1H), 7.63 (d, 1H), 7.41-7.40 (m, 2H), 7.15 (dd, 1H), 7.03 (dd, 1H), 6.96-6.94 (m, 1H), 4.98 (dd, 1H), 4.58-4.53 (m, 1H), 4.44 (q, 2H), 4.30 (t, 2H), 3.95-3.89 (m, 1H), 3.38 (bs, 2H), 2.95-2.93 (m, 2H), 2.92-2.85 (m, 3H), 2.76 (bs, 2H), 2.63-2.56 (m, 2H), 2.15-2.10 (m, 2H), 2.08-2.03 (m, 1H), 1.94-1.91 (m, 2H), 1.73-1.66 (m, 2H), 1.57-1.50 (m, 2H), 1.43 (t, 3H).

Compound 192: MS: m/z 858.3 (M⁺+23); ¹H NMR (DMSO-d₆) δ 11.06 (s, 1H), 8.61 (d, 1H), 7.87-7.83 (m, 2H), 7.63 (d, 1H), 7.39 (d, 1H), 7.36-7.33 (m, 2H), 7.14 (dd, 1H), 7.02 (d, 1H), 4.98 (dd, 1H), 4.56-4.51 (m, 1H), 4.44 (q, 2H), 4.10-4.08 (m, 1H), 4.00-3.97 (m, 1H), 3.89-3.82 (m, 1H), 3.71 (bs, 4H), 2.92-2.86 (m, 1H), 2.64-2.56 (m, 2H), 2.54-2.48 (m, 4H), 2.30-2.24 (m, 2H), 2.12-2.08 (m, 3H), 2.07-2.02 (m, 1H), 1.91-1.87 (m, 2H), 1.67-1.61 (m, 2H), 1.54-1.49 (m, 2H), 1.42 (t, 3H), 1.07 (d, 3H).

Compound 193: MS: m/z 871.9 (M⁺+23); ¹H NMR (DMSO-d₆) δ 11.07 (s, 1H), 8.52 (d, 1H), 7.85 (d, 1H), 7.64-7.61 (m, 2H), 7.36 (d, 1H), 7.34 (d, 1H), 7.03 (dd, 1H), 6.95 (d, 1H), 6.84 (dd, 1H), 4.99 (dd, 1H), 4.44 (q, 2H), 4.11-4.09 (m, 1H), 4.00-3.98 (m, 1H), 3.89-3.82 (m, 1H), 3.81-3.71 (m, 1H), 3.72 (bs, 4H), 2.93-2.86 (m, 1H), 2.86 (s, 3H), 2.63-2.58 (m, 1H), 2.58-2.46 (m, 6H), 2.30-2.24 (m, 2H), 2.08-2.03 (m, 1H), 1.96-1.90 (m, 2H), 1.79-1.72 (m, 2H), 1.71-1.64 (m, 4H), 1.43 (t, 3H), 1.08 (d, 3H).

Compound 194: MS: m/z 899.0 (M⁺+23); ¹H NMR (DMSO-d₆) δ 11.07 (s, 1H), 8.58 (d, 1H), 7.86 (d, 1H), 7.82 (d, 1H), 7.64 (d, 1H), 7.39 (d, 1H), 7.37-7.31 (m, 1H), 7.29 (bs, 1H), 7.14 (dd, 1H), 7.01 (dd, 1H), 4.98 (dd, 1H), 4.56-4.52 (m, 1H), 4.16 (bs, 2H), 3.97 (s, 3H), 3.89-3.83 (m, 1H), 3.72 (bs, 4H), 2.93-2.86 (m, 1H), 2.63-2.56 (m, 1H), 2.55-2.46 (m, 5H), 2.46-2.31 (m, 2H), 2.14-2.10 (m, 2H), 2.06-2.00 (m, 1H), 1.99-1.88 (m, 4H), 1.67-1.42 (m, 12H).

Compound 195: MS: m/z 885.4 (M⁺+23); ¹H NMR (DMSO-d₆) δ 11.06 (s, 1H), 8.59 (d, 1H), 7.86 (d, 1H), 7.81 (d, 1H), 7.63 (bs, 1H), 7.39 (d, 1H), 7.37-7.31 (m, 2H), 7.14 (dd, 1H), 7.03 (bs, 1H), 4.98 (dd, 1H), 4.56-4.52 (m, 1H), 4.21 (bs, 2H), 3.97 (s, 3H), 3.89-3.83 (m, 1H), 3.72 (bs, 4H), 2.93-2.86 (m, 1H), 2.78 (bs, 2H), 2.63-2.56 (m, 2H), 2.55-2.48 (m, 4H), 2.14-2.10 (m, 2H), 2.06-2.01 (m, 1H), 1.93-1.88 (m, 2H), 1.73-1.45 (m, 12H).

Compound 196: MS: m/z 805.6 (M⁺+1); ¹H NMR (DMSO-d₆) δ 11.06 (s, 1H), 9.04 (d, 1H), 8.12-8.07 (m, 2H), 7.86 (d, 1H), 7.62 (d, 1H), 7.40 (d, 1H), 7.29 (d, 1H), 7.15 (dd, 1H), 7.02 (dd, 1H), 6.95-6.93 (m, 1H), 4.98 (dd, 1H), 4.58-4.52 (m, 1H), 4.18 (t, 2H), 3.95 (s, 3H), 3.94-3.89 (m, 1H), 3.26 (bs, 2H), 2.92-2.86 (m, 1H), 2.74 (bs, 4H), 2.66-2.64 (m, 2H), 2.62-2.56 (m, 2H), 2.15-2.10 (m, 2H), 2.06-2.01 (m, 3H), 1.96-1.91 (m, 2H), 1.73-1.66 (m, 2H), 1.57-1.50 (m, 2H).

Compound 197: MS: m/z 791.3 (M⁺+1); ¹H NMR (DMSO-d₆) δ 11.06 (s, 1H), 9.04 (d, 1H), 8.12-8.09 (m, 2H), 7.86 (d, 1H), 7.63 (d, 1H), 7.40 (d, 1H), 7.35 (d, 1H), 7.15 (dd, 1H), 7.04 (dd, 1H), 6.96-6.94 (m, 1H), 4.98 (dd, 1H), 4.58-4.52 (m, 1H), 4.30 (t, 2H), 3.97 (s, 3H), 3.94-3.89 (m, 1H), 3.38 (bs, 2H), 2.95-2.94 (m, 2H), 2.92-2.83 (m, 3H), 2.76 (bs, 2H), 2.63-2.56 (m, 1H), 2.55-2.50 (m, 1H), 2.14-2.10 (m, 2H), 2.06-2.02 (m, 1H), 1.95-1.90 (m, 2H), 1.73-1.66 (m, 2H), 1.56-1.50 (m, 2H).

Compound 198: MS: m/z 890.0 (M⁺+1); ¹H NMR (DMSO-d₆) δ 11.07 (s, 1H), 8.48 (d, 1H), 7.82 (d, 1H), 7.68-7.59 (m, 2H), 7.40-7.26 (m, 2H), 7.02 (d, 1H), 6.95 (d, 1H), 6.83 (dd, 1H), 4.99 (dd, 1H), 4.18 (bs, 2H), 3.97 (s, 3H), 3.88-3.75 (m, 2H), 3.73 (bs, 4H), 2.93-2.85 (m, 1H), 2.86 (s, 3H), 2.63-2.55 (m, 2H), 2.56-2.50 (m, 4H), 2.45-2.32 (m, 2H), 2.07-2.01 (m, 1H), 1.99-1.89 (m, 4H), 1.79-1.73 (m, 2H), 1.73-1.63 (m, 6H), 1.63-1.44 (m, 6H).

Compound 199: MS: m/z 875.6 (M⁺+1); ¹H NMR (DMSO-d₆) δ 11.06 (s, 1H), 8.48 (d, 1H), 7.81 (d, 1H), 7.65-7.60 (m, 2H), 7.35-7.31 (m, 2H), 7.01 (d, 1H), 6.95 (d, 1H), 6.83 (dd, 1H), 4.98 (dd, 1H), 4.21 (bs, 2H), 3.97 (s, 3H), 3.88-3.75 (m, 2H), 3.71 (bs, 4H), 2.93-2.85 (m, 1H), 2.85 (s, 3H), 2.78 (bs, 2H), 2.63-2.56 (m, 2H), 2.56-2.50 (m, 4H), 2.06-2.01 (m, 1H), 1.94-1.89 (m, 2H), 1.79-1.72 (m, 2H), 1.71-1.62 (m, 4H), 1.61-1.45 (m, 8H).

Compound 200: MS: m/z 819.8 (M⁺+1); ¹H NMR (DMSO-d₆) δ 11.06 (s, 1H), 9.04 (d, 1H), 8.12-8.08 (m, 2H), 7.86 (d, 1H), 7.62 (d, 1H), 7.40 (d, 1H), 7.27 (d, 1H), 7.15 (dd, 1H), 7.03 (dd, 1H), 6.95-6.93 (m, 1H), 4.98 (dd, 1H), 4.58-4.52 (m, 1H), 4.12-4.09 (m, 1H), 4.00-3.96 (m, 1H), 3.95 (s, 3H), 3.94-3.89 (m, 1H), 3.26 (bs, 2H), 2.92-2.86 (m, 1H), 2.78-2.66 (m, 4H), 2.62-2.55 (m, 3H), 2.40-2.35 (m, 1H), 2.34-2.28 (m, 1H), 2.15-2.10 (m, 2H), 2.06-2.01 (m, 1H), 1.95-1.89 (m, 2H), 1.73-1.67 (m, 2H), 1.56-1.50 (m, 2H), 1.08 (d, 3H).

Compound 201: MS: m/z 832.7 (M⁺+1); ¹H NMR (DMSO-d₆) δ 11.06 (s, 1H), 8.95 (d, 1H), 8.13-8.09 (m, 2H), 7.63-7.61 (m, 2H), 7.35 (d, 1H), 7.02 (dd, 1H), 6.97-6.94 (m, 2H), 6.84 (dd, 1H), 4.98 (dd, 1H), 4.43 (q, 2H), 4.18 (t, 2H), 3.95-3.88 (m, 1H), 3.82-3.78 (m, 1H), 3.27 (bs, 2H), 2.92-2.86 (m, 1H), 2.86 (s, 3H), 2.74 (bs, 4H), 2.68-2.63 (m, 2H), 2.63-2.54 (m, 2H), 2.07-2.02 (m, 3H), 1.97-1.92 (m, 2H), 1.81-1.68 (m, 6H), 1.42 (t, 3H).

Compound 202: MS: m/z 818.6 (M⁺+1); ¹H NMR (DMSO-d₆) δ 11.06 (s, 1H), 8.94 (d, 1H), 8.13-8.09 (m, 2H), 7.64-7.60 (m, 2H), 7.41 (d, 1H), 7.04 (dd, 1H), 6.97-6.94 (m, 2H), 6.84 (dd, 1H), 4.98 (dd, 1H), 4.44 (q, 2H), 4.30 (t, 2H), 3.95-3.88 (m, 1H), 3.82-3.78 (m, 1H), 3.39-3.37 (m, 2H), 2.95-2.93 (m, 2H), 2.92-2.84 (m, 1H), 2.86 (s, 3H), 2.76 (bs, 2H), 2.63-2.56 (m, 1H), 2.55-2.50 (m, 3H), 2.08-2.03 (m, 1H), 1.97-1.92 (m, 2H), 1.81-1.68 (m, 6H), 1.43 (t, 3H).

Compound 203: MS: m/z 876.3 (M⁺+1); ¹H NMR (DMSO-d₆) δ 11.06 (s, 1H), 8.58 (d, 1H), 7.86 (d, 1H), 7.81 (d, 1H), 7.62 (d, 1H), 7.40-7.36 (m, 2H), 7.33 (d, 1H), 7.14 (dd, 1H), 7.02 (d, 1H), 4.98 (dd, 1H), 4.60-4.51 (m, 2H), 4.51-4.46 (m, 2H), 4.43 (q, 2H), 3.89-3.83 (m, 1H), 3.03-3.00 (m, 2H), 2.92-2.86 (m, 1H), 2.72 (bs, 2H), 2.63-2.56 (m, 2H), 2.32-2.22 (m, 2H), 2.21-2.16 (m, 2H), 2.14-2.08 (m, 2H), 2.07-1.96 (m, 3H), 1.93-1.87 (m, 2H), 1.87-1.80 (m, 2H), 1.73-1.59 (m, 5H), 1.55-1.49 (m, 2H), 1.41 (t, 3H), 1.16-1.07 (m, 2H).

Compound 204: MS: m/z 876.8 (M⁺+1); ¹H NMR (DMSO-d₆) δ 11.06 (s, 1H), 8.52 (d, 1H), 7.86 (d, 1H), 7.83 (d, 1H), 7.62 (d, 1H), 7.43-7.32 (m, 2H), 7.14 (d, 1H), 7.02 (bs, 1H), 6.94 (d, 1H), 4.98 (dd, 1H), 4.69-4.50 (m, 3H), 4.50-4.38 (m, 6H), 3.89-3.83 (m, 1H), 3.60-3.58 (m, 1H), 3.18-3.06 (m, 1H), 2.92-2.86 (m, 1H), 2.83-2.72 (m, 2H), 2.63-2.56 (m, 2H), 2.37-2.25 (m, 2H), 2.23-2.15 (m, 2H), 2.15-2.08 (m, 2H), 2.08-1.97 (m, 3H), 1.95-1.87 (m, 2H), 1.73-1.59 (m, 7H), 1.57-1.49 (m, 2H), 1.58-1.38 (m, 3H).

Compound 205: MS: m/z 821.5 (M⁺+1); ¹H NMR (DMSO-d₆) δ 11.06 (s, 1H), 9.02 (d, 1H), 8.13 (bs, 1H), 7.86 (d, 1H), 7.84 (d, 1H), 7.64 (d, 1H), 7.40 (d, 1H), 7.36 (bs, 1H), 7.14 (dd, 1H), 7.02 (dd, 1H), 4.98 (dd, 1H), 4.57-4.52 (m, 1H), 4.44 (q, 2H), 4.18 (bs, 2H), 3.95-3.89 (m, 1H), 3.13-2.96 (m, 1H), 2.92-2.86 (m, 1H), 2.63-2.56 (m, 2H), 2.56-2.50 (m, 6H), 2.18-2.08 (m, 4H), 2.08-2.03 (m, 2H), 1.98-1.84 (m, 5H), 1.73-1.66 (m, 2H), 1.56-1.50 (m, 2H), 1.43 (t, 3H).

Compound 206: MS: m/z 807.2 (M⁺+1); ¹H NMR (DMSO-d₆) δ 11.06 (s, 1H), 9.03 (d, 1H), 8.11 (d, 1H), 7.87-7.83 (m, 2H), 7.63 (d, 1H), 7.43-7.36 (m, 2H), 7.14 (dd, 1H), 7.03 (d, 1H), 4.98 (dd, 1H), 4.57-4.52 (m, 1H), 4.45 (q, 2H), 4.25 (bs, 2H), 3.95-3.89 (m, 1H), 3.13 (bs, 2H), 3.06-2.95 (m, 1H), 2.93-2.77 (m, 3H), 2.63-2.52 (m, 2H), 2.31-2.18 (m, 2H), 2.14-2.08 (m, 2H), 2.08-2.00 (m, 1H), 1.98-1.79 (m, 6H), 1.72-1.66 (m, 2H), 1.56-1.50 (m, 2H), 1.43 (t, 3H).

Compound 207: MS: m/z 807.6 (M⁺+1); ¹H NMR (DMSO-d₆) δ 11.07 (s, 1H), 9.03 (d, 1H), 8.13 (bs, 1H), 7.87-7.83 (m, 2H), 7.64 (d, 1H), 7.40 (d, 1H), 7.30 (bs, 1H), 7.15 (dd, 1H), 7.03 (d, 1H), 4.98 (dd, 1H), 4.57-4.52 (m, 1H), 4.19 (bs, 2H), 3.97 (s, 3H), 3.95-3.89 (m, 1H), 3.13-2.97 (m, 1H), 2.93-2.87 (m, 1H), 2.63-2.59 (m, 1H), 2.58-2.50 (m, 7H), 2.15-2.07 (m, 5H), 2.06-2.01 (m, 2H), 1.97-1.85 (m, 4H), 1.73-1.66 (m, 2H), 1.56-1.50 (m, 2H).

Compound 208: MS: m/z 793.1 (M⁺+1); ¹H NMR (DMSO-d₆) δ 11.06 (s, 1H), 9.03 (d, 1H), 8.11 (d, 1H), 7.87-7.83 (m, 2H), 7.63 (d, 1H), 7.40 (d, 1H), 7.35 (bs, 1H), 7.15 (dd, 1H), 7.03 (d, 1H), 4.98 (dd, 1H), 4.57-4.52 (m, 1H), 4.25 (bs, 2H), 3.97 (s, 3H), 3.95-3.89 (m, 1H), 3.13 (bs, 2H), 3.04-2.97 (m, 1H), 2.92-2.79 (m, 3H), 2.63-2.59 (m, 1H), 2.58-2.52 (m, 1H), 2.31-2.22 (m, 2H), 2.15-2.10 (m, 2H), 2.06-2.01 (m, 1H), 1.97-1.83 (m, 6H), 1.72-1.66 (m, 2H), 1.56-1.50 (m, 2H).

Compound 209: MS: m/z 858.6 (M⁺+23); ¹H NMR (DMSO-d) δ 11.06 (s, 1H), 8.61 (d, 1H), 7.87-7.82 (m, 2H), 7.62 (d, 1H), 7.39 (d, 1H), 7.36-7.34 (m, 2H), 7.14 (dd, 1H), 7.02 (dd, 1H), 4.98 (dd, 1H), 4.76-4.72 (m, 1H), 4.56-4.51 (m, 1H), 4.43 (q, 2H), 3.89-3.83 (m, 1H), 3.70 (bs, 4H), 2.92-2.86 (m, 1H), 2.64-2.55 (m, 2H), 2.54-2.48 (m, 6H), 2.12-2.08 (m, 2H), 2.07-2.02 (m, 1H), 1.91-1.81 (m, 4H), 1.67-1.61 (m, 2H), 1.54-1.49 (m, 2H), 1.41 (t, 3H), 1.33 (d, 3H).

Compound 210: MS: m/z 870.9 (M⁺+23); ¹H NMR (DMSO-d₆) δ 11.06 (s, 1H), 8.55 (d, 1H), 7.87-7.82 (m, 2H), 7.62 (d, 1H), 7.39-7.37 (m, 2H), 7.14 (dd, 1H), 7.02 (dd 1H), 6.86 (d, 1H), 4.98 (dd, 1H), 4.64-4.51 (m, 2H), 4.44 (q, 2H), 4.27-4.20 (m, 2H), 3.89-3.78 (m, 3H), 3.10-3.00 (m, 1H), 2.92-2.86 (m, 1H), 2.80-2.63 (m, 4H), 2.63-2.56 (m, 1H), 2.52-2.49 (m, 1H), 2.38-2.75 (m, 2H), 2.12-2.07 (m, 2H), 2.08-1.96 (m, 3H), 1.95-1.87 (m, 2H), 1.75-1.61 (m, 4H), 1.55-1.49 (m, 2H), 1.41 (t, 3H).

Compound 211: MS: m/z 835.2 (M⁺+1); ¹H NMR (DMSO-d₆) δ 11.06 (s, 1H), 9.02 (d, 1H), 8.14 (bs, 1H), 7.86 (d, 1H), 7.82 (d, 1H), 7.64 (d, 1H), 7.40 (d, 1H), 7.38 (bs, 1H), 7.14 (dd, 1H), 7.03 (dd, 1H), 4.98 (dd, 1H), 4.82-4.72 (m, 1H), 4.57-4.52 (m, 1H), 4.44 (q, 2H), 3.95-3.88 (m, 1H), 3.08-2.94 (m, 1H), 2.92-2.86 (m, 1H), 2.63-2.56 (m, 2H), 2.56-2.50 (m, 6H), 2.24-2.08 (m, 5H), 2.08-2.02 (m, 2H), 2.00-1.82 (m, 4H), 1.72-1.66 (m, 2H), 1.57-1.50 (m, 2H), 1.42 (t, 3H), 1.34 (d, 3H).

Compound 212: MS: m/z 926.8 (M⁺+23).

Compound 213: MS: m/z 926.6 (M⁺+23); ¹H NMR (DMSO-d₆) δ 11.06 (s, 1H), 8.24 (d, 1H), 7.91 (d, 1H), 7.82 (d, 1H), 7.62 (d, 1H), 7.41-7.36 (m, 2H), 7.25 (d, 1H), 7.05-7.01 (m, 2H), 4.98 (dd, 1H), 4.64-4.55 (m, 1H), 4.54-4.41 (m, 4H), 4.47 (s, 1H), 4.01 (d, 1H), 3.05-3.01 (m, 2H), 2.92-2.86 (m, 1H), 2.72 (bs, 2H), 2.63-2.56 (m, 2H), 2.34-2.15 (m, 4H), 2.09-1.96 (m, 3H), 1.94-1.81 (m, 3H), 1.73-1.63 (m, 2H), 1.41 (t, 3H), 1.29-1.22 (m, 2H), 1.23 (s, 6H), 1.15 (s, 6H).

Compound 214: MS: m/z 924.9 (M⁺+23); ¹H NMR (DMSO-d₆) δ 11.06 (s, 1H), 7.91 (d, 1H), 7.75 (d, 2H), 7.62 (d, 1H), 7.39-7.37 (m, 2H), 7.21 (d, 1H), 7.03-7.00 (m, 2H), 6.54 (d, 2H), 4.98 (dd, 1H), 4.63-4.54 (m, 1H), 4.44 (q, 2H), 4.32 (s, 1H), 4.06 (d, 1H), 3.52-3.49 (m, 1H), 3.40-3.37 (m, 1H), 3.30-3.25 (m, 1H), 2.95-2.85 (m, 2H), 2.76 (bs, 1H), 2.62-2.59 (m, 1H), 2.59-2.54 (m, 1H), 2.44-2.37 (m, 1H), 2.34-2.24 (m, 3H), 2.19-2.13 (m, 1H), 2.07-1.97 (m, 3H), 1.74-1.57 (m, 5H), 1.41 (t, 3H), 1.29-1.22 (m, 2H), 1.22 (s, 6H), 1.13 (s, 6H).

Compound 215: MS: m/z 884.3 (M⁺+23); ¹H NMR (DMSO-d₆) δ 11.06 (s, 1H), 8.59 (d, 1H), 7.86 (d, 1H), 7.81 (d, 1H), 7.61 (d, 1H), 7.39 (d, 1H), 7.34-7.31 (m, 2H), 7.14 (dd, 1H), 7.02 (d, 1H), 4.98 (dd, 1H), 4.59-4.51 (m, 2H), 4.50-4.48 (m, 2H), 3.96 (s, 3H), 3.90-3.83 (m, 1H), 3.04-2.99 (m, 2H), 2.93-2.86 (m, 1H), 2.72 (bs, 2H), 2.63-2.55 (m, 2H), 2.30-2.22 (m, 2H), 2.21-2.16 (m, 2H), 2.13-2.08 (m, 2H), 2.07-1.98 (m, 3H), 1.94-1.86 (m, 2H), 1.86-1.80 (m, 2H), 1.73-1.62 (m, 5H), 1.54-1.49 (m, 2H), 1.15-1.09 (m, 2H).

Compound 216: MS: m/z 884.5 (M⁺+23); ¹H NMR (DMSO-d₆) δ 11.06 (s, 1H), 8.53 (d, 1H), 7.86 (d, 1H), 7.82 (d, 1H), 7.62 (d, 1H), 7.39 (d, 1H), 7.31 (s, 1H), 7.14 (dd, 1H), 7.02 (d, 1H), 6.94 (d, 1H), 4.98 (dd, 1H), 4.60-4.51 (m, 2H), 3.96 (s, 3H), 3.90-3.83 (m, 1H), 3.83-3.57 (m, 2H), 3.49-3.40 (m, 1H), 3.17-3.07 (m, 1H), 2.92-2.86 (m, 1H), 2.76 (bs, 2H), 2.63-2.55 (m, 2H), 2.43-2.38 (m, 1H), 2.37-2.22 (m, 2H), 2.21-2.16 (m, 1H), 2.15-2.08 (m, 2H), 2.06-1.98 (m, 3H), 1.94-1.88 (m, 2H), 1.72-1.61 (m, 7H), 1.55-1.49 (m, 2H).

Compound 217: MS: m/z 834.4 (M⁺+1); ¹H NMR (DMSO-d₆) δ 11.06 (s, 1H), 8.55 (d, 1H), 7.87-7.82 (m, 2H), 7.61 (d, 1H), 7.39 (d, 1H), 7.31 (bs, 1H), 7.14 (dd, 1H), 7.02 (dd, 1H), 6.86 (d, 1H), 4.98 (dd, 1H), 4.59-4.51 (m, 2H), 4.24-4.21 (m, 2H), 3.96 (s, 3H), 3.89-3.83 (m, 1H), 3.82-3.79 (m, 2H), 3.07-3.02 (m, 1H), 2.92-2.86 (m, 1H), 2.75 (bs, 2H), 2.70-2.64 (m, 2H), 2.63-2.56 (m, 1H), 2.52-2.49 (m, 1H), 2.35-2.29 (m, 2H), 2.12-2.09 (m, 2H), 2.06-2.00 (m, 3H), 1.92-1.89 (m, 2H), 1.71-1.61 (m, 4H), 1.55-1.49 (m, 2H).

Compound 218: MS: m/z 848.6 (M⁺+1); ¹H NMR (DMSO-d₆) δ 11.07 (s, 1H), 8.52 (d, 1H), 7.87 (d, 1H), 7.82 (d, 1H), 7.62 (d, 1H), 7.40 (d, 1H), 7.32 (d, 1H), 7.14 (dd, 1H), 7.03 (dd, 1H), 6.96 (d, 1H), 4.98 (dd, 1H), 4.62-4.51 (m, 2H), 3.96 (s, 3H), 3.90-3.83 (m, 1H), 3.79-3.58 (m, 2H), 3.56-3.46 (m, 1H), 3.30-3.20 (m, 1H), 2.93-2.86 (m, 1H), 2.85-2.72 (m, 2H), 2.66-2.57 (m, 2H), 2.44-2.39 (m, 2H), 2.38-2.26 (m, 2H), 2.20-2.10 (m, 4H), 2.07-1.98 (m, 3H), 1.96-1.90 (m, 2H), 1.80-1.74 (m, 1H), 1.74-1.62 (m, 4H), 1.55-1.50 (m, 2H), 1.15-1.09 (m, 2H).

Compound 219: MS: m/z 910.8 (M⁺+23); ¹H NMR (DMSO-d₆) δ 11.06 (s, 1H), 7.91 (d, 1H), 7.75 (d, 2H), 7.62 (d, 1H), 7.38 (d, 1H), 7.31 (bs, 1H), 7.21 (d, 1H), 7.03-7.00 (m, 2H), 6.54 (d, 2H), 4.98 (dd, 1H), 4.61-4.54 (m, 1H), 4.33 (s, 1H), 4.06 (d, 1H), 3.96 (s, 3H), 3.52-3.49 (m, 1H), 3.40-3.36 (m, 1H), 3.30-3.26 (m, 1H), 2.95-2.86 (m, 2H), 2.76 (bs, 1H), 2.62-2.59 (m, 1H), 2.59-2.54 (m, 1H), 2.44-2.37 (m, 1H), 2.34-2.23 (m, 3H), 2.19-2.14 (m, 1H), 2.07-1.97 (m, 3H), 1.75-1.59 (m, 5H), 1.29-1.22 (m, 2H), 1.22 (s, 6H), 1.13 (s, 6H).

Compound 220: MS: m/z 822.6 (M⁺+1); ¹H NMR (DMSO-d₆) δ 11.06 (s, 1H), 8.77 (s, 2H), 8.14 (d, 1H), 7.86 (d, 1H), 7.62 (d, 1H), 7.39 (d, 1H), 7.34 (d, 1H), 7.15 (dd, 1H), 7.01 (dd, 1H), 4.98 (dd, 1H), 4.58-4.53 (m, 1H), 4.44 (q, 2H), 4.18-4.15 (m, 2H), 3.87-3.77 (m, 5H), 2.92-2.86 (m, 1H), 2.61-2.50 (m, 6H), 2.49-2.45 (m, 2H), 2.12-2.08 (m, 2H), 2.07-2.03 (m, 1H), 2.00-1.95 (m, 2H), 1.95-1.90 (m, 2H), 1.54-1.46 (m, 4H), 1.42 (t, 3H).

Compound 221: MS: m/z 889.7 (M⁺+1); ¹H NMR (DMSO-d₆) δ 11.06 (s, 1H), 8.62 (s, 1H), 7.94-7.90 (m, 2H), 7.62-7.57 (m, 2H), 7.31 (s, 1H), 7.21 (s, 1H), 7.03-7.00 (m, 2H), 6.85 (d, 1H), 4.98 (dd, 1H), 4.60-4.52 (m, 1H), 4.42-4.40 (m, 2H), 4.30 (s, 1H), 4.05 (d, 1H), 3.95 (s, 3H), 2.92-2.86 (m, 3H), 2.72 (bs, 2H), 2.63-2.55 (m, 2H), 2.30-2.21 (m, 2H), 2.21-2.14 (m, 2H), 2.07-1.97 (m, 3H), 1.86-1.78 (m, 3H), 1.73-1.63 (m, 2H), 1.28-1.22 (m, 2H), 1.22 (s, 6H), 1.12 (s, 6H).

Compound 222: MS: m/z 889.5 (M⁺+1); ¹H NMR (DMSO-d₆) δ 11.06 (s, 1H), 8.62 (d, 1H), 7.94 (d, 1H), 7.91 (d, 1H), 7.62 (d, 1H), 7.55 (d, 1H), 7.32 (bs, 1H), 7.21 (d, 1H), 7.03-7.00 (m, 2H), 6.47 (d, 2H), 4.98 (dd, 1H), 4.61-4.52 (m, 1H), 4.31 (s, 1H), 4.06 (d, 1H), 3.96 (s, 3H), 3.75-3.66 (m, 1H), 3.41-3.33 (m, 1H), 3.08-3.01 (m, 1H), 2.93-2.86 (m, 1H), 2.81-2.67 (m, 2H), 2.64-2.56 (m, 2H), 2.43-2.36 (m, 1H), 2.34-2.22 (m, 2H), 2.19-2.13 (m, 1H), 2.08-1.98 (m, 3H), 1.75-1.58 (m, 5H), 1.28-1.22 (m, 2H), 1.22 (s, 6H), 1.12 (s, 6H).

Compound 223: MS: m/z 794.4 (M⁺+1); ¹H NMR (DMSO-d₆) δ 11.06 (s, 1H), 8.77 (s, 2H), 8.14 (d, 1H), 7.87 (d, 1H), 7.62 (d, 1H), 7.39 (d, 1H), 7.34 (d, 1H), 7.15 (dd, 1H), 7.03 (dd, 1H), 4.98 (dd, 1H), 4.56-4.53 (m, 1H), 4.26-4.24 (m, 2H), 3.97 (s, 3H), 3.86-3.84 (m, 4H), 3.83-3.77 (m, 1H), 2.93-2.86 (m, 1H), 2.84-2.82 (m, 2H), 2.61-2.56 (m, 5H), 2.52-2.49 (m, 1H), 2.13-2.07 (m, 2H), 2.06-2.01 (m, 1H), 1.94-1.90 (m, 2H), 1.54-1.48 (m, 4H).

In addition, compound ARV-110 was purchased from BLD Pharmatech Ltd. (Cat. No.: BD01398519; Lot No.: CKA112, Purity: 97%) for the following assays. ARV-110 is a well-known bifunctional compound for AR degradation.

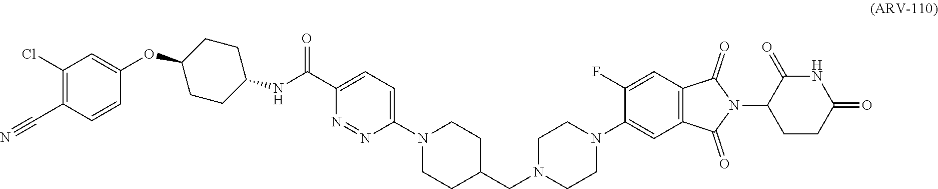 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

### Androgen Receptor Degradation Assay Using Western Blot Analysis

LNCaP.FGC cells (Cat. 60088, Bioresource Collection and Research Center, HsinChu City, Taiwan R.O.C.) grown in RPMI 1640 (Cat. 31800022, Thermo Fisher Scientific, Waltham, Massachusetts, United States) medium supplemented with 10% FBS (Cat. 10437028, Thermo Fisher Scientific, Waltham, Massachusetts, United States), 10 mM HEPES (Cat. 15630080, Thermo Fisher Scientific, Waltham, Massachusetts, United States) and 1 mM sodium pyruvate (Cat. 11360070, Thermo Fisher Scientific, Waltham, Massachusetts, United States) were seeded at 2×10⁵ cells per well in 24-well tissue culture plates. Cells were incubated at 37° C., 5% CO₂ for 24 hours (hr), then treated with 100 nanomolar concentrations (nM) any of the compounds 1 to 223 and ARV-110 for 24 hr. After the treatment, the cells were harvested, washed by PBS, and lysed with RIPA lysis and extraction buffer (Cat. 89900, Thermo Fisher Scientific, Waltham, Massachusetts, United States) supplemented with Halt Protease Inhibitor Cocktail (Cat. 78430, Thermo Fisher Scientific, Waltham, Massachusetts, United States) to collect protein samples. Cells not treated with any of the above-mentioned compounds are used as a negative control.

The protein samples were separated by polyacrylamide gel electrophoresis, and then transferred to a piece of Immuno-Blot PVDF membrane (Cat. 1620177, Bio-Rad Laboratories, Hercules, California, United States). The presence of androgen receptor in the protein samples was detected by standard Western blotting procedure using an anti-AR antibody (1:2000 dilution) (Cat. 5153, Cell Signaling Technology Inc., Danvers, Massachusetts, United States) and a goat anti-rabbit HRP-conjugated secondary antibody (1:5000 dilution) (C04003, Croyez Bioscience Co., Ltd., Taipei City, Taiwan R.O.C.). The internal loading control GAPDH was detected using a mouse monoclonal antibody (1:5000) (GTX627408, GeneTex International Corp., HsinChu City, Taiwan R.O.C.) and a goat anti-mouse HRP-conjugated secondary antibody (1:5000 dilution) (C04001, Croyez Bioscience Co., Ltd., Taipei City, Taiwan R.O.C.). Chemiluminescence signals were developed using Clarity Western ECL substrate (Cat. 1705061, Bio-Rad Laboratories, Hercules, California, United States) and detected with digital imager iBright FL1500 (Invitrogen Corp., Carlsbad, California, United States).

AR remaining percentage was determined by the intensity difference of AR signals (each was normalized by the corresponding internal loading control GAPDH) between the protein samples from the untreated cells (100%) and those from the treated cells. The results of compounds 1 to 169 are shown in Table 2. “Cpd. #” indicates the compound number hereinabove.

In addition, several compounds were selected to prepare a treatment stock solutions at 100 nM in RPMI medium. The treatment stock solutions were separately serially diluted (10×) with RPMI medium for treating LNCaP.FGC cells seeded with 2×10⁵ cells per well in 24-well tissue culture plates. A calibration curve was determined by the serial diluted samples for each compound, and the concentration needed for 50% AR degradation (AR DC₅₀) for each compound was calculated. The results are shown in Table 3. In Table 3, the result marks with “A” when AR DC₅₀ of the compound is less than 10 nM; the result marks with “B” when AR DC₅₀ of the compound is equal or greater than 10 nM and less than 25 nM; the result marks with “C” when AR DC₅₀ of the compound is equal or greater than 25 nM and less than 100 nM and the result marks with “D” when AR DC₅₀ of the compound is equal or greater than 100 nM.

From Tables 2 and 3, it is clear that the compounds of the present invention have good effect in AR degradation. Under the same operation conditions, many compounds of the present invention result in greater AR degradation than ARV-100 at the same concentration (100 nM), and they generally have a lower AR DC₅₀ (nM).

TABLE 2
Androgen Receptor Remaining (%) of Compounds
of the Present Invention at 100 nM

| Cpd. No. | % |
|---|---|
| 1 | 47 |
| 2 | 56 |
| 3 | 32 |
| 4 | 66 |
| 5 | 51 |
| 6 | 31 |
| 7 | 45 |
| 8 | 22 |
| 9 | 73 |
| 10 | 37 |
| 11 | 73 |
| 12 | 42 |
| 13 | 155 |
| 14 | 70 |
| 15 | 106 |
| 16 | 96 |
| 17 | 127 |
| 18 | 99 |
| 19 | 73 |
| 20 | 63 |
| 21 | 92 |
| 22 | 89 |
| 23 | 88 |
| 24 | 62 |
| 25 | 80 |
| 26 | 75 |
| 27 | 107 |
| 28 | — |
| 29 | 31 |
| 30 | 56 |
| 31 | 47 |
| 32 | 59 |
| 33 | 27 |
| 34 | 75 |
| 35 | 75 |
| 36 | — |
| 37 | 55 |
| 38 | — |
| 39 | 16 |
| 40 | 29 |
| 41 | 81 |
| 42 | 30 |
| 43 | 26 |
| 44 | — |
| 45 | — |
| 46 | 20 |
| 47 | 19 |
| 48 | 20 |
| 49 | 36 |
| 50 | 45 |
| 51 | 20 |
| 52 | 42 |
| 53 | 77 |
| 54 | 72 |
| 55 | 40 |
| 56 | 4 |
| 57 | 53 |
| 58 | 65 |
| 59 | 66 |
| 60 | 57 |
| 61 | 96 |
| 62 | 65 |
| 63 | — |
| 64 | 40 |
| 65 | 32 |
| 66 | 100 |
| 67 | 58 |
| 68 | 81 |
| 69 | 51 |
| 70 | 62 |
| 71 | 77 |
| 72 | 26 |
| 73 | 41 |
| 74 | 40 |
| 75 | 26 |
| 76 | 78 |
| 77 | 92 |
| 78 | 57 |
| 79 | 60 |
| 80 | 22 |
| 81 | 9 |
| 82 | 35 |
| 83 | 41 |
| 84 | 62 |
| 85 | 20 |
| 86 | 20 |
| 87 | 21 |
| 88 | 10 |
| 89 | 7 |
| 90 | 28 |
| 91 | 0 |
| 92 | 6 |
| 93 | 11 |
| 94 | 89 |
| 95 | 1 |
| 96 | 1 |
| 97 | 0 |
| 98 | 26 |
| 99 | 39 |
| 100 | 44 |
| 101 | 40 |
| 102 | 44 |
| 103 | 52 |
| 104 | 42 |
| 105 | 42 |
| 106 | 119 |
| 107 | 15 |
| 108 | 19 |
| 109 | 29 |
| 110 | 34 |
| 111 | 39 |
| 112 | 32 |
| 113 | 26 |
| 114 | 21 |
| 115 | 26 |
| 116 | 37 |
| 117 | 56 |
| 118 | 80 |
| 119 | 72 |
| 120 | 48 |
| 121 | 48 |
| 122 | 33 |
| 123 | 33 |
| 124 | 57 |
| 125 | 57 |
| 126 | 58 |
| 127 | 58 |
| 128 | 63 |
| 129 | 63 |
| 130 | 51 |
| 131 | 51 |
| 132 | 7 |
| 133 | 1 |
| 134 | 2 |
| 135 | 17 |
| 136 | 17 |
| 137 | 1 |
| 138 | 39 |
| 139 | 52 |
| 140 | 32 |
| 141 | 49 |
| 142 | 49 |
| 143 | 86 |
| 144 | 86 |
| 145 | 62 |
| 146 | 62 |
| 147 | 46 |
| 148 | 30 |
| 149 | 11 |
| 150 | 40 |
| 151 | 57 |
| 152 | 45 |
| 153 | 109 |
| 154 | 33 |
| 155 | 19 |
| 156 | 37 |
| 157 | 18 |
| 158 | 13 |
| 159 | 8 |
| 160 | 17 |
| 161 | 31 |
| 162 | 29 |
| 163 | 41 |
| 164 | 31 |
| 165 | 77 |
| 166 | 47 |
| 167 | 78 |
| 168 | 20 |
| 169 | 27 |
| ARV 110 | 31 |

TABLE 3
AR DC₅₀ (nM) of Compounds of the Present Invention

| Cpd. | AR DC₅₀ |
|---|---|
| No. | (nM) |
| 1 | B |
| 3 | C |
| 6 | C |
| 7 | C |
| 8 | B |
| 9 | D |
| 10 | C |
| 12 | C |
| 28 | C |
| 29 | B |
| 31 | C |
| 33 | B |
| 36 | B |
| 38 | C |
| 39 | B |
| 40 | D |
| 42 | B |
| 43 | C |
| 44 | C |
| 45 | C |
| 46 | B |
| 47 | B |
| 48 | A |
| 49 | C |
| 50 | C |
| 51 | C |
| 52 | B |
| 55 | C |
| 56 | C |
| 63 | B |
| 64 | C |
| 65 | C |
| 72 | A |
| 73 | C |
| 74 | C |
| 75 | B |
| 78 | B |
| 80 | B |
| 81 | A |
| 82 | C |
| 83 | B |
| 85 | C |
| 86 | C |
| 87 | C |
| 88 | B |
| 89 | A |
| 90 | C |
| 91 | B |
| 92 | C |
| 93 | C |
| 95 | B |
| 96 | B |
| 97 | B |
| 98 | C |
| 99 | C |
| 100 | C |
| 101 | C |
| 102 | C |
| 104 | C |
| 105 | C |
| 109 | B |
| 110 | B |
| 111 | A |
| 112 | B |
| 113 | B |
| 114 | B |
| 115 | C |
| 116 | C |
| 120 | C |
| 121 | C |
| 122 | D |
| 123 | D |
| 132 | B |
| 133 | B |
| 134 | B |
| 135 | C |
| 136 | C |
| 137 | C |
| 138 | C |
| 139 | C |
| 140 | C |
| 141 | C |
| 142 | C |
| 147 | B |
| 148 | B |
| 152 | C |
| 153 | B |
| 154 | B |
| 155 | B |
| 156 | C |
| 157 | B |
| 158 | B |
| 159 | C |
| 160 | A |
| 161 | B |
| 162 | C |
| 164 | B |
| 168 | B |
| 169 | B |
| 170 | C |
| 171 | C |
| 172 | C |
| 173 | C |
| 174 | C |
| 175 | B |
| 176 | C |
| 177 | C |
| 178 | B |
| 179 | C |
| 180 | A |
| 181 | A |
| 182 | A |
| 183 | B |
| 184 | C |
| 185 | C |
| 186 | B |
| 187 | C |
| 188 | C |
| 189 | B |
| 190 | B |
| 191 | A |
| 192 | C |
| 193 | C |
| 194 | B |
| 195 | B |
| 196 | A |
| 197 | B |
| 198 | A |
| 199 | B |
| 200 | B |
| 201 | B |
| 202 | C |
| 203 | A |
| 204 | B |
| 205 | A |
| 206 | B |
| 207 | A |
| 208 | B |
| 209 | B |
| 210 | B |
| 211 | A |
| 212 | C |
| 213 | B |
| 214 | D |
| 215 | B |
| 216 | A |
| 217 | B |
| 218 | B |
| 219 | C |
| 220 | C |
| 221 | B |
| 222 | B |
| 223 | C |
| ARV 110 | C |

### In Vivo Hair Regrowth Efficacy of Compounds of the Present Invention in Androgenetic Alopecia (AGA) Mice Model

Seven-week old male C57BL/6 mice (with black fur) purchased from BioLASCO Taiwan Co., Ltd. were grouped 6 to 8 mice per cage, and intraperitoneally administered with dihydrotestosterone (DHT) (1 mg/day, dissolved in corn oil) or corn oil alone once daily for four days. DHT is an agonist to androgen receptors, which can be used to induce symptoms of androgenetic alopecia.

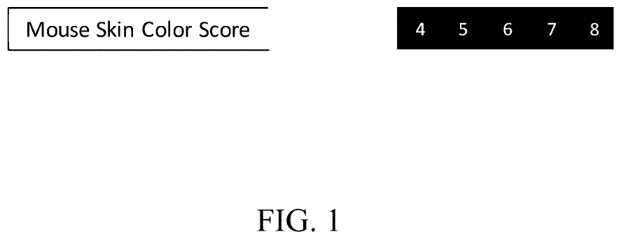

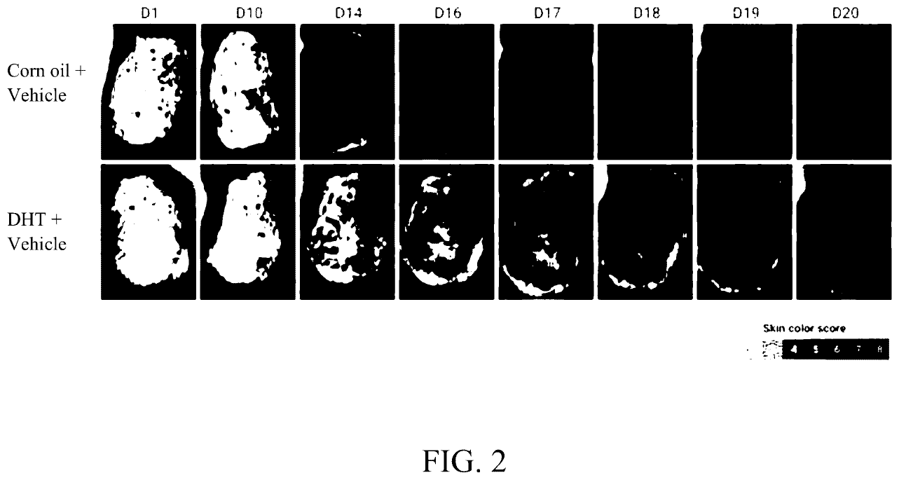

Four days later, the dorsal region of the mice was depilated with hair removal cream (Nair Sensitive Hair Removal Cream, 100 mg to 200 mg per mouse). After hair removal, the skin color was recorded by photographs with a resolution of 4032*3024 pixel (day 1, D1). On the next day (D2), the test solutions of the compound 29 and compound 33 in a vehicle were prepared (3.5 μg/cm²/day), and each test solution or vehicle alone was topically administered on the depilated region once daily for 20 days. The hair removal cream, the test solution and the vehicle were separately rubbed into the dorsal region of mice. The photographs underwent analysis using a self-developed software that utilized auto-image segmentation, primarily focusing on the border of the depilation area. This segmentation process involved classifying different regions within the images. The software could identify the border of the depilation area on the photographs, and mixed and transformed the colors in the depilation area on each photograph into a single color block. Subsequently, the photographs were transformed into a skin color score. As shown in the color chart of FIG. 1, the score ranges from 1 to 8, as score 1 corresponds to depilated skin (skin color), and score 8 corresponds to fully regrowth hair (black color), and scores 2 to 7 sequentially correspond to different stages between scores 1 and 8. FIG. 2 shows the photographs of the depilation area in the Corn oil+Vehicle group and the DHT+Vehicle group on D1, D10, D14, D16, D17, D18, D19 and D20, the color change corresponds the hair regrowth efficacy. The hair regrowth efficacy could be evaluated by the photographs and/or the skin color score.

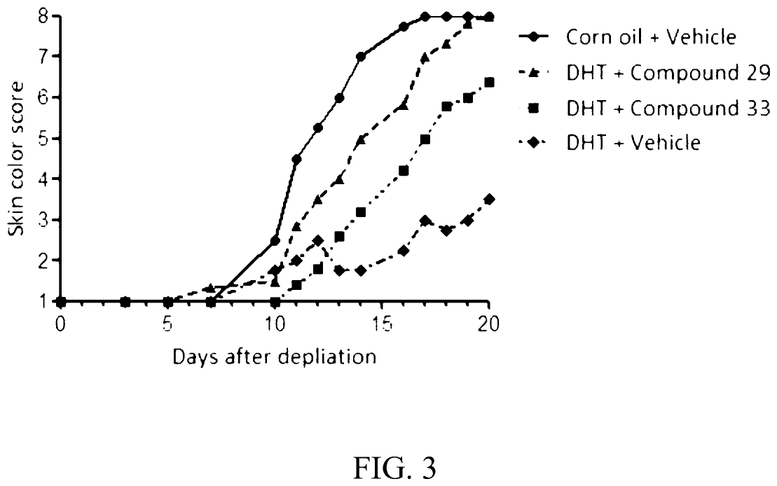

As shown in FIG. 3, it can be found that the average skin color score at day 15 is nearly 8 in the corn oil+vehicle group. DHT pre-treatment obviously damages the hair regrowth capability, and the compounds of the present invention can recover the damaged hair regrowth capability. The average skin color score reaches 8 at day 20 in the DHT+compound 29 group, and the average skin color score reaches 6.5 at day 20 in the DHT+compound 33 group, both are much higher than the average skin color score that reaches 3.5 at day 20 in the DHT+vehicle group.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1.-19. (canceled)

20. A bifunctional compound, or a pharmaceutically acceptable salt, hydrate, solvate, metabolite or prodrug thereof, wherein the bifunctional compound is represented by Formula (I):
ABM-L-CLM (I);
wherein:
ABM is an androgen receptor binding moiety;
-L- is a linking moiety; and
CLM is a cereblon E3 ubiquitin ligase binding moiety represented by Formula (II)-1: 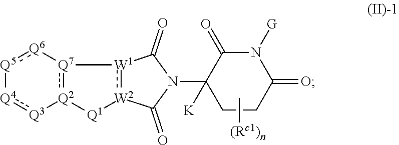
wherein: [symbol]
represents a single bond or a double bond;
one end of the -L- is covalently joined to Q₃, Q₄, Q₅ or Q₆; and the other end of the -L- is covalently joined to the ABM;
W¹ and W² are each independently CR^{C2} or N when a single bond is present between W¹ and W²; or, W¹ and W² are each C when a double bond is present between W¹ and W²;
G is selected from the group consisting of —H, —OH, —CH₂OH, —R^{C3}OC(═O)OR^{C4}, —R^{C3}OC(═O)NR^{C4}R^{C5}, and 2-(trimethylsilyl)ethoxymethyl group;
Q¹ is NR^{C6};
Q² and Q⁷ are each independently N or CR^{C2} when a single bond is present between Q² and Q⁷; or, Q² and Q⁷ are each C when a double bond is present between Q² and Q⁷;
when the one end of the -L- is covalently joined to any one atom selected from Q₃, Q₄, Q₅ and Q₆; the atom covalently joined with the -L- is CR^{C2} when two single bonds are each independently present between the C atom of CR^{C2} and its two adjacent atoms on the ring, or the atom covalently joined with the -L- is C when a double bond is present between the C atom and one of its two adjacent atoms on the ring; and, the other atoms selected from Q³, Q⁴, Q⁵ and Q⁶ which are not covalently joined with the -L- are each independently O, S, C(R^{C2})₂ or NR^{C2} when two single bonds are each independently present between the O atom, S atom, C atom of C(R^{C2})₂ or N atom of NR^{C2} and its two adjacent atoms on the ring, or the other atoms selected from Q³, Q⁴, Q⁵ and Q⁶ which are not covalently joined with the -L- are each independently CR^{C2} when a double bond is present between the C atom of CR^{C2} and one of its two adjacent atoms on the ring;
K is selected from the group consisting of —H, an unsubstituted alkyl group, an alkyl group substituted by R^{C7}, an unsubstituted cycloalkyl group, and a cycloalkyl group substituted by R^{C7}; K is bound to the 6-membered ring with a stereospecific bond or a non-stereospecific bond;
R^{C1} is selected from the group consisting of an unsubstituted alkyl group, an alkyl group substituted by R^{C8}, an unsubstituted aryl group, an aryl group substituted by R^{C8}, an unsubstituted alkyl-aryl group, an alkyl-aryl group substituted by R^{C8}, an unsubstituted alkoxyl group, and an alkoxyl group substituted by R^{C8};
R^{C2} is selected from the group consisting of —H, -D, a halo group, —CH₂OH, —NR^{C4}R^{C5}, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, and an aryl group substituted by one or more halo groups;
R^{C3} is selected from the group consisting of an unsubstituted alkylene group, and an alkylene group substituted by R^{C7};
R^{C4} and R^{C5} are each independently selected from the group consisting of —H, an unsubstituted alkyl group, an alkyl group substituted by R^{C9}, an unsubstituted cycloalkyl group, a cycloalkyl group substituted by R^{C9}, an unsubstituted heterocyclyl group, a heterocyclyl group substituted by R^{C9}, an unsubstituted aryl group, an aryl group substituted by R^{C9}, an unsubstituted heteroaryl group, and a heteroaryl group substituted by R^{C9};
R^{C6} is selected from the group consisting of —CH₂OH, 2-(trimethylsilyl)ethoxymethyl, an unsubstituted C₁₋₆ alkyl group, a C₁₋₆ alkyl group substituted by one or more halo groups, an unsubstituted cycloalkyl group, and a cycloalkyl group substituted by one or more halo groups;
R^{C7} is selected from the group consisting of a halo group, —CH₂OH, —NR^{C4}R^{C5}, 2-(trimethylsilyl)ethoxymethyl, an alkoxyl group, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C8} is selected from the group consisting of a halo group, —CH₂OH, —NR^{C4}R^{C5}, 2-(trimethylsilyl)ethoxymethyl, an unsubstituted cycloalkyl group, a cycloalkyl group substituted by one or more halo groups, an unsubstituted heteroaryl group, a heteroaryl group substituted by one or more halo groups, an unsubstituted heterocyclyl group, and a heterocyclyl group substituted by one or more halo groups;
R^{C9} is selected from the group consisting of a halo group, —CH₂OH, 2-(trimethylsilyl)ethoxymethyl, and an alkoxyl group; and
n is 0, 1, 2, 3 or 4; and
the heteroatom is selected from N, O and S;
wherein the -L- is a linking moiety represented by Formula (III): 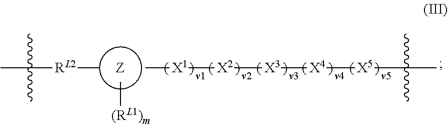
wherein
Z is selected from the group consisting of a 3- to 8-membered monocyclic ring, a 5- to 12-membered bicyclic ring, a 8- to 15-membered tricyclic ring and a 6- to 12-membered spiro bicyclic ring, each independently having 0 to 4 heteroatoms and 0 or 1 double bond;
R^{L1} is selected from the group consisting of an unsubstituted C₁₋₆ alkyl group, a C₁₋₆ alkyl group substituted by a C₁₋₆ alkoxyl group, a C₁₋₆ alkyl group substituted by one or more halo groups, a halo group, an unsubstituted C₁₋₆ alkoxyl group, a keto group, and an oxide group;
R^{L2} is a bond or an ethynylene group;
X¹ is a methylene group or an ethylene group, each of which is unsubstituted or substituted by an alkyl or a cycloalkyl;
X² and X⁵ are each independently selected from the group consisting of an unsubstituted methylene group, a methylene group substituted by an alkyl or a cycloalkyl, CO, COCO, CONH, NHCO, NHCONR^{L3}, NHCOCONR^{L3}—, NHSO₂NR^{L3}, SO₂NH, CHR^{L3}, NR^{L4}, O and S;
X³ is selected from the group consisting of a C₁₋₈ alkylene group, a C₁₋₈ heteroalkylene group, a 5- to 8-membered arylene group, a 5- to 8-membered heteroarylene group having 1 to 3 hetero atoms, a 3- to 7-membered cyclic alkylene group, a 3- to 7-membered heterocyclic alkylene group having 1 to 2 hetero atoms, and a 6- to 12-membered spiro bicyclic ring having 0 to 4 heteroatoms, each of which is unsubstituted or substituted by an alkyl or a cycloalkyl;
X⁴ is an unsubstituted C₁₋₆ alkylene group, or a C₁₋₆ alkylene group substituted by an alkyl or a cycloalkyl;
R^{L3} and R^{L4} are each independently selected from the group consisting of —H, an unsubstituted C₁₋₆ alkyl group, a C₁₋₆ alkyl group substituted by a C₁₋₆ alkoxyl group, and a C₁₋₆ alkyl group substituted by one or more halo groups;
m is 0, 1, 2, 3, 4, 5 or 6; v1 is 0 or 1; v2 is 0 or 1; v3 is 1; v4 is 0 or 1; and v5 is 1.

21. The bifunctional compound, or the pharmaceutically acceptable salt, hydrate, solvate, metabolite or prodrug thereof as claimed in claim 20, wherein the CLM is represented by Formula (II)-2: 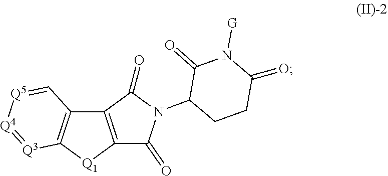
wherein
one end of the -L- is covalently joined to Q₃, Q₄ or Q₅;
G is selected from the group consisting of —H, —OH, —CH₂OH and 2-(trimethylsilyl)ethoxymethyl group;
Q₁ is NR^{C6};
when the one end of the -L- is covalently joined to any one atom selected from Q₃, Q₄ and Q₅; the atom attached with the -L- is C; and, the other atoms selected from Q³, Q⁴ and Q⁵ which are not attached with the -L- are each independently CR^{C2};
R^{C2} is selected from the group consisting of —H, -D, a halo group, an unsubstituted alkyl group, and an alkyl group substituted by one or more halo groups; and
R^{C6} is selected from the group consisting of —CH₂OH, 2-(trimethylsilyl)ethoxymethyl, an unsubstituted C₁₋₆ alkyl group, and a C₁₋₆ alkyl group substituted by one or more halo groups.

22. The bifunctional compound, or the pharmaceutically acceptable salt, hydrate, solvate, metabolite or prodrug thereof as claimed in claim 20, wherein the -L is a linking moiety represented by Formula (III): 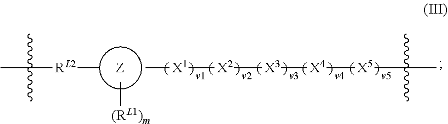
wherein
Z is selected from the group consisting of a 3- to 8-membered monocyclic ring, a 6- to 10-membered bicyclic ring and a 8- to 11-membered spiro bicyclic ring, each independently having 1 to 2 heteroatoms and 0 or 1 double bond;
R^{L1} is selected from the group consisting of an unsubstituted linear C₁₋₃ alkyl group, and a keto group;
R^{L2} is a bond or an ethynylene group;
X¹ is a methylene group or an ethylene group, each of which is unsubstituted or substituted by an alkyl or a cycloalkyl;
X² is selected from the group consisting of an unsubstituted methylene group, a methylene group substituted by an alkyl or a cycloalkyl, CO, NHCO, NR^{L4}, and O;
X³ is selected from the group consisting of a C₁₋₆ alkylene group, a 3- to 7-membered cyclic alkylene group, and a 3- to 7-membered heterocyclic alkylene group having 0 to 2 hetero atoms, each of which is unsubstituted or substituted by an alkyl or a cycloalkyl;
X⁴ is an unsubstituted C₁₋₃ alkylene group, or a C₁₋₃ alkylene group substituted by an alkyl or a cycloalkyl;
X⁵ is selected from the group consisting of an unsubstituted methylene group, a methylene group substituted by an alkyl or a cycloalkyl, CONH and O;
R^{L4} is hydrogen;
m is 0, 1, 2 or 3; v1 is 0 or 1; v2 is 0 or 1; v3 is 1; v4 is 0 or 1; and v5 is 1.

23. The bifunctional compound, or the pharmaceutically acceptable salt, hydrate, solvate, metabolite, or prodrug thereof as claimed in claim 20, wherein the -L- is 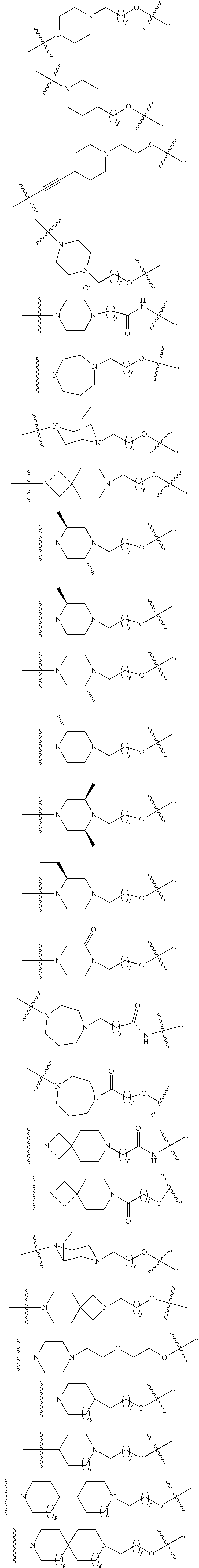 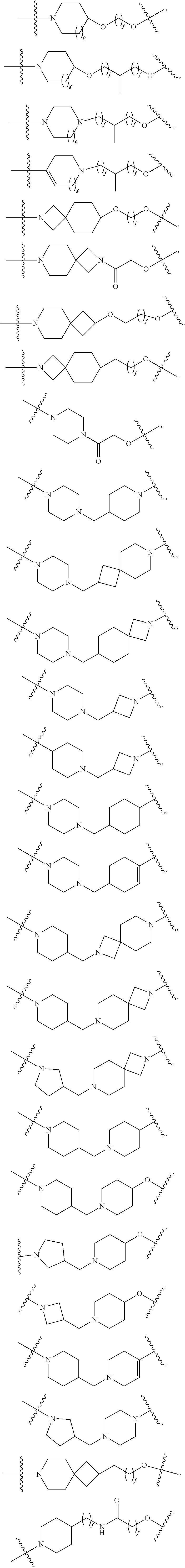 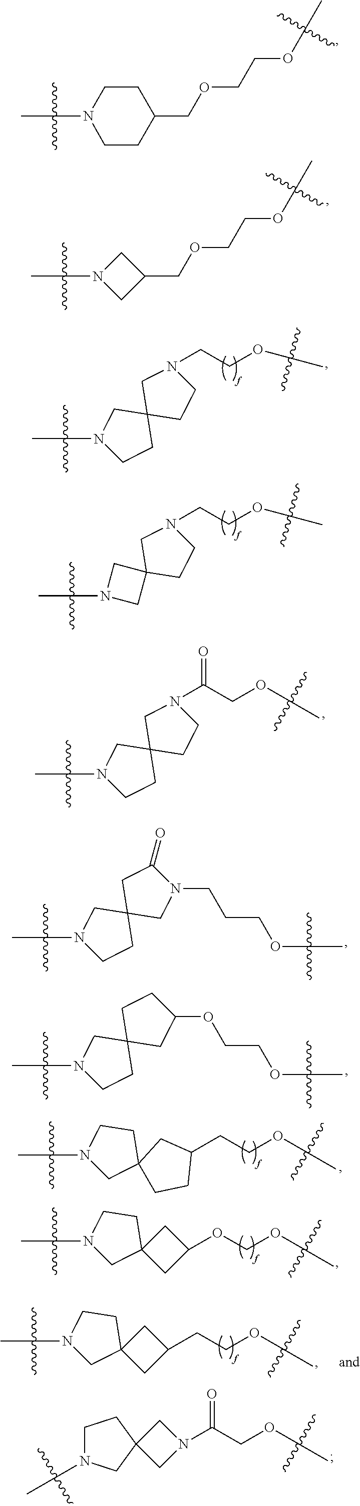
wherein
f is an integer of 0, 1, 2, 3 or 4; and
g is an integer of 0, 1, 2 or 3.

24. The bifunctional compound, or the pharmaceutically acceptable salt, hydrate, solvate, metabolite or prodrug thereof as claimed in claim 20, wherein the ABM is an androgen receptor binding moiety represented by 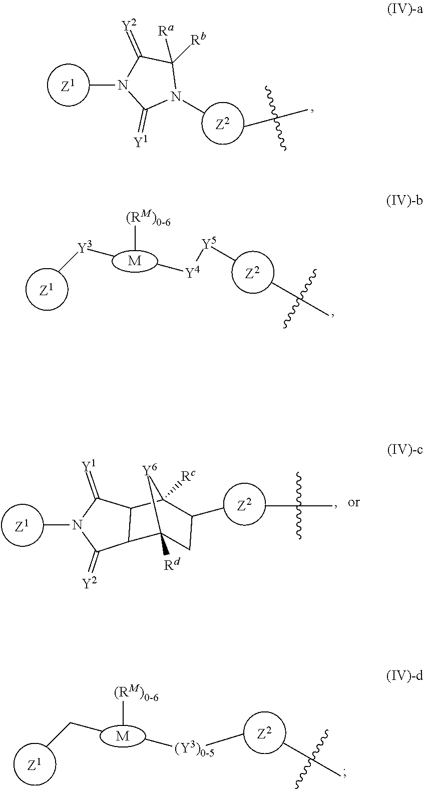
wherein:
Z¹ is selected from the group consisting of an aryl group, a heteroaryl group, a bicyclic group, or a bi-heterocyclic group, each independently substituted by one or more substituents independently selected from the group consisting of a halo group, a hydroxyl group, a nitro group, —CN, —C≡CH, an unsubstituted C₁₋₆ alkyl group, a C₁₋₆ alkyl group substituted by a C₁₋₆ alkoxyl group, a C₁₋₆ alkyl group substituted by one or more halo groups, an unsubstituted C₁₋₆ alkoxyl group, a C₁₋₆ alkoxyl group substituted by one or more halo groups, an unsubstituted C₂₋₆ alkenyl, a C₂₋₆ alkenyl substituted by one or more halo groups, an unsubstituted C₂₋₆ alkynyl, a C₃₋₆ alkynyl substituted by one or more halo groups, and any combinations thereof;
Y¹ and Y² are each independently NR^{Y1}, O or S;
Y³, Y⁴ and Y⁵ are each independently selected from the group consisting of a bond,
—O—, —NR^{Y2}—, —C(—R^{Y1})(—R^{Y2})—, —C(═O)—, —C(═S)—, —S(═O)—, —SO₂—, a heteroarylene group, and an arylene group;
Y⁶ is —N(—R^{Y1})—, —O— or —S—;
M is a 3- to 6-membered ring having 0 to 4 heteroatoms, which is unsubstituted or substituted by 0 to 6 R^{M} groups;
each R^{M} group is independently selected from the group consisting of an unsubstituted C₁₋₄ alkyl group, a C₁₋₆ alkyl group substituted by a C₁₋₆ alkoxyl group, a C₁₋₆ alkyl group substituted by one or more halo groups, a halo group, and a C₁₋₆ alkoxyl group; or two R^{M} groups are taken together with the atom they are attached to and form a 3- to 8-membered ring system containing 0 to 2 heteroatoms;
R^{a}, R^{b}, R^{c}, R^{d}, R^{Y1} and R^{Y2} are each independently selected from the group consisting of —H, an unsubstituted C₁₋₆ alkyl group, a C₁₋₆ alkyl group substituted by a C₁₋₆ alkoxyl group, a C₁₋₆ alkyl group substituted by one or more halo groups, a halo group, a C₁₋₆ alkoxyl group, a cyclic group, and a heterocyclic group; or R^{a}, R^{b} are taken together with the atom they are attached to and form a 3- to 8-membered ring system containing 0 to 2 heteroatoms;
Z² is selected from the group consisting of a bond, a C_{1,6} alkylene group, a C₁₋₆ heteroalkylene group, —O—, an arylene group, a heteroarylene group, an alicyclic bivalent group, a heterocyclic bivalent group, a heterobicyclic bivalent group, a bicyclic arylene group, and a bicyclic heteroarylene group, each or which is unsubstituted or substituted by 1 to 10 R^{Z2} groups;
each R^{Z2} group is independently selected from the group consisting of —H, a halo group, an unsubstituted C₁₋₆ alkyl group, a C₁₋₆ alkyl group substituted by one or more —F, —OR^{Z2A}, a C₃₋₆ cycloalkyl group, a C₄₋₆ cycloheteroalkyl group, an unsubstituted C₁₋₆ alkyl group, a C₁₋₆ alkyl group substituted by a C₁₋₃ alkyl group, a C₁₋₆ alkyl group substituted by a C₁₋₆ alkoxyl group, a C₁₋₆ alkyl group substituted by one or more halo groups, an unsubstituted heterocyclic group, a heterocyclic group substituted by a C₁₋₃ alkyl group, a heterocyclic group substituted by a C₁₋₆ alkoxyl group, a heterocyclic group substituted by one or more halo groups, an unsubstituted aryl group, an aryl group substituted by a C₁₋₃ alkyl group, an aryl group substituted by a C₁₋₆ alkoxyl group, an aryl group substituted by one or more halo groups, an unsubstituted heteroaryl group, a heteroaryl group substituted by a C₁₋₃ alkyl group, a heteroaryl group substituted by a C₁₋₆ alkoxyl group, a heteroaryl group substituted by one or more halo groups, a bicyclic hereoaryl group, an unsubstituted C₁₋₃ alkoxyl group, and a C₁₋₃ alkoxyl substituted by one or more groups selected from —F, —OH, —NH₂, —NR^{Y1}R^{Y2} and —CN; and
R^{Z2A} is selected from the group consisting of H, a C₁₋₆ alkyl group, and a C₁₋₆ heteroalkyl group, each of which is unsubstituted or substituted by a cycloalkyl group, a cycloheteroalkyl group, an aryl group, a heterocyclic group, a heteroaryl group, a halo group, or a C₁₋₃alkoxyl group.

25. The bifunctional compound, or the pharmaceutically acceptable salt, hydrate, solvate, metabolite or prodrug thereof as claimed in claim 24, wherein Z¹ is selected from the group consisting of

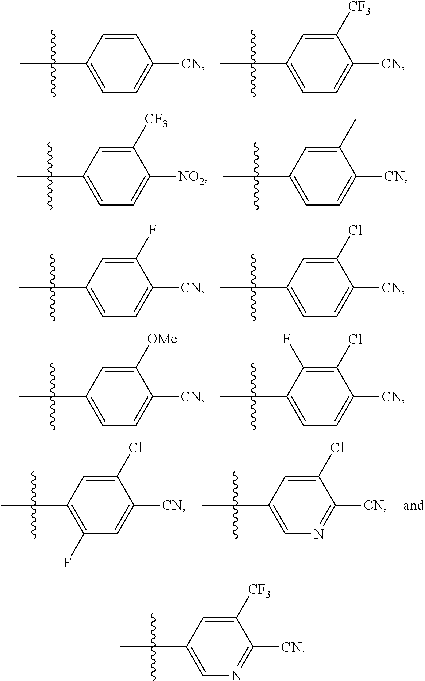

26. The bifunctional compound, or the pharmaceutically acceptable salt, hydrate, solvate, metabolite or prodrug thereof as claimed in claim 24, wherein Z² is selected from the group consisting of

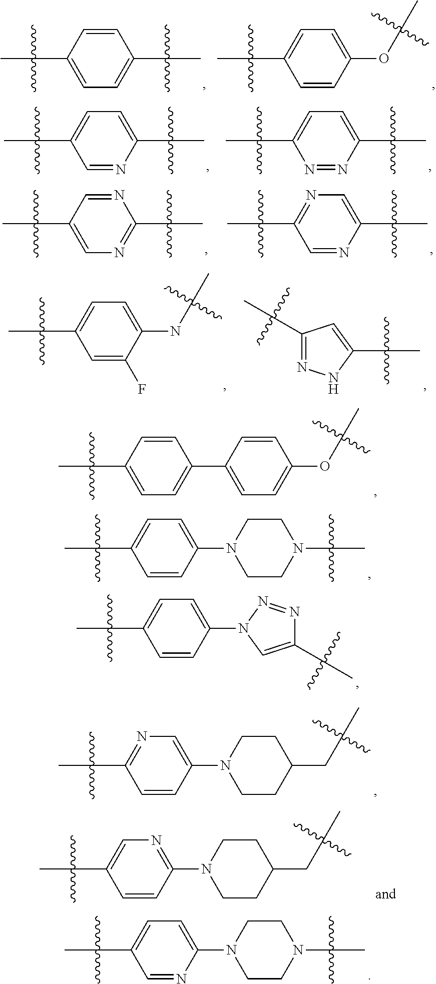

27. The bifunctional compound, or the pharmaceutically acceptable salt, hydrate, solvate, metabolite or prodrug thereof as claimed in claim 24, wherein the ABM is selected from the group consisting of 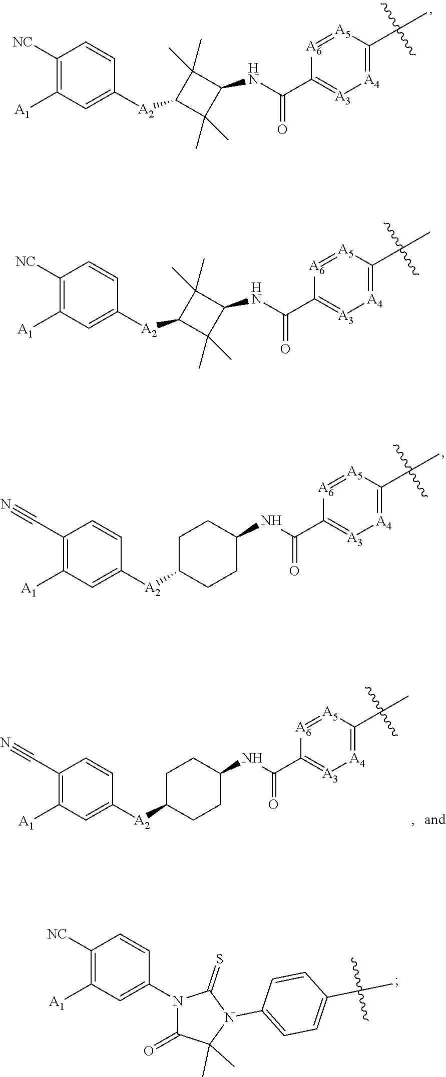
wherein:
A₁ is selected from —Cl, —F, —Br or —CF₃;
A₂ is selected from —O—, —NH—, —NCH₃— or —NCH₂CH₃—; and
A₃, A₄, A₅ and A₆ are each independently —CH— or —N—.

28. The bifunctional compound, or the pharmaceutically acceptable salt, hydrate, solvate, metabolite or prodrug thereof as claimed in claim 20, wherein the bifunctional compound is selected from the group consisting of:

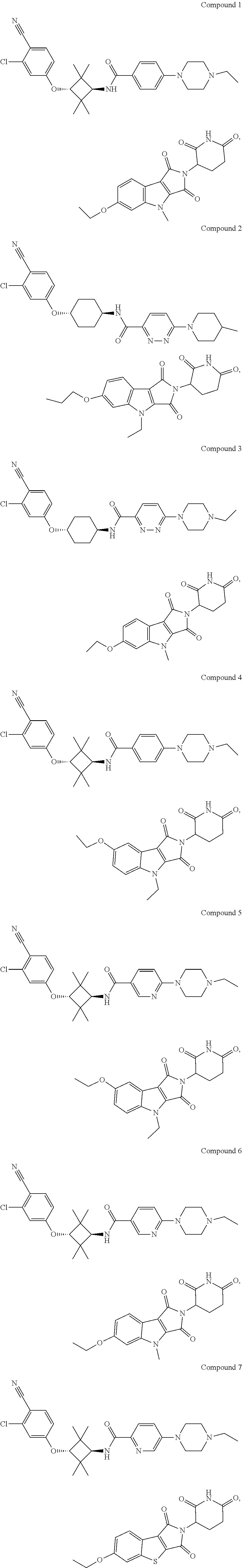

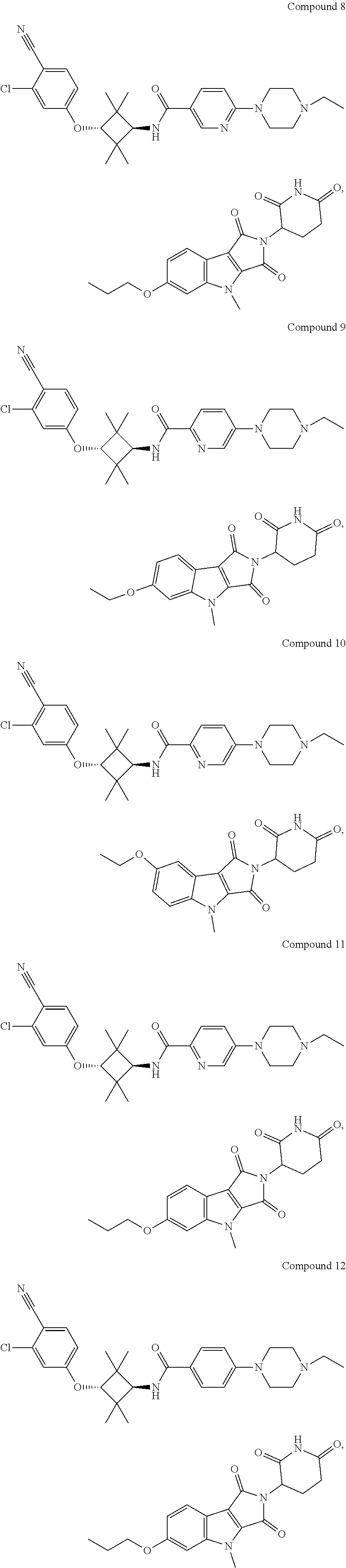

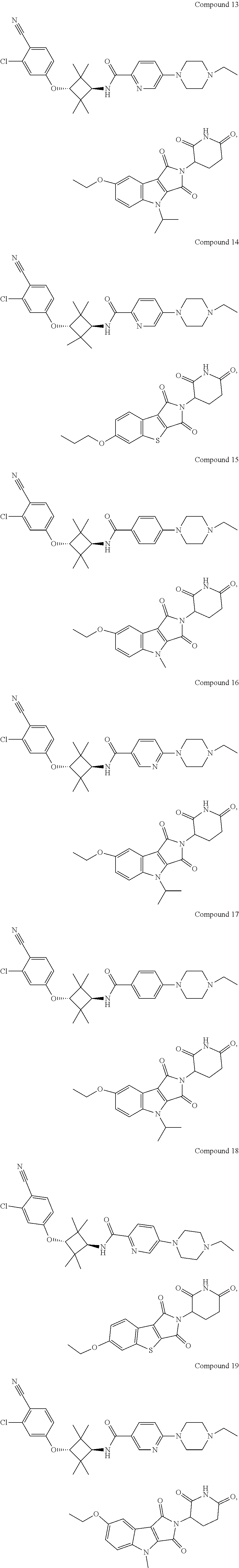

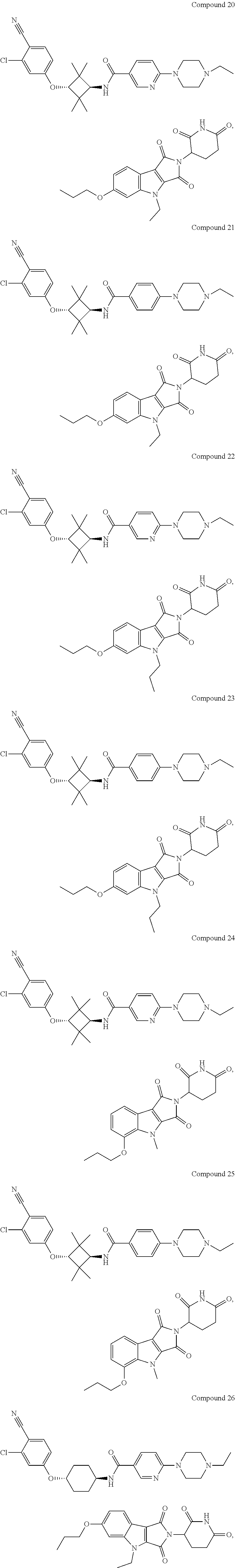

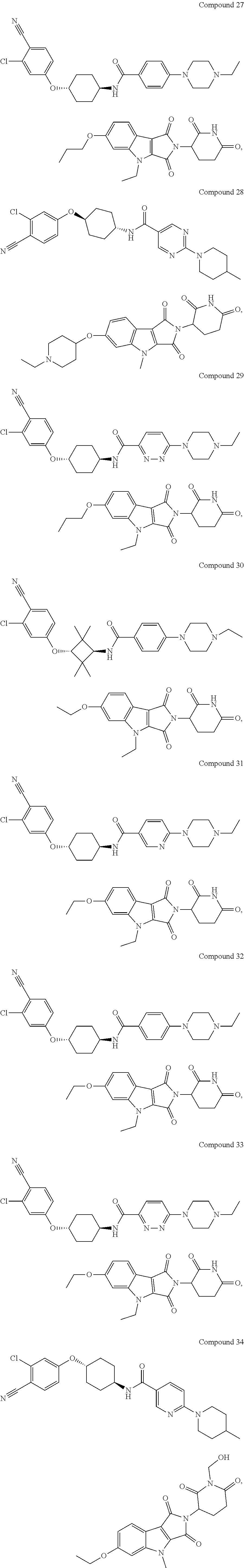

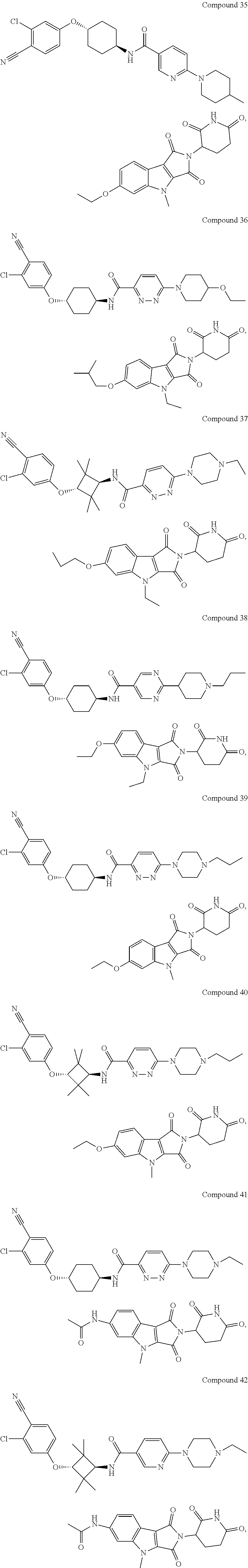

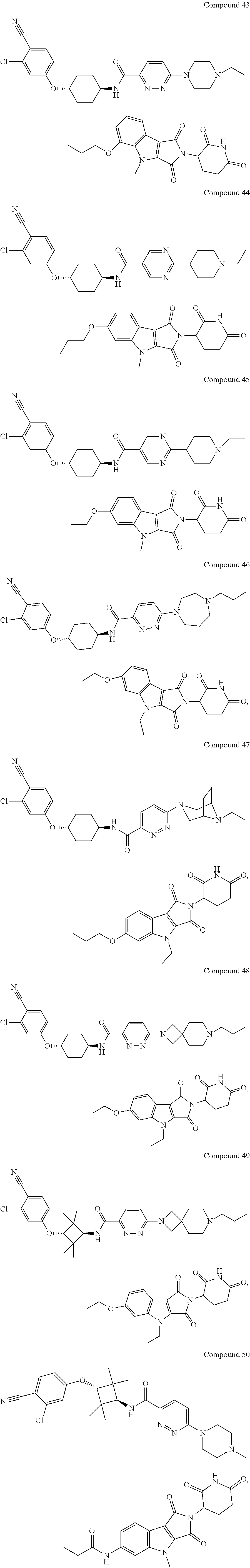

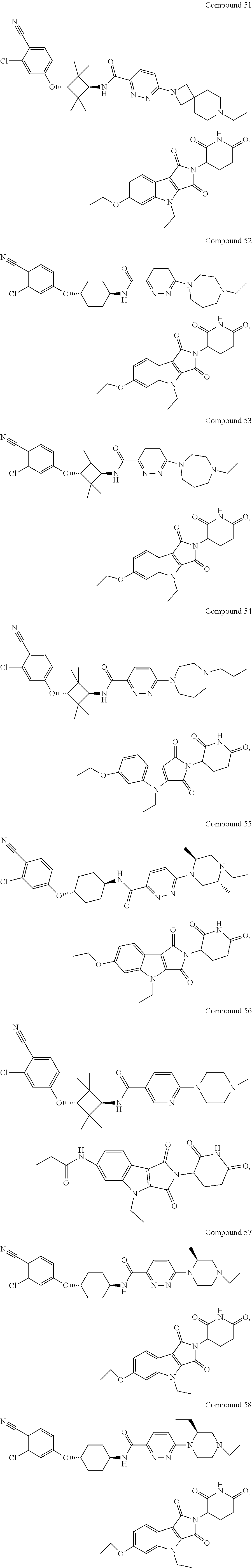

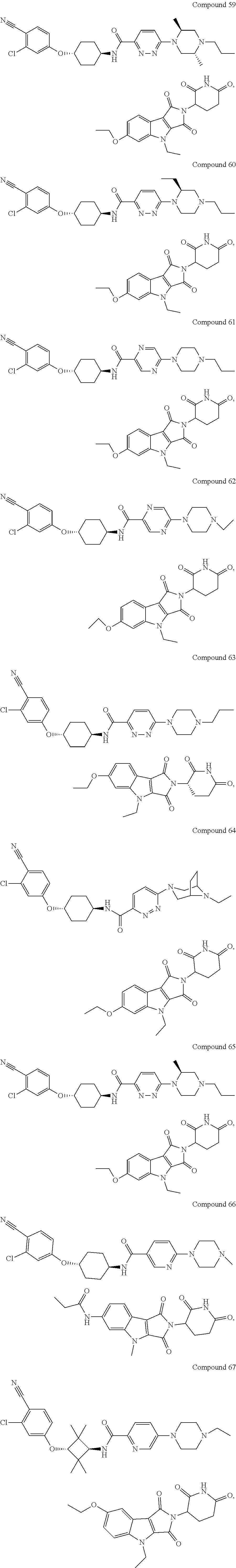

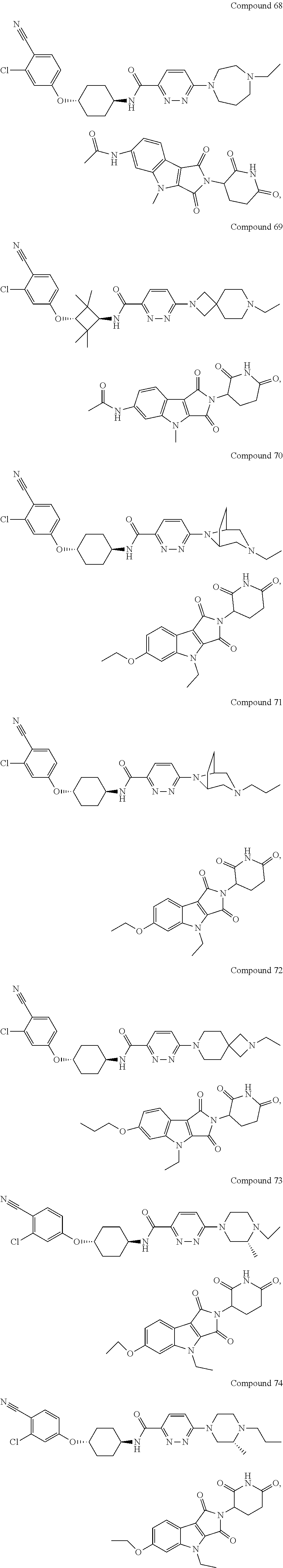

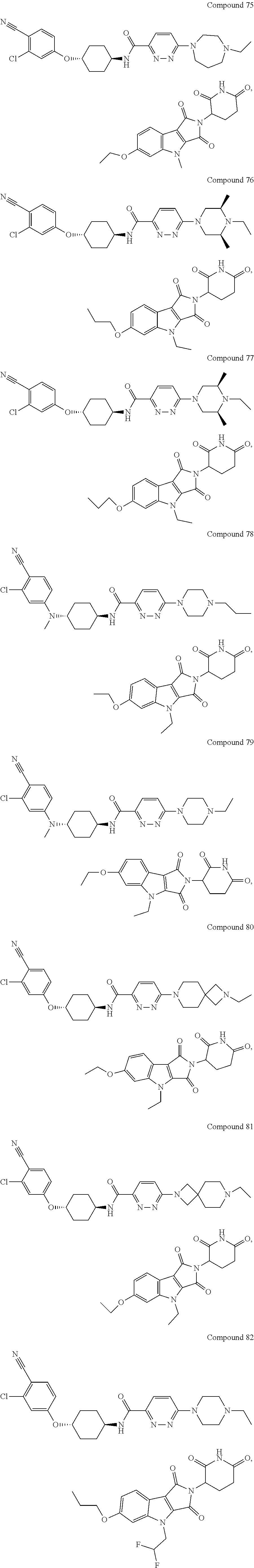

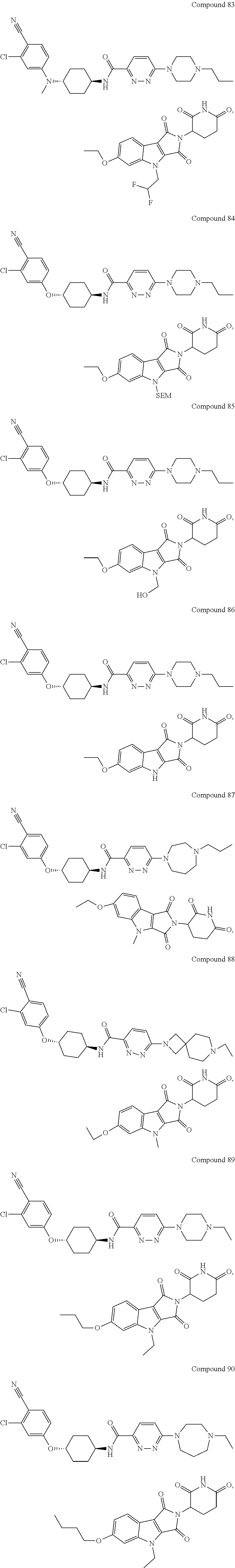

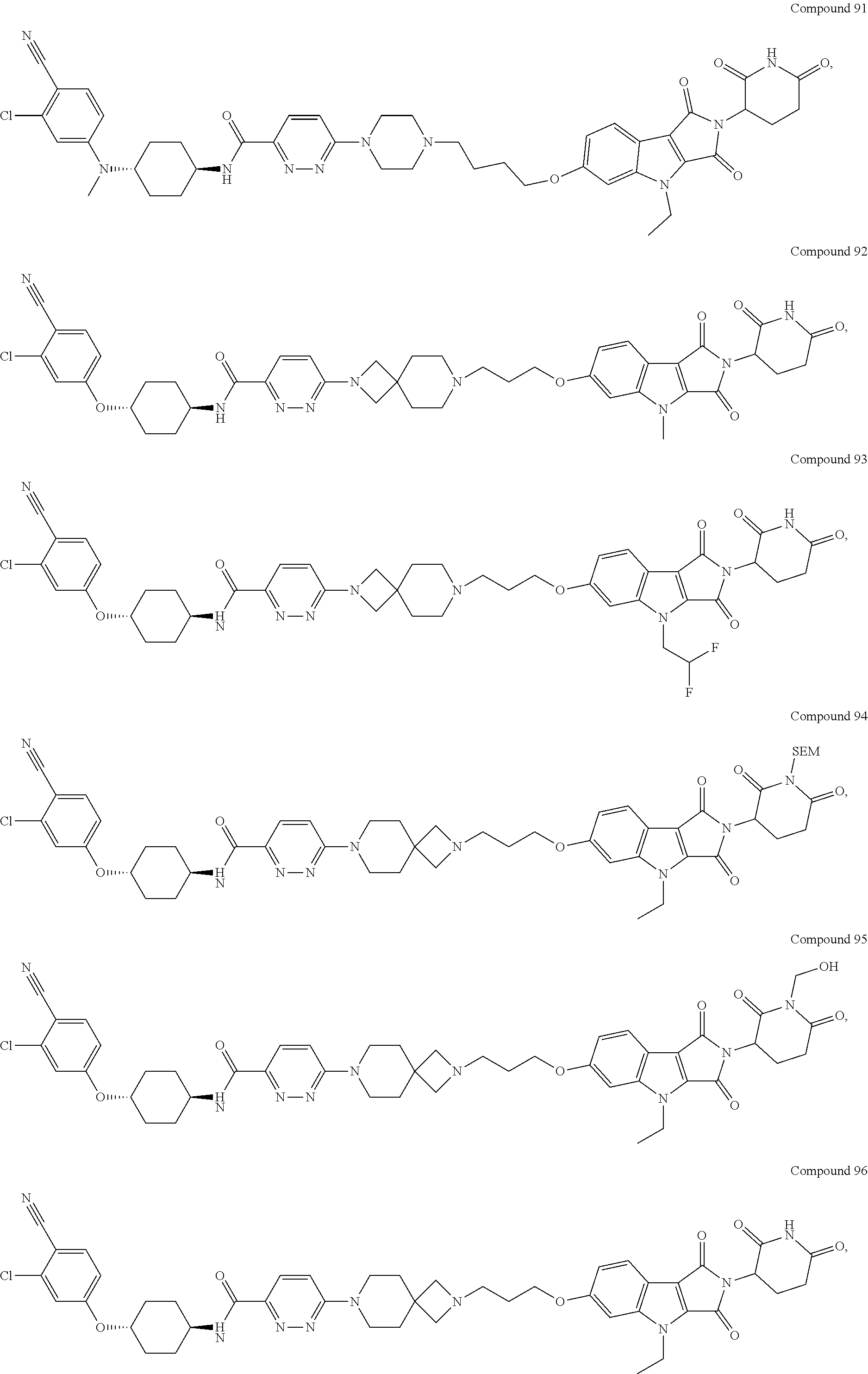

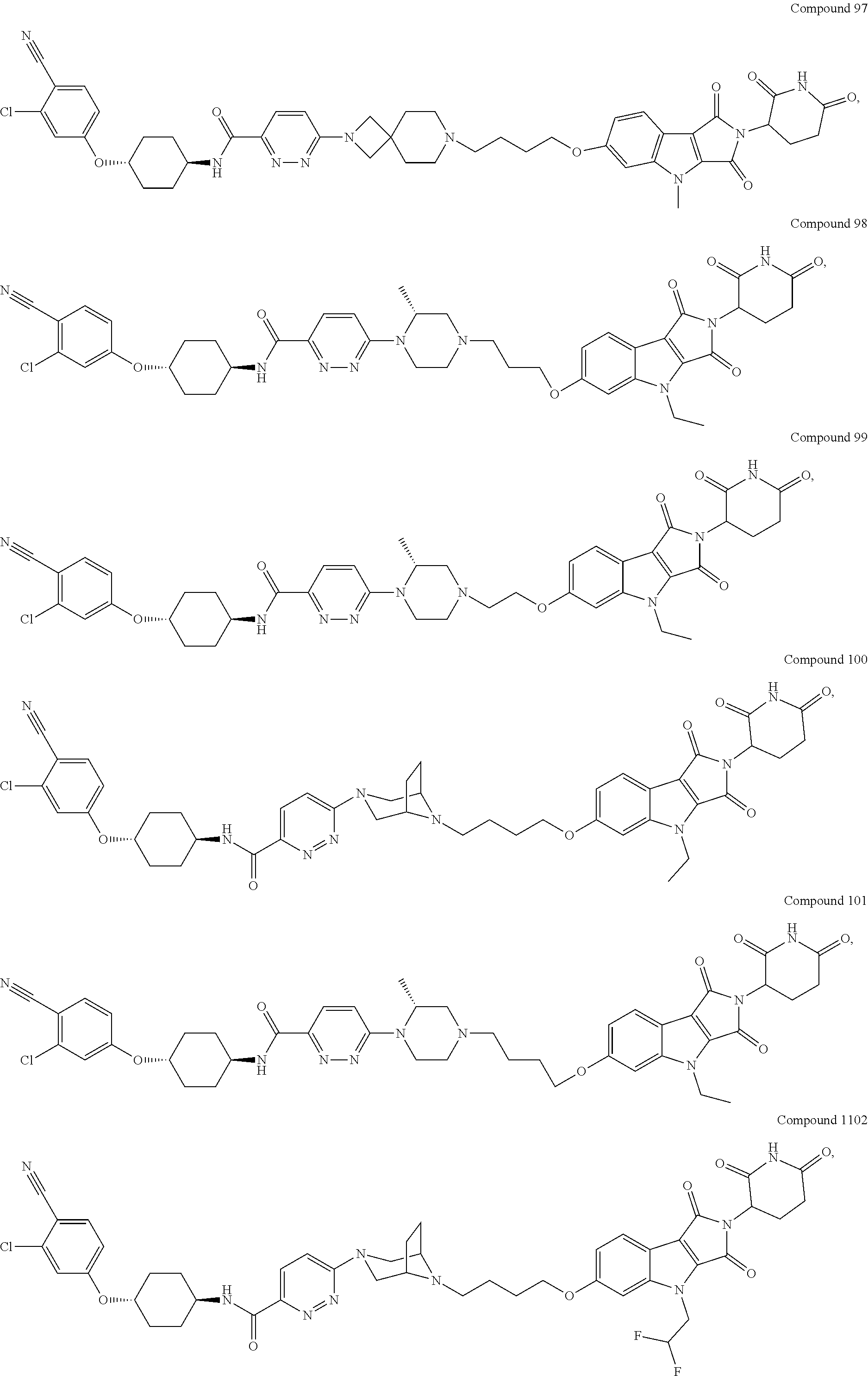

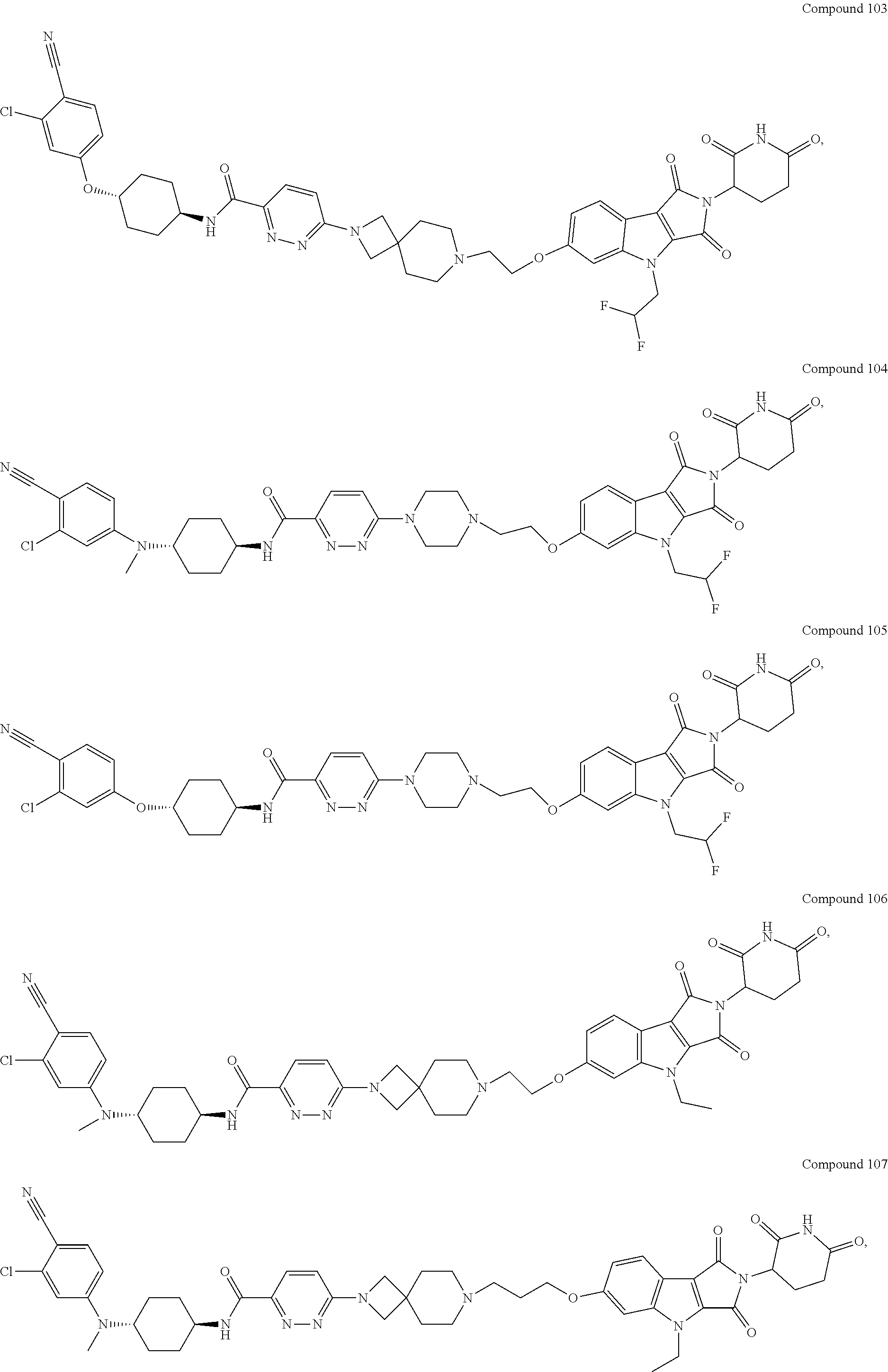

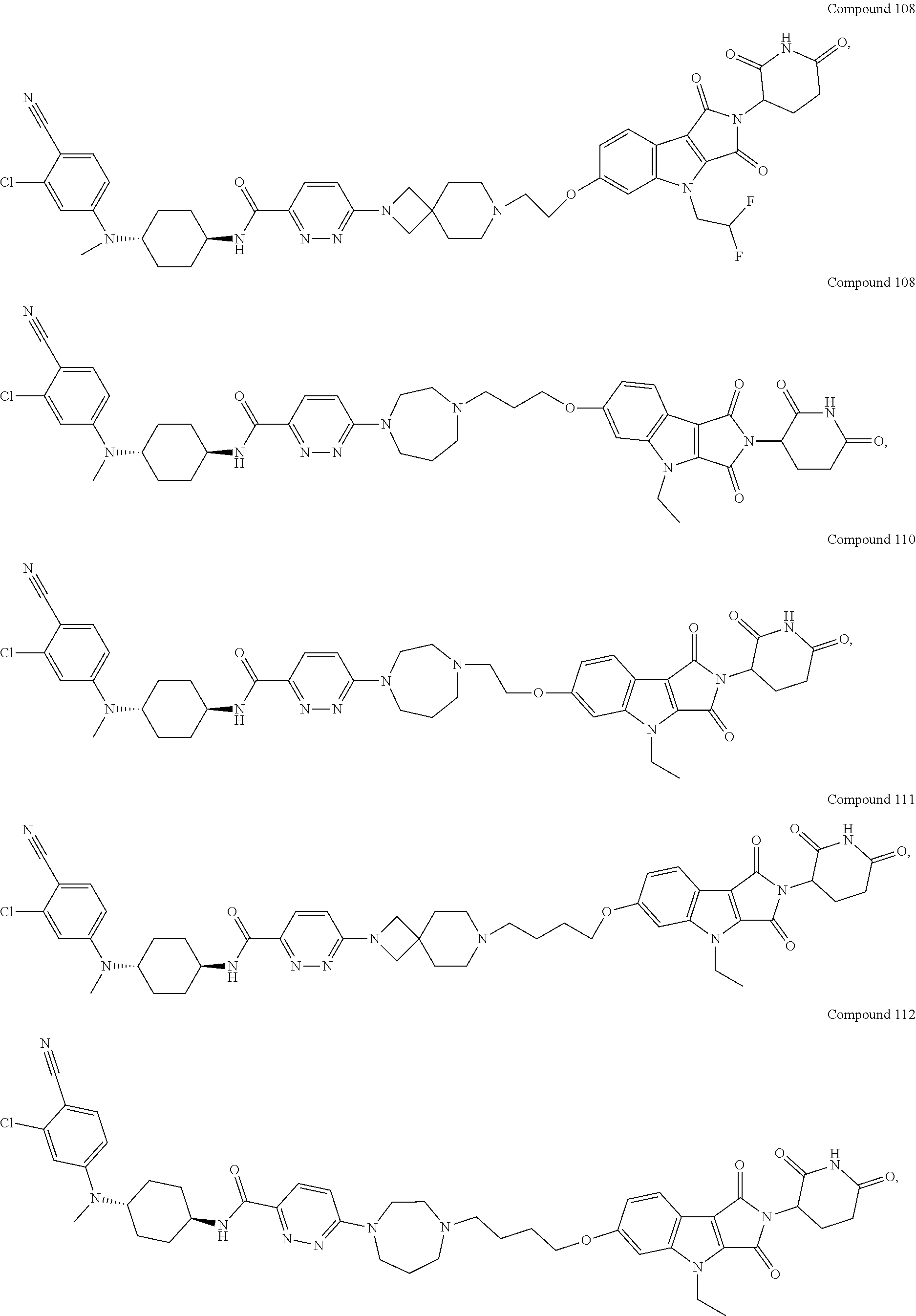

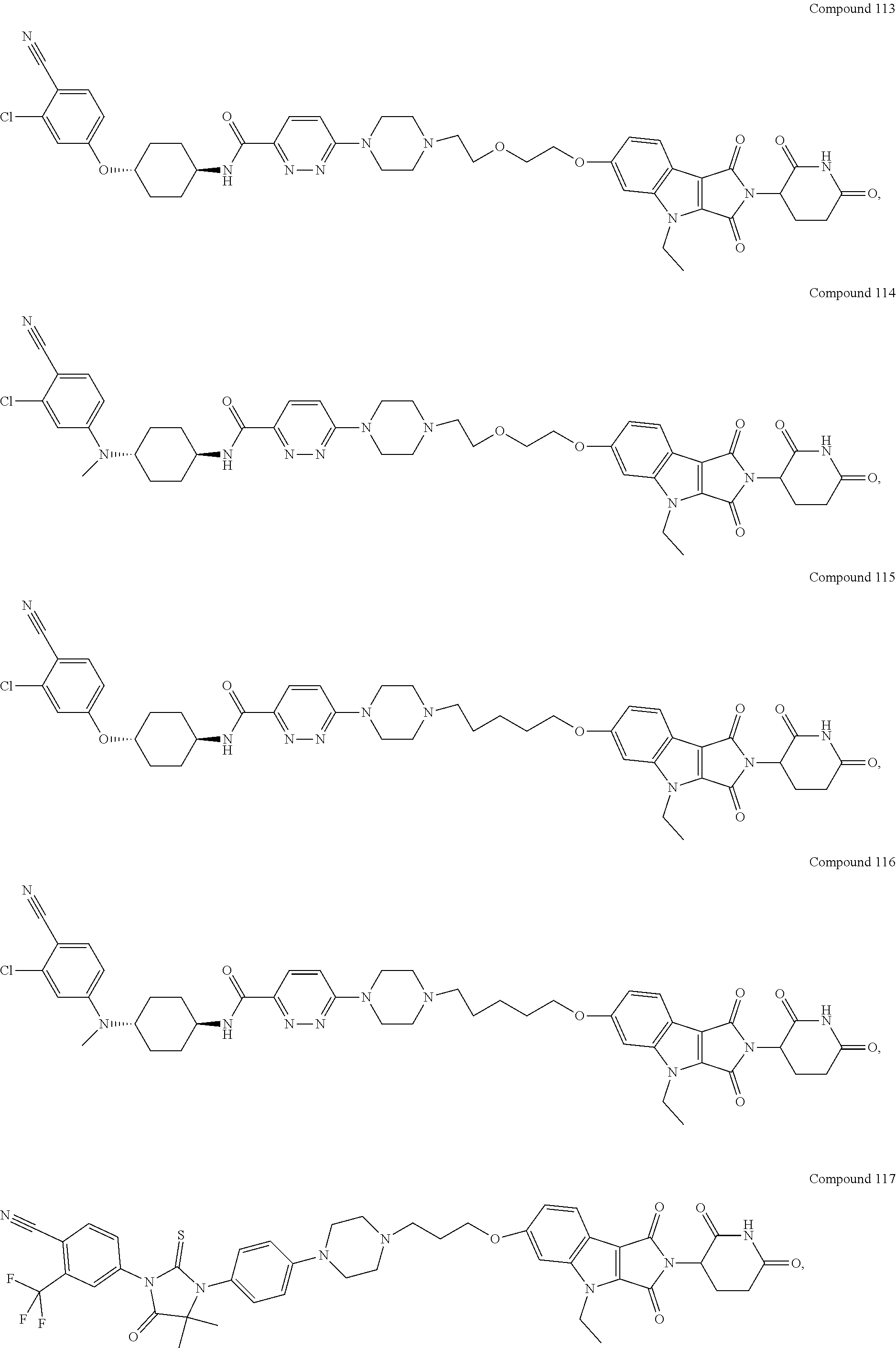

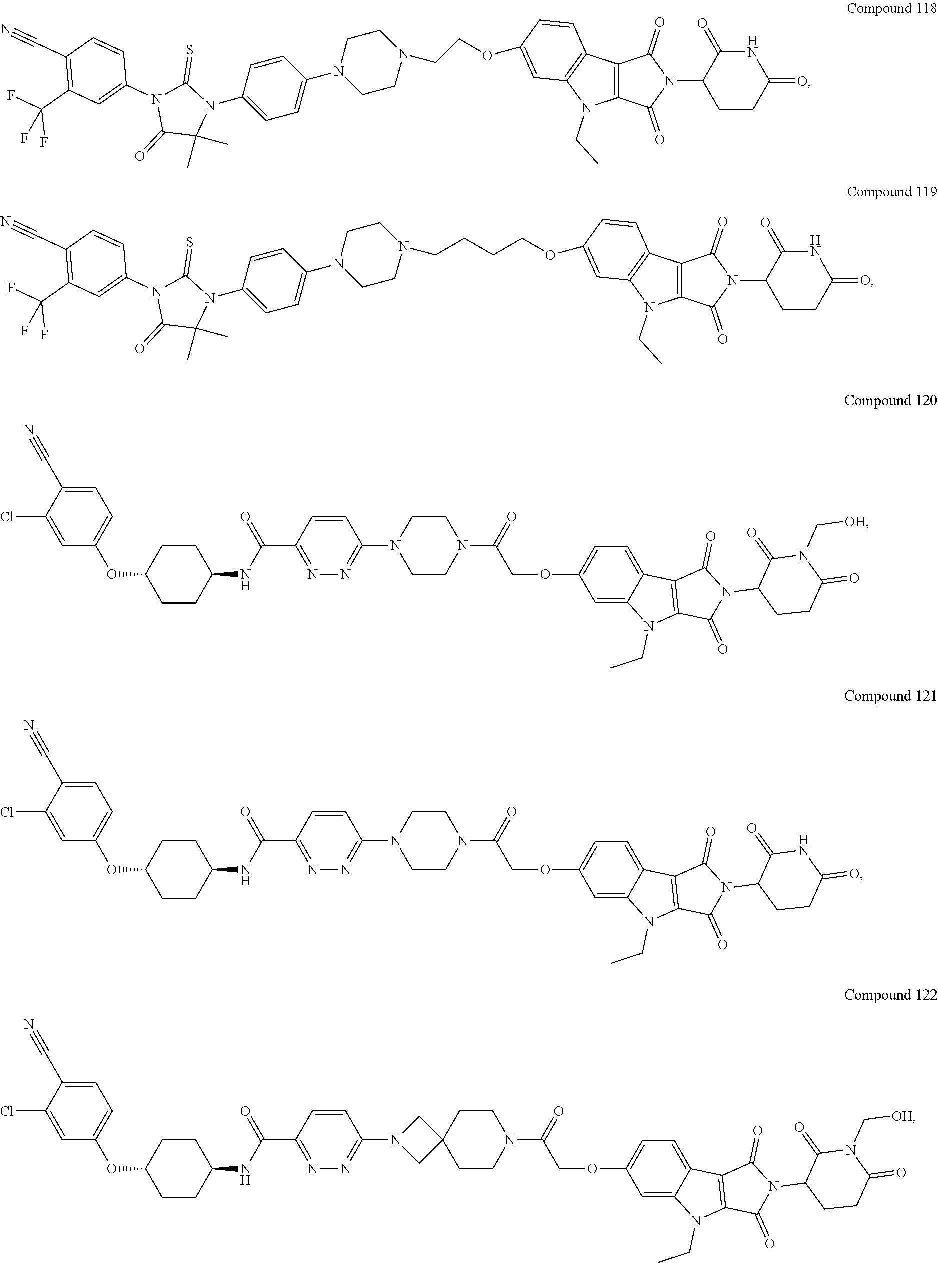

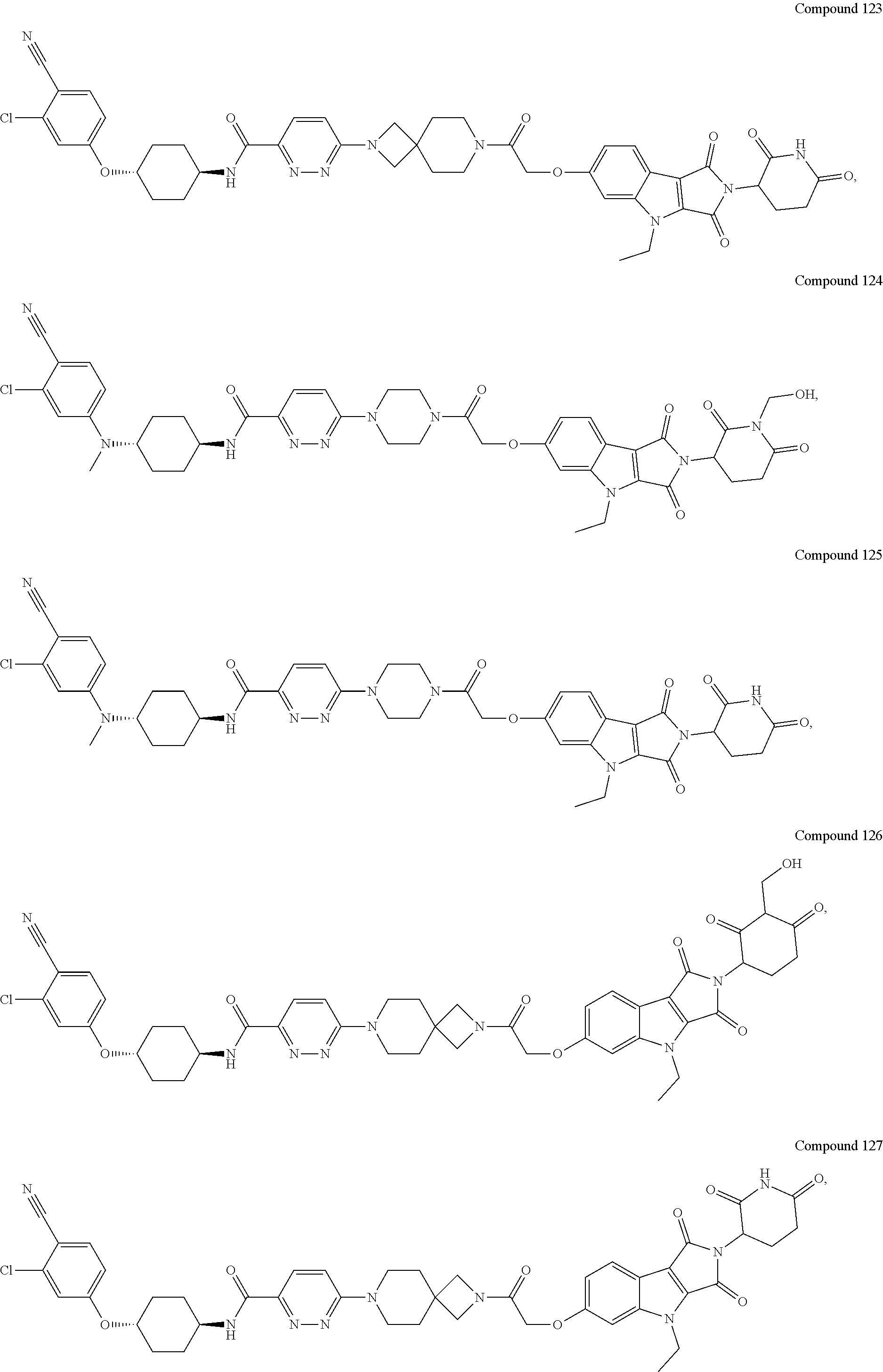

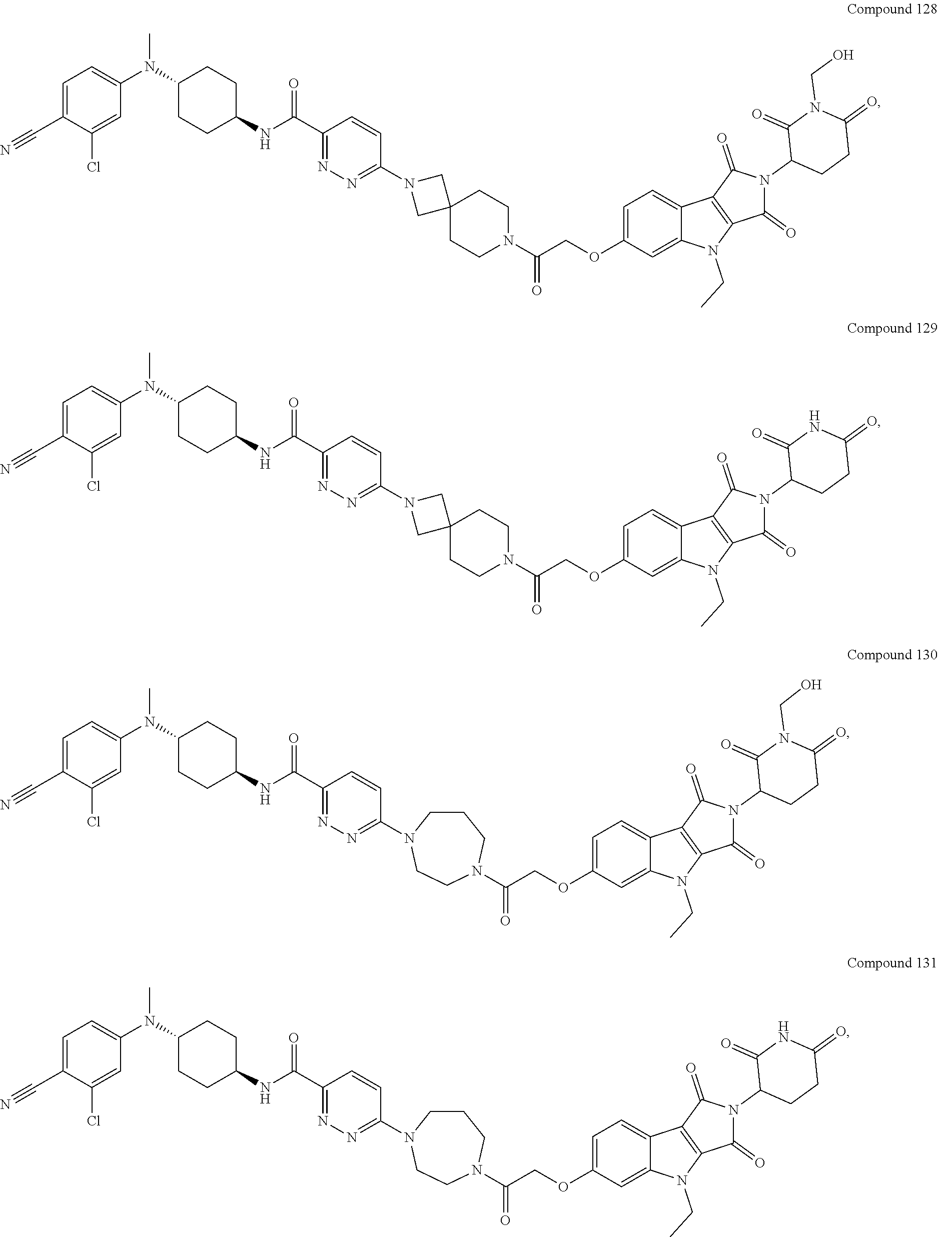

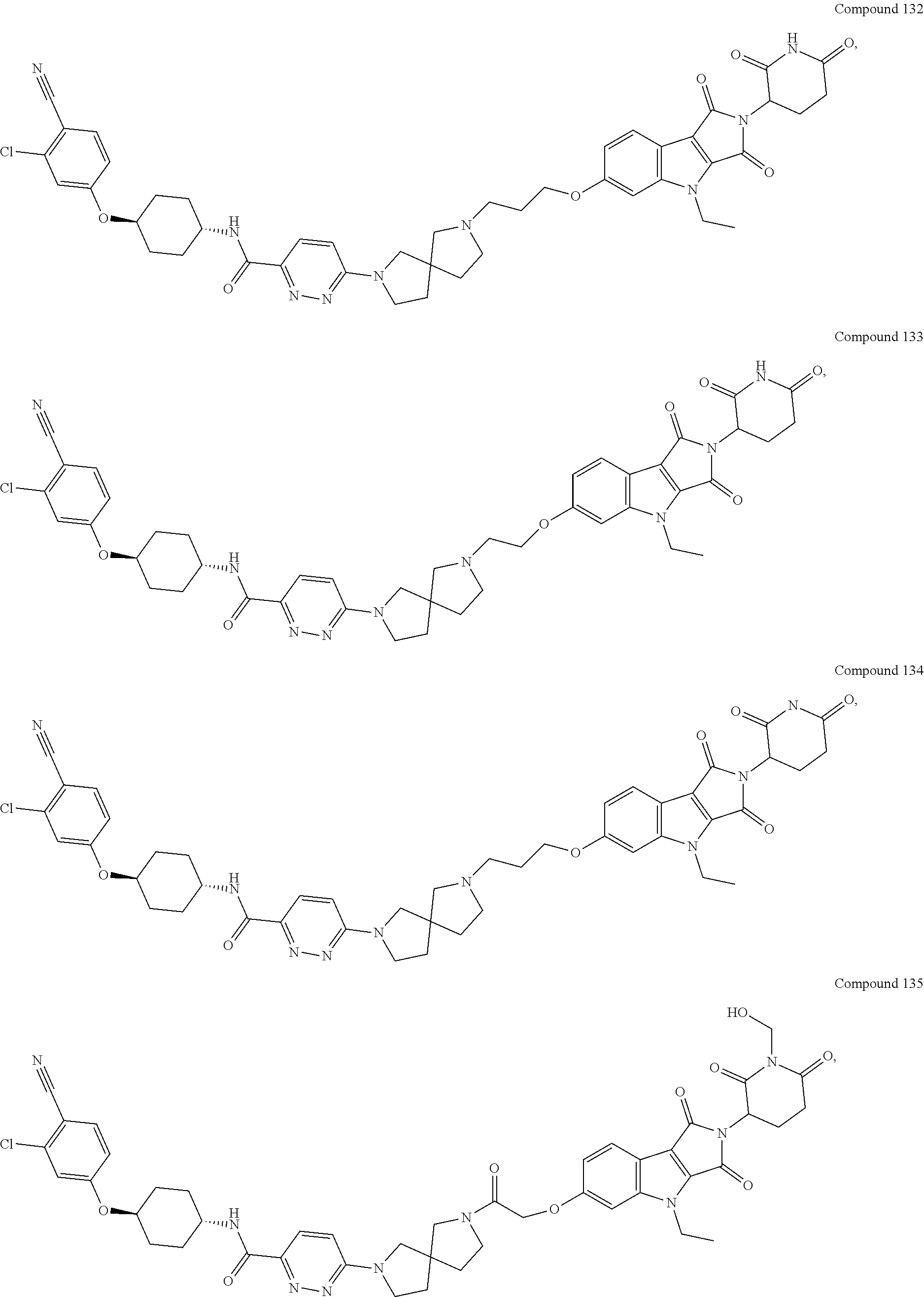 [SMILES: CCn1c2c(c3ccc(OCCCN4CCC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O ; CCn1c2c(c3ccc(OCCN4CCC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O ; CCn1c2c(c3ccc(OCCCN4CCC5(CCN(c6ccc(C(=O)N[C@H]7CC[C@H](Oc8ccc(C#N)c(Cl)c8)CC7)nn6)C5)C4)cc31)C(=O)N(C1CCC(=O)NC1=O)C2=O ; CCn1c2c(c3ccc(OCC(=O)N4CCC5(CCN(c6ccc(C(=O)N[C@H]7CC[C@H](Oc8ccc(C#N)c(Cl)c8)CC7)nn6)C5)C4)cc31)C(=O)N(C1CCC(=O)N(CO)C1=O)C2=O]

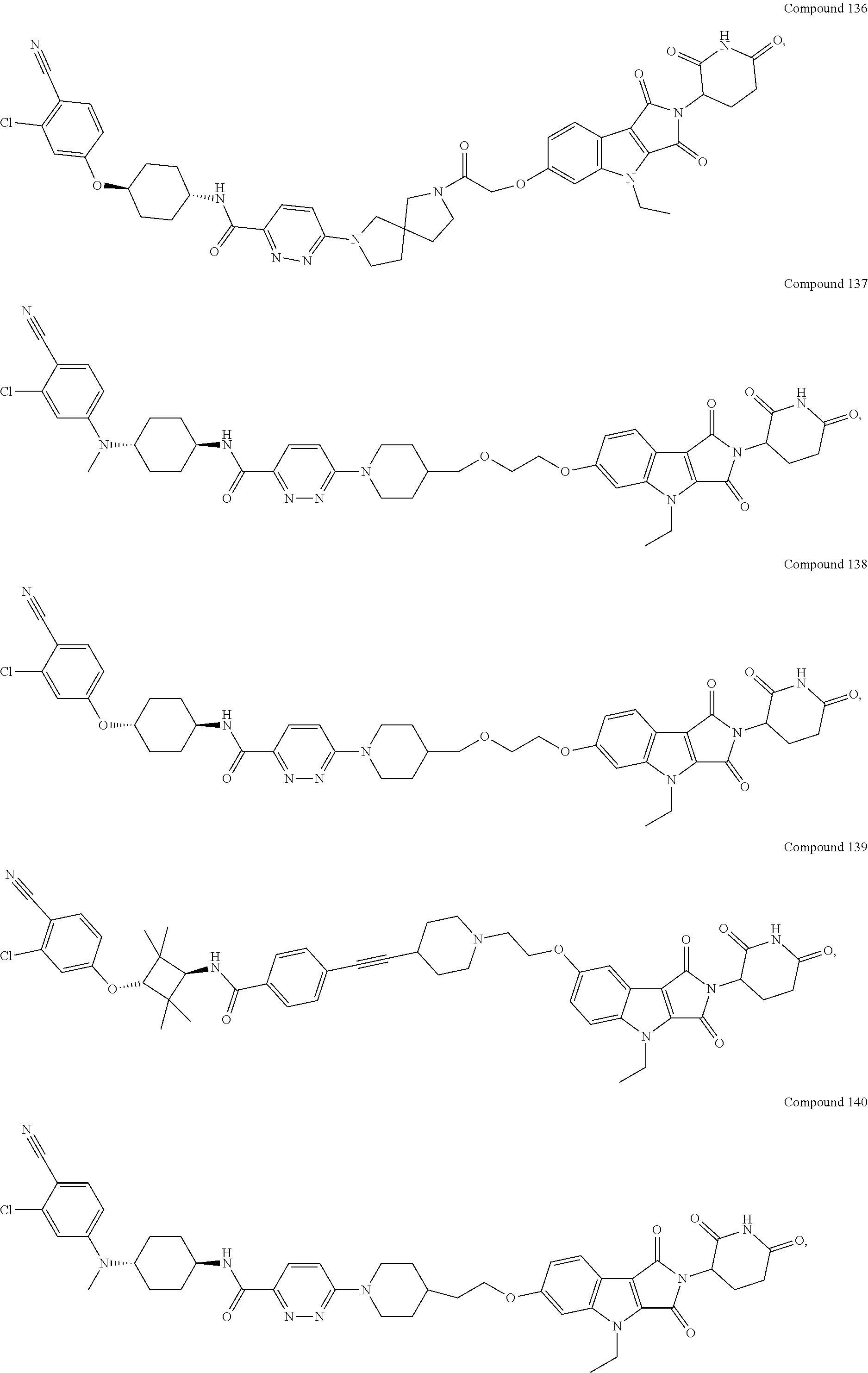

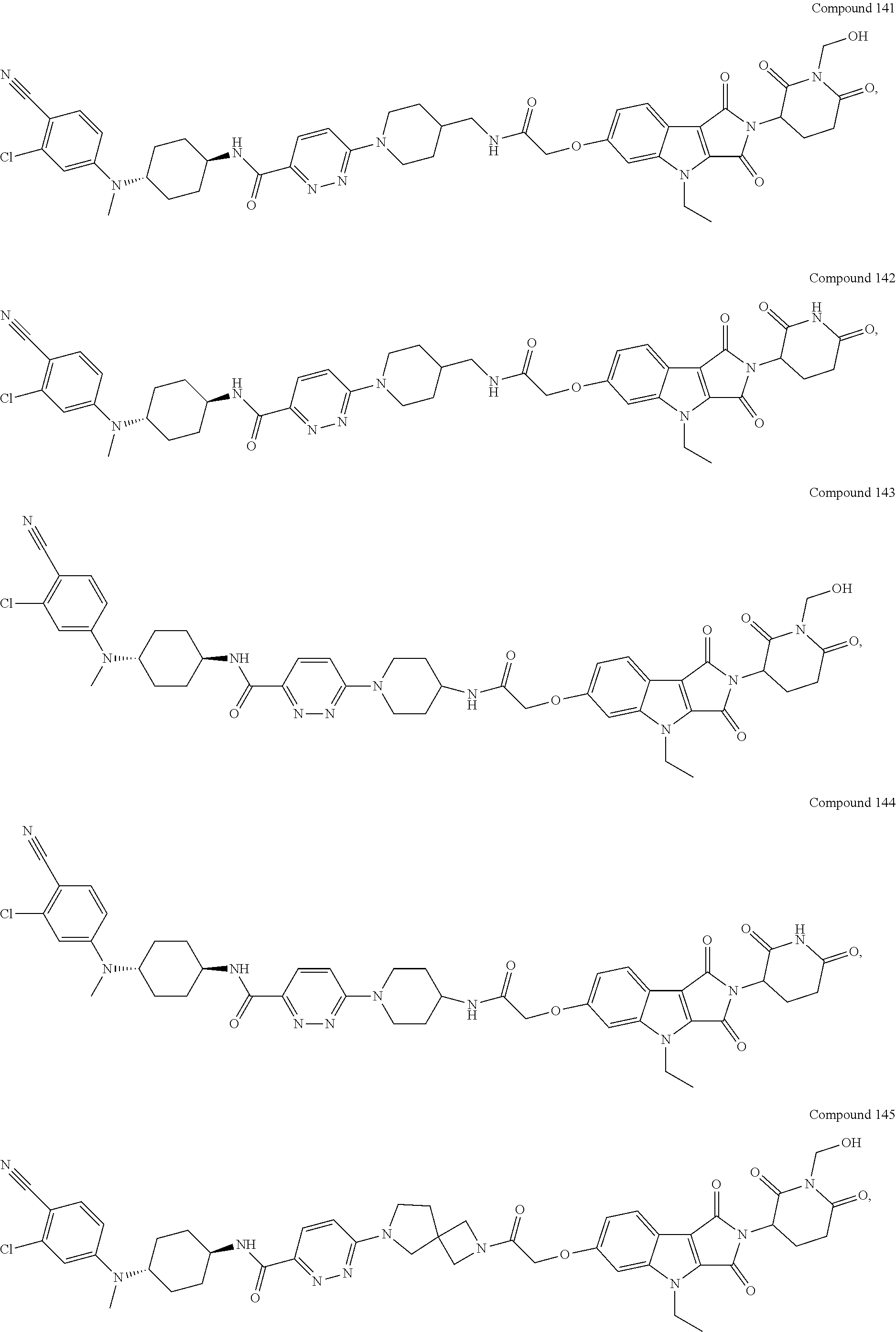

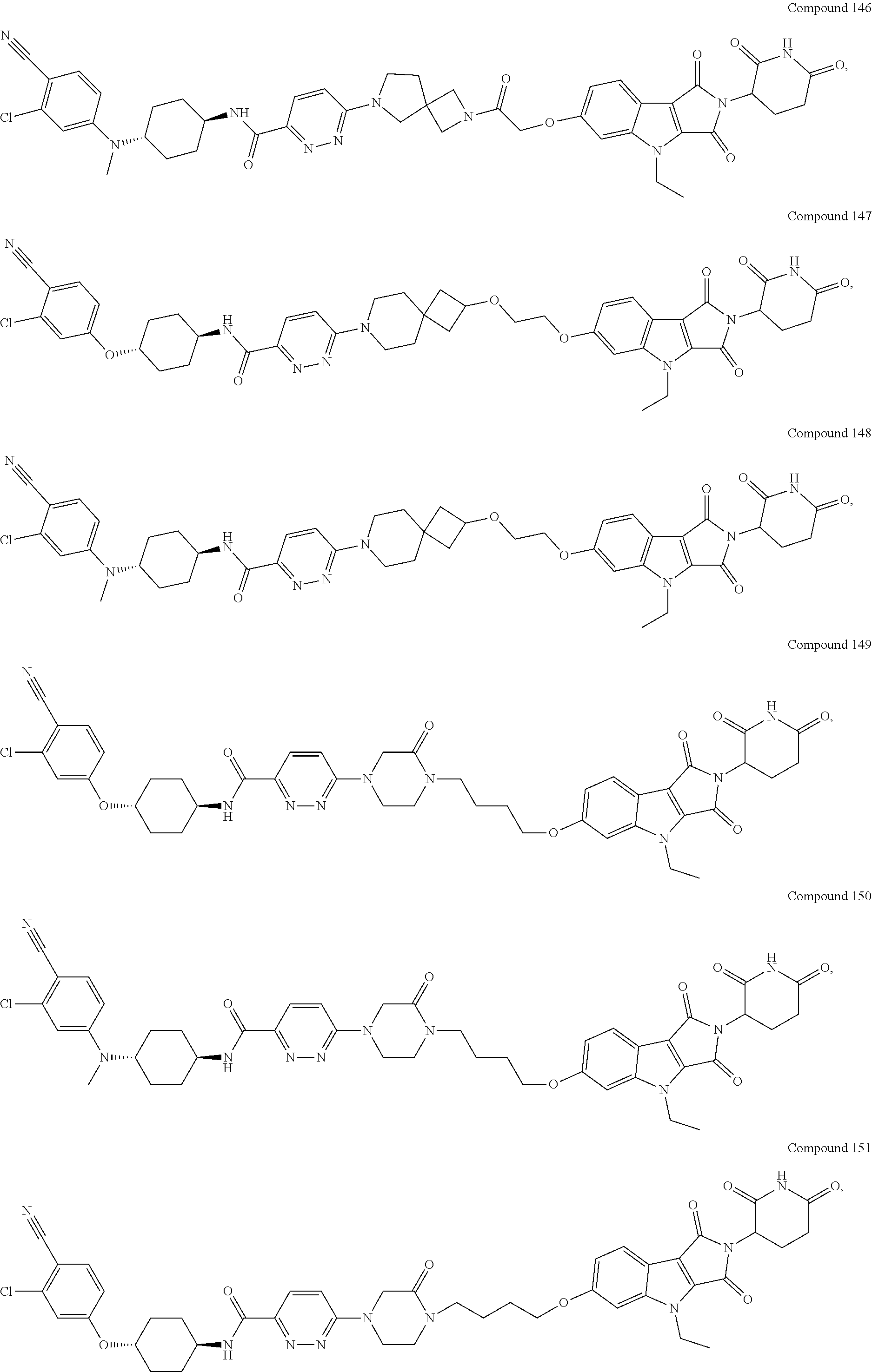

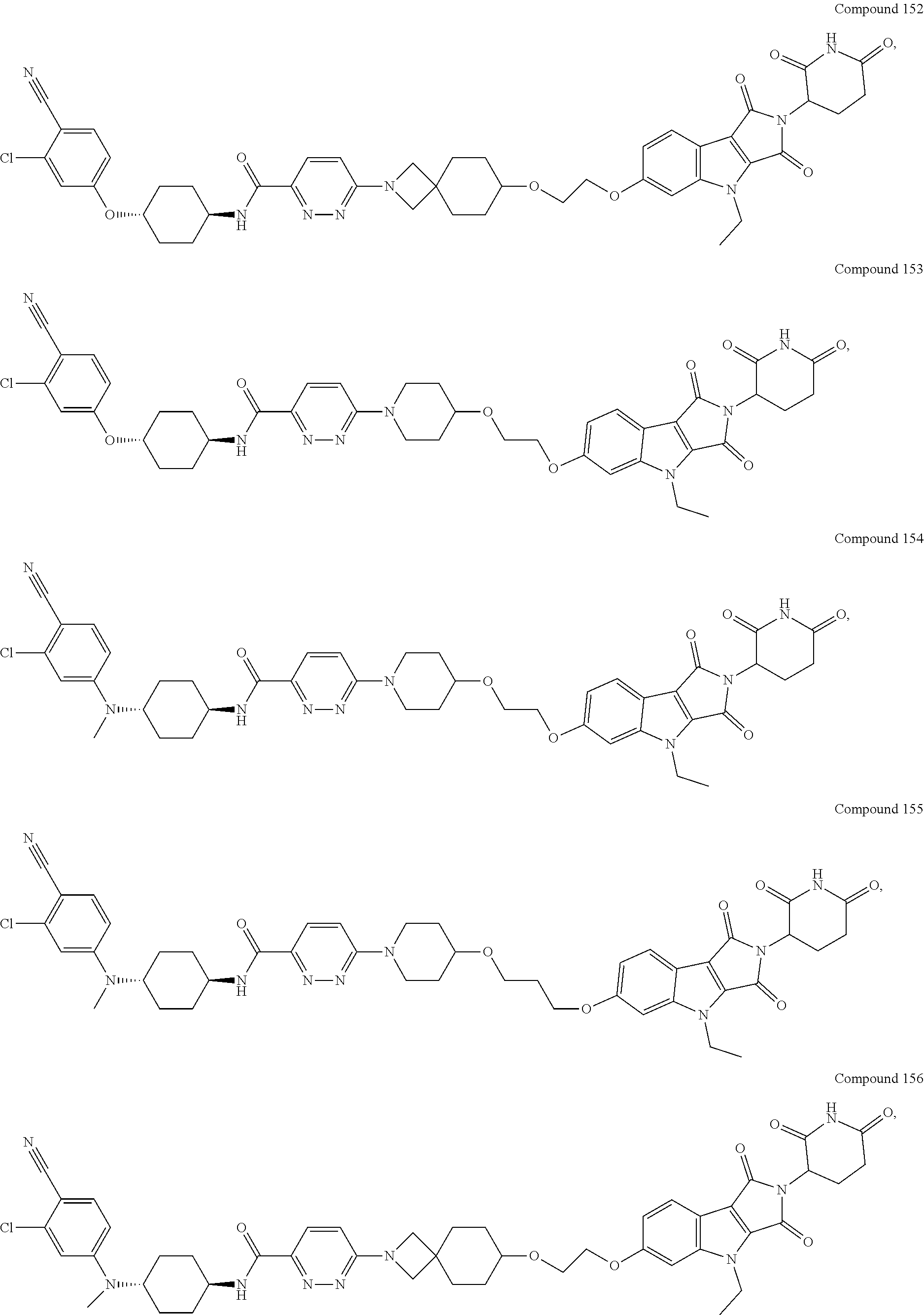

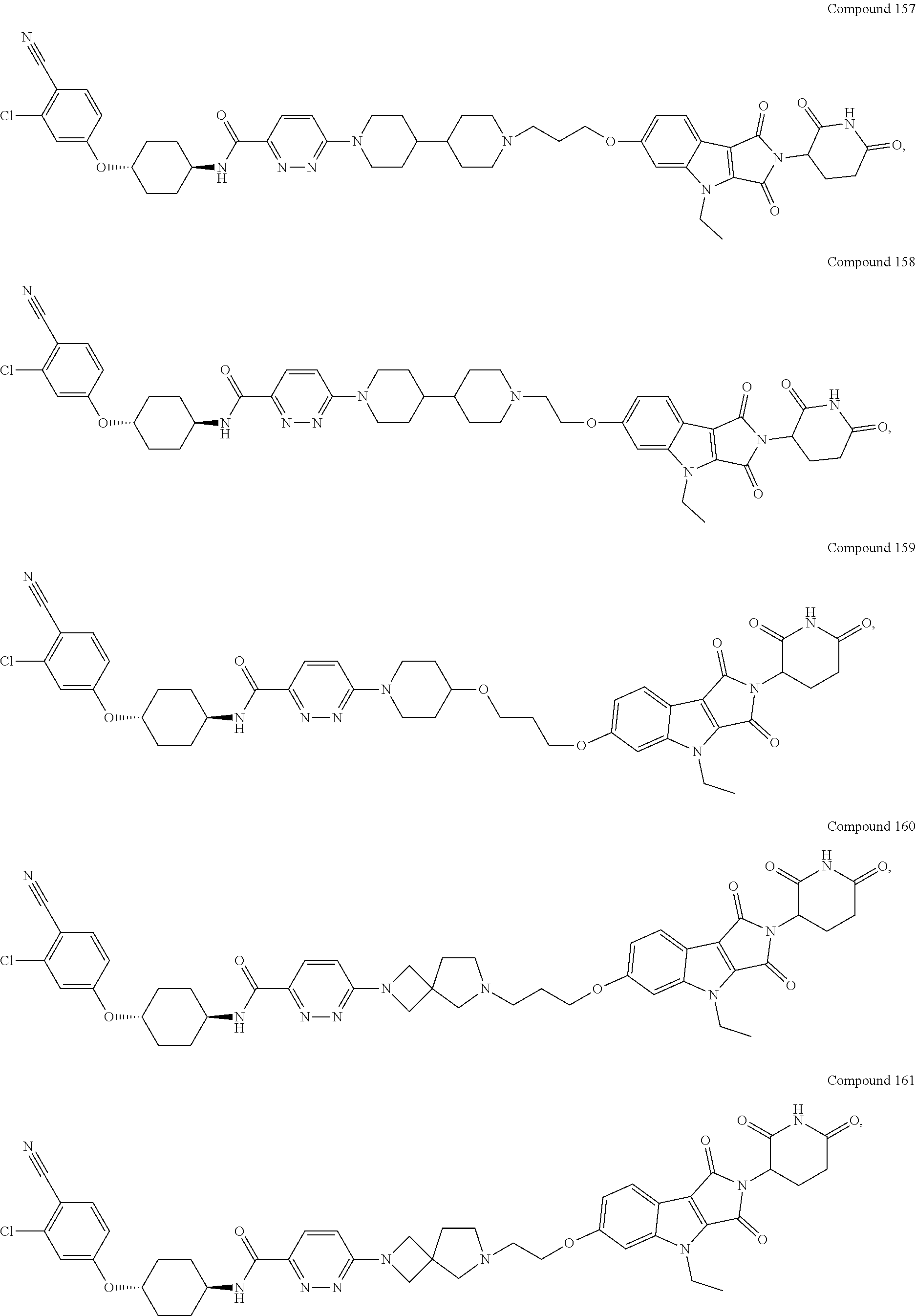

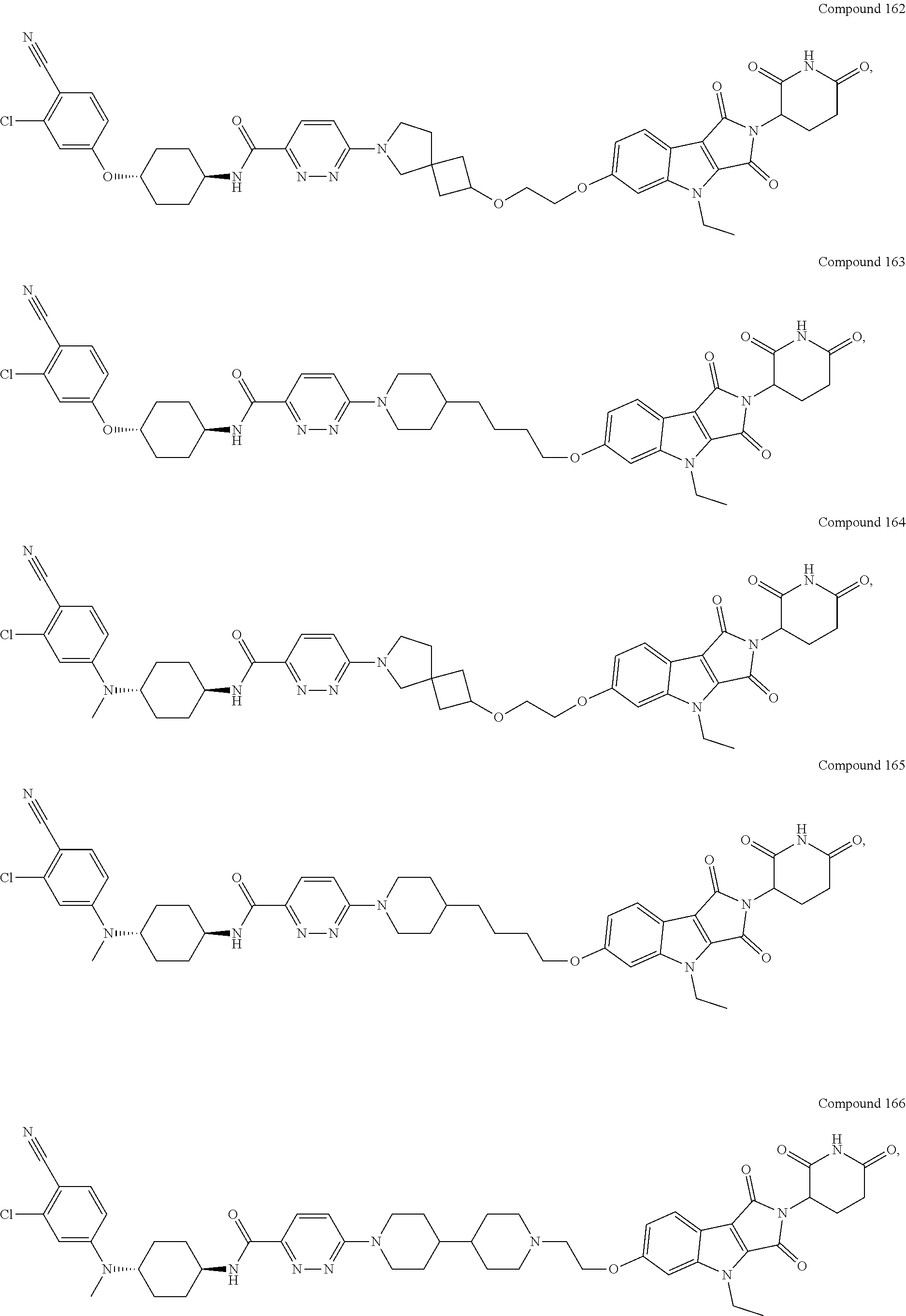

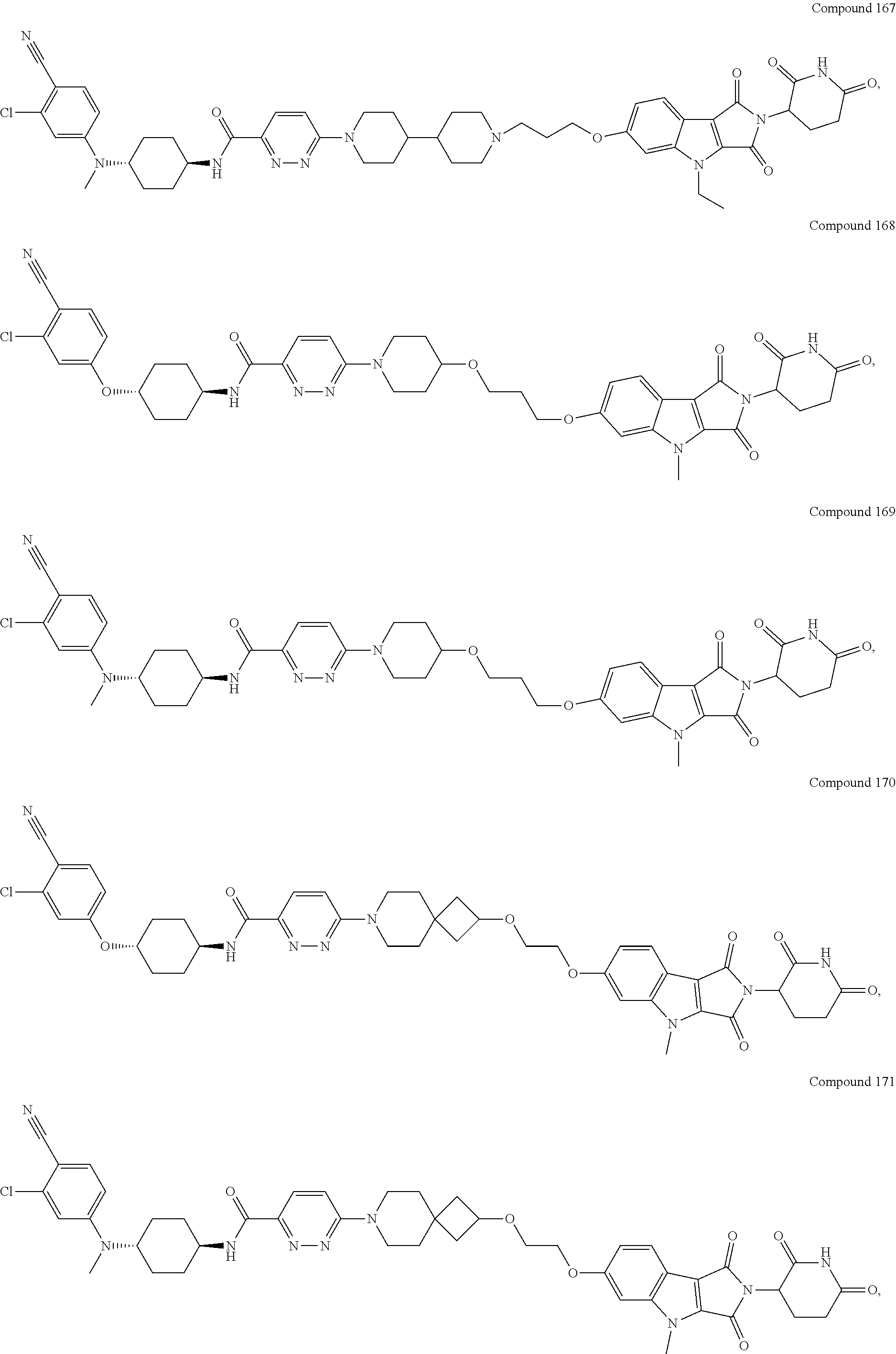

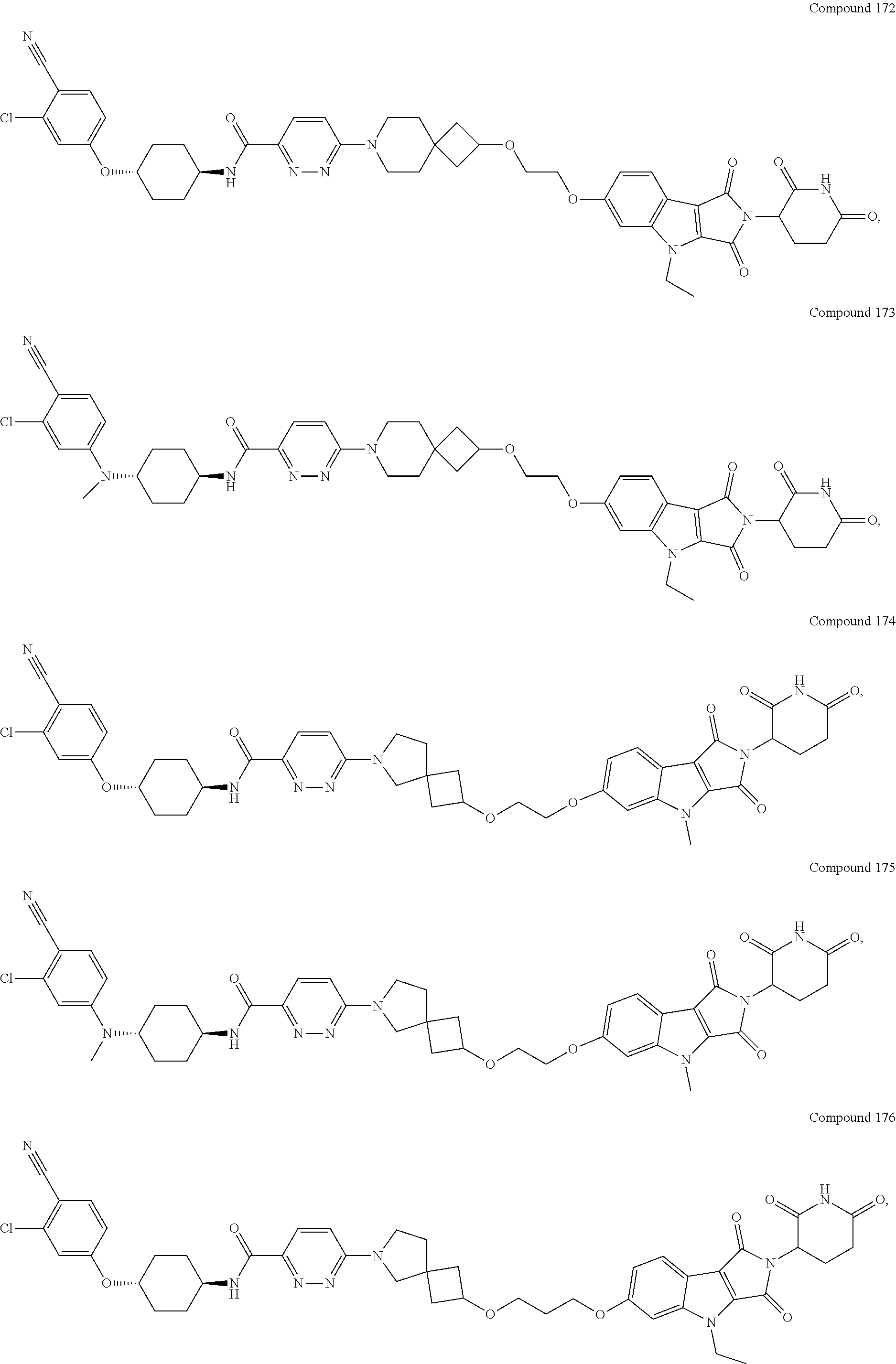

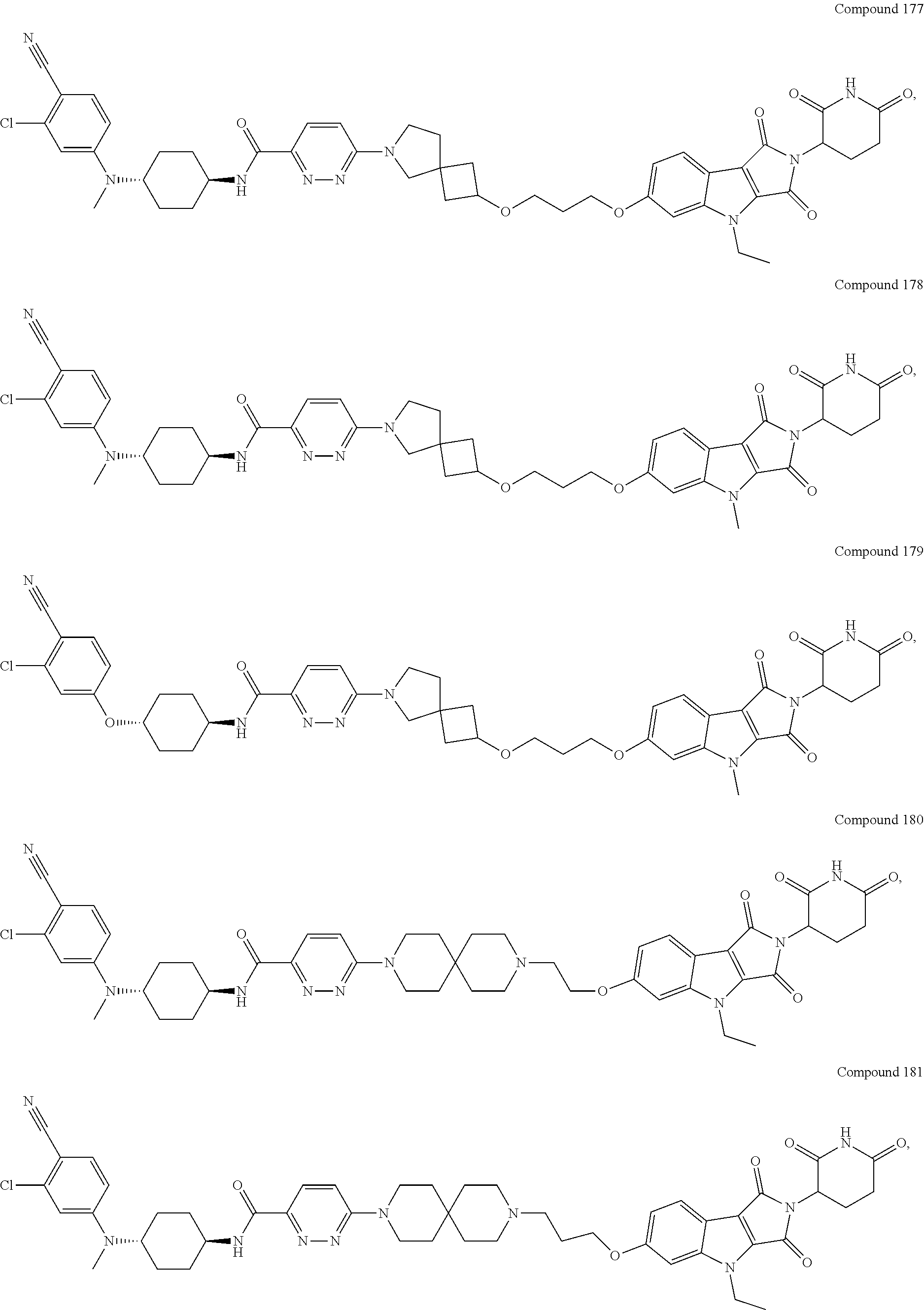

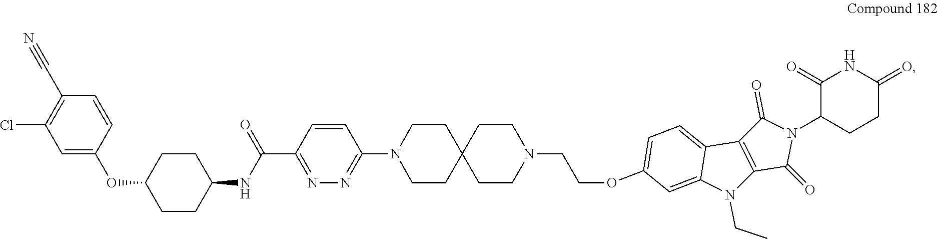 [SMILES: CCn1c2c(c3ccc(OCCN4CCC5(CC4)CCN(c4ccc(C(=O)N[C@H]6CC[C@H](Oc7ccc(C#N)c(Cl)c7)CC6)nn4)CC5)cc31)C(=O)N(C1CCC(=O)NC1=O)C2=O]

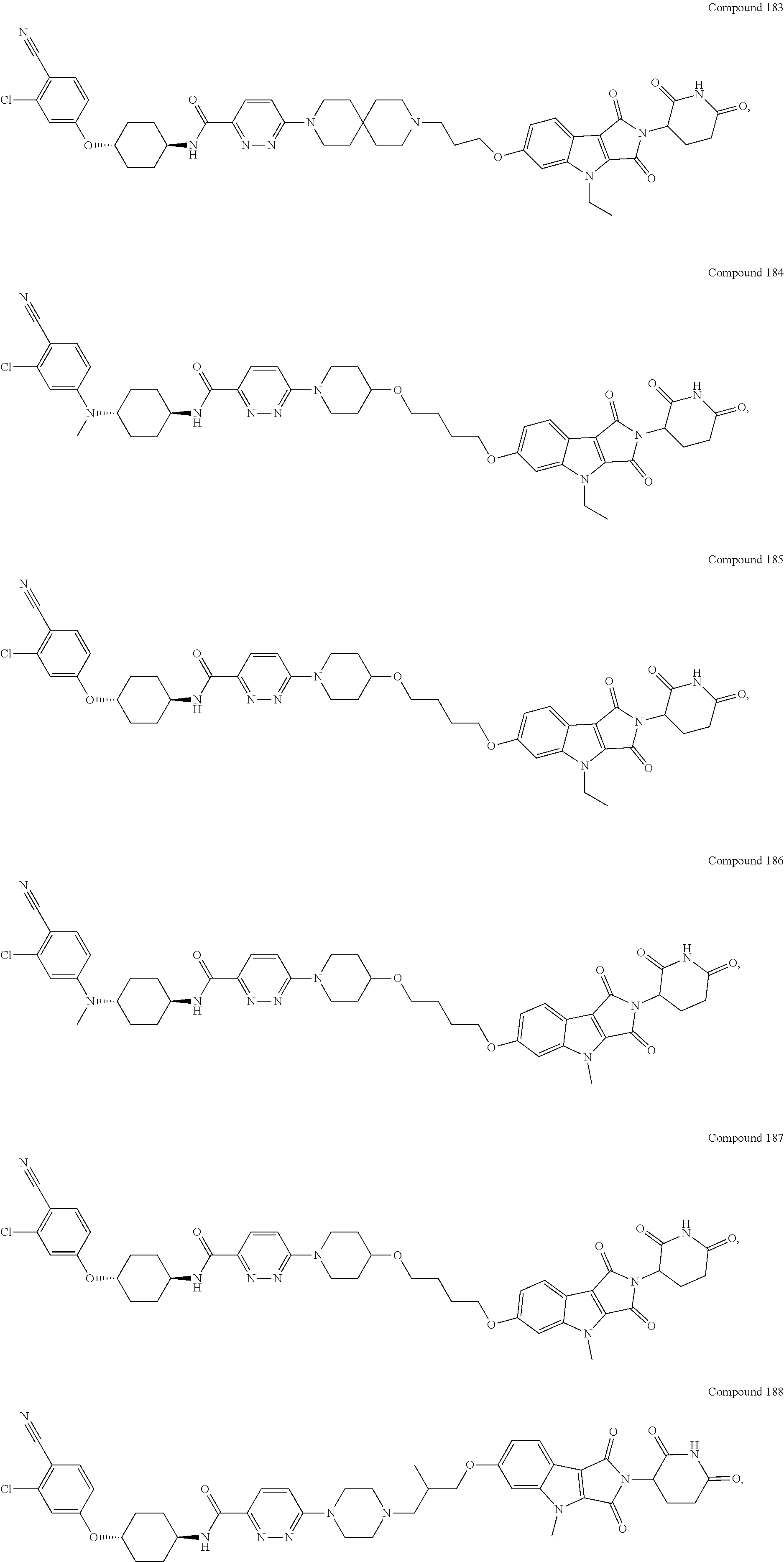

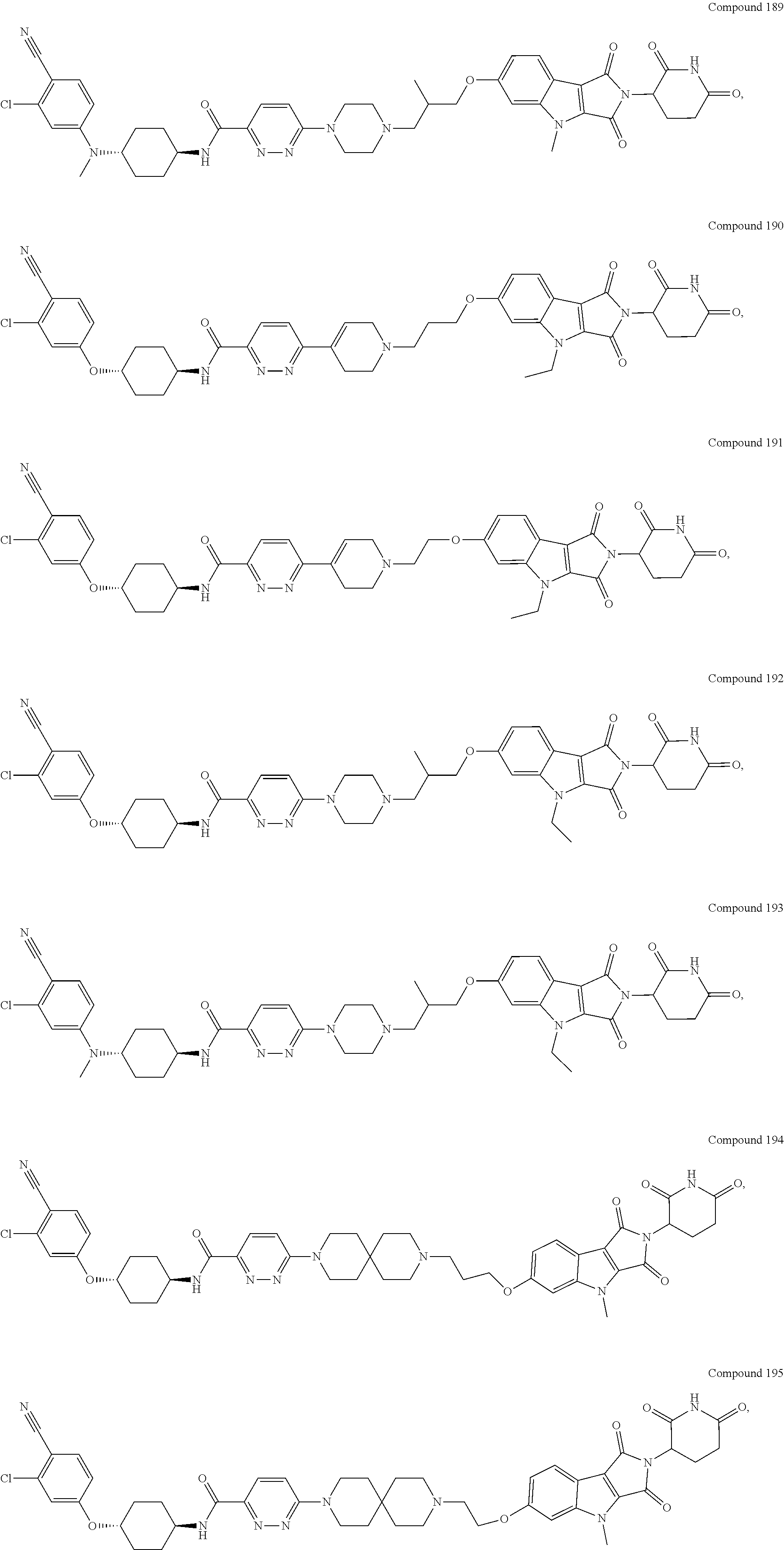

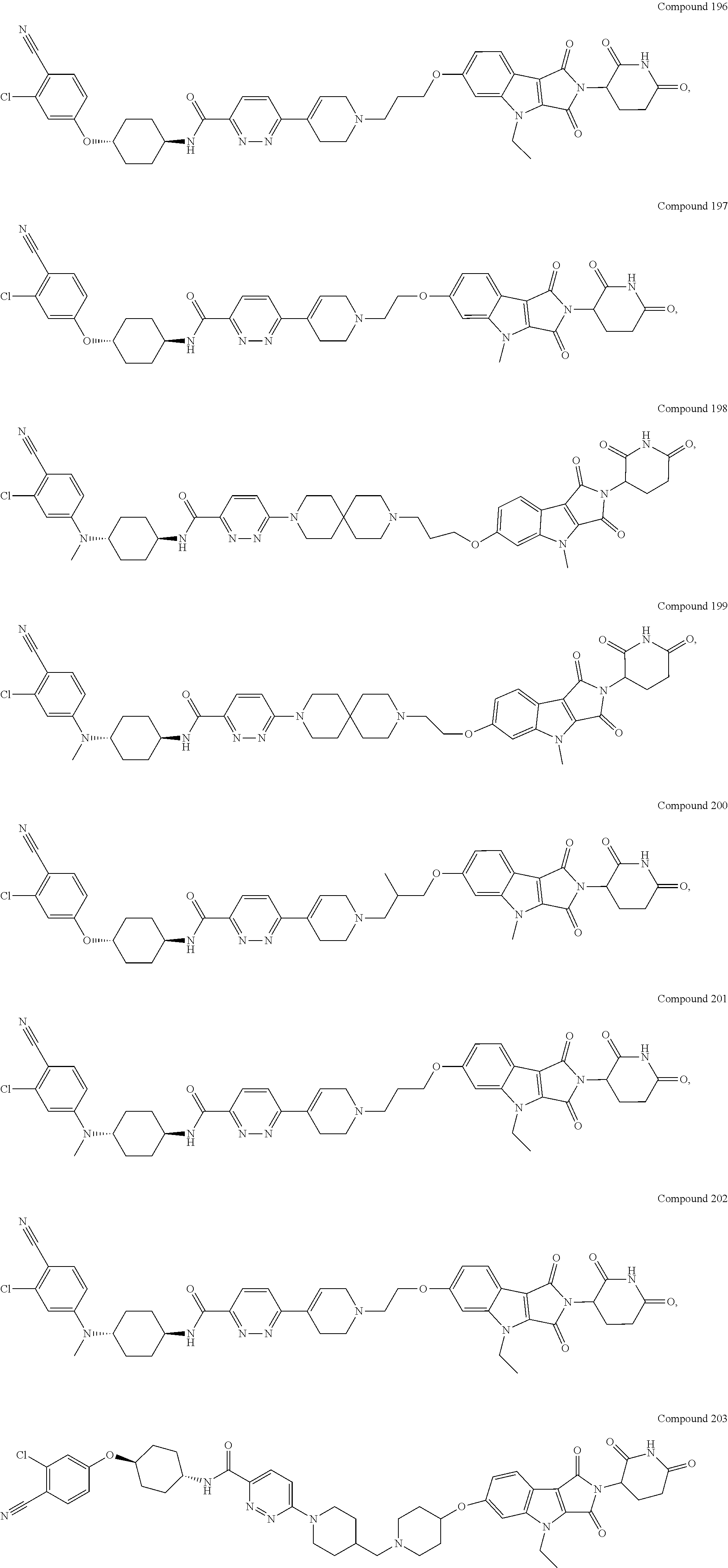

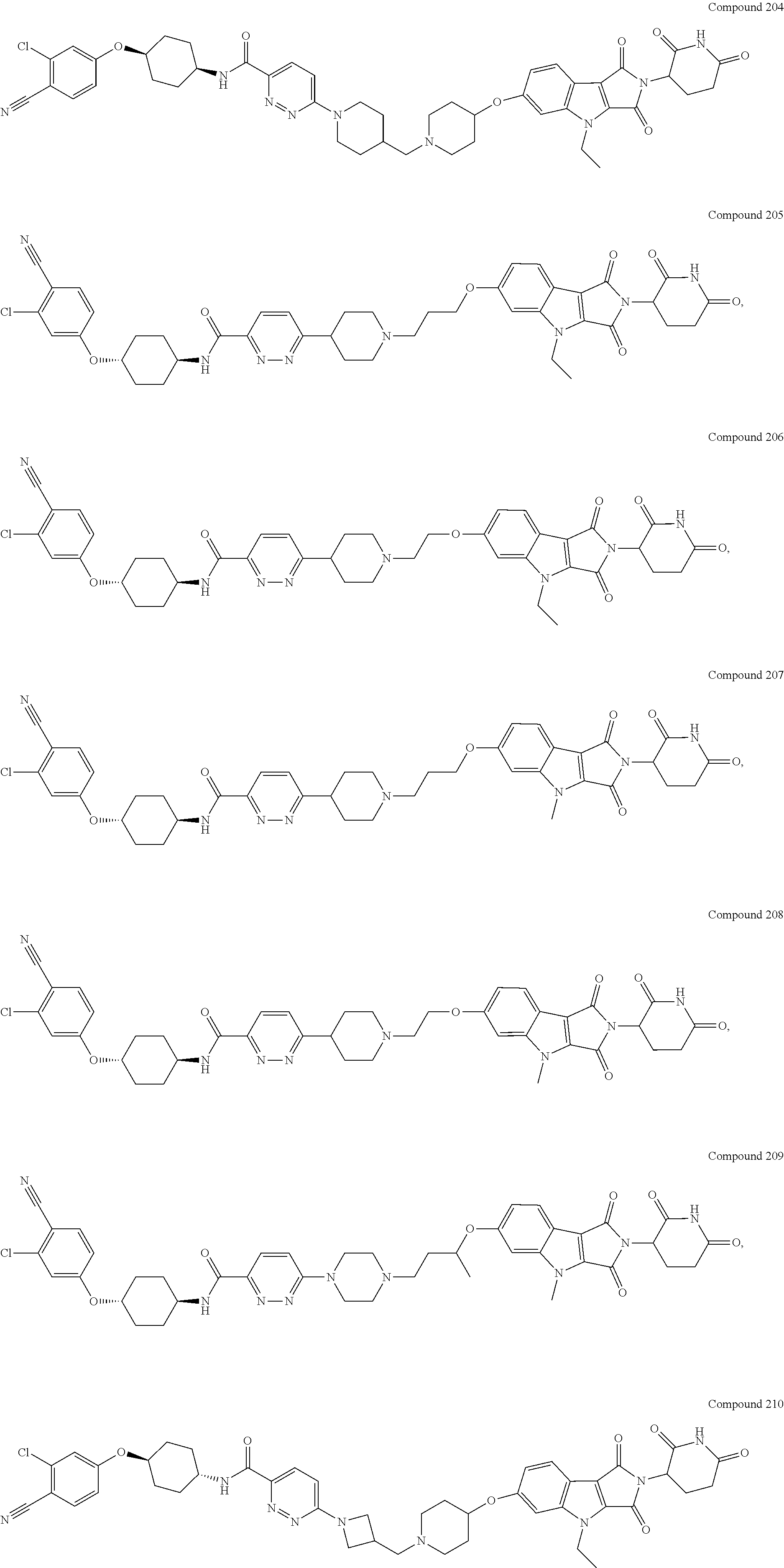

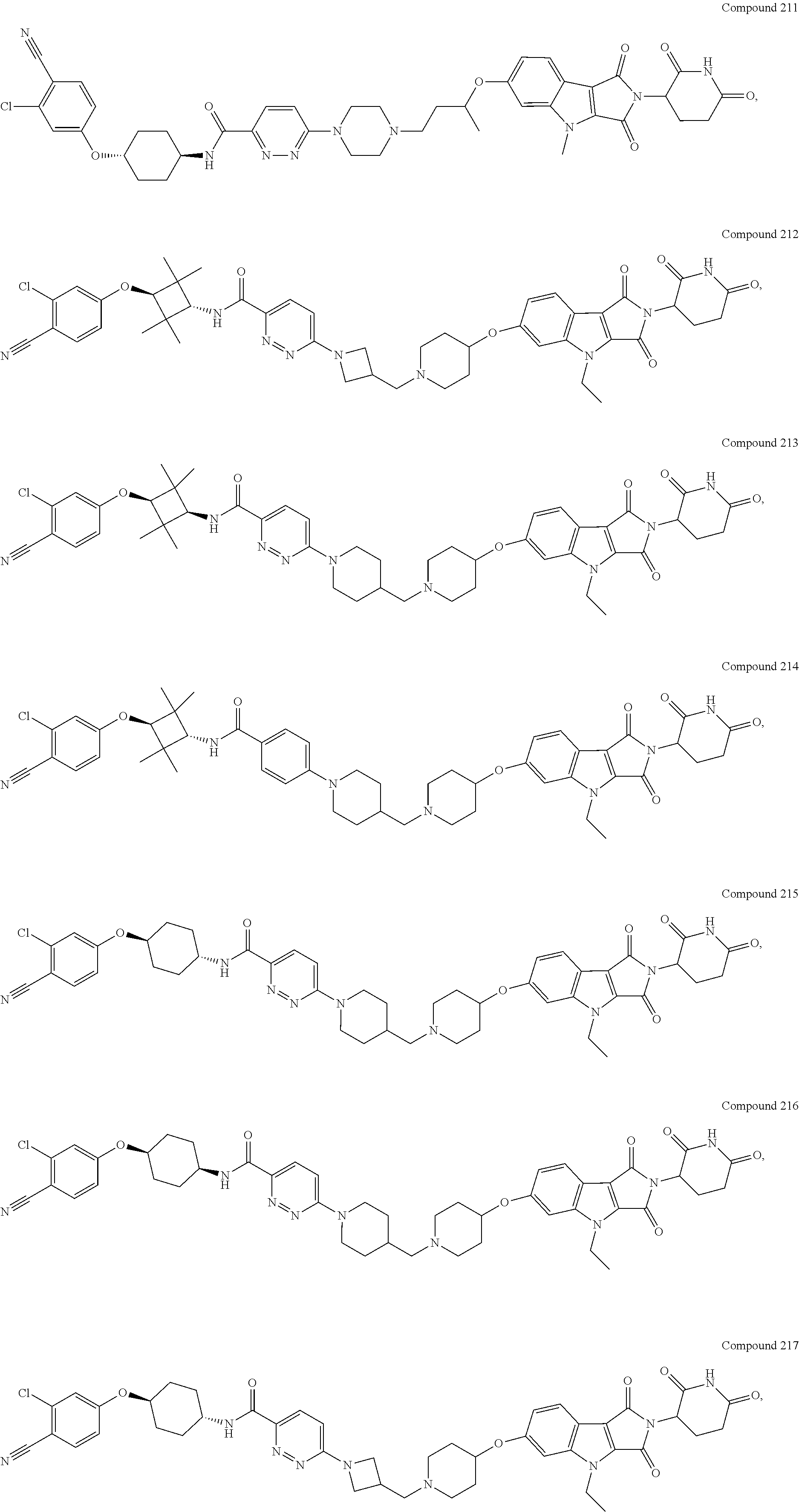

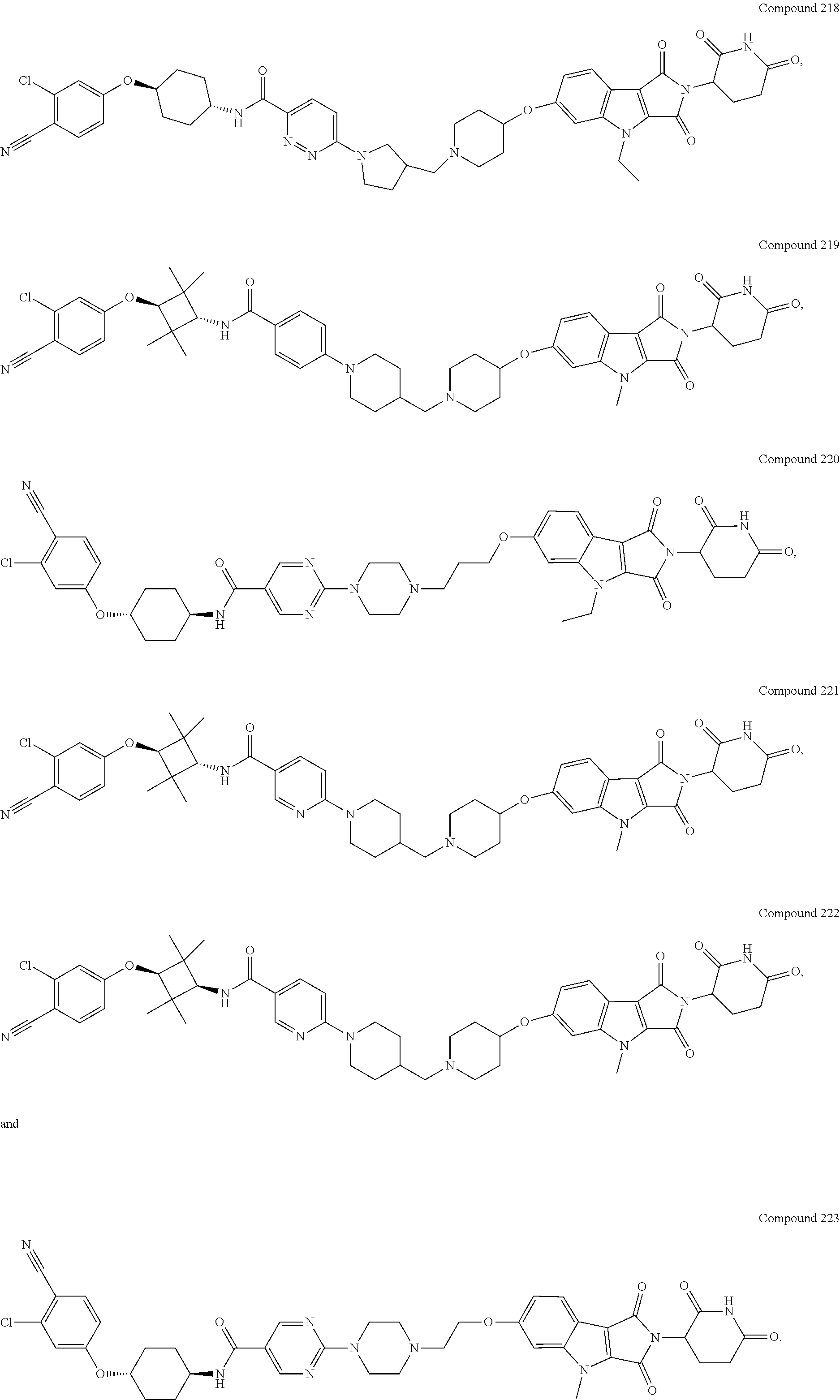

29. A pharmaceutical composition comprising an effective amount of the compound, or the pharmaceutically acceptable salt, hydrate, solvate, metabolite or prodrug thereof as claimed in claim 20; and a pharmaceutically acceptable carrier.

30. The pharmaceutical composition of claim 29, further comprising a second therapeutic agent.

31. A method for treating an androgen receptor related disease or disorder in a subject in need thereof, comprising administering an effective amount of the compound, or the pharmaceutically acceptable salt, metabolite or prodrug thereof as claimed in claim 20 to the subject.

32. A method for treating an androgen receptor related disease or disorder in a subject in need thereof, comprising administering an effective amount of the pharmaceutical composition as claimed in claim 29 to the subject.

33. The method of claim 31, wherein the androgen receptor related disease is an androgen receptor related cancer or an androgen receptor related skin disease.

34. The method of claim 33, wherein the androgen receptor related cancer is breast cancer or prostate cancer.

35. The method of claim 34, wherein the prostate cancer is castration-resistant prostate cancer.

36. The method of claim 34, wherein the breast cancer is triple negative breast cancer.

37. The method of claim 31, wherein the androgen receptor related skin disease is androgenetic alopecia, acne, hidradenitis suppurativa, hirsutism, or atopic dermatitis.

38. The method of claim 31, further comprising administering an effective amount of a second therapeutic agent.
